(19) United States
(12) Patent Application Publication
(10) Pub. No.: US 2007/0043010 A1
(43) Pub. Date: Feb. 22, 2007

(54) Quinazoline derivatives

(75) Inventors: Robert Hugh Bradbury, Cheshire (GB); Christopher Thomas Halsall, Cheshire (GB); Jason Grant Kettle, Cheshire (GB); Alleyn Plowright, Cheshire (GB); Laurent Francois Andre Hennequin, Reims (FR)

Correspondence Address: MORGAN LEWIS & BOCKIUS LLP, 1111 PENNSYLVANIA AVENUE NW, WASHINGTON, DC, 20004, US

(73) Assignee: ASTRAZENECA UK LIMITED, London (GB)

(21) Appl. No.: 10/573,352

(22) PCT Filed: Sep. 22, 2004
(86) PCT No.: PCT/GB04/04085

§ 371 Date: Mar. 24, 2006

(30) Foreign Application Priority Data
Sep. 25, 2003 (GB) 0322409.4
Sep. 26, 2003 (GB) 0322534.9

(51) Int. Cl. A61K 31/33

(52) U.S. Cl. 514/183

(57) ABSTRACT

The invention concerns a quinazoline derivative of the Formula (I): wherein each of the variables have any of the meanings defined in the description; processes for their preparation, pharmaceutical compositions containing them and their use in the manufacture of a medicament for use as an antiproliferative agent in the prevention or treatment of tumours which are sensitive to inhibition of erbB receptor tyrosine kinases.

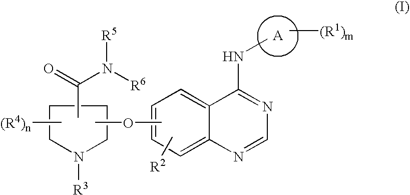

The invention concerns certain novel quinazoline derivatives, or pharmaceutically-acceptable salts thereof, which possess anti-tumour activity and are accordingly useful in methods of treatment of the human or animal body. The invention also concerns processes for the manufacture of said quinazoline derivatives, to pharmaceutical compositions containing them and to their use in therapeutic methods, for example in the manufacture of medicaments for use in the prevention or treatment of solid tumour disease in a warm-blooded animal such as man.

Many of the current treatment regimes for diseases resulting from the abnormal regulation of cellular proliferation such as psoriasis and cancer, utilise compounds that inhibit DNA synthesis and cellular proliferation. To date, compounds used in such treatments are generally toxic to cells however their enhanced effects on rapidly dividing cells such as tumour cells can be beneficial Alternative approaches to these cytotoxic anti-tumour agents are currently being developed, for example selective inhibitors of cell signaling pathways. These types of inhibitors are likely to have the potential to display an enhanced selectivity of action against tumour cells and so are likely to reduce the probability of the therapy possessing unwanted side effects.

Eukaryotic cells are continually responding to many diverse extracellular signals that enable communication between cells within an organism. These signals regulate a wide variety of physical responses in the cell including proliferation, differentiation, apoptosis and motility. The extracellular signals take the form of a diverse variety of soluble factors including growth factors as well as paracrine and endocrine factors. By binding to specific transmembrane receptors, these ligands integrate the extracellular signal to the intracellular signaling pathways, therefore transducing the signal across the plasma membrane and allowing the individual cell to respond to its extracellular signals. Many of these signal transduction processes utilise the reversible process of the phosphorylation of proteins that are involved in the promotion of these diverse cellular responses. The phosphorylation status of target proteins is regulated by specific kinases and phosphatases that are responsible for the regulation of about one third of all proteins encoded by the mammalian genome. As phosphorylation is such an important regulatory mechanism in the signal transduction process, it is therefore not surprising that aberrations in these intracellular pathways result in abnormal cell growth and differentiation and so promote cellular transformation (reviewed in Cohen et al, Curr Opin Chem Biol, 1999, 3, 459-465).

It has been widely shown that a number of these tyro sine kinases are mutated to constitutively active forms and/or when over-expressed result in the transformation of a variety of human cells. These mutated and over-expressed forms of the kinase are present in a large proportion of human tumours (reviewed in Kolibaba et al, Biochimica et Biophysica Acta, 1997, 133, F217-F248). As tyrosine kinases play fundamental roles in the proliferation and differentiation of a variety of tissues, much focus has centred on these enzymes in the development of novel anti-cancer therapies. This family of enzymes is divided into two groups—receptor and non-receptor tyrosine kinases e.g. EGF Receptors and the SRC family respectively. From the results of a large number of studies including the Human Genome Project, about 90 tyrosine kinase have been identified in the human genome, of this 58 are of the receptor type and 32 are of the non-receptor type. These can be compartmentalised in to 20 receptor tyrosine kinase and 10 non-receptor tyrosine kinase sub-families (Robinson et al, Oncogene, 2000, 19, 5548-5557).

The receptor tyrosine kinases are of particular importance in the transmission of mitogenic signals that initiate cellular replication. These large glycoproteins, which span the plasma membrane of the cell possess an extracellular binding domain for their specific ligands (such as Epidermal Growth Factor (EGF) for the EGF Receptor). Binding of ligand results in the activation of the receptor's kinase enzymatic activity that is encoded by the intracellular portion of the receptor. This activity phosphorylates key tyrosine amino acids in target proteins, resulting in the transduction of proliferative signals across the plasma membrane of the cell. It is known that the erbB family of receptor tyrosine kinases, which include EGFR, erbB2, erbB3 and erbB4, are frequently involved in driving the proliferation and survival of tumour cells (reviewed in Olayioye et al., EMBO J., 2000, 19, 3159). One mechanism in which this can be accomplished is by over-expression of the receptor at the protein level, generally as a result of gene amplification. This has been observed in many common human cancers (reviewed in Kiapper et al., Adv. Cancer Res., 2000, 77, 25) such as breast cancer (Sainsbury et al., Brit. J. Cancer, 1988, 58, 458; Guerin et al., Oncogene Res., 1988, 3, 21; Slamon et al., Science, 1989, 244, 707; Klijn et al., Breast Cancer Res. Treat., 1994, 29, 73 and reviewed in Salomon et al., Crit. Rev. Oncol. Hematol., 1995, 19, 183), non-small cell lung cancers (NSCLCs) including adenocarcinomas (Cemy et al., Brit. J. Cancer, 1986, 54, 265; Reubi et al. Int. J. Cancer, 1990, 4, 269; Rusch et al., Cancer Research, 1993, 53, 2379; Brabender et al, Clin. Cancer Res., 2001, 7, 1850) as well as other cancers of the lung (Hendler et al., Cancer Cells, 1989, 7, 347; Ohsaki et al., Oncol. Rep., 2000, 7, 603), bladder cancer (Neal et al, Lancet, 1985, 366; Chow et al., Clin. Cancer Res., 2001, 7, 1957, Zhau et al, Mol Carcinogy 3, 254), oesophageal cancer (Mukaida et al., Cancer, 1991, 68, 142), gastrointestinal cancer such as colon, rectal or stomach cancer (Bolen et al, Oncogene Res., 1987, 1, 149; Kapitanovic et al., Gastroenterology, 2000, 112, 1103; Ross et al, Cancer Invest., 2001, 19, 554), cancer of the prostate (Visakorpi et al., Histochem J., 1992, 24 481; Kumar et al., 2000, 32 73; Scher et al., J. Natl. Cancer Inst., 2000, 92, 1866), leukemia (Konaka et al, Cell, 1984, 37, 1035, Martin-Subero et al., Cancer Genet Cytogenet., 2001, 127, 174), ovarian (Helistrom et al., Cancer Res., 2001, 61, 2420), head and neck (Shiga et al., Head Neck, 2000, 22, 599) or pancreatic cancer (Ovotny et al., Neoplasma, 2001, 48, 188). As more human tumour tissues are tested for expression of the erbB family of receptor tyrosine kinases it is expected that their widespread prevalence and importance will be further enhanced in the future.

As a consequence of the mis-regulation of one or more of these receptors, it is widely believed that many tumours become clinically more aggressive and so correlate with a poorer prognosis for the patient (Braberder et al, Clin, Cancer Res., 2001, 77 1850; Ross et al, Cancer Investigation, 2001, 19, 554, Yu et al., Bioessays, 2000, 22.7, 673). In addition to these clinical findings, a wealth of pre-clinical information suggests that the erbB family of receptor tyrosine kinases are involved in cellular transformation This includes the observations that many tumour cell lines over express one or more of the erbB receptors and that EGFR or erbB2 when transfected into non-tumour cells have the ability to transform these cells. This tumourigenic potential has been further verified as transgenic mice that over express erbB2 spontaneously develop tumours in the mammary gland. In addition to this, a number of pre-clinical studies have demonstrated that anti-proliferative effects can be induced by knocking out one or more erbB activities by small molecule inhibitors, dominant negatives or inhibitory antibodies (reviewed in Mendelsobn et al., Oncolgene, 2000, 19, 6550). Thus it has been recognised that inhibitors of these receptor tyrosine kinases should be of value as a selective inhibitor of the proliferation of mammalian cancer cells (Yaish et al. Science, 1988, 242, 933, Kolibaba et al, Biochimica et Biophysica Acta, 1997, 133, F217-F248; Al-Obeidi et al, 2000, Oncogene, 19, 5690-5701; Mendelsohn et al, 2000, Oncogene, 19 6550-6565). In addition to this pre-clinical data, findings using inhibitory antibodies against EGFR and erbB2 (c-225 and trastuzumab respectively) have proven to be beneficial in the clinic for the treatment of selected solid tumours (reviewed in Mendelsohn et al, 2000, Oncogene, 19, 6550-6565).

Amplification and/or activity of members of the erbB type receptor tyrosine kinases have been detected and so have been implicated to play a role in a number of non-malignant proliferative disorders such as psoriasis (Ben-Bassat, Curr. Pharm Des., 2000, 6, 933; Elder et al, Science, 1989, 243, 811), benign prostatic hyperplasia (BPH) (Kunar et al., Int. Urol. Nerol., 2000, 32, 73), atherosclerosis and restenosis (Bokemeyer et al., Kidney Int., 2000, 58, 549). It is therefore expected that inhibitors of erbb type receptor tyrosine kinases will be useful in the treatment of these and other non-malignant disorders of excessive cellular proliferation.

European patent application EP 566 226 discloses certain 4-anilinoquinazolines that are receptor tyrosine kinase inhibitors.

International patent applications WO 96/33977, WO 96/33978, WO 96/33979, WO 96/33980, WO 96/33981, WO 97/30034, WO 97/38994 disclose that certain quinazoline derivatives, which bear an anilino substituent at the 4-position and a substituent at the 6- and/or 7-position, possess receptor tyrosine kinase inhibitory activity.

European patent application EP 837 063 discloses aryl substituted 4-aninoquinazoline derivatives carrying moiety containing an aryl or heteroaryl group at the 6-or 7-position on the quinazoline ring. The compounds are stated to be useful for treating hyperproliferative disorders.

International patent applications WO 97/30035 and WO 98/13354 disclose certain 4-anilinoquinazolines substituted at the 7-position are vascular endothelial growth factor receptor tyrosine kinase inhibitors.

WO 00/55141 discloses 6,7-substituted 4-aninoquinazoline compounds characterised in that the substituents at the 6-and/or 7-position carry an ester linked moiety (RO—CO).

WO 00/56720 discloses 6,7-dialkoxy-4-aninoquinazoline compounds for the treatment of cancer or allergic reactions.

WO 02/41882 discloses 4-anilinoquinazoline compounds substituted at the 6- and/or 7-position by a substituted pyrrolidinyl-alkoxy or piperidinyl-alkoxy group.

We have now surprisingly found that certain pyrrolidinyloxyquinazoline derivatives possess potent anti-tumour activity and in general have good physical properties, for example good solubility.

Without wishing to imply that the compounds disclosed in the present invention possess pharmacological activity only by virtue of an effect on a single biological process, it is believed that the compounds provide an anti-tumour effect by way of inhibition of one or more of the erbB family of receptor tyrosine kinases that are involved in the signal transduction steps which lead to the proliferation of tumour cells. In particular, it is believed that the compounds of the present invention provide an anti-tumour effect by way of inhibition of EGFR and/or erbB2 receptor tyrosine kinases.

Generally the compounds of the present invention possess potent inhibitory activity against the erbB receptor tyrosine kinase family, for example by inhibition of EGFR and/or erbB2 and/or erbB4 receptor tyrosine kinases, whilst possessing less potent inhibitory activity against other kinases. Furthermore, certain compounds of the present invention possess substantially better potency against the EGFR over that of the erbB2 tyrosine kinase. The invention also includes compounds that are active against all or a combination of EGFR, erbB2 and erbB4 receptor tyrosine kinases, thus potentially providing treatments for conditions mediated by one or more of these receptor tyrosine kinases.

Generally the compounds of the present invention exhibit favourable physical properties such as a high solubility in physiological fluids whilst retaining high antiproliferative activity. Furthermore, many of the compounds according to the present invention are inactive or only weakly active in a hERG assay.

According to a first aspect of the invention there is provided a quinazoline derivative of the Formula (I): 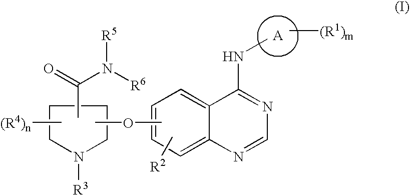
wherein:
either R² is in the 6-position and the substituted-pyrrolidinyloxy group is in the 7-position of the quinazoline ring or R² is in the 7-position and the substituted-pyrrolidinyloxy group is in the 6-position of the quinazoline ring;
A is phenyl or pyridyl;
each R¹ is a substituent on a ring carbon atom in ring A and is independently selected from halogeno, cyano, nitro, hydroxy, carboxy, trifluoromethyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkoxycarbonyl ureido, N-(1-6C)alkylureido, N,N-di-[(1-6C)alkyl]ureido, —NR^{a}R^{b}, —SO₂NR^{a}R^{b} and a group of the formula —CONR^{a}R^{b} (wherein R^{a} is hydrogen or (1-6C)alkyl and R^{b} selected from hydrogen, (1-6C)alkyl, phenyl, benzyl, heterocyclyl, heterocyclyl(1-3C)alkyl, heteroaryl, heteroaryl(1-3C)akyl, (3-7)cycloalkyl and (3-7)cycloalkyl(1-3C)alkyl wherein any alkyl, heterocyclyl, heteroaryl and cycloalkyl groups in R^{a} and R^{b} are optionally substituted by 1, 2 or 3 substituents selected from (1-4C)alkyl, halogeno, hydroxy and (1-4C)alkoxy;
or R^{a} and R^{b} together with the nitrogen atom to which they are attached form a 4, 5 or 6-membered ring which optionally contains an additional ring heteroatom selected from nitrogen, oxygen and sulphur and which is optionally substituted by 1 or 2 substituents on an available ring carbon atom, independently selected from halogeno, hydroxy, (1-4C)alkyl and (1-3C)alkylenedioxy and optionally substituted on any available zing nitrogen by a substituent selected from (1-4C)alkyl and (2-4C)alkanoyl (provided the ring is not thereby quaternised), and wherein any (1-4C)alkyl or (2-4C)alkanoyl group present as a substituent on the ring formed by R^{a} and R^{b} together with the nitrogen atom to which they are attached is optionally substituted by 1, 2 or 3 substituents independently selected from halogeno, hydroxyl, (1-4C)alkyl and (1-4C)alkoxy;
or, when two R¹ groups are attached to adjacent carbon atoms, they may, together with the carbon atoms to which they are attached, form a pyrrole ring, wherein the pyrrole ring is optionally substituted by 1 or 2 substituents independently selected from (1-6C)alkyl, halogeno, cyano, nitro, hydroxy, amino, carbamoyl, sulfamoyl and trifluoromethyl;
or, when two R¹ groups are attached to adjacent carbon atoms, they may, together form a (1-3C)alkylenedioxy group [—O(CH₂),₁₋₃];
m is 0, 1, 2 or 3;
each R² is selected from hydrogen, (1-6C)akyl, (3-6C)cycloalkyl (3-6C)cycloalkyl(1-3C)alkyl and a group of the formula R⁷O—, wherein R⁷ is (1-6C)alkyl optionally substituted by 1, 2 or 3 substituents independently selected from hydroxy and a group of the formula R⁸O— (wherein R⁸ is (1-3C)alkyl);
R³ is selected from hydrogen, (1-6C)alkyl, (3-6C)cycloalkyl, (3-6C)cycloalkyl(1-3C)alkyl (1-6C)alkylthio, (1-6C)alkylsulfinyl, (1-6C)alkylsulfonyl, (2-6C)alkanoyl, carbamoyl(1-6C)alkyl, N-(1-6C)alkylcarbamoyl(1-6C)alkyl, N,N-di-[(1-6C)alkyl]carbamoyl(1-6C)alkyl, sulfamoyl(1 -6C)alkyl, N-(1-6C)alkylsulfamoyl(1-6C)alkyl, N,N-di-[(1-6C)alkyl]sulfamoyl(1-6C)alkyl and (2-6C)alkanoyl(1-6C)alkyl,
and wherein any (1-6C)alkyl or (2-6C)alkanoyl group within R³ is optionally substituted by 1, 2 or 3 substituents independently selected from halogeno, hydroxy and (1-6C)alkyl and/or optionally a substituent selected from cyano, nitro, (2-8C)alkenyl, (2-8C)alkynyl, (1-6C)alkoxy and NR^{c}R^{d}, wherein R^{c} is hydrogen or (1-4C)alkyl and R^{d} is hydrogen or (1-4C)alkyl, and wherein any (1-4C)alkyl in R^{c} or R^{d} is optionally substituted by 1, 2 or 3 substituents independently selected from halogeno and hydroxy and/or optionally a substituent selected from cyano, nitro and (1-4C)alkoxy,

or R^{c} and R^{d} together with the nitrogen atom to which they are attached form a 4, 5 or 6 membered ring which optionally contains an additional ring heteroatom selected from nitrogen, oxygen and sulphur and which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised), and wherein any (1-4C)alkyl or (2-4C)alkanoyl group present as a substituent on the ring formed by R^{c} and R^{d} together with the nitrogen atom to which they are attached is optionally substituted by 1, 2 or 3 substituents independently selected from halogeno and hydroxy and/or optionally a substituent selected from (1-4C)alkyl and (1-4C)alkoxy;
each R⁴ is independently selected from (1-4C)alkyl, (1-4C)alkoxy, cyano, halogeno, hydroxyl and oxo;
n is 0, 1 or 2;
R⁵ is hydrogen or (1-6C)alkyl;
R⁶ is selected from hydrogen, (1-6C)alkyl, (2-6C)alkenyl, (2-6C)alkynyl, (1-6C)alkoxy, (3-7)cycloalkyl, (1-6C)alkysulfonyl, heterocyclyl, heteroaryl, (3-7)cycloalkyl(1-3C)alkyl, (3-7)heterocyclyl(1-3C)alkyl and heteroaryl(1-3C)alkyl,
and wherein any (1-3C)akyl, (1-6C)alkyl, (3-7)cycloalkyl, heteroaryl or heterocyclyl group within R⁵ or R⁶ is optionally substituted (on any available carbon atoms) by 1, 2 or 3 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heterocyclyl group within R⁶ is optionally substituted on any available ring nitrogen (provided the ring is not thereby quaternised) by (1-4C)alkyl or (2-4C)alkanoyl, or R⁵ and R⁶ together with the nitrogen atom to which they are attached form a 4, 5 or 6 membered ring which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised), and wherein any (1-4C)alkyl or (2-4C)alkanoyl group present as a substituent on the ring formed by R⁵ and R⁶ together with the nitrogen atom to which they are attached is optionally substituted by 1, 2 or 3 substituents independently selected from halogeno and hydroxy and/or optionally a substituent selected from (1-4C)alkyl and (1-4C)akoxy;

provided that when the pyrrolidinyloxy group is linked to the 6-position of the quinazoline ring, m is 2 and substituents R¹ are both halogeno and attached to the 2- and 3-positions of the ring A, then R⁶ is selected from substituted-(1-6C)alkyl (wherein substituted-(1-6C)alkyl is (1-6C)alkyl substituted by 1, 2 or 3 substituents independently selected from halogeno, hydroxy(1-6C)alkyl, (1-6C)alkoxycarbonyl, carbamoyl, (2-6C)alkanoylamino, (1-6C)alkylamino, di-[(1-6C)alkyl]amino and hydroxy and/or optionally a substituent selected from oxo, cyano, nitro and (1-4C)alkoxy), (2-6C)alkenyl, (2-6C)alkynyl, (1-6C)alkoxy, (3-7)cycloalkyl, (1-6C)alkylsulfonyl, (3-7)heterocyclyl, heteroaryl, (3-7)cycloalkyl(1-6C)alkyl, (3-7)heterocyclyl(1-6C)alkyl and heteroaryl( 1-6C)alkyl,
and wherein any (3-7)cycloalkyl, heteroaryl or (3-7)heterocyclyl group within R⁶ is optionally substituted (on any available carbon atoms) by 1, 2 or 3 substituents independently selected from halogeno, hydroxy, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heteroaryl or heterocyclyl group within R⁶ is optionally substituted on any available ring nitrogen (provided the ring is not thereby quaternised) by (1-4C)alkyl or (2-4C)alkanoyl or
R⁵ and R⁶ together with the nitrogen atom to which they are attached form a 4, 5 or 6 membered ring which contains one or two nitrogen atoms as the only heteroatom(s) present in the ring and is optionally substituted by 1 or 2 substituents on an available ring carbon atom, independently selected from hydroxy, carbamoyl, (1-4C)alkyl, and (1-3C)alkylenedioxy; and wherein any 4, 5 or 6 membered heterocyclic ring formed by R⁵ and R⁶ is optionally substituted on any available ring nitrogen (provided the ring is not thereby quaternised) by (1-4C)alkyl or (2-4C)alkanoyl;

or a pharmaceutically-acceptable salt thereof.

The definitions of the variables given hereinabove will be referred to as definition ‘I’ for the purposes of defining classes of compound in Table A hereinbelow.

According to a second aspect of the invention there is provided a quinazoline derivative of the Formula (I): 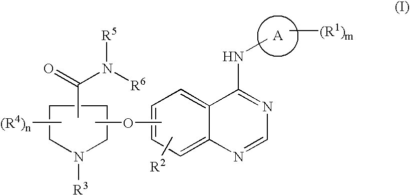
wherein:
either R² is in the 6position and the substituted-pyrrolidinyloxy group is in the 7-position of the quinazoline ring or R² is in the 7-position and the substituted-pyrrolidinyloxy group is in the 6-position of the quinazoline ring;
A is phenyl or pyridyl;
each R¹ is a substituent on a ring carbon atom in ring A and is independently selected from halogeno, cyano, nitro, hydroxy, carboxy, trifluoromethyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkoxycarbonyl, ureido, N-(1-6C)alkylureido, N,N-di-[(1-6C)alkyl]ureido, —NR^{a}R^{b}, —SO₂NR^{a}R^{b} and a group of the formula —CONR^{a}R^{b} (wherein R^{a} is hydrogen or (1-6C)alkyl and R^{b} selected from hydrogen, (1-6C)alkyl, phenyl, benzyl, heterocyclyl, heterocyclyl(1-3C)alkyl, heteroaryl, heteroaryl(1-3C)alkyl, (3-7)cycloalkyl and (3-7)cycloalkyl(1-3C)alkyl wherein any alkyl, heterocyclyl, heteroaryl and cycloalkyl groups in R^{a} and R^{b} are optionally substituted by 1, 2 or 3 substituents selected from (1-4C)alkyl, halogeno, hydroxy and (1-4C)alkoxy;
or R^{a} and R^{b} together with the nitrogen atom to which they are attached form a 4, 5 or 6-membered ring which optionally contains an additional ring hetero atom selected from nitrogen, oxygen and sulphur and which is optionally substituted by 1 or 2 substituents on an available ring carbon atom, independently selected from halogeno, hydroxy, (1-4C)alkyl and (1-3C)alkylenedioxy and optionally substituted on any available ring nitrogen by a substituent selected from (1-4C)alkyl and (2-4C)alkanoyl (provided the ring is not thereby quaternised), and wherein any (1-4C)alkyl or (2-4C)alkanoyl group present as a substituent on the ring formed by R^{a} and R^{b} together with the nitrogen atom to which they are attached is optionally substituted by 1, 2 or 3 substituents independently selected from halogeno, hydroxyl (1-4C)alkyl and (1-4C)alkoxy;
or, when two R¹ groups are attached to adjacent carbon atoms, they may, together with the carbon atoms to which they are attached, form a pyrrole ring, wherein the pyrrole ring is optionally substituted by 1 or 2 substituents independently selected from (1-6C)alkyl, halogeno, cyano, nitro, hydroxy, amino, carbamoyl, sulfamoyl and trifluoromethyl;
or, when two R¹ groups are attached to adjacent carbon atoms, they may, together form a (1-3C)alkylenedioxy group [—O(CH₂)₁₋₃O];
m is 0, 1, 2 or 3; each R² is selected from hydrogen, (1-6C)alkyl, (3-6C)cycloalkyl, (3-6C)cycloalkyl(1-3C)alkyl and a group of the formula R⁷O—, wherein R⁷ is (1-6C)alkyl optionally substituted by 1, 2 or 3 substituents independently selected from hydroxy and a group of the formula R⁸O— (wherein R⁸ is (1-3C)alkyl);
R³ is selected from hydrogen, (1-6C)alkyl, (3-6C)cycloalkyl, (3-6C)cycloalkyl(1-3C)alkyl, (1-6C)alkylthio, (1-6C)alkylsulfinyl, (1-6C)alkylsulfonyl, (2-6C)alkanoyl, carbamoyl(1-6C)alkyl, N-(1-6C)alkylcarbamoyl(1-6C)alkyl, N,N-di-[(1-6C)alkyl]carbamoyl(1-6C)alkyl, sulfamoyl(1-6C)alkyl, N-(1-6C)alkylsulfamoyl(1-6C)alkyl, N,N-di-[( 1-6C)alkyl]sulfamoyl(1-6C)alkyl and (2-6C)alkanoyl(1-6C)alkyl,
and wherein any (1-6C)alkyl or (2-6C)alkanoyl group within R³ is optionally substituted by 1, 2 or 3 substituents independently selected from halogeno, hydroxy and (1-6C)alkyl and/or optionally a substituent selected from cyano, nitro, (2-8C)alkenyl, (2-8C)alkynyl, (1-6C)alkoxy and NR^{c}R^{d}, wherein R^{c} is hydrogen or (1-4C)alkyl and R^{d} is hydrogen or (1-4C)alkyl, and wherein any (1-4C)alkyl in R^{c} or R^{d} is optionally substituted by 1, 2 or 3 substituents independently selected from halogeno and hydroxy and/or optionally a substituent selected from cyano, nitro and (1-4C)alkoxy,

or R^{c} and R^{d} together with the nitrogen atom to which they are attached form a 4, 5 or 6 membered ring which optionally contains an additional ring heteroatom selected from nitrogen, oxygen and sulphur and which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4)alkyl and (2-4C)alkanoyl (provided the ring is not thereby quaternised), and wherein any (1-4C)alkyl or (2-4C)alkanoyl group present as a substituent on the ring formed by R^{c} and R^{d} together with the nitrogen atom to which they are attached is optionally substituted by 1, 2 or 3 substituents independently selected from halo geno and hydroxy and/or optionally a substituent selected from (1-4C)alkyl and (1-4C)alkoxy;
each R⁴ is independently selected from (1-4C)alkyl, (1-4C)alkoxy, cyano, halogeno, hydroxyl and oxo;
n is 0, 1 or2;
R⁵ is hydrogen or (1-6C)alkyl;
R⁶ is selected from hydrogen, (1-6C)alkyl, (2-6C)alkenyl, (2-6C)alkynyl, (1-6C)alkoxy, (3-7)cycloalkyl, (1-6C)alkylsulfonyl, heterocyclyl, heteroaryl, (3-7)cycloalkyl(1-3C)alkyl, (3-7)heterocyclyl(1-3C)alkyl and heteroaryl(1-3C)alkyl,
and wherein any (1-3C)alkyl, (1-6C)alkyl, (3-7)cycloalkyl, heteroaryl or heterocyclyl group within R⁵ or R⁶ is optionally substituted (on any available carbon atoms) by 1, 2 or 3 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heterocyclyl group within R⁶ is optionally substituted on any available ring nitrogen (provided the ring is not thereby quaternised) by (1-4C)alkyl or (2-4C)alkanoyl, or R⁵ and R⁶ together with the nitrogen atom to which they are attached form a 4, 5 or 6 membered ring which is optionally substituted by 1 or 2 substituents on an available ring carbon atom, independently selected from halo geno, hydroxy, ( 1-4C)alkyl and (1-3C)alkylenedioxy, and optionally substituted on any available ring nitrogen by a substituent selected from (1-4C)alkyl and (2-4C)alkanoyl (provided the ring is not thereby quaternised), and wherein any (1-4C)alkyl or (2-4C)alkanoyl group present as a substituent on the ring formed by R⁵ and R⁶ together with the nitrogen atom to which they are attached is optionally substituted by 1, 2 or 3 substituents independently selected from halogeno and hydroxy and/or optionally a substituent selected from (1-4C)alkyl and (1-4C)alkoxy;

provided that when the pyrrolidinyloxy group is linked to the 6-position of the quinazoline ring, m is 2 and substituents R¹ are both halogeno and attached to the 2- and 3-positions of the ring A, then R⁶ is selected from substituted-(1-6C)alkyl (wherein substituted-(1-6C)alkyl is (1-6C)alkyl substituted by 1, 2 or 3 substituents independently selected from (1-6C)alkoxycarbonyl, carbamoyl, (2-6C)alkanoylamino, and oxo or a (1-6C)alkoxycarbonyl together with a hydroxy group), (1-6C)alkoxy, (1-6C)alkylsulfonyl, (3-7)heterocyclyl (wherein the heterocyclyl is carbon linked), heteroaryl, (3-7)heterocyclyl( 1-6C)alkyl (wherein the heterocyclyl is carbon linked to the (1-6C)alkyl moiety) and heteroaryl(1-6C)alkyl, and wherein any heteroaryl or (3-7)heterocyclyl group within R⁶ is optionally substituted (on any available carbon atoms) by 1, 2 or 3 substituents independently selected from halogeno, hydroxy, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heteroaryl or heterocyclyl group within R⁶ is optionally substituted on any available ring nitrogen (provided the ring is not thereby quaternised) by (1-4C)alkyl or (2-4C)alkanoyl, or
R⁵ and R⁶ together with the nitrogen atom to which they are attached form a 4, 5 or 6 membered ring which contains one or two nitrogen atoms as the only heteroatom(s) present in the ring and which is substituted on an available ring carbon atom by 1 or 2 substituents independently selected from carbamoyl and (1-3C)alkylenedioxy;

or a pharmaceutically-acceptable salt thereof.

In this specification the generic term “alkyl” includes both straight-chain and branched-chain alkyl groups such as propyl, isopropyl and tert-butyl, and (3-7C)cycloalkyl groups such as cyclopropyl, cyclobutyl, cyclopentyl, cyclohexyl and cycloheptyl. However references to individual alkyl groups such as “propyl” are specific for the straight-chain version only, references to individual branched-chain alkyl groups such as “isopropyl” are specific for the branched-chain version only and references to individual cycloalkyl groups such as “cyclopentyl” are specific for that 5-membered ring only. An analogous convention applies to other generic terms, for example (1-6C)alkoxy includes methoxy, ethoxy, cyclopropyloxy and cyclopentyloxy, (1-6C)alkylamino includes methylamino, ethylamino, cyclobutylamino and cyclohexylamino, and di-[(1-6C)alkyl]amino includes dimethylamino, diethylamino, N-cyclobutyl-N-methylamino and N-cyclohexyl-N-ethylamino.

It is to be understood that, insofar as certain of the compounds of Formula I defined above may exist in optically active or racemic forms by virtue of one or more asymmetrically substituted carbon and/or sulfur atoms, and accordingly may exist in, and be isolated as enantiomerically pure, a mixture of diastereoisomers or as a racemate. The present invention includes in its definition any racemic, optically-active, enantiomerically pure, mixture of diastereoisomers, stereoisomeric form of the compound of Formula (I), or mixtures thereof, which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

The invention relates to all tautomeric forms of the compounds of the Formula I that possess antiproliferative activity.

It is also to be understood that certain compounds of the Formula I may exist in solvated as well as unsolvated forms such as, for example, hydrated forms. It is to be understood that the invention encompasses all such solvated forms, which possess antiproliferative activity.

It is also to be understood that certain compounds of the Formula I may exhibit polymorphism, and that the invention encompasses all such forms which possess antiproliferative activity.

Suitable values for the generic radicals referred to above include those set out below.

A suitable value for (3-7C)cycloalkyl is, for example, cyclopropyl, cyclobutyl, cyclopentyl, cyclohexyl cycloheptyl or bicyclo [2.2. 1]heptyl.

A heterocyclyl group is a non-aromatic saturated (i.e. with the maximum degree of saturation) or partially saturated (i.e. ring systems retaining some, but not the fill, degree of unsaturation) 3 to 7 membered monocyclic ring with up to 3 heteroatoms selected from oxygen, nitrogen and sulfur (but not containing any O—O, O—S or S—S bonds), and linked via a ring carbon atom, or a ring nitrogen atom (provided the ring is not thereby quaternised). Suitable values for heterocyclyl include for example, oxiranyl, oxetanyl, azetidinyl, tetrahydrofuranyl, tetrahydropyranyl, oxepanyl, oxazepanyl pyrrolinyl pyrrolidinyl, morpholinyl, tetrahydro-1,4-thiazinyl, 1,1-dioxotetrahydro-1,4-thiazinyl, piperidinyl, homopiperidinyl, piperazinyl, homopiperazinyl, dihydropyridinyl, tetrahydropyridimyl, dihydropyrimidinyl, tetrahydropyrimidinyl, tetrahydrothienyl, tetrahydrothiopyranyl, thiomorpholinyl, more specifically including for example, tetrahydrofuran-3-yl, tetrahydrofuran-2-yl-, tetrahydropyran-4-yl, tetrahydrothienyl-3-yl, tetrahydrothiopyran-4-yl, pyrroldin-3-yl, pyrrolidin-2-yl, 3-pyrrolin-3yl, morpholo,
1,1-dioxotetrahydro-4H-1,4-thiazin-4-yl, piperidino, piperidin-4-yl, piperidin-3-yl, piperidin-2-yl, homopiperidin-3-yl, homopiperidin-4-yl, piperazin-1-yl, 1,4-oxazepanyl, or 1,2,3,6tetrahydropyridin-4-yl. A nitrogen or sulfur atom within a heterocyclyl group may be oxidized to give the corresponding N or S oxide(s), for example 1,1-dioxotetrahydrothienyl, 1-oxotetrahydrothienyl, 1,1-dioxotetrahydrothiopyranyl or 1-oxotetrahydrothiopyxanyl. A suitable value for such a group which bears 1 or 2 oxo or thioxo substituents is, for example, 2-oxopyrrolidinyl, 2-oxopiperazinyl, 2-thioxopyrrolidinyl, 2-oxopiperidinyl, 2,5-dioxopyrrolidinyl or 2,6-dioxopiperidinyl.

Particular values for heterocyclyl include, for example, non-aromatic saturated or partially saturated 3 to 7 membered monocyclic heterocyclyl rings with 1 ring nitrogen or sulfur heteroatom and optionally 1 or 2 heteroatoms selected from nitrogen, oxygen and sulfur. Examples of such rings include azetidinyl, oxazepanyl, pyrrolinyl, pyrrolidinyl, morpholinyl, tetrahydro-1,4-thiazinyl, piperidinyl homopiperidinyl piperazinyl homopiperazinyl, dihydropyridinyl, tetrahydropyridinyl, dihydropyrimidinyl, tetrahydropyrimidinyl, tetrahydrothienyl, tetrahydrothiopyranyl or thiomorpholinyl,

Further particular values for heterocyclyl include, for example, morpholino, or 4, 5 or 6 membered heterocyclyl rings containing 1 nitrogen atom and optionally 1 heteroatom selected from nitrogen and sulfur such as piperazinyl, pyrrolidinyl, piperidinyl, particularly pyrrolidin-1-yl, pyrrolidin-2-yl, piperazin-1-yl, piperidino, morpholino or piperazino.

Illustrative examples of carbon-linked heterocyclyl groups include tetrahydrofuran-2-yl, tetrahydrofuran-3-yl, tetrahydropyran-4-yl, pyrrolidin-2-yl, pyrrolidin-3-yl, piperidin-2-yl, piperidin-3-yl, and piperidin-4-yl.

The term “heterocyclyl-alkyl” refers to substituent groups comprising a heterocyclyl group that is linked via an alkyl moiety.

Illustrative examples of carbon linked (3-7)heterocyclyl(1-6C)alkyl groups include tetrahydrofuran-2-ylmethyl, tetrahydrofuran-3-ylmethyl, tetrahydropyran-4-ylmethyl, pyrrolidin-2-ylmethyl, pyrrolidin-3-ylmethyl, piperidin-2-ylmethyl, piperidin-3-ylmethyl, and piperidin-4-ylmethyl.

When R^{a} and R^{b} or R^{c} and R^{d} or R⁵ and R⁶, together with the nitrogen atoma to which they are attached form a 4, 5 or 6 membered ring, the ring is a saturated or partially saturated non-aromatic heterocyclyl ring containing 1 nitrogen and optionally 1 heteroatom selected from oxygen, sulfur and Estrogen and wherein the ring so formed is linked via a ring nitrogen atom to the group to which the ring is attached. Suitable values for R^{a} and R^{b} or R^{c} and R^{d} or R⁵ and R⁶, when together with the nitrogen atom to which they are attached form a 4, 5 or 6 membered ring include, for example, azetidin-1-yl, pyrrolin-1-yl, 1,2,3,6-tetrahydropyridin-1-yl, pyrrolidin-1-yl, piperidin-1-yl, piperazin-1-yl and morpholino.

A heteroaryl ring is a monocyclic, 5- or 6-membered aryl ring containing 1, 2 or 3 heteroatoms independently selected from nitrogen, oxygen and sulphur. Examples of heteroaryl rings include pyrazolyl, thienyl, oxazolyl, isooxazolyl imidazolyl, pyridinyl, pyridazinyl, pyrazinyl, pyrimidyl, furanyl, pyrazolyl, thiazolyl, isothiazolyl and thiadiazolyl Illustrative examples of heteroaryl(1-6C)alkyl groups include pyrazol-5-ylmethyl, thien-3-ylmethyl, isoxazol-3-ylmethyl, imidazol-1-ylmethyl, imidazol-2-ylmethyl, imidazol-4 ylmethyl, pyridin-2-ylmethyl, pyrimidin-3-ylmethyl, furan-2-ylmethyl, pyrazol-5-ylmethyl, 2-(pyrazol-5-yl)ethyl, 2-(thien-3-yl)ethyl, 2-(isoxazol-3-yl)ethyl, 2-(imidazol-1-yl)ethyl, 2-(imidazol-2-yl)ethyl, 2-(imidazol-4-yl)ethyl, 2-(pyridin-2-yl)ethyl, 2-(pyrimidin-3-yl)ethyl, 2(furan-2-yl)ethyl, 2-(pyrazol-5-yl)ethyl.

Suitable values for any of the R¹, R², R³, R⁴, R⁵, R⁶ or for various groups within them as defined hereinbefore or hereafter in this specification include:
for halogeno: fluoro, chloro, bromo and iodo;
for (1-6C)alkyl: methyl, ethyl, propyl, isopropyl, tert-butyl pentyl and hexyl;
for (1-4C)alkyl: methyl, ethyl, propyl, isopropyl and tert-butyl;
for (1-6C)alkoxy: methoxy, ethoxy, propoxy, isopropoxy and butoxy;
for (2-8C)alkenyl: vinyl, isopropenyl, allyl and but-2-enyl;
for (2-8C)alkynyl: ethynyl, 2-propynyl and but-2-ynyl;
for (2-6C)alkenyloxy: vinyloxy and allyloxy;
for (2-6C)alkynyloxy: ethynyloxy and 2-propynyloxy;
for (1-6C)alkylthio: methylthio, ethylthio and propylthio;
for (1-6C)alkylsulfinyl: methylsulfinyl and ethylsulfinyl;
for (1-6C)alkylsulfonyl: methylsulfonyl and ethylsulfonyl;
for (1-6C)alkoxycarbonyl: methoxycarbonyl ethoxycarbonyl propoxycarbonyl and tert-butoxycarbonyl;
for (2-6C)alkanoyl: acetyl, propionyl and isobutyryl;
for (2-6C)alkanoyloxy: acetoxy and propionyloxy;
for (2-6C)alkanoylamino: acetamido and propionamido;
for N-(1-6C)alkyl-(2-6C)alkanoylamino: N-methylacetaIimido and N-methylpropionamido;
for hydroxy-(1-6C)alkyl: hydroxymethyl 2-hydroxyethyl, 1-hydroxyethyl and 3-hydroxypropyl;
for hydroxy-(1-6C)alkoxy: hydroxymethoxy, 2-hydroxyethoxy, 1-hydroxyethoxy and 3-hydroxypropoxy;
for (1-6C)alkoxy-(1-6C)alkyl: methoxymethyl, ethoxymethyl, 1-methoxyethyl, 2-methoxyethyl, 2-ethoxyethyl and 3-methoxypropyl;
for carbamoyl(1-6C)alkyl: carbonylmethyl, 1-carbamoylethyl, 2-carbamoylethyl and 3-carbamoylpropyl;
for N-(1-6C)alkylcarbamoyl(1-6C)alkyl: N-methylcarbamoylmethyl, N-ethylcarbamoybmethyl, N-propylcarbamoylmethyl, 1-(N-methylcarbamoyl)ethyl, 2-(N-methylcarbamoyl)ethyl and 3-(N-methylcarbamoyl)propyl;
for N,N di-(1-6C)alkylcarbamoyl(1-6C)alkyl: N,N-dimetiylcarbamoylmethyl, N,N-diethylcarbamoylmethyl, N methyl,N-ethylcarbamoylmethyl, 1-( N,N-dim-ethylcarbamoyl)ethyl, 1-(N,N-diethylcarbamoyl) ethyl, 2-(N,N-dimethylcarbamoyl)ethyl, 2-(N,N-diethylcarbamoyl)ethyl and 3-(N,N-dimethylcarbamoyl)propyl;
for sulfamoyl(1-6C)alkyl: sulfamoylmethyl, 1-sulfamoylmethyl, 2-sulfamoylethyl and 3-sulfamoylpropyl;
for N-(1-6C)alkylsulfamoyl(1-6C)alkyl: N-methylsulfamoylmethyl, N-ethylsulfamoylmethyl, N-propylsulfamoylmethyl, 1-N-methylsulfamoyl)ethyl, 2-(N-methylsulfamoyl)ethyl and 3-N-methylsulfamoyl)propyl;
for N,N di-(1-6C)alkylsulfamoyl(1-6C)alkyl: N,N-dimethylsulfamoylmethyl, N,N-diethylsulfamoylmethyl, N methyl,N-ethylsulfamoylmethyl, 1-( N,N-dimethylsulfamoyl)ethyl, 1-(N,N-diethylsulfamoyl)ethyl, 2-(N,N-dimethylsulfamoyl)ethyl, 2-(N,N-diethylsulfamoyl)ethyl and 3-(N,N-dimethylsulfamoyl)propyl;
for (2-6C)alkanoyl(1-6C)alkyl: acetylmethyl, propionylmethyl, 2-acetylethyl and 2-propionylethyl;
for N-(1-6C)alkylureido: N-methylureido, N-ethylureido and N-propylureido;
for N,N-[di(1-6C)]alkylureido: N,N-(dimethyl)ureido, N-methyl-N-ethylureido and N-methyl-N-propylureido;
for (3-7C)cycloakyl: cyclopropyl, cyclobutyl, cyclopentyl and cyclohexyl; and
for (3-7C)cycloakyl(1-3C)alkyl: cyclopropylmethyl, 2-cyclopropylmethyl, cyclobutylmethyl, cyclopentylmethyl and cyclohexylethyl.

Examples of suitable groups for —CONR^{a}R^{b} in R¹ are: carbamoyl, N-methylcarbamoyl, N-ethylcarbamoyl, N-propylcarbamoyl and N-isopropylcarbamoyl, N-isobutylcarbamoyl, N,N-dimethylcarbamoyl, N-ethyl-N-methylcarbamoyl, N,N-diethylcarbamoyl, N-isobutyl-N-methylcarbamoyl, N-phenylcarbamoyl N-phenyl-N-methylcarbamoyl, N-cyclopentylcarbamoyl; N-cyclohexylyl-N-methylcarbamoyl; N-(2-methoxyethyl)-N-methylcarbamoyl, 2-hydroxypyrrolidin-1-ylcarbonyl, morpholinocarbonyl and 1,2,36-tetrahydropyridin-1-ylcarbonyl.

Examples of suitable groups for —SO₂NR^{a}R^{b} in R¹ are: sulfamoyl, N-methylsulfamoyl, N-ethylsulfamoyl, N-propylsulfamoyl and N-isopropylsulfamoyl, N-isobutylsulfamoyl, N,N-dimethylsulfamoyl, N-ethyl-N-methylsulfamoyl, N,N-diethylsulfamoyl, N-isobutyl-N-methylsulfamoyl, N-phenylsulfamoyl, N-phenyl-N-methylsulfamoyl, N-cyclopentylsulfamoyl; N-cyclohexylyl-N-methylsulfamoyl; N-(2-methoxyethyl)-N-methylsulfamoyl, 2-hydroxypyrrolidin-1-ylsulfonyl, morpholinosulfonyl and 1,2,36-tetrahydropyridin-1-ylsulfonyl,

Examples of suitable groups for NR^{a}R^{b} in R¹ include amino, methylamino, ethylamino, propylamino, isopropylamino, butylamino, isobutylamino, dimethylamino, diethylamino, N-ethyl-N-methylamino, diisopropylamino, N-isobutyl-N-methylamino, N-phenylamino, N-phenyl-N-methylamino, N-cyclopentylamino, N-cyclopentyl-N-methylamino, N-cyclohexylamino, N-cyclohexyl-N-methylamino, N-cyclohexyl-N-methylamino, N-[2-(hydroxyethyl)]amino, N-[2-(hydroxyethyl)]-N-methylamino, N-(furan-2-yl)amino, N-(furan-2-yl)-N-methylamino, azetidin-1-yl, pyrrolin-1-yl, pyrrolidin-1-yl, piperidino, morpholino and piperazino ring.

A suitable value for a (1-3C)alkylenedioxy group which may be present as a substituent formed by 2 R¹ groups on ring A or on the ring formed by R^{a} and R^{b} or R⁵ and R⁶ together with the nitrogen atom to which they are attached is, for example, methylenedioxy, ethylidenedioxy, isopropylidenedioxy or ethylenedioxy and the oxygen atoms thereof occupy adjacent ring positions. A particular value for a (1-3C)alkylenedioxy group which may be present as a substituent formed by two R¹ groups on ring A or on the ring formed by R^{a} and R^{b} or R⁵ and R⁶ together with the nitrogen atom to which they are attached is methylenedioxy.

It is to be understood that when, R¹ is a group (1-6C)alkyl substituted by, for example amino to give for example a 2-aminoethyl group, it is the (1-6C)alkyl group that is attached to ring A, An analogous convention applies to the other groups defined herein.

When in this specification reference is made to a (1-4C)alkyl group it is to be understood that such groups refer to alkyl groups containing up to 4 carbon atoms. A skilled person will realise that representative examples of such groups are those listed above under (1-6C)alkyl that contain up to 4 carbon atoms, such as methyl, ethyl, propyl, isopropyl, butyl and tert-butyl. Similarly, reference to a (1-3C)alkyl group refers to alkyl groups containing up to 3 carbon atoms such as methyl ethyl, propyl and isopropyl. A similar convention is adopted for the other groups listed above such as (1-4C)alkoxy, (2-4C)alkenyl, (2-4C)akyl and (2-4C)alkanoyl.

A suitable pharmaceutically-acceptable salt of a compound of the Formula I is, for example, an acid-addition salt of a compound of the Formula I, for example an acid-addition salt with an inorganic or organic acid such as hydrochloric, hydrobromic, sulfuric, trifluoroacetic, citric or maleic acid; or, for example, a salt of a compound of the Formula I which is sufficiently acidic, for example an alkali or alkaline earth nietal salt such as a calcium or magnesium salt, or an ammonium salt, or a salt with an organic base such as methylamine, dimethylamine, trimethylamine, piperidine, morpholine or tris-(2-hydroxyethyl)amine.

Particular novel compounds of the invention include, for example, quinazoline derivatives of the Formula I, or pharmaceutically-acceptable salts thereof, wherein, unless otherwise stated, each of m, R¹, R², R³, R⁴, R⁵, R⁶, A, m and n has any of the meanings defined hereinbefore or in paragraphs listed hereinafter:

1. Definitions of m and R¹

(a) m is 0, 1, 2 or 3 and R¹ is independently selected from halogeno, cyano, nitro, hydroxy, trifluoromethyl, (1-6C)alkyl, (1-6C)alkoxy, (1-6C)alkylthio, (1-6C)alkylsulfinyl, (1-6C)alkylsulfonyl, ureido, N-(1-6C)alkylureido, N,N-di-[(1-6C)alkylureido, —NR^{a}R^{b}, —SO₂NR^{a}R^{b} and a group of the formula —CONR^{a}R^{b} (wherein R^{a} and R^{b} are as hereinabove defined);
or, when two R¹ groups are attached to adjacent carbon atoms, they may, together with the carbon atoms to which they are attached, form a pyrrole ring, wherein the pyrrole ring is optionally substituted by 1 or 2 substituents independently selected from (1-6C)alkyl, halogeno, cyano, nitro, hydroxy, amino, carbamoyl, sulfamoyl and trifluoromethyl; or, when two R¹ groups are attached to adjacent carbon atoms, they may, together form a (1-3C)alkylenedioxy group.
(b) m is 0, 1, 2 or 3 and R¹ is independently selected from halogeno, cyano, nitro, hydroxy, trifluoromethyl, (1-6C)alkyl, (1-6C)alkoxy, (1-6C)alkylthio, (1-6C)alkylsulfinyl, (1-6C)alkylsulfonyl, ureido, N-(1-6C)alkylureido, N,N-di-[(1-6C)alkyl]ureido, —NR^{a}R^{b}, —SO₂NR^{a}R^{b} and a group of the formula —CONR^{a}R^{b} (wherein RI is hydrogen or (1-6C)alkyl and R^{b} selected from hydrogen, (1-6C)alkyl, (3-7)cycloalkyl, heteroaryl and wherein any alkyl, (3-7)cycloalkyl, heteroaryl in R^{a} and R^{b} are optionally substituted by 1 or 2 substituents selected from hydroxy and (1-4C)alkoxy;
or R^{a} and R^{b} together with the nitrogen atom to which they are attached form a 4, 5 or 6-membered ring which optionally contains an additional ring heteroatom selected from nitrogen, oxygen and sulphur and which is optionally substituted by 1 or 2 substituents on an available ring carbon atom, independently selected from halogeno, hydroxy, (1-4C)alkyl and (1-3C)alkylenedioxy and optionally substituted on any available ring nitrogen by a substituent selected from (1-4C)alkyl and (2-4C)alkanoyl (provided the ring is not thereby quaternised), and wherein any (1-4C)alkyl or (2-4C)alkanoyl group present as a substituent on the ring formed by R^{a} and R^{b} together with the nitrogen atom to which they are attached is optionally substituted by 1, 2 or 3 substituents independently selected from hydroxyl and (1-4C)alkoxy); or, when two R¹ groups are attached to adjacent carbon atoms, they may, together with the carbon atoms to which they are attached, form a pyrrole ring, wherein the pyrrole ring is optionally substituted by 1 or 2 substituents independently selected from hydroxy; or, when two R¹ groups are attached to adjacent carbon atoms, they may, together form a (1-3C)alkylenedioxy group.
(c) mis 0, 1, 2 or 3 and R¹ is independently selected from halogeno, (1-6C)alkyl, trifluoromethyl, hydroxyl, (1-6C)alkylthio, (1-6C)alkylsulfinyl, (1-6C)alkylsulfonyl, ureido, —NR^{a}R^{b}, —SO₂NR^{a}R^{b} and a group of the formula —CONR^{a}R^{b} (wherein R^{a} is hydrogen or (1-6C)alkyl and R^{b} selected from hydrogen, (1-6C)alkyl, (3-7)cycloalkyl, heteroaryl and wherein any alkyl, (3-7)cycloalkyl, heteroaryl in R^{a} and R^{b} are optionally substituted by 1 or 2 substituents selected from hydroxy and (1-4C)alkoxy;

or R^{a} and R^{b} together with the nitrogen atom to which they are attached form a azetidin-1-yl, pyrrolin-1-yl, 1,2,3,6-tetrahydropyridin-1-yl, pyrrolidin-1-yl, piperidino, piperazin-1-yl or morpholino ring, which is optionally substituted by 1 or 2 substituents on an available ring carbon atom, independently selected from hydroxyl and optionally substituted on any available ring nitrogen by a substituent selected from (1-4C)alkyl and (2-4C)alkanoyl (provided the ring is not thereby quaternised),
or, when two R¹ groups are attached to adjacent carbon atoms, they may, together with the carbon atoms to which they are attached, form a pyrrole ring, wherein the pyrrole ring is optionally substituted by 1 or 2 substituents independently selected from hydroxy; or, when two R¹ groups are attached to adjacent carbon atoms, they may, together form a (1-3C)alkylenedioxy group.
(d) m is 0, 1 or 2 and R¹ is independently selected from fluoro, chloro, methoxy, methyl, hydroxyl, methylthio, isobutylthio, sulfamoyl, and a group of the formula —CONR^{a}R^{b} (wherein R^{a} is hydrogen or methyl and R^{b} selected from hydrogen, methyl, ethyl isobutyl, furanyl, cyclopentyl and cyclohexyl, wherein any alkyl, (3-7)cycloalkyl, heteroaryl in R^{a} and R^{b} are optionally substituted by 1 or 2 substituents selected from hydroxy and methoxy;

or R^{a} and R^{b} together with the nitrogen atom to which they are attached form a 1,2,3,6-tetrahydropyridin-1-yl, pyrrolidin-1-yl, piperidino, piperazin-1-yl or morpholino ring, which is optionally substituted by 1 or 2 substituents on an available ring carbon atom, independently selected from hydroxyl and optionally substituted on any available ring nitrogen by a substituent selected from methyl and acetyl (provided the ring is not thereby quaternised), or, when two R¹ groups are attached to adjacent carbon atoms, they may, together with the carbon atoms to which they are attached, form a pyrrole ring, wherein the pyrrole ring is optionally substituted by 1 or 2 substituents independently selected from hydroxy; or, when two R¹ groups are attached to adjacent carbon atoms, they may, together form a (1-3C)alkylenedioxy group.
(e) m is 0, 1 or 2 and R¹ is independently selected from fluoro, chloro, cyano, trifluoromethyl, methyl, methoxy, methylthio, isobutylthio, sulfamoyl, and a group of the formula —CONR^{a}R^{b} (wherein R^{a} is hydrogen or methyl and R^{b} selected from hydrogen, methyl ethyl, isobutyl, furanyl, cyclopentyl and cyclohexyl, wherein any alkyl, (3-7)cycloalkyl, heteroaryl in R^{a} and R^{b} are optionally substituted by 1 or 2 substituents selected from hydroxy and methoxy;

or R^{a} and R^{b} together with the nitrogen atom to which they are attached form a 1,2,3,6-tetrahydropyridin-1-yl, pyrrolidin-1-yl, piperidino, piperazin-1-yl or morpholino ring, which is optionally substituted by 1 or 2 substituents on an available ring carbon atom, independently selected from hydroxyl and optionally substituted on any available ring nitrogen by a substituent selected from methyl and acetyl (provided the ring is not thereby quaternised), or, when two R¹ groups are attached to adjacent carbon atoms, they may, together with the carbon atoms to which they are attached, form a pyrrole ring, wherein the pyrrole ring is optionally substituted by 1 or 2 substituents independently selected from hydroxy; or, when two R¹ groups are attached to adjacent carbon atoms, they may, together form a (1-3C)alkylenedioxy group.
(f) m is 2 and R¹ is positioned in the 2- and 3-positions of ring A and R¹ is independently selected from fluoro and chloro.
(g) m is 2 and R1 is 2-fluoro and 3-chloro.
   2. Definitions of A
(a) A is phenyl or pyridin-3-yl.
(b) A is phenyl.
   3. Definitions of R²
(a) R² is selected from hydrogen, (1-6C)alkyl and a group of the formula R⁷O—, wherein R⁷ is (1-6C)alkyl optionally substituted by 1 or 2 substituents independently selected from hydroxy and a group of the formula R⁸O— (wherein R⁸ is (1-3C)alkyl).
(b) R² is selected from hydrogen, methyl, ethyl and a group of the formula R⁷O—, wherein R⁷ is methyl or ethyl.
(c) R² is methoxy.
(d) R² is hydrogen
   4. Position of R² on the Quinazoline Ring
(a) R² is in the 6-position and the substituted-pyrrolidinyloxy group is in the 7-position of the quinazoline ring.
(b) R² is in the 7-position and the substituted-pyrrolidinyloxy group is in the 6-position of the quinazoline ring.
   5. Definitions of R³
(a) R³ is selected from hydrogen, (1-6C)alkyl, (3-6C)cycloalkyl, (3-6C)cycloalkyl(1-3C)alkyl (2-6C)alkanoyl;

and wherein any (1-6C)alkyl or (2-6C)alkanoyl group within R³ is optionally substituted by 1 or 2 substituents independently selected from halogeno, hydroxy and (1-6C)alkyl and/or optionally a substituent selected from cyano, nitro, (2-8C)alkenyl, (2-8C)alkynyl, (1-6C)alkoxy and NR^{c}R^{d}, wherein R^{c} is hydrogen or (1-4C)alkyl and R_{d} is hydrogen or (1-4C)alkyl.
(b) R³ is selected from hydrogen, (1-6C)alkyl and (2-6C)alkanoyl;

and wherein any (1-6C)alkyl or (2-6C)alkanoyl group within R³ is optionally substituted by 1 or 2 substituents independently selected from halogeno, hydroxy and (1-4C)alkyl and/or optionally a substituent selected from cyano, nitro, (1-4C)alkoxy and NR^{c}R^{d}, wherein R^{c} is hydrogen or (1-4C)alkyl and R^{d} is hydrogen or (1-4C)alkyl.
(c) R³ is selected from hydrogen, methyl, ethyl, acetyl and propionyl;

and wherein any (1-6C)alkyl or (2-6C)alkanoyl group within R³ is optionally substituted by substituent independently selected from NR^{c}R^{d}, wherein R^{c} is hydrogen or methyl and R^{d} is hydrogen or methyl.
(d) R³ is hydrogen or methyl.
(e) R³ is methyl.
   6. Definitions of n and R⁴
(a) n is 0, 1 or 2 and R⁴ is independently selected from methyl, ethyl, methoxy, ethoxy, hydroxyl and oxo.
(b) n is 0 or 1 and R⁴ is independently selected from methyl, ethyl, methoxy, ethoxy, hydroxyl and oxo.
(c) n is 0.
   7. Position of the —CONR⁵R⁶ Group on the Pyrrolidine Ring
(a) The —CONR⁵R⁶ group is in the 2-position of the pyrrolidine ring.
   8. Position of the Substituted-Quinazolinyloxy Group on the Pyrrolidine Ring
(a) The substituted-quinazolinyloxy group is in the 3-position or the 4position of the pyrrolidine ring.
(b) The substituted-quinazolinyloxy group is in the 3-position of the pyrrolidine ring.
   9. Definitions of R⁵ and R⁶
(a) R⁵ is hydrogen or (1-6C)alkyl and R⁶ is selected from hydrogen, (1-6C)alkyl, (2-6C)alkenyl, (2-6C)alkynyl, (1-6C)alkoxy, (3-7)cycloalkyl, heterocyclyl, heteroaryl, (3-7)cycloalkyl(1-3C)alkyl, (3-7)heterocyclyl(1-3C)alkyl and heteroaryl(1-3C)alkyl, and wherein any (1-3C)alkyl, (1-6C)alkyl, (3-7)cycloalkyl, heteroaryl or heterocyclyl group within R⁵ or R⁶ is optionally substituted (on any available carbon atoms) by 1 or 2 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano and (1-4C)alkoxy,
and wherein any heterocyclyl group within R⁶ is optionally substituted on any available ring nitrogen (provided the ring is not thereby quaternised) by (1-4C)alkyl or (2-4C)alkanoyl, or R⁵ and R⁶ together with the nitrogen atom to which they are attached form a 4, 5 or 6 membered ring which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised),

and wherein any (1-4C)alkyl or (2-4C)alkanoyl group present as a substituent on the ring formed by R⁵ and R⁶ together with the nitrogen atom to which they are attached is optionally substituted by 1 or 2 substituents independently selected from halogeno and hydroxy and/or optionally a substituent selected from (1-4C)alkyl and (1-4C)alkoxy;
provided that when the pyrrolidinyloxy group is liked to the 6-position of the quinazoline ring, m is 2 and substituents R¹ are both halogeno and attached to the 2- and 3-positions of the ring A, then R⁶ is selected from substituted-(1-6C)alkyl (wherein substituted-(1-6C)alkyl is (1-6C)alkyl substituted by 1 or 2 substituents independently selected from halogeno, hydroxy(1-6C)alkyl, (1-6C)alkoxycarbonyl, carbamoyl, (2-6C)alkanoylamino, (1-6C)alkylamino, di-[(1-6C)alkyl]amino and hydroxy and/or optionally a substituent selected from oxo, cyano and (1-4C)alkoxy), (2-6C)alkenyl, (2-6C)alkynyl, (1-6C)alkoxy, (3-7)cycloalkyl, (1-6C)alkylsulfonyl, (3-7)heterocyclyl, heteroaryl, (3-7)cycloalkyl(1-6C)alkyl, (3-7)heterocyclyl(1-6C)alkyl and heteroaryl(1-6C)alkyl, and wherein any (3-7)cycloalkyl, heteroaryl or (3-7)heterocyclyl group within R⁶ is optionally substituted (on any available carbon atoms) by 1 or 2 substituents independently selected from halogeno, hydroxy, (1-6C)alkyl, hydroxy(1-6C)alkyl, (1-6C)alkoxycarbonyl, carbamoyl (2-6C)alkanoylamino and hydroxy and/or optionally a substituent selected from oxo, cyano, nitro and (1-4C)alkoxy,
and wherein any heteroaryl or heterocyclyl group within R⁶ is optionally substituted on any available ring nitrogen (provided the ring is not thereby quaternised) by (1-4C)alkyl or (2-4C)alkanoyl, or
R⁵ and R⁶ together with the nitrogen atom to which they are attached form a 4, 5 or 6 membered ring which contains one or two nitrogen atoms as the only heteroatom(s) present in the ring and which is optionally substituted by 1 or 2 substituents on an available ring carbon atom, independently selected from hydroxy, carbamoyl, (1-4C)alkyl, and (1-3C)alkylenedioxy; and wherein any 4, 5 or 6 membered heterocyclic ring formed by R⁵ and R⁶ is optionally substituted on any available ring nitrogen (provided the ring is not thereby quaternised) by (1-4C)alkyl or (2-4C)alkanoyl;
(b) R⁵ is hydrogen, methyl, ethyl propyl, isopropyl or isobutyl and R⁶ is selected from hydrogen, methyl, ethyl propyl isopropyl, isobutyl, vinyl, isopropenyl, allyl but-2-enyl ethynyl, 2-propynyl, butynyl, methoxy, ethoxy propoxy, isopropoxy, cyclopropyl, cyclopentyl, cyclohexyl azetidinyl, oxazepanyl pyrrolinyl, pyrrolidinyl, morpholinyl, tetrahydro-1,4thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl tetrahydrothiopyranyl, thiomorpholinyl, pyrazolyl, thienyl, oxazolyl, isooxazolyl, imidazolyl, pyridinyl, pyridazinyl, pyrazinyl pyrimidyl, furanyl, pyrazolyl, thiazolyl, isothiazolyl, thiadiazolyl, cyclopropylmethyl, cyclopentylmethyl, cyclohexylmethyl 2-cyclopropylethyl, 2-cyclopentylethyl, 2-cyclohexylethyl, azetidinylmethyl, oxazolylmethyl, pyrrolinylmethyl, pyrrolidinylmethyl, miorpholinylmethyl, tetrahydro-1,4thiazinylmethyl, piperidinylmethyl homopiperidinylmethyl, piperazinylmethyl, homopiperazinylnethyl, dihydropyridinylmethyl, tetrahydropyridinylmethyl, dihydropyrimidinylmethyl tetrahydropyrimidinylmethyl, tetrahydrothienylmethyl, tetrahydrothiopyranylmethyl, thiomorpholinylmethyl pyrazolylmethyl, thienylmethyl, oxazolylmethyl, isoxazolylmethyl imidazolylmethyl, pyridinylmethyl, pyridazinylmethyl, pyrazinylmethyl pyrimidylmethyl, furanylmethyl pyrazolylmethyl thiazolylmethyl isothiazolylmethyl, thiadiazolylmethyl, 2-(azetidinyl)ethyl, 2-(oxazepanyl)ethyl, 2-(pyrrolinyl)ethyl, 2-(pyrrolidinyl)ethyl, 2-(morpholinyl)ethyl 2-(tetrahydro-1,4thiazinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrothienyl)ethyl, 2-(tetrahydrothiopyranyl)ethyl, 2-(thiomorpholinyl)ethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl, 2-(isothiazolyl)ethyl and 2-(thiadiazolyl)ethyl,
and wherein any alkyl, cycloalkyl, heteroaryl or heterocyclyl group within R⁵ or R⁶ is optionally substituted (on any available carbon atom) by 1 or 2 substituents independently selected from fluoro, chloro, bromo, hydroxymethyl, 2-hydroxyethyl, methoxycarbonyl, ethoxycarbonyl, carbamoyl, acetamido, propionamido and hydroxy and/or optionally a substituent selected from oxo, cyano, methoxy and ethoxy,
and wherein any heterocyclyl group within R⁶ is optionally substituted on any available ring nitrogen (provided the ring is not thereby quaternised) by methyl, ethyl, acetyl or propionyl, or R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, bromo, hydroxy, methyl, ethyl and propylenedioxy, and optionally substituted on any available ring nitrogen by a substituent selected from methyl, ethyl, acetyl and propionyl (provided the ring is not thereby quaternised),
and wherein any alkyl or alkanoyl group present as a substituent on the ring formed by R⁵ and R⁶ together with the nitrogen atom to which they are attached is optionally substituted by 1 or 2 substituents independently selected from fluoro, chloro, bromo and hydroxy and/or optionally a substituent selected from methyl, ethyl, methoxy and ethoxy;

provided that when the pyrrolidinyloxy group is linked to the 6-position of the quinazoline ring, m is 2 and substituents R¹ are both halogeno and attached to the 2- and 3-positions of the ring A, then R⁶ is selected from substituted-methyl, substituted-ethyl substituted-propyl, substituted-isopropyl, substituted-isobutyl, (wherein the substituted groups are substituted by 1 or 2 substituents independently selected from fluoro, chloro, bromo, hydroxymethyl, 2-hydroxyethyl, methoxycarbonyl, ethoxycarbonyl, carbamoyl, acetamido, propionamido, di-methylamino and hydroxy and/or optionally a substituent selected from oxo, cyano, methoxy and ethoxy) vinyl, isopropenyl allyl but-2-enyl ethynyl, 2-propynyl, butynyl, methoxy, ethoxy propoxy, isopropoxy, cyclopropyl, cyclopentyl cyclohexyl, azetidinyl, oxazepanyl, pyrrolinyl pyrrolidinyl, morpholinyl, tetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl tetrahydrothiopyranyl thiomorpholinyl, pyrazolyl, thienyl, oxazolyl, isoxazolyl, imidazolyl, pyridinyl pyridazinyl, pyrazinyl, pyrimidyl, furanyl, pyrazolyl, thiazolyl, isothiazolyl, thiadiazolyl cyclopropylmethyl, cyclopentylmethyl, cyclohexylmethyl, 2-cyclopropylethyl, 2-cyclopentylethyl, 2-cyclohexylethyl azetidinylmethyl, oxazolylmethyl, pyrrolinylmethyl, pyrrolidinylmethyl, morpholinylmethyl, tetrahydro-1,4-thiazinyhmethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl tetrahydrothienylmethyl, tetrahydrotbiopyranylmethyl, thiomorpholinylmethyl, pyrazolylmethyl thienylmethyl, oxazolylmethyl, isoxazolylmethyl, imidazolylmethyl, pyridinylmethyl, pyridazinylmethyl, pyrazinylmethyl, pyrimidylmethyl, furanylmethyl, pyrazolylmethyl, thiazolylmethyl isothiazolylmethyl, thiadiazolylmethyl, 2-(azetidinyl)ethyl, 2-(oxazepanyl)ethyl, 2-(pyrrolinyl)ethyl, 2-(pyrrolidinyl)ethyl, 2-(morpholinyl)ethyl, 2-(tetrahydro-1,4-thiazinyl)ethyl, 2-(piperidinyl)ethyl, 2-homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyridinyl)ethyl, 2-(tetrahydrothienyl)ethyl, 2-(tetrahydrothiopyranyl)ethyl, 2-(thiomorpholinyl)ethyl 2-(pyrazolyl)ethyl, 2-(thienyl)ethyl, 2-(oxazolyl)ethyl 2-(isoxazolyl)ethyl 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl, 2-(isotbiazolyl)ethyl and 2-(thiadiazolyl)ethyl,
and wherein any cycloalkyl, heteroaryl or heterocyclyl group within R⁶ is optionally substituted (on any available carbon atoms) by 1 or 2 substituents independently selected from fluoro, chloro, bromo, hydroxy, methyl, ethyl, hydroxymethyl, 2-hydroxyethyl, methoxycarbonyl, ethoxycarbonyl, carbamoyl, acetamido, propionamido, and hydroxy and/or optionally a substituent selected from oxo, cyano, methoxy and ethoxy,
and wherein any heteroaryl or heterocyclyl group within R⁶ is optionally substituted on any available ring nitrogen (provided the ring is not thereby quaternised) by methyl, ethyl, acetyl or propionyl, or R⁵ and R⁶ together with the nitrogen atom to which they are attached form a azetidin-1-yl, pyrrolin-1-yl, pyrrolidin-1-yl, piperidino, or piperazino ring which is optionally substituted on any available carbon atom by 1 or 2 substituents selected from hydroxy, carbamoyl, methyl or ethyl, or substituted on adjacent ring carbon atoms by a propylenedioxy group, or optionally substituted on any available ring nitrogen by a substituent selected from methyl, ethyl, acetyl and propionyl (provided the ring is not thereby quaternised).
(c) R⁵ is hydrogen, methyl or ethyl and R⁶ is selected from hydrogen, methyl, ethyl propyl, isopropyl, isobutyl, vinyl, isoprop-2-enyl, allyl, but-2-enyl ethynyl, 2-prop-2-ynyl, but-3-ynyl, methoxy, ethoxy, cyclopropyl, cyclopentyl, cyclohexyl, azetidinyl, pyrrolinyl, pyrrolidinyl, morpholinyl, piperidinyl, piperazinyl, tetrahydropyridinyl thiomorpholinyl, 1,2,3,6-tetrahydropyridin-1-yl, pyrazolyl thienyl, oxazolyl, isoxazolyl, imidazolyl, pyridinyl, pyridazinyl, pyrazinyl pyrimidyl, furanyl, pyrazolyl thiazolyl isothiazolyl, cyclopropylmethyl, cyclopentylmethyl, cyclohexylmethyl, 2-cyclopropylethyl, 2-cyclopentylethyl, 2-cyclohexylethyl, azetidinylmethyl, pyrrolinylmethyl, pyrrolidinylmethyl morpholinylmethyl, piperidinylmethyl piperazinylmethyl tetrahydropyridinylmethyl, thiomorpholinylmethyl, pyrazolylmethyl, thienylmethyl, oxazolylmethyl, isoxazolylmethyl, imidazolylnethyl, pyridinylmethyl, pyridazinylmethyl, pyrazinylmethyl, pyridylmethyl, furanylmethyl pyrazolylmethyl, thiazolylmethyl, isothiazolylmethyl, 2-(azetidinyl)ethyl2-(pyrrolinyl)ethyl, 2-(pyrrolidinyl)ethyl, 2-(morpholinyl)ethyl, 2-(piperidinyl)ethyl, 2-(piperazinyl)ethyl, 2-(tetrahydropyridinyl)ethyl, 2-(thiomorpholinyl)ethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl)ethyl,
and wherein any alkyl, cycloalkyl, heteroaryl or heterocyclyl group within R⁵ or R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl carbamoyl, acetamido and hydroxy and/or optionally a substituent selected from oxo, cyano, methoxy and ethoxy,
and wherein any heterocyclyl group within R⁶ is optionally substituted on any available ring nitrogen (provided the ring is not thereby quaternised) by methyl, ethyl, acetyl or propionyl, or R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, hydroxy, methyl, ethyl and propylenedioxy, and optionally substituted on any available ring nitrogen by a substituent selected from methyl, ethyl, acetyl and propionyl (provided the ring is not thereby quaternised),
and wherein any alkyl or alkanoyl group present as a substituent on the ring formed by R⁵ and R⁶ together with the nitrogen atom to which they are attached is optionally substituted by 1 or 2 substituents independently selected from fluoro, chloro and hydroxy and/or optionally a substituent selected from methyl, ethyl, methoxy and ethoxy;

provided that when the pyrrolidinyloxy group is linked to the 6-position of the quinazoline ring, m is 2 and substituents R¹ are both halogeno and attached to the 2- and 3-positions of the ring A, then R⁶ is selected from substituted-methyl, substituted-ethyl substituted-propyl, substituted-isopropyl, substituted-isobutyl, (wherein the substituted groups are substituted by 1 or 2 substituents independently selected from fluoro, chloro, bromo, hydroxymethyl, 2-hydroxyethyl methoxycarbonyl, ethoxycarbonyl, carbamoyl, acetamido, dimethylamino and hydroxy and/or optionally a substituent selected from oxo, cyano, niethoxy and ethoxy), vinyl, isoprop-2-enyl, allyl, but-2-enyl ethynyl, 2-prop-2-ynyl, but-3-ynyl, methoxy, ethoxy, cyclopropyl cyclopentyl, cyclohexyl, azetidinyl, pyrrolinyl pyrrolidinyl, morpholinyl, piperidinyl, piperazinyl, tetrahydropyridinyl, thiomorpholinyl, 1,2,3,6-tetrahydropyridin-1-yl, pyrazolyl, thienyl, oxazolyl, isoxazolyl, imidazolyl, pyridinyl, pyridazinyl, pyrazinyl, pyrimidyl, furanyl, pyrazolyl, thiazolyl, isotbiazolyl, cyclopropylmethyl, cyclopentylmethyl, cyclohexylmethyl, 2-cyclopropylethyl, 2-cyclopentylethyl, 2-cyclohexylethyl, azetidinylmethyl, pyrrolinylmethyl, pyrrolidinylmethyl, morpholinylmethyl, piperidinylmethyl, piperazinylmethyl, tetrahydropyridinylmethyl, thiomorpholinylmethyl, pyrazolylmethyl, thienylmethyl, oxazolylmethyl isoxazolylmethyl, imidazolylmethyl, pyridinylmethyl, pyridazinylmethyl, pyrazinylmethyl pyrimidylmethyl, furanylmethyl, pyrazolylmethyl, thiazolylmethyl, isothiazolylmethyl, 2-(azetidinyl)ethyl2-(pyrrolinyl)ethyl, 2-(pyrrolidinyl)ethyl, 2-(morpholinyl)ethyl, 2-(piperidinyl)ethyl, 2-(piperazinyl)ethyl, 2-(tetrahydropyridinyl)ethyl, 2-(thiomorpholinyl)ethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl)ethyl,
and wherein any cycloalkyl, heteroaryl or heterocyclyl group within R⁶ is optionally substituted (on any available carbon atoms) by 1 or 2 substituents independently selected from fluoro, chloro, bromo, hydroxy, methyl, hydroxymethyl, 2-hydroxyethyl, methoxycarbonyl, ethoxycarbonyl, carbamoyl, acetamido and hydroxy and/or optionally a substituent selected from oxo, cyano, methoxy and ethoxy,
and wherein any heteroaryl or heterocyclyl group within R⁶ is optionally substituted on any available ring nitrogen (provided the ring is not thereby quaternised) by methyl, ethyl, or acetyl or
R⁵ and R⁶ together with the nitrogen atom to which they are attached form a azetidin-1-yl, pyrrolin-1-yl, pyrrolidin-1-yl, piperidino, or piperazino ring which is optionally substituted by a substituent selected from hydroxy, carbamoyl, methyl or ethyl, or substituted on adjacent ring carbon atoms by a propylenedioxy group, and optionally substituted on any available ring nitrogen by a substituent selected from methyl, ethyl, acetyl and propionyl (provided the ring is not thereby quaternised).
(d) R⁵ is hydrogen or methyl and R⁶ is selected from hydrogen, methyl, ethyl, propyl, isopropyl, vinyl, isoprop-2-enyl, allyl, but-2-enyl ethynyl, 2-propynyl, but-3-ynyl, methoxy, cyclopropyl, cyclopentyl 1-(hydroxymethyl)cyclopentyl, cyclohexyl, 4hydroxycyclohexyl, cyclopropylmethyl, cyclopentylmethyl, methoxymethyl, 2-(methoxy)ethyl, 2-(ethoxy)ethyl, carbamoylmethyl, 2-(acetyl)ethyl, cyanomethyl, 2-(cyano)ethyl, 2,3-dihydroxypropyl, 2-hydroxyl)-1,1-dimethylethyl, 2,2,2-trifluoroethyl, 1-(ethoxycarbonyl)-2-hydroxyethyl, 2-acetamido)ethyl, tetrahydrofaran-2-ylmethyl, imidazol-2-ylmethyl, 1-methylpyrazol-5-yl, 1-methylpyrazol-5-yl, 3-methylpyrazol-5-yl, imidazol-1-ylmethyl, 2-(imidazol-1-yl)ethyl, furan-2-ylmethyl, 2-(furan-2-yl)ethyl, 5-methylisoxazol-3-ylmethyl, thien-3yl, morpholino, piperidin-4-yl, 1-methylpiperidin-4yl, tetrahydro-2H-pyran-4-yl and 3-oxotetrahydrofuran-4-yl, or R⁵ and R⁶ together with the nitrogen atom to which they are attached form a 3-hydroxyazetidin-1-yl, 2-carbamoylazetidin-1-yl, pyrrolin-1-yl, pyrrolidin-1-yl, 3-hydroxy, pyrrolidin-1-yl, piperidino, morpholino or piperazino group;

provided that when the pyrrolidinyloxy group is linked to the 6-position of the quinazoline ring, m is 2 and substituents R¹ are both halogeno and attached to the 2- and 3-positions of the ring A, then R⁶ is selected from vinyl, isoprop-2-enyl, allyl, but-2-enyl ethynyl, 2-propynyl, but-3-ynyl, methoxy, cyclopropyl, cyclopentyl, 1-(hydroxymethyl)cyclopentyl, cyclohexyl, 4-hydroxycyclohexyl, cyclopropylmethyl, cyclopentyhmethyl, methoxymethyl, 2-(methoxy)ethyl, 2-(ethoxy)ethyl, carbamoylmethyl 2-(acetyl)ethyl, 2-(acetylamino)ethyl, cyanomethyl, 2-(dimethylamino)ethyl 2-(cyano)ethyl, 2,3-dihydroxypropyl, 2-(hydroxyl)- 1,1-dimethylethyl, 2,2,2-trifluoroethyl, 2-(hydroxy)-1-(methoxycarbonyl)ethyl, 1-(ethoxycarbonyl)-2-hydroxyethyl, 2-(acetamido)ethyl, tetrahydrofuran-2-ylmethyl, imidazol-2-ylmethyl, 1-methylpyrazol-5-yl, 3-methylpyrazol-5-yl, imidazol-1-ylmethyl 1H-imidazol-2-ylmethyl, 2-(imidazol-1-yl)ethyl, 2-(1H-imidazol-4-yl)ethyl, furan-2-ylmethyl, 2-(furan-2-yl)ethyl, 5-methylisoxazol-3-ylmethyl, thien-3yl, morpholino, piperidin-4-yl, 1-methylpiperidin-4-yl, tetrahydropyran-4-yl, tetrahydro-2H-pyran-4-ylmethyl, 4-hydroxytetrahydrofuran-3-yl, and 3-oxotetrahydrofuran-4-yl or R⁵ and R⁶ together with the nitrogen atom to which they are attached form an azetidin-1-yl ring substituted in the 2 position by a carbamoyl group.
(e) R⁵ is hydrogen or (1-6C)alkyl and R⁶ is selected from hydrogen, (1-6C)alkyl, (2-6C)alkenyl, (2-6C)alkyl, (1-6C)alkoxy, (3-7)cycloalkyl, heterocyclyl, heteroaryl, (3-7)cycloalkyl(1-3C)alkyl, (3-7)heterocyclyl(1-3C)alkyl and heteroaryl(1-3C)alkyl,
and wherein any (1-3C)alkyl, (1-6C)alkyl, (3-7)cycloalkyl, heteroaryl or heterocyclyl group within R⁵ or R⁶ is optionally substituted (on any available carbon atoms) by 1 or 2 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano and (1-4C)alkoxy,
and wherein any heterocyclyl group within R⁶ is optionally substituted on any available ring nitrogen (provided the ring is not thereby quaternised) by (1-4C)alkyl or (2-4C)alkanoyl, or R⁵ and R⁶ together with the nitrogen atom to which they are attached form a 4, 5 or 6 membered ring which is optionally substituted by 1 or 2 substituents on an available ring carbon atom, independently selected from halogeno, hydroxy, (1-4C)alkyl and
(1-3C)alkylenedioxy, and optionally substituted on any available ring nitrogen by a substituent selected from (1-4C(alkyl and (2-4C)alkanoyl (provided the ring is not thereby quaternised), and wherein any (1-4C)alkyl or (2-4C)alkanoyl group present as a substituent on the ring formed by R⁵ and R⁶ together with the nitrogen atom to which they are attached is optionally substituted by 1 or 2 substituents independently selected from halogeno and hydroxy and/or optionally a substituent selected from (1-4C)alkyl and (1-4C)alkoxy;

provided that when the pyrrolidinyloxy group is linked to the 6-position of the quinazoline ring, m is 2 and substituents R¹ are both halogeno and attached to the 2- and 3-positions of the ring A, then R⁶ is (3-7)cycloalkyl optionally substituted by 1 or 2 substituents independently selected from halogeno, hydroxy, (1-6C)alkyl, hydroxy(1-6C)alkyl, (1-6C)alkoxycarbonyl, carbamoyl (2-6C)alkanoylamino and hydroxy and/or optionally a substituent selected from oxo, cyano, nitro and (1-4C)alkoxy.
(f) R⁵ is hydrogen, methyl, ethyl propyl, isopropyl or isobutyl and R⁶ is selected from hydrogen, methyl, ethyl propyl, isopropyl, isobutyl, vinyl, isopropenyl allyl, but-2-enyl ethynyl 2-propynyl, butynyl methoxy, ethoxy propoxy, isopropoxy, cyclopropyl, cyclopentyl, cyclohexyl, azetidinyl, oxazepanyl, pyrrolinyl, pyrrolidinyl, morpholinyl tetrahydro-1,4-thiazinyl, piperidinyl homopiperidinyl, piperazinyl, homopiperazinyl, dihydropyridinyl, tetrahydropyridinyl, dihydropyrimidinyl, tetrahydropyrimidinyl, tetrahydrothienyl tetrahydrothiopyranyl thiomorpholinyl, pyrazolyl thienyl oxazolyl, isoxazolyl imidazolyl, pyridinyl, pyridazinyl, pyrazinyl, pyrimidyl, furanyl, pyrazolyl, thiazolyl, isothiazolyl, thiadiazolyl, cyclopropyhlethyl, cyclopentylmethyl, cyclohexylmethyl, 2-cyclopropylethyl, 2-cyclopentylethyl, 2-cyclohexylethyl, azetidinylmethyl, oxazolylmethyl, pyrro inylmethyl pyrrolidinylmethyl, morpholinylmethyl tetrahydro- 1,4-thiazinylmethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl tetrahydrothienylmethyl, tetrahydrothiopyranylimethyl, thiomorpholiiylmethyl, pyrazolylmethyl, thienylmethyl oxazolylmethyl, isoxazolylmethyl, imidazolylmethyl, pyridinylmethyl, pyridazinylmethyl, pyrazinylmethyl pyrimidylmethyl, furanylmethyl, pyrazolylmethyl, thiazolylmethyl isothiazolylmethyl, thiadiazolylmethyl, 2-(azetidinyl)ethyl 2-(oxazepanyl)ethyl, 2-(pyrrolinyl)ethyl, 2-(pyrrolidinyl)ethyl, 2-(morpholinyl)ethyl, 2-(tetrahydro-1,4thiazinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrothienyl)ethyl, 2-(tetrahydrothiopyranyl)ethyl, 2-(thiomorpholinyl)ethyl, 2-(pyrazolyl)ethyl, 2-(thienyl)ethyl, 2-(oxazolyl)ethyl 2-(isoxazolyl)ethyl 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl, 2-(isothiazolyl)ethyl and 2-(thiadiazolyl)ethyl,
and wherein any alkyl, cycloalkyl heteroaryl or heterocyclyl group within R⁵ or R⁶ is optionally substituted (on any available carbon atoms) by 1 or 2 substituents independently selected from fluoro, chloro, bromo, hydroxymethyl, 2-hydroxyethyl, methoxycarbonyl ethoxycarbonyl, carbamoyl, acetamido, propionamido and hydroxy and/or optionally a substituent selected from oxo, cyano, methoxy and ethoxy,
and wherein any heterocyclyl group within R⁶ is optionally substituted on any available ring nitrogen (provided the ring is not thereby quaternised) by methyl, ethyl acetyl or propionyl, or R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, bromo, hydroxy, methyl, ethyl and propylenedioxy, and optionally substituted on any available ring nitrogen by a substituent selected from methyl, ethyl, acetyl and propionyl (provided the ring is not thereby quaternised),

and wherein any alkyl or alkanoyl group present as a substituent on the ring formed by R⁵ and R⁶ together with the nitrogen atom to which they are attached is optionally substituted by 1 or 2 substituents independently selected from fluoro, chloro, bromo and hydroxy and/or optionally a substituent selected from methyl, ethyl, methoxy and ethoxy;
provided that when the pyrrolidinyloxy group is linked to the 6-position of the quinazoline ring, m is 2 and substituents R¹ are both halogeno and attached to the 2- and 3-positions of the ring A, then R⁶ is selected from cyclopropyl, cyclopentyl, and cyclohexyl, and wherein said cyclopropyl, cyclopentyl, and cyclohexyl group is optionally substituted by 1 or 2 substituents independently selected from fluoro, chloro, bromo, hydroxy, methyl, ethyl, hydroxymethyl, 2-hydroxyethyl, methoxycarbonyl ethoxycarbonyl, carbamoyl, acetamido, propionamido and hydroxy and/or optionally a substituent selected from oxo, cyano, methoxy and ethoxy.
(g) R⁵ is hydrogen, methyl or ethyl and R⁶ is selected from hydrogen, methyl, ethyl propyl, isopropyl isobutyl, vinyl, isoprop-2-enyl, allyl, but-2-enyl ethynyl, 2-prop-2-ynyl, but-3-ynyl, methoxy, ethoxy, cyclopropyl, cyclopentyl, cyclohexyl azetidinyl, pyrrolinyl, pyrrolidinyl, morpholinyl, piperidinyl, piperazinyl, tetrahydropyridinyl thiomorpholinyl 1,2,3,6-tetrahydropyridin-1-yl, pyrazolyl, thienyl, oxazolyl isoxazolyl imidazolyl, pyridinyl, pyridazinyl, pyrazinyl, pyrimidyl, furanyl, pyrazolyl thiazolyl, isothiazolyl, cyclopropylmethyl, cyclopentylmethyl, cyclohexylmethyl, 2-cyclopropylethyl, 2-cyclopentylethyl, 2-cyclohexylethyl, azetidinylmethyl, pyrrolinylmethyl, pyrrolidinylmethyl, morpholinylmethyl, piperidinylmethyl, piperazinylmethyl, tetrahydropyridinylmethyl, thiomorpholinylnethyl, pyrazolylmethyl thienylmethyl, oxazolylmethyl, isoxazolylmethyl, imidazolylmethyl, pyridinylmethyl, pyndazinylmethyl, pyrazinylmethyl pyrimidyblmethyl, furanylmethyl, pyrazolylmethyl, thiazolylmethyl, isothiazolylmethyl, 2-(azetidinyl)ethyl2-(pyrrolinyl)ethyl, 2-(pyrrolidinyl)ethyl, 2-(morpholinyl)ethyl, 2-(piperidinyl)ethyl, 2-(piperazinyl)ethyl, 2-(tetrahydropyridinyl)ethyl, 2-(thiomorpholinyl)ethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl)ethyl,
and wherein any alkyl, cycloalkyl, heteroaryl or heterocyclyl group within R⁵ or R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido and hydroxy and/or optionally a substituent selected from oxo, cyano, methoxy and ethoxy,
and wherein any heterocyclyl group within R⁶ is optionally substituted on any available ring nitrogen (provided the ring is not thereby quaternised) by methyl, ethyl, acetyl or propionyl, or R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, hydroxy, methyl, ethyl and propylenedioxy, and optionally substituted on any available ring nitrogen by a substituent selected from methyl ethyl, acetyl and propionyl (provided the ring is not thereby quaternised),

and wherein any alkyl or alkanoyl group present as a substituent on the ring formed by R⁵ and R⁶ together with the nitrogen atom to which they are attached is optionally substituted by 1 or 2 substituents independently selected from fluoro, chloro and hydroxy and/or optionally a substituent selected from methyl, ethyl, methoxy and ethoxy;

provided that when the pyrrolidinyloxy group is linked to the 6position of the quinazoline ring, m is 2 and substituents R¹ are both halogeno and attached to the 2- and 3-positions of the ring A, then R⁶ is selected from cyclopropyl cyclopentyl and cyclohexyl, and wherein said cyclopropyl, cyclopentyl, and cyclohexyl group is optionally substituted by 1 or 2 substituents independently selected from hydroxy and hydroxymethyl.
(h) R⁵ is hydrogen or methyl and R⁶ is selected from hydrogen, methyl, ethyl, propyl, isopropyl vinyl isoprop-2-enyl, allyl, but-2-enyl ethynyl, 2-propynyl, but-3-ynyl, methoxy, cyclopropyl, cyclopentyl 1-(hydroxymethyl)cyclopentyl, cyclohexyl, 4-hydroxycyclohexyl, cyclopropylmethyl, cyclopentylmethyl, methoxymethyl, 2-(methoxy)ethyl, 2-(ethoxy)ethyl, carbamoylmethyl 2-(acetyl)ethyl, cyanomethyl, 2-(cyano)ethyl, 2,3-dihydroxypropyl, 2-(hydroxyl)-1,1-dimethylethyl, 2,2,2-trifluoroethyl, 1-(ethoxycarbonyl)-2-hydroxyethyl, 2-acetamido)ethyl, tetrahydrofuran-2-ylmethyl, imidazol-2-ylmethyl, 1-methylpyrazol-5-yl, 1-methylpyrazol-5-yl, 3-methylpyrazol-5-yl, imidazol-1-ylmethyl, 2-(imidazol-1-yl)ethyl, furan-2-ylmethyl, 2-(furan-2-yl)ethyl, 5-methyhsoxazol-3-ylmethyl, thien-3yl, morpholino, piperidin-4-yl, 1-methylpiperidin-4-yl, tetrahydro-2H-pyran-4-yl and 3-oxotetrahydrofuran-4-yl,

or R⁵ and R⁶ together with the nitrogen atom to which they are attached form a 3-hydroxyazetidin-1-yl, 2-carbamoylazetidin-1-yl, pyrrolin-1-yl, pyrrolidin-1-yl, 3-hydroxy, pyrrolidin-1-yl, piperidino, morpholino or piperazino group;

provided that when the pyrrolidinyloxy group is linked to the 6-position of the quinazoline ring, m is 2 and substituents R¹ are both halogeno and attached to the 2- and 3-positions of the ring A, then R⁶ is selected from cyclopropyl, cyclopentyl, 1-(hydroxymethyl)cyclopentyl, cyclohexyl, and 4-hydroxycyclohexyl.
(i) R⁵ is hydrogen or (1-6C)alkyl and R6 is selected from hydrogen, (1-6C)alkyl, (2-6C)alkenyl, (2-6C)alkynyl, (1-6C)alkoxy, (3-7)cycloalkyl, heterocyclyl, heteroaryl, (3-7)cycloalkyl(1-3C)alkyl, (3-7)heterocyclyl(1-3C)alkyl and heteroaryl(1-3C)alkyl, and wherein any (1-3C)alkyl, (1-6C)allyl (3-7)cycloalkyl, heteroaryl or heterocyclyl group within R⁵ or R⁶ is optionally substituted (on any available carbon atoms) by 1 or 2 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano and (1-4C)alkoxy,

and wherein any heterocyclyl group within R⁶ is optionally substituted on any available ring nitrogen (provided the ring is not thereby quaternised) by (1-4C)alkyl or (2-4C)alkanoyl, or
R⁵ and R⁶ together with the nitrogen atom to which they are attached form a 4, 5 or 6 membered ring which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised),

and wherein any (1-4C)alkyl or (2-4C)alkanoyl group present as a substituent on the ring formed by R⁵ and R⁶ together with the nitrogen atom to which they are attached is optionally substituted by 1 or 2 substituents independently selected from halogeno and hydroxy and/or optionally a substituent selected from (1-4C)alkyl and (1-4C)alkoxy;

provided that when the pyrrolidinyloxy group is linked to the 6-position of the quinazoline ring, m is 2 and substituents R¹ are both halogeno and attached to the 2- and 3-positions of the ring A, then R⁶ is a (3-7)cycloalkyl(1-6C)alkyl group, wherein the (3-7)cycloalkyl moiety is optionally substituted (on any available carbon atoms) by 1 or 2 substituents independently selected from halogeno, hydroxy, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy.
(j) R⁵ is hydrogen, methyl, ethyl propyl, isopropyl or isobutyl and R⁶ is selected from hydrogen, methyl, ethyl propyl, isopropyl, isobutyl, vinyl, isopropenyl, allyl, but-2-enyl ethynyl, 2-propynyl, butynyl methoxy, ethoxy propoxy, isopropoxy, cyclopropyl, cyclopentyl cyclohexyl, azetidinyl, oxazepanyl pyrrolinyl pyrrolidinyl, morpholinyl tetrahydro-1,4thiazinyl, piperidinyl, homopiperidinyl piperazinyl, homopiperazinyl, dihydropyridinyl tetrahydropyridinyl, dihydropyrimidinyl, tetrahydropyrimidinyl, tetrahydrothienyl, tetrahydrothiopyranyl, thiomorpholinyl, pyrazolyl, thienyl oxazolyl, isoxazolyl imidazolyl, pyridinyl pyridazinyl, pyrazinyl pyrimidyl, furanyl, pyrazolyl, thiazolyl, isothiazolyl, thiadiazolyl, cyclopropylmethyl, cyclopentylmethyl cyclohexylmethyl, 2-cyclopropylethyl, 2-cyclopentylethyl, 2-cyclohexylethyl, azetidinylmethyl, oxazolylmethyl, pyrrolinylmethyl, pyrrolidinylmethyl, morpholinylmethyl, tetrahydro-1,4t-iazinylmethyl, piperidinylmethyl, homopiperidinylmethyl piperazinylmethyl homopiperazinylmethyl dihydropyridinylmethyl, tetrahydropyridinylmethyl, dihydropyrimidinylmethyl, tetrahydropyrimidinylmethyl, tetrahydrothienylmethyl, tetrahydrothiopyranylimethyl thiomorpholinylmethyl, pyrazolylmethyl, thienylmethyl, oxazolylmethyl, isoxazolylmethyl, imidazolylmethyl, pyridinylmethyl, pyridazinylmethyl, pyrazinylmethyl, pyrinidylmethyl, furanylmethyl, pyrazolylmethyl, thiazolylmethyl, isothiazolylmethyl, thiadiazolylmethyl, 2-(azetidinyl)ethyl, 2-(oxazepanyl)ethyl, 2-(pyrrolinyl)ethyl, 2-(pyrrolidinyl)ethyl, 2-(morpholinyl)ethyl, 2-(tetrahydro-1,4thiazinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrothienyl)ethyl, 2-(tetrahydrothiopyranyl)ethyl, 2-(thiomorpholinyl)ethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl, 2-(isothiazolyl)ethyl and 2-(thiadiazolyl)ethyl,
and wherein any alkyl, cycloalkyl, heteroaryl or heterocyclyl group within R⁵ or R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido, propionamido and hydroxy and/or optionally a substituent selected from oxo, cyano, methoxy and ethoxy,
and wherein any heterocyclyl group within R⁶ is optionally substituted on any available ring nitrogen (provided the ring is not thereby quaternised) by methyl, ethyl, acetyl or propionyl, or R⁵ and R⁶ together with the nitrogen atom to which they are attached form a azetidin-1-yl, pyrrolin-1-yl, pyrrolidin- 1-yl, piperidino, morpholino or piperazino ring which is optionally substituted by 1 or 2 substituents on an available ring carbon atom, independently selected from fluoro, chloro, bromo, hydroxy, methyl, ethyl and propylenedioxy, and optionally substituted on any available ring nitrogen by a substituent selected from methyl, ethyl, acetyl and propionyl (provided the ring is not thereby quaternised),

and wherein any alkyl or alkanoyl group present as a substituent on the ring formed by R⁵ and R⁶ together with the nitrogen atom to which they are attached is optionally substituted by 1 or 2 substituents independently selected from fluoro, chloro, bromo and hydroxy and/or optionally a substituent selected from methyl, ethyl, methoxy and ethoxy; provided that when the pyrrolidinyloxy group is linked to the 6-position of the quinazoline ring, m is 2 and substituents R¹ are both halogeno and attached to the 2- and 3-positions of the ring A, then R⁶ is selected from cyclopropylmethyl cyclobutylmethyl, cyclopentylmethyl and cyclohexylmethyl,
and wherein said cyclopropyl cyclobutyl cyclopentyl and cyclohexyl group is optionally substituted by 1 or 2 substituents independently selected from fluoro, chloro, bromo, hydroxy, methyl, ethyl, hydroxymethyl 2-hydroxyethyl, methoxycarbonyl ethoxycarbonyl carbamoyl, acetamido, propionamido and hydroxy and/or optionally a substituent selected from oxo, cyano, methoxy and ethoxy.
(k) R⁵ is hydrogen, methyl or ethyl and R⁶ is selected from hydrogen, methyl, ethyl propyl, isopropyl, isobutyl, vinyl, isoprop-2-enyl, allyl, but-2-enyl ethynyl, 2-prop-2-ynyl, but-3-ynyl, methoxy, ethoxy, cyclopropyl, cyclopentyl, cyclohexyl, azetidinyl, pyrrolinyl, pyrrolidinyl, morpholinyl piperidinyl, piperazinyl, tetrahydropyridinyl thiomorpholinyl, 1,2,3,6-tetrahydropyridin-1-yl, pyrazolyl thienyl, oxazolyl, isoxazolyl imidazolyl, pyridinyl, pyridazinyl, pyrazinyl, pyrimidyl, furanyl, pyrazolyl, thiazolyl, isothiazolyl, cyclopropylmethyl, cyclopentylmethyl, cyclohexylmethyl, 2-cyclopropylethyl, 2-cyclopentylethyl, 2-cyclohexylethyl, azetidinylmethyl, pyrrolinylmethyl, pyrrolidinylmethyl, morpholinylmethyl, piperidinylmethyl piperazinylmethyl, tetrahydropyridinylmethyl, thiomorpholinylnethyl, pyrazolylmethyl, thienylmethyl, oxazolylmethyl, isoxazolylnethyl, imidazolylmethyl, pyridinylmethyl, pyridazinylmethyl, pyrazinylmethyl, pyrimidylmethyl, furanylmethyl, pyrazolyhlethyl, thiazolylmethyl, isothiazolylmethyl, 2-(azetidinyl)ethyl2-(pyrrolinyl)ethyl, 2-(pyrrolidinyl)ethyl, 2-(,morpholinyl)ethyl 2-piperidinyl)ethyl, 2-(piperazinyl)ethyl, 2-(tetrahydropyridinyl)ethyl, 2-(thiomorpholinyl)ethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl)ethyl,
and wherein any alkyl, cycloalkyl, heteroaryl or heterocyclyl group within R⁵ or R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl carbamoyl acetamido and hydroxy and/or optionally a substituent selected from oxo, cyano, methoxy and ethoxy,
and wherein any heterocyclyl group within R⁶ is optionally substituted on any available ring nitrogen (provided the ring is not thereby quaternised) by methyl ethyl acetyl or propionyl or R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, hydroxy, methyl, ethyl and propylenedioxy, and optionally substituted on any available ring nitrogen by a substituent selected from methyl, ethyl, acetyl and propionyl (provided the ring is not thereby quaternised),
and wherein any alkyl or alkanoyl group present as a substituent on the ring formed by R⁵ and R⁶ together with the nitrogen atom to which they are attached is optionally substituted by 1 or 2 substituents independently selected from fluoro, chloro and hydroxy and/or optionally a substituent selected from methyl ethyl, methoxy and ethoxy;

provided that when the pyrrolidinyloxy group is linked to the 6-position of the quinazoline ring, m is 2 and substituents R¹ are both halogeno and attached to the 2- and 3-positions of the ring A, then R⁶ is selected from cyclopropylmethyl, cyclobutylmethyl and cyclpentylmethyl,
and wherein said cyclopropyl cyclobutylethyl or cyclpentylmethyl group is optionally substituted (on any available carbon atoms) by 1 or 2 substituents independently selected from halo and hydroxy.
(l) R⁵ is hydrogen or methyl and R⁶ is selected from hydrogen, methyl, ethyl, propyl, isopropyl vinyl, isoprop-2-enyl, allyl, but-2-enyl ethynyl, 2-propynyl, but-3-ynyl, methoxy, cyclopropyl, cyclopentyl, 1-(hydroxymethyl)cyclopentyl, cyclohexyl, 4-hydroxycyclohexyl, cyclopropylmethyl, cyclopentylmethyl, methoxymethyl, 2-(methoxy)ethyl, 2-(ethoxy)ethyl, carbamoylmethyl, 2-(acetyl)ethyl, cyanomethyl 2-(cyano)ethyl, 2,3-dihydroxypropyl, 2-(hydroxyl)-1,1-dimethylethyl, 2,2,2-trifluoroethyl, 1-(ethoxycarbonyl)-2-hydroxyethyl, 2-acetamido)ethyl, tetrahydrofuran-2-ylmethyl, imidazol-2-ylmethyl, 1-methylpyrazol-5-yl, 1-methylpyrazol-5-yl, 3-methylpyrazol-5-yl, imidazol-1-ylmethyl, 2-(imidazol-1-yl)ethyl, furan-2-ylmethyl, 2-(furan-2-yl)ethyl, 5-methylisoxazol-3-ylmethyl, thien-3yl, morpholino, piperidin-4yl, 1-methylpiperidin-4yl, tetrahydro-2H-pyran-4yl and 3-oxotetrahydrofuran-4yl,

or R⁵ and R⁶ together with the nitrogen atom to which they are attached form a 3-hydroxyazetidin-1-yl, 2-carbamoylazetidin-1-yl, pyrrolin-1-yl, pyrrolidin-1-yl, 3-hydroxy, pyrrolidin-1-yl, piperidino, morpholino or piperazino group; provided that when the pyrrolidinyloxy group is linked to the 6position of the quinazoline ring, m is 2 and substituents R¹ are both halogeno and attached to the 2- and 3-positions of the ring A, then R⁶ is cyclopropylmethyl or cyclopentylmethyl,
(m) R⁵ is hydrogen or (1-6C)alkyl and R⁶ is selected from hydrogen, (1-6C)alkyl, (2-6C)alkenyl, (2-6C)alkynyl, (1-6C)alkoxy, (3-7)cycloalkyl, (1-6C)alkylsulfonyl, heterocyclyl, heteroaryl, (3-7)cycloalkyl(1-3C)alkyl (3-7)heterocyclyl(1-3C)alkyl and heteroaryl(1-3C)alkyl, and wherein any (1-3C)alkyl, (1-6C)alkyl, (3-7)cycloalkyl, heteroaryl or heterocyclyl group within R⁵ or R⁶ is optionally substituted (on any available carbon atoms) by 1, 2 or 3 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heterocyclyl group within R⁶ is optionally substituted on any available ring nitrogen (provided the ring is not thereby quaternised) by (1-4C)alkyl or (2-4C)alkanoyl, or R⁵ and R⁶ together with the nitrogen atom to which they are attached form a 4, 5 or 6 membered ring which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised),

and wherein any (1-4C)alkyl or (2-4C)alkanoyl group present as a substituent on the ring formed by R⁵ and R⁶ together with the nitrogen atom to which they are attached is optionally substituted by 1, 2 or 3 substituents independently selected from halogeno and hydroxy and/or optionally a substituent selected from (1-4C)alkyl and (1-4C)alkoxy;

provided that when the pyrrolidinyloxy group is linked to the 6-position of the quinazoline ring, m is 2 and substituents R¹ are both halogeno and attached to the 2- and 3-positions of the ring A, then R⁶ is selected from substituted-(1-6C)alkyl (wherein substituted-(1-6C)alkyl is (1-6C)alkyl substituted by 1, 2 or 3 substituents independently selected from (1-6C)alkoxycarbonyl, carbamoyl, (2-6C)alkanoylamino, and oxo or a (1-6C)alkoxycarbonyl together with a hydroxy group), (1-6C)alkoxy, (1-6C)alkylsulfonyl, (3-7)heterocyclyl (wherein the heterocyclyl is carbon linked), heteroaryl, (3-7)cycloalkyl(1-6C)alkyl, (3-7)heterocyclyl(1-6C)alkyl (wherein the heterocyclyl is carbon linked to the (1-6C)alkyl moiety) and heteroaryl(1-6C)alkyl,
and wherein any (3-7)cycloalkyl, heteroaryl or (3-7)heterocyclyl group within R⁶ is optionally substituted (on any available carbon atoms) by 1, 2 or 3 substituents independently selected from halogeno, (1-6C)alkyl hydroxy(1-6C)alkyl, (1-6C)alkoxycarbonyl carbamoyl (2-6C)alkanoylamino and hydroxy and/or optionally a substituent selected from oxo, cyano, nitro and (1-4C)alkoxy,
and wherein any heteroaryl or heterocyclyl group within R⁶ is optionally substituted on any available ring nitrogen (provided the ring is not thereby quaternised) by (1-4C)alkyl or (2-4C)alkanoyl, or
R⁵ and R⁶ together with the nitrogen atom to which they are attached form a 4, 5 or 6 membered ring which contains one or two nitrogen atoms as the only heteroatom(s) present in the ring and which is substituted on an available ring carbon atom by 1 or 2 substituents independently selected from carbamoyl and (1-3C)alkylenedioxy.
(n) R⁵ is hydrogen or (1-6C)alkyl and R⁶ is selected from hydrogen, (1-6C)alkyl, (2-6C)alkenyl (2-6C)alkynyl, (1-6C)alkoxy, (3-7)cycloalkyl, (1-6C)akylsulfonyl, heterocyclyl, heteroaryl, (3-7)cycloalkyl(1-3C)alkyl, (3-7)heterocyclyl(1-3C)alkyl and heteroaryl(1-3C)alkyl, and wherein any (1-3C)alkyl, (1-6C)alkyl, (3-7)cycloalkyl, heteroaryl or heterocyclyl group within R⁵ or R⁶ is optionally substituted (on any available carbon atoms) by 1, 2 or 3 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,

and wherein any heterocyclyl group within R⁶ is optionally substituted on any available ring nitrogen (provided the ring is not thereby quaternised) by (1-4C)alkyl or (2-4C)alkanoyl, or
R⁵ and R⁶ together with the nitrogen atom to which they are attached form a 4, 5 or 6 membered ring which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised),

and wherein any (1-4C)alkyl or (2-4C)akanoyl group present as a substituent on the ring formed by R⁵ and R⁶ together with the nitrogen atom to which they are attached is optionally substituted by 1, 2 or 3 substituents independently selected from halogeno and hydroxy and/or optionally a substituent selected from (1-4C)alkyl and (1-4C)alkoxy;

provided that when the pyrrolidinyloxy group is linked to the 6-position of the quinazoline ring, m is 2 and substituents R¹ are both halogeno and attached to the 2- and 3-positions of the ring A, then R⁶ is selected from substituted-(1-6C)alkyl (wherein substituted-(1-6C)alkyl is (1-6C)alkyl substituted by 1, 2 or 3 substituents independently selected from (1-6C)alkoxycarbonyl, carbamoyl, (2-6C)alkanoylamino, and oxo or a (1-6C)alkoxycarbonyl together with a hydroxy group), (1-6C)alkoxy, (1-6C)alkylsulfonyl, (3-7)heterocyclyl (wherein the heterocyclyl is carbon linked), heteroaryl, (3-7)heterocyclyl(1-6C)alkyl (wherein the heterocyclyl is carbon linked to the (1-6C)alkyl moiety) and heteroaryl(l-6C)alkyl, and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,

and wherein any heteroaryl or heterocyclyl group within R⁶ is optionally substituted on any available ring nitrogen (provided the ring is not thereby quaternised) by (1-4C)alkyl or (2-4C)alkanoyl, or
R⁵ and R⁶ together with the nitrogen atom to which they are attached form a 4, 5 or 6 membered ring which contains one or two nitrogen atoms as the only heteroatom(s) present in the ring and which is substituted on an available ring carbon atom by 1 or 2 substituents independently selected from carbamoyl and (1-3C)alkylenedioxy.
(o) R⁵ is hydrogen, methyl ethyl propyl, isopropyl or isobutyl and R⁶ is selected from hydrogen, methyl, ethyl propyl, isopropyl, isobutyl vinyl, isopropenyl, alkyl, but-2-enyl ethynyl, 2-propynyl, butynyl, methoxy, ethoxy propoxy, isopropoxy, cyclopropyl, cyclopentyl, cyclohexyl azetidinyl, oxazepanyl, pyrrolinyl, pyrrolidinyl, morpholinyl, tetrahydro-1,4thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl, tetrahydrothiopyranyl, thiomorpholinyl, pyrazolyl, thienyl, oxazolyl, isoxazolyl, imidazolyl, pyridinyl, pyridazinyl, pyrazinyl, pyrimidyl, furanyl, pyrazolyl, thiazolyl, isothiazolyl, thiadiazolyl, cyclopropylmethyl, cyclopentylmethyl, cyclohexylmethyl, 2-cyclopropylethyl, 2-cyclopentylethyl, 2-cyclohexylethyl, azetidinylmethyl, oxazolylmethyl, pyrrolinylmethyl, pyrrolidinylmethyl morpholinyl methyl, tetrahydro-1,4thiayznyethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl, tetrahydrothienylmethyl, tetrahydrothiopyranylimethyl, thiomorpholinylmethyl, pyrazolylmethyl, thienylmethyl, oxazolylmethyl, isoxazolylmethyl, imidazolylmethyl pyridinylmethyl, pyridazinylmethyl, pyrazinylmethyl, pyrimidylmethyl, furanylmethyl, pyrazolylmethyl, thiazolylmethyl, isothiazolylmethyl, thiadiazolylmethyl, 2-(azetidinyl)ethyl, 2-(oxazepanyl)ethyl, 2-(pyrrolinyl)ethyl, 2-(pyrrolidinyl)ethyl, 2-(morpholinyl)ethyl, 2-(tetrahydro-1,4-thiazinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrothienyl)ethyl, 2-(tetrahydrothiopyranyl)ethyl, 2-(thiomorpholinyl)ethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl 2-(pyrazolyl)ethyl, 2-(thiazolyl)ethyl, 2-(isotbiazolyl)ethyl and 2-(thiadiazolyl)ethyl,

and wherein any alkyl cycloalkyl, heteroaryl or heterocyclyl group within R⁵ or R⁶ is optionally substituted (on any available carbon atoms) by 1 or 2 substituents independently selected from fluoro, chloro, bromo, hydroxymethyl 2-hydroxyethyl, methoxycarbonyl, ethoxycarbonyl, carbamoyl, acetamido, propionamido and hydroxy and/or optionally a substituent selected from oxo, cyano, methoxy and ethoxy,
and wherein any heterocyclyl group within R⁶ is optionally substituted on any available ring nitrogen (provided the ring is not thereby quaternised) by methyl, ethyl, acetyl or propionyl or R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, bromo, hydroxy, methyl, ethyl and propylenedioxy, and optionally substituted on any available ring nitrogen by a substituent selected from methyl, ethyl, acetyl and propionyl (provided the ring is not thereby quaternised),

and wherein any alkyl or alkanoyl group present as a substituent on the ring formed by R⁵ and R⁶ together with the nitrogen atom to which they are attached is optionally substituted by 1 or 2 substituents independently selected from fluoro, chloro, bromo and hydroxy and/or optionally a substituent selected from methyl, ethyl, methoxy and ethoxy; provided that when the pyrrolidinyloxy group is linked to the 6-position of the quinazoline ring, m is 2 and substituents R¹ are both halogeno and attached to the 2- and 3-positions of the ring A, then R⁶ is selected from substituted-methyl, substituted-ethyl substituted-propyl, substituted-isopropyl, substituted-isobutyl, (wherein the substituted groups are substituted by 1 or 2 substituents independently selected from methoxycarbonyl, ethoxycarbonyl, carbamoyl, acetamido, propionamido and oxo or a methoxycarbonyl group together with a hydroxy group or an ethoxycarbonyl group together with a hydroxy group) methoxy, ethoxy, propoxy, isopropoxy,

a carbon linked heterocyclyl group selected from azetidinyl, oxazepanyl, pyrrolinyl, pyrrolidinyl, morpholinyl, tetrahydrofuranyl, tetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl, tetrahydropyranyl, tetrahydrothiopyranyl, thiomorpholinyl,

a heteroaryl group selected from pyrazolyl, thienyl, oxazolyl, isoxazolyl, imidazolyl, pyridinyl, pyridazinyl, pyrazinyl, pyrimidyl, furanyl, thiazolyl, isothiazolyl, thiadiazolyl,

a (3-7)heterocyclyl(1-6C)alkyl group (wherein the heterocyclyl is carbon linked to the (1-6C)alkyl moiety) selected from azetidinylmethyl, oxazolylmethyl, pyrrolinylmethyl, pyrrolidinylmethyl, morpholinylmethyl, tetrahydro-1,4-thiazinylmethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl, tetrahydrofuranylmethyl, tetrahydrothienylmethyl, tetrahydropyranylmethyl, tetrahydrothiopyranylmethyl, thiomorpholinylmethyl 2-(azetidinyl)ethyl, 2-(oxazepanyl)ethyl, 2-(pyrrolinyl)ethyl, 2-(pyrrolidinyl)ethyl, 2-(morpholinyl)ethyl, 2-(tetrahydro-1,4-thiazinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrofuranyl)ethyl, 2-(tetrahydrothienyl)ethyl, 2-(tetrahydropyranyl)ethyl, 2-(tetrahydrothiopyranyl)ethyl, 2-(thiomorpholinyl)ethyl,

a heteroaryl(1-6C)alkyl group selected from pyrazolylmethyl, thienylmethyl, oxazolylmethyl, isoxazolylmethyl, imidazolylmethyl, pyridinylmethyl, pyridazinylmethyl, pyrazinylmethyl, pyrimidylmethyl, furanylmethyl, pyrazolylmethyl, thiazolylmethyl, isothiazolylmethyl, thiadiazolylmethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl, 2-(isothiazolyl)ethyl and 2-(thiadiazolylmethyl,
and wherein any heteroaryl or heterocyclyl group within R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido, propionamido and hydroxy and/or optionally a substituent selected from oxo, cyano, methoxy and ethoxy,

and wherein any heteroaryl or heterocyclyl group within R⁶ is optionally substituted on any available ring nitrogen (provided the ring is not thereby quaternised) by methyl, ethyl, acetyl or propionyl
or R⁵ and R⁶ together with the nitrogen atom to which they are attached form an azetidin-1-yl, pyrrolidin-1-yl, or piperidin-1-yl ring which is substituted on an available carbon atom by a substituent selected from carbamoyl or (1-3C)alkylenedioxy.
(p) R⁵ is hydrogen, methyl or ethyl and R⁶ is selected from hydrogen, methyl, ethyl propyl, isopropyl, isobutyl, vinyl, isoprop-2-enyl, allyl but-2-enyl ethynyl, 2-prop-2-ynyl, but-3-ynyl, methoxy, ethoxy, cyclopropyl, cyclopentyl, cyclohexyl, azetidinyl, pyrrolinyl, pyrrolidinyl, morpholinyl, piperidinyl, piperazinyl, tetrahydropyridinyl thiomorpholinyl, 1,2,3,6-tetrahydropyridin-1-yl, pyrazolyl, thienyl, oxazolyl, isoxazolyl, imidazolyl, pyridinyl, pyridazinyl, pyrazinyl, pyrimidyl, furanyl pyrazolyl, thiazolyl, isothiazolyl, cyclopropylethyl, cyclopentylmethyl, cyclohexylmethyl, 2-cyclopropylethyl, 2-cyclopentylethyl, 2-cyclohexylethyl, azetidinylmethyl, pyrrolinylmethyl, pyrrolidinylmethyl, morpholinylmethyl, piperidinylmethyl, piperazinylmethyl, tetrahydropyridinylmethyl, thiomorpholinylmethyl, pyrazolylmethyl, thienylmethyl, oxazolylmethyl, isoxazolylmethyl, imidazolylmethyl, pyridinylmethyl, pyridazinylmethyl, pyrazinylmethyl, pyrimidylmethyl, furanylmethyl, pyrazolylmethyl, thiazolylmethyl, isothiazolylmethyl, 2-(azetidinyl)ethyl2-(pyrrolinyl)ethyl, 2-(pyrrolidinyl)ethyl, 2-(morpholinyl)ethyl, 2-(piperidinyl)ethyl, 2-(piperazinyl)ethyl, 2-(tetrahydropyridinyl)ethyl, 2-(thiomorpholinyl)ethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl)ethyl,

and wherein any alkyl cycloalkyl, heteroaryl or heterocyclyl group within R⁵ or R⁶ is optionally substituted (on any available carbon atoms) by 1 or 2 substituents independently selected from fluoro, chloro, bromo, hydroxymethyl 2-hydroxyethyl, methoxycarbonyl ethoxycarbonyl, carbamoyl acetamido and hydroxy and/or optionally a substituent selected from oxo, cyano, methoxy and ethoxy,

and wherein any heterocyclyl group within R⁶ is optionally substituted on any available ring nitrogen (provided the ring is not thereby quaternised) by methyl, ethyl, acetyl or propionyl, or
R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, hydroxy, methyl, ethyl and propylenedioxy, and optionally substituted on any available ring nitrogen by a substituent selected from methyl, ethyl acetyl and propionyl (provided the ring is not thereby quaternised),

and wherein any alkyl or alkanoyl group present as a substituent on the ring formed by R⁵ and R⁶ together with the nitrogen atom to which they are attached is optionally substituted by 1 or 2 substituents independently selected from fluoro, chloro and hydroxy and/or optionally a substituent selected from methyl, ethyl, methoxy and ethoxy; provided that when the pyrrolidinyloxy group is linked to the 6-position of the quinazoline ring, m is 2 and substituents R¹ are both halogeno and attached to the 2- and 3-positions of the ring A, then R⁶ is selected from substituted-methyl, substituted-ethyl substituted-propyl, substituted-isopropyl, substituted-isobutyl (wherein the substituted groups are substituted by 1 or 2 substituents independently selected from methoxycarbonyl, ethoxycarbonyl, carbamoyl, acetamido and oxo or a methoxycarbonyl group together with a hydroxy group), methoxy, ethoxy,

a carbon linked heterocyclyl group selected from azetidinyl, pyrrolinyl, pyrrolidinyl, morpholinyl, tetrahydrofuranyl, piperidinyl, piperazinyl, tetrahydropyridinyl, tetrahydropyranyl, thiomorpholinyl,

a heteroaryl group selected from pyrazolyl, thienyl, oxazolyl, isoxazolyl, imidazolyl, pyridinyl, pyridazinyl, pyrazinyl, pyrimidyl, furanyl, pyrazolyl thiazolyl, isothiazolyl,

a (3-7)heterocyclyl(1-6C)alkyl group (wherein the heterocyclyl is carbon linked to the (1-6C)alkyl moiety) selected from azetidinylmethyl, pyrrolinylmethyl, pyrrolidinylmethyl, morpholinylmethyl, piperidinylethyl, piperazinylmethyl, tetrahydrofuranylmethyl, tetrahydropyranylmethyl, tetrahydropyridinylmethyl, thiomorpholinylmethyl, 2-(azetidinyl)ethyl, 2-(pyrrolinyl)ethyl, 2-(pyrrolidinyl)ethyl, 2-(morpholinyl)ethyl, 2-(piperidinyl)ethyl, 2-(piperazinyl)ethyl, 2-(tetrahydrofuranyl)ethyl, 2-(tetrahydropyranyl)methyl, 2-(tetrahydropyridinyl)ethyl, 2-(thiomorpholinyl)ethyl,

a heteroaryl(1-6C)alkyl group selected from pyrazolylmethyl thienylmethyl, oxazolylmethyl, isoxazolylmethyl, imidazolylmethyl, pyridinylmethyl, pyridazinyhmethyl, pyrazinylmethyl, pyrimidylmethyl, furanylmethyl, pyrazolylmethyl, thiazolylmethyl, isothiazolylmethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl)ethyl,
and wherein any heteroaryl or heterocyclyl group within R⁶ is optionally substituted (on any available carbon atoms) by 1 or 2 substituents independently selected from fluoro, chloro, bromo, hydroxymethyl, 2-hydroxyethyl, methoxycarbonyl ethoxycarbonyl, carbamoyl, acetamido and hydroxy and/or optionally a substituent selected from oxo, cyano, methoxy and ethoxy,

and wherein any heteroaryl or heterocyclyl group within R⁶ is optionally substituted on any available ring nitrogen (provided the ring is not thereby quaternised) by methyl ethyl, acetyl or propionyl;
or R⁵ and R⁶ together with the nitrogen atom to which they are attached form an azetidin-1-yl ring which is substituted on an available carbon atom by a carbamoyl group.
(q) R⁵ is hydrogen or methyl and R⁶ is selected from hydrogen, methyl, ethyl propyl, isopropyl, vinyl, isoprop-2-enyl, allyl but-2-enyl ethynyl, 2-propynyl, but-3-ynyl, methoxy, cyclopropyl, cyclopentyl, 1-(hydroxymethyl)cyclopentyl, cyclohexyl 4-hydroxycyclohexyl, cyclopropylmethyl, cyclopentyhmethyl, methoxymethyl, 2-(methoxy)ethyl, 2-(ethoxy)ethyl, carbamoylmethyl, 2-(acetyl)ethyl, cyanomethyl 2-(cyano)ethyl, 2,3-dihydroxypropyl, 2-hydroxyl)-1,1-dimethylethyl, 2,2,2-trifluoroethyl, 1-(ethoxycarbonyl)-2-hydroxyethyl, 2-acetamido)ethyl, tetrahydrofuran-2-ylmethyl, imidazol-2-ylmethyl, 1-methylpyrazol-5-yl, 1-methylpyrazol-5-yl, 3-methylpyrazol-5-yl, imidazol-1-ylmethyl, 2-(imidazol-1-yl)ethyl, furan-2-ylmethyl, 2-(furan-2-yl)ethyl, 5-methylisoxazol-3-ylmethyl, thien-3yl, morpholino, piperidin-4-yl, 1-methylpiperidin-4-yl, tetrahydro-2H-pyran-4yl and 3-oxotetrahydrofuran-4-yl,

or R⁵ and R⁶ together with the nitrogen atom to which they are attached form a 3-hydroxyazetidin-1-yl, 2-carbamoylazetidin-1-yl, pyrrolin-1-yl, pyrrolidin-1-yl, 3-hydroxy, pyrrolidin-1-yl piperidino, morpholino or piperazino group;
provided that when the pyrrolidinyloxy group is linked to the 6-position of the quinazoline ring, m is 2 and substituents R¹ are both halogeno and attached to the 2- and 3-positions of the ring A, then R⁶ is selected from methoxy, carbamoylmethyl 2-(hydroxy)-1-(methoxycarbonyl)ethyl, 1-(ethoxycarbonyl)-2-hydroxyethyl 2-(acetamido)ethyl, piperidin-4-yl, 1-methylpiperidin-4-yl, tetrahydropyran-4-yl, 4-hydroxytetrahydrofuran-3-yl, 3-oxotetrahydrofuran-4-yl, 1-methylpyrazol-5-yl, thien-3yl, 3-methylpyrazol-5-yl, tetrahydrofaran-2-ylmethyl, tetrahydropyran-4-ylmethyl, furan-2-ylmethyl, 2-(furan-2-yl)ethyl, imidazol-1-ylmethyl, imidazol-2-ylmethyl, imidazol-2-ylmethyl, 2-(imidazol-1-yl)ethyl, 2-(imidazol-4-yl)ethyl and 5-methylisoxazol-3-ylmethyl or R⁵ and R⁶ together with the nitrogen atom to which they are attached form an azetidin-1-yl ring which is substituted in the 2 position by a carbamoyl group.
(r) R⁵ is hydrogen or (1-6C)alkyl and R⁶ is selected from hydrogen, (1-6C)alkyl, (2-6C)alkenyl, (2-6C)alkynyl, (1-6C)alkoxy, (3-7)cycloalkyl, (1-6C)alkylsulfonyl, heterocyclyl, heteroaryl, (3-7)cycloalkyl(1-3C)alkyl, (3-7)heterocyclyl(1-3C)alkyl and heteroaryl(1-3C)alkyl, and wherein any (1-3C)alkyl, (1-6C)alkyl, (3-7)cycloalkyl, heteroaryl or heterocyclyl group within R⁵ or R⁶ is optionally substituted (on any available carbon atoms) by 1, 2 or 3 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heterocyclyl group within R⁶ is optionally substituted on any available ring nitrogen (provided the ring is not thereby quaternised) by (1-4C)alkyl or (2-4C)alkanoyl, or R⁵ and R⁶ together with the nitrogen atom to which they are attached form a 4, 5 or 6 membered ring which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised),

and wherein any (1-4C)alkyl or (2-4C) alkanoyl group present as a substituent on the ring formed by R⁵ and R⁶ together with the nitrogen atom to which they are attached is optionally substituted by 1, 2 or 3 substituents independently selected from halogeno and hydroxy and/or optionally a substituent selected from (1-4C)alkyl and (1-4C)alkoxy;

provided that when the pyrrolidinyloxy group is linked to the 6-position of the quinazoline ring, m is 2 and substituents R¹ are both halogeno and attached to the 2- and 3-positions of the ring A, then R⁶ is selected from (3-7)heterocyclyl (wherein the heterocyclyl is carbon linked), heteroaryl, (3-7)heterocyclyl(1-6C)alkyl (wherein the heterocyclyl is carbon linked to the (1-6C)alkyl moiety) and heteroaryl(1-6C)alkyl,
and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy, and wherein any heteroaryl or heterocyclyl group within R⁶ is optionally substituted on any available ring nitrogen (provided the ring is not thereby quaternised) by (1-4C)alkyl or (2-4C)alkanoyl.
(s) R⁵ is hydrogen, methyl, ethyl propyl, isopropyl or isobutyl and R⁶ is selected from hydrogen, methyl, ethyl propyl isopropyl, isobutyl, vinyl, isopropenyl alkyl but-2-enyl ethynyl, 2-propynyl, butynyl, methoxy, ethoxy propoxy, isopropoxy, cyclopropyl, cyclopentyl, cyclohexyl, azetidinyl, oxazepanyl, pyrrolinyl, pyrrolidinyl, morpholinyl, tetrahydro-1,4-thiazinyl, piperidinyl, homopiperidinyl, piperazinyl, homopiperazinyl, dihydropyridinyl, tetrahydropyridinyl, dihydropyrimidinyl tetrahydropyrimidinyl, tetrahydrothienyl, tetrahydrothiopyranyl, thiomorpholinyl, pyrazolyl, thienyl, oxazolyl, isoxazolyl, imidazolyl, pyridinyl, pyridazinyl, pyrazinyl, pyrimidyl, furanyl, pyrazolyl, thiazolyl, isothiazolyl, thiadiazolyl, cyclopropylmethyl, cyclopentylmethyl, cyclohexylmethyl 2-cyclopropylethyl, 2-cyclopentylethyl, 2-cyclohexylethyl, azetidinylmethyl, oxazolylmethyl, pyrrolinylmethyl, pyrrolidinylmethyl, morpholinylmethyl, tetrahydro-1,4-thiazinylmethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl, tetrahydrothienylmethyl, tetrahydrothiopyranylmethyl, thiomorpholinylmethyl, pyrazolylmethyl, thienylmethyl, oxazolylmethyl isoxazolylmethyl, imidazolylmethyl, pyridinylmethyl, pyridazinylmethyl, pyrazinylmethyl, pyrimidylmethyl, furanylmethyl, pyrazolylmethyl, thiazolylmethyl, isothiazolylmethyl, thiadiazolylmethyl, 2-(azetidinyl)ethyl, 2-(oxazepanyl)ethyl, 2-(pyrrolinyl)ethyl, 2-(pyrrolidinyl)ethyl, 2-(morpholinyl)ethyl, 2-(tetrahydro-1,4thiazinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrothienyl)ethyl, 2-(tetrahydrothiopyranyl)ethyl, 2-(thiomorpholinyl)ethyl, 2-(pyrazolyl)ethyl, 2-(thienyl)methyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl, 2-(isothiazolyl)ethyl and 2-(thiadiazolyl)ethyl,
and wherein any alkyl, cycloalkyl, heteroaryl or heterocyclyl group within R⁵ or R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido, propionamido and hydroxy and/or optionally a substituent selected from oxo, cyano, methoxy and ethoxy,
and wherein any heterocyclyl group within R⁶ is optionally substituted on any available ring nitrogen (provided the ring is not thereby quaternised) by methyl, ethyl, acetyl or propionyl or R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, bromo, hydroxy, methyl, ethyl and propylenedioxy, and optionally substituted on any available ring nitrogen by a substituent selected from methyl, ethyl, acetyl and propionyl (provided the ring is not thereby quaternised),

and wherein any alkyl or alkanoyl group present as a substituent on the ring formed by R⁵ and R⁶ together with the nitrogen atom to which they are attached is optionally substituted by 1 or 2 substituents independently selected from fluoro, chloro, bromo and hydroxy and/or optionally a substituent selected from methyl ethyl, methoxy and ethoxy;
provided that when the pyrrolidinyloxy group is linked to the 6position of the quinazoline ring, m is 2 and substituents R¹ are both halogeno and attached to the 2- and 3-positions of the ring A, then R⁶ is selected from.

a carbon linked heterocyclyl group selected from azetidinyl, oxazepanyl, pyrrolinyl, pyrrolidinyl, morpholinyl, tetrahydrofuranyl tetrahydro-1,4thiazinyl, piperidinyl homopiperidinyl, piperazinyl, homopiperazinyl dihydropyridinyl, tetrahydropyridinyl, dihydropyrimidinyl, tetrahydropyrimidinyl, tetrahydrothienyl, tetrahydropyranyl, tetrahydrothiopyranyl, thiomorpholinyl;

a heteroaryl group selected from pyrazolyl, thienyl, oxazolyl, isoxazolyl, imidazolyl, pyridinyl, pyridazinyl, pyrazinyl, pyrimidyl, furanyl, thiazolyl, isothiazolyl, thiadiazolyl;

a (3-7)heterocyclyl(1-6C)alkyl group (wherein the heterocyclyl is carbon linked to the (1-6C)alkyl moiety) selected from azetidinylmethyl, oxazolylmethyl, pyrrolinylmethyl, pyrrolidinylmethyl, morpholinylmethyl, tetrahydro-1,4-thiazinylmethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl, tetrahydrofuranylmethyl, tetrahydrothienylmethyl, tetrahydropyranylmethyl, tetrahydrothiopyranylmethyl, thiomorpholinylmethyl, 2-(azetidinyl)ethyl, 2-(oxazepanyl)ethyl, 2-(pyrrolinyl)ethyl, 2-(pyrrolidinyl)ethyl, 2-(morpholinyl)ethyl, 2-(tetrahydro-1,4-thiazinyl)ethyl, 2-(piperidinyl)ethyl, 2-homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrofuranyl)ethyl, 2-(tetrahydrothienyl)ethyl, 2-(tetrahydropyranyl)ethyl, 2-(tetrahydrothiopyranyl)ethyl, 2-(thiomorpholinyl)ethyl;

a heteroaryl(1-6C)alkyl group selected from pyrazolylmethyl, thienylmethyl, oxazolylmethyl, isoxazolylmethyl, imidazolylmethyl, pyridinylmethyl, pyridazinylmethyl, pyrazinylmethyl, pyrinidylmethyl, furanylmethyl, pyrazolylmethyl, thiazolylmethyl, isothiazolylmethyl, thiadiazolylmethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl, 2-(isothiazolyl)ethyl and 2-(thiadiazolyl)ethyl;
and wherein any heteroaryl or heterocyclyl group within R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido, propionamido and hydroxy and/or optionally a substituent selected from oxo, cyano, methoxy and ethoxy,
and wherein any heteroaryl or heterocyclyl group within R⁶ is optionally substituted on any available ring nitrogen (provided the ring is not thereby quaternised) by methyl ethyl, acetyl or propionyl.
(t) R⁵ is hydrogen, methyl or ethyl and R⁶ is selected from hydrogen, methyl, ethyl propyl isopropyl, isobutyl, vinyl isoprop-2-enyl, allyl, but-2-enyl ethynyl 2-prop-2-ynyl, but-3-ynyl, methoxy, ethoxy, cyclopropyl cyclopentyl, cyclohexyl, azetidinyl, pyrrolinyl, pyrrolidinyl, morpholinyl, piperidinyl, piperazinyl, tetrahydropyridinyl, thiomorpholinyl 1,2,3,6-tetrahydropyridin-1-yl, pyrazolyl, thienyl, oxazolyl, isoxazolyl, imidazolyl, pyridinyl, pyridazinyl, pyrazinyl, pyrimidyl, furanyl, pyrazolyl, thiazolyl, isothiazolyl, cyclopropylmethyl, cyclopentylmethyl, cyclohexylmethyl, 2-cyclopropylethyl, 2-cyclopentylethyl, 2-cyclohexylethyl, azetidinylmethyl, pyrrolinylmethyl, pyrrolidinylmethyl, morpholinylmethyl, piperidinylmethyl, piperazinylmethyl, tetrahydropyridinylmethyl, thiomorpholinylmethyl, pyrazolylmethyl, thienylmethyl oxazolylmethyl, isoxazolylmethyl, imidazolylmethyl, pyridinylmethyl, pyridazinylmethyl, pyrazinylmethyl, pyrimidylmethyl, furanylmethyl, pyrazolylmethyl, thiazolylmethyl, isothiazolylmethyl, 2-(azetidinyl)ethyl2-(pyrrolinyl)ethyl, 2-(pyrrolidinyl)ethyl, 2-(morpholinyl)ethyl, 2-(piperidinyl)ethyl, 2-(piperazinyl)ethyl, 2-(tetrahydropyridinylmethyl, 2-(thiomorpholinyl)ethyl, 2-(pyrazolyl)ethyl, 2-(thienyl)ethyl, 2-(oxazolyl)ethyl 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl)ethyl,
and wherein any alkyl cycloalkyl, heteroaryl or heterocyclyl group within R⁵ or R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido and hydroxy and/or optionally a substituent selected from oxo, cyano, methoxy and ethoxy,

and wherein any heterocyclyl group within R⁶ is optionally substituted on any available ring nitrogen (provided the ring is not thereby quaternised) by methyl, ethyl, acetyl or propionyl, or R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, hydroxy, methyl, ethyl and propylenedioxy, and optionally substituted on any available ring nitrogen by a substituent selected from methyl, ethyl, acetyl and propionyl (provided the ring is not thereby quaternised),

and wherein any alkyl or alkanoyl group present as a substituent on the ring formed by R⁵ and R⁶ together with the nitrogen atom to which they are attached is optionally substituted by 1 or 2 substituents independently selected from fluoro, chloro and hydroxy and/or optionally a substituent selected from methyl ethyl methoxy and ethoxy;

provided that when the pyrrolidinyloxy group is linked to the 6-position of the quinazoline ring, m is 2 and substituents R¹ are both halogeno and attached to the 2- and 3-positions of the ring A, then R⁶ is selected from

a carbon linked heterocyclyl group selected from azetidinyl, pyrrolinyl, pyrrolidinyl, morpholinyl, tetrahydrofuranyl, piperidinyl, piperazinyl tetrahydropyridinyl, tetrahydropyranyl, thiomorpholinyl;

a heteroaryl group selected from pyrazolyl, thienyl, oxazolyl, isoxazolyl, imidazolyl, pyridinyl, pyridazinyl, pyrazinyl, pyrimidyl, furanyl, pyrazolyl, thiazolyl, isothiazolyl;

a (3-7)heterocyclyl(1-6C)alkyl group (wherein the heterocyclyl is carbon linked to the (1-6C)alkyl moiety) selected from azetidinylmethyl, pyrrolinylmethyl, pyrrolidinylmethyl, morpholinylmethyl, piperidinylmethyl, piperazinylmethyl, tetrahydrofuranylmethyl, tetrahydropyranylmethyl, tetrahydropyridinylmethyl, thiomorpholinylmethyl, 2-(azetidinyl)ethyl, 2-(pyrrolinyl)ethyl, 2-(pyrrolidinyl)ethyl, 2-(morpholinyl)ethyl, 2-(piperidinyl)ethyl, 2-(piperazinylmethyl, 2-(tetrahydrofuranyl)ethyl, 2-(tetrahydropyranyl)methyl, 2-(tetrahydropyridinyl)ethyl, 2-(thiomorpholinyl)ethyl;

a heteroaryl(1-6C)alkyl group selected from pyrazolylmethyl, thienylmethyl, oxazolylmethyl, isoxazolylmethyl, imidazolylmethyl, pyridinylmethyl, pyridazinylmethyl, pyrazinylmethyl, pyrinidylmethyl, furanylmethyl, pyrazolylmethyl, thiazolylmethyl, isothiazolyl)methyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl) ethyl;
and wherein any heteroaryl or heterocyclyl group within R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido and hydroxy and/or optionally a substituent selected from oxo, cyano, methoxy and ethoxy,
and wherein any heteroaryl or heterocyclyl group within R⁶ is optionally substituted on any available ring nitrogen (provided the ring is not thereby quaternised) by methyl, ethyl acetyl or propionyl.
(u) R⁵ is hydrogen or methyl and R⁶ is selected from hydrogen, methyl ethyl propyl, isopropyl, vinyl isoprop-2-enyl, allyl but-2-enyl ethynyl 2-propynyl, but-3-ynyl, methoxy, cyclopropyl cyclopentyl, 1-(hydroxymethyl)cyclopentyl, cyclohexyl, 4-hydroxycyclohexyl, cyclopropylmethyl, cyclopentylmethyl, methoxymethyl, 2-(methoxy)ethyl, 2-(ethoxy)ethyl, carbamoylmethyl, 2-(acetyl)ethyl, cyanomethyl, 2-(cyano)ethyl, 2,3-dihydroxypropyl, 2-(hydroxyl)-1,1-dimethylethyl, 2,2,2-trifluoroethyl, 1-(ethoxycarbonyl)-2-hydroxyethyl, 2-acetamido)ethyl, tetrahydrofuran-2-ylmethyl, imidazol-2-ylmethyl, 1-methylpyrazol-5-yl, 1-methylpyrazol-5-yl, 3-methylpyrazol-5-yl, imidazol-1-ylmethyl, 2-(imidazol-1-yl)ethyl, furan-2-ylmethyl, 2-(furan-2-yl)ethyl, 5-methylisoxazol-3-ylmethyl, thien-3yl, morpholino, piperidin-4-yl, 1-methylpiperidin-4yl, tetrahydro-2H-pyran-4-yl and 3-oxotetrahydrofuran-4-yl,

or R⁵ and R⁶ together with the nitrogen atom to which they are attached form a 3-hydroxyazetidin-1-yl, 2-carbamoylazetidin-1-yl, pyrrolin-1-yl, pyrrolidin-1-yl, 3-hydroxy, pyrrolidin-1-yl, piperidino, morpholino or piperazino group;
provided that when the pyrrolidinyloxy group is linked to the 6-position of the quinazoline ring, m is 2 and substituents R¹ are both halogeno and attached to the 2- and 3-positions of the ring A, then R⁶ is selected from piperidin-4yl, 1-methylpiperidin-4-yl, tetrahydropyran-4-yl, 4-hydroxytetrahydrofuran-3-yl, 3-oxotetrahydrofuran-4-yl, 1-methylpyrazol-5-yl, thien-3yl, 3-methylpyrazol-5-yl, tetrahydrofuran-2-ylmethyl, tetrahydropyran-4-ylmethyl, furan-2-ylmethyl, 2-(furan-2-yl)ethyl, imidazol-1-ylmethyl, imidazol-2-ylmethyl, 2-(imidazol-2-yl)ethyl, 2-(imidazol-1-yl)ethyl, 2-(imidazol-4-yl)ethyl and 5-methylisoxazol-3-ylmethyl.
(v) R⁵ is hydrogen or (1-6C)alkyl and R⁶ is selected from substituted-(1-6C)alkyl (wherein substituted-(1-6C)alkyl is (1-6C)alkyl substituted by 1, 2 or 3 substituents independently selected from (1-6C)alkoxycarbonyl, carbamoyl, (2-6C)alkanoylamino, and oxo or a (1-6C)alkoxycarbonyl group together with a hydroxy group), (1-6C)alkoxy, (1-6C)alkylsulfonyl, (3-7)heterocyclyl (wherein the heterocyclyl is carbon linked), heteroaryl, (3-7)heterocyclyl(1-6C)alkyl (wherein the heterocyclyl is carbon linked to the (1-6C)alkyl moiety) and heteroaryl(1-6C)alkyl,
and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heteroaryl or heterocyclyl group within R⁶ is optionally substituted on any available ring nitrogen (provided the ring is not thereby quaternised) by (1-4C)alkyl or (2-4C)alkanoyl, or

R⁵ and R⁶ together with the nitrogen atom to which they are attached form a 4, 5 or 6 membered ring which contains one or two nitrogen atoms as the only heteroatom(s) present in the ring and which is substituted on an available ring carbon atom by 1 or 2 substituents independently selected from carbamoyl and (1-3C)alkylenedioxy.
(w) R⁵ is hydrogen, methyl ethyl propyl, isopropyl or isobutyl and R⁶ is selected from substituted-methyl substituted-ethyl substituted-propyl, substituted-isopropyl, substituted-isobutyl, (wherein the substituted groups are substituted by 1 or 2 substituents independently selected from methoxycarbonyl, ethoxycarbonyl, carbamoyl, acetamido, propionamido and oxo or a methoxycarbonyl group together with a hydroxy group or an ethoxycarbonyl group together with a hydroxy group) methoxy, ethoxy, propoxy, isopropoxy,

a carbon linked heterocyclyl group selected from azetidinyl, oxazepanyl, pyrrolinyl, pyrrolidinyl, morpholinyl tetrahydrofuranyl, tetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl tetrahydrothienyl, tetrahydropyranyl, tetrahydrothiopyranyl thiomorpholinyl,

a heteroaryl group selected from pyrazolyl, thienyl oxazolyl isoxazolyl, imidazolyl, pyridinyl, pyridazinyl, pyrazinyl, pyrimidyl, furanyl, thiazolyl, isothiazolyl thiadiazolyl,

a (3-7)heterocyclyl(1-6C)alkyl group (wherein the heterocyclyl is carbon linked to the (1-6C)alkyl moiety) selected from azetidinylmethyl, oxazolylmethyl, pyrrolinylmethyl, pyrrolidinylmethyl, morpholinylmethyl, tetrahydro-1,4-thiazinylmethyl, piperidinylmethyl, homopiperidinylmethyl, piperazinylmethyl, homopiperazinylmethyl, dihydropyridinylethyl, tetrahydropyridinylmethyl, dihydropyrimidinylmethyl, tetrahydropyrimidinylmethyl, tetrahydrofuranylmethyl, tetrahydrothienylmethyl, tetrahydropyranylmethyl, tetrahydrothiopyranylmethyl, thiomorpholinylmethyl, 2-(azetidinyl)ethyl, 2-(oxazepanyl)ethyl, 2-(pyrrolinyl)ethyl, 2-(pyrrolidinyl)ethyl, 2-(morpholinyl)ethyl, 2-(tetrahydro-1,4-thiazinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrofuranyl)ethyl, 2-(tetrahydrothienyl)ethyl, 2-(tetrahydropyranyl)ethyl, 2-(tetrahydrothiopyranyl)ethyl, 2-(thiomorpholinyl)ethyl,

a heteroaryl(1-6C)alkyl group selected from pyrazolylmethyl, thienylmethyl, oxazolylmethyl, isoxazolylmethyl, imidazolylmethyl, pyridinylmethyl, pyridazinylmethyl, pyrazinylmethyl, pyrimidylmethyl, furanylmethyl, pyrazolylmethyl, thiazolylmethyl, isothiazolylmethyl, thiadiazolylmethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl, 2-(isothiazolyl)ethyl and 2-(thiadiazolyl)ethyl,
and wherein any heteroaryl or heterocyclyl group within R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido, propionamido and hydroxy and/or optionally a substituent selected from oxo, cyano, methoxy and ethoxy,
and wherein any heteroaryl or heterocyclyl group within R⁶ is optionally substituted on any available ring nitrogen (provided the ring is not thereby quaternised) by methyl, ethyl, acetyl or propionyl
or R⁵ and R⁶ together with the nitrogen atom to which they are attached form an azetidin-1-yl ring which is substituted on an available ring carbon atom by a substituent selected from carbamoyl or (1-3C)alkylenedioxy.
(x) R⁵ is hydrogen, methyl or ethyl and R⁶ is selected from substituted-methyl substituted-ethyl substituted-propyl, substituted-isopropyl, substituted-isobutyl, (wherein the substituted groups are substituted by 1 or 2 substituents independently selected from methoxycarbonyl, ethoxycarbonyl, carbamoyl acetamido and oxo or a methoxycarbonyl group together with a hydroxy group), methoxy, ethoxy,

a carbon liked heterocyclyl group selected from azetidinyl, pyrrolinyl, pyrrolidinyl, morpholinyl, tetrahydrofuranyl, piperidinyl, piperazinyl, tetrahydropyridinyl, tetrahydropyranyl, thiomorpholinyl,

a heteroaryl group selected from pyrazolyl, thienyl, oxazolyl, isoxazolyl, imidazolyl, pyridinyl, pyridazinyl, pyrazinyl, pyrimidyl, furanyl, pyrazolyl, thiazolyl, isothiazolyl,

a (3-7)heterocyclyl(1-6C)alkyl group (wherein the heterocyclyl is carbon linked to the (1-6C)alkyl moiety) selected from azetidinylmethyl, pyrrolinylmethyl, pyrrolidinylmethyl, morpholinylmethyl, piperidinylmethyl, piperazinylmethyl, tetrahydrofuranylmethyl, tetrahydropyranylmethyl, tetrahydropyridinylmethyl, thiomorpholinylnethyl, 2-(azetidinyl)ethyl, 2-(pyrrolinyl)ethyl, 2-(pyrrolidinyl)ethyl, 2-(morpholinyl)ethyl, 2-(piperidinyl)ethyl, 2-(piperazinyl)ethyl, 2-(tetrahydrofuranyl)ethyl, 2-(tetrahydropyranyl)methyl, 2-(tetrahydropyridinyl)ethyl, 2-(thiomorpholinyl)ethyl,

a heteroaryl(1-6C)alkyl group selected from pyrazolylmethyl, thienylmethyl, oxazolylmethyl, isoxazolylmethyl, imidazolylmethyl, pyridinylmethyl, pyridazinylmethyl, pyrazinylmethyl, pyrimidylmethyl, furanylmethyl, pyrazolylmethyl, thiazolylmethyl, isothiazolylmethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl)ethyl,
and wherein any heteroaryl or heterocyclyl group within R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido and hydroxy and/or optionally a substituent selected from oxo, cyano, methoxy and ethoxy,
and wherein any heteroaryl or heterocyclyl group within R⁶ is optionally substituted on any available ring nitrogen (provided the ring is not thereby quaternised) by methyl, ethyl, acetyl or propionyl;
or R⁵ and R⁶ together with the nitrogen atom to which they are attached form an azetidin-1-yl ring which is substituted by a carbamoyl group.
(y) R⁵ is hydrogen or methyl and R⁶ is selected from methoxy, carbamoylmethyl, 2-(hydroxy)-1-(methoxycarbonyl)ethyl, 1-(ethoxycarbonyl)-2-hydroxyethyl, 2-(acetamido)ethyl, piperidin-4yl, 1-methylpiperidin-4yl, tetrahydropyran-4yl, 4hydroxytetrahydrofuran-3-yl, 3-oxotetrahydrofuran-4yl, 1-methylpyrazol-5-yl, thien-3yl, 3-methylpyrazol-5-yl, tetrahydrofuran-2-ylmethyl, tetrahydropyran-4ylmethyl, furan-2-ylmethyl, 2-(furan-2-yl)ethyl, imidazol-1-ylmethyl, imidazol-2-ylmethyl, imidazol-2-ylmethyl, 2-(imidazol-1-yl)ethyl, 2-(imidazol-4yl)ethyl and 5-methylisoxazol-3-ylmethyl or R⁵ and R⁶ together with the nitrogen atom to which they are attached form an azetidin- 1-yl ring which is substituted in the 2 position by a carbamoyl group.

Preferably, R² is in the 7-position and the substituted-pyrrolidinyloxy group is in the 6-position of the quinazoline ring.

When the pyrrolidinyloxy group is linked to the 6-position of the quinazoline ring in the compounds of formula I, and m is 2 and substituents R¹ are both halogeno and attached to the 2- and 3-positions of the ring A, then R⁶ is suitably as defined paragraphs 9(n) or 9(v) above (ie. R⁶ is selected from a substituted-(1-6C)alkyl (wherein substituted-(1-6C)alkyl is (1-6C)alkyl substituted by 1, 2 or 3 substituents independently selected from (1-6C)alkoxycarbonyl, carbamoyl, (2-6C)alkanoylamino, and oxo or a (1-6C)alkoxycarbonyl together with a hydroxy group), (1-6C)alkoxy, (1-6C)alkylsulfonyl, (3-7)heterocyclyl (wherein the heterocyclyl is carbon linked), heteroaryl, (3-7)heterocyclyl(1-6C)alkyl (wherein the heterocyclyl is carbon linked to the (1-6C)alkyl moiety) and heteroaryl(1-6C)alkyl, and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy, wherein any heteroaryl or heterocyclyl group within R⁶ is optionally substituted on any available ring nitrogen (provided the ring is not thereby quaternised) by (1-4C)alkyl or (2-4C)alkanoyl, or
R⁵ and R⁶ together with the nitrogen atom to which they are attached form a 4, 5 or 6 membered ring which is substituted by 1 or 2 substituents on an available ring carbon atom, independently selected from carbamoyl and (1-3C)alkylenedioxy).

More particularly, when the pyrrolidinyloxy group is linked to the 6-position of the quinazoline ring, m is 2 and substituents R¹ are both halogeno and attached to the 2- and 3-positions of the ring A, then R⁶ has any one of the definitions set out in paragraphs 9(o), 9(p), 9(q), (or 9(w), 9(x) or 9(y)) above.

In a particular group of compounds of the invention in which the pyrrolidinyloxy group is linked to the 6-position of the quinazoline ring, m is 2 and substituents R¹ are both halogeno and attached to the 2- and 3-positions of the ring A, R⁶ is as defined in paragraph 9(r) above [ie. R⁶ is selected from (3-7)heterocyclyl (wherein the heterocyclyl is carbon linked), heteroaryl, (3-7)heterocyclyl(1-6C)alkyl (wherein the heterocyclyl is carbon linked to the (1-6C)alkyl moiety) and heteroaryl(1-6C)alkyl, and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy, and wherein any heteroaryl or heterocyclyl group within R⁶ is optionally substituted on any available ring nitrogen (provided the ring is not thereby quaternised) by (1-4C)alkyl or (2-4C)alkanoyl], or any one of paragraphs 9(s), 9(t), and 9(u) above.

A particular class of compounds of the present invention have the sub-formula A1 shown below: 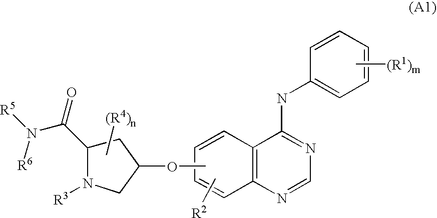
wherein:
either R² is in the 6-position and the substituted-pyrrolidinyloxy group is in the 7-position of the quinazoline ring or R² is in the 7-position and the substituted-pyrrolidinyloxy group is in the 6-position of the quinazoline ring;
and m, n, R¹, R², R³, R⁴, R⁵ and R⁶ have any of the definitions set out above.

A more particular class of compounds of the invention have the sub-formula A2 shown below: 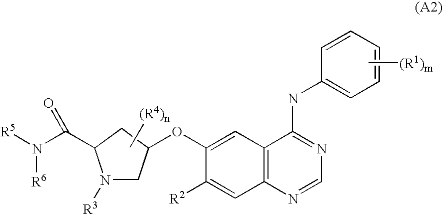
wherein m, n, R¹, R², R³, R⁴, R⁵ and R⁶ have any of the definitions set out above.

In a particular group of compounds of Formula A1 or A2 above:
m and R¹ have any one of the definitions set out in paragraphs 1(a) to (g) above;
R² has any one of the definitions set out in paragraphs 3(a) to (d) above;
R³ has any one of the definitions set out in paragraphs 5(a) to (e) above;
n and R⁴ have any one of the definitions set out in paragraphs 6(a) to (c) above;
and R⁵ and R⁶ have any one of the definitions set out in paragraphs 9(e) to (y) above.

In a preferred group of compounds of Formula A1 or A2 above: m and R¹ have any of the definitions set out in paragraphs 1(f) or 1(g) above;
R² is methoxy;
R³ has any one of the definitions set out in paragraphs 5(d) or 5(e) above;
n is 0;
and R⁵ and R⁶ have any one of the definitions set out in paragraphs 9(n) to 9(y) above.

In a particularly preferred group of compounds of Formula A2 above:
m is 2 and R¹ is 2-fluoro and 3-chloro;
R² is methoxy;
R³ is methyl;
n is 0;
R⁵ is hydrogen or (1-6C)alkyl and R⁶ is selected from substituted-(1-6C)alkyl (wherein substituted-(1-6C)alkyl is (1-6C)alkyl substituted by 1, 2 or 3 substituents independently selected from (1-6C)alkoxycarbonyl, carbamoyl, (2-6C)alkanoylamino, and oxo or a (1-6C)alkoxycarbonyl group together with a hydroxy group), (1-6C)alkoxy, (1-6C)alkylsulfonyl, (3-7)heterocyclyl (wherein the heterocyclyl is carbon linked), heteroaryl, (3-7)heterocyclyl(1-6C)alkyl (wherein the heterocyclyl is carbon linked to the (1-6C)alkyl moiety) and heteroaryl(1-6C)alkyl,
and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy, and wherein any heteroaryl or heterocyclyl group within R⁶ is optionally substituted on any available ring nitrogen (provided the ring is not thereby quaternised) by (1-4C)alkyl or (2-4C)alkanoyl, or

R⁵ and R⁶ together with the nitrogen atom to which they are attached form a 4, 5 or 6 membered ring which is substituted by 1 or 2 substituents on an available ring carbon atom, independently selected from carbamoyl and (1-3C)alkylenedioxy.

In a further preferred group of compounds of Formula A2 above:
m is 2 and R¹ is 2-fluoro and 3-chloro;
R² is methoxy;
R³ is methyl;
n is 0;
R⁵ is hydrogen or (1-6C)alkyl and R⁶ is selected from (3-7)heterocyclyl (wherein the heterocyclyl is carbon linked), heteroaryl, (3-7)heterocyclyl(1-6C)alkyl (wherein the heterocyclyl is carbon linked to the (1-6C)alkyl moiety) and heteroaryl(1-6C)alkyl, and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(I-6C)alkyl, (1-6C)alkoxycarbonyl, carbamoyl, (2-6C)alkanoylamino and hydroxy and/or optionally a substituent selected from oxo, cyano, nitro and (1-4C)alkoxy, and wherein any heteroaryl or heterocyclyl group within R⁶ is optionally substituted on any available ring nitrogen (provided the ring is not thereby quaternised) by (1-4C)alkyl or (2-4C)alkanoyl.

Further classes of particular compounds of the invention are disclosed in Table A using combinations of the definitions described hereinabove. For example, ‘a’ in the column headed R² in the table refers to definition (a) given for R² hereinabove.

TABLE A

| | n and | | | Position | | n and | Position of | Position of | R⁵ and |
|---|---|---|---|---|---|---|---|---|---|
| Class | R¹ | A | R² | of R² | R³ | R⁴ | —CONR⁵R⁶ | quinazolinyloxy | R⁶ |
| 1 | A | a | I | I | I | a | I | I | I |
| 2 | A | a | A | I | a | a | a | a | I |
| 3 | B | b | A | I | a | a | a | a | a |
| 4 | C | b | A | I | b | b | a | b | a |
| 5 | D | b | B | b | b | c | a | b | b |
| 6 | E | b | B | b | c | c | a | b | c |
| 7 | F | b | D | a | d | c | a | b | I |
| 8 | F | b | B | b | d | c | a | b | c |
| 9 | F | b | C | b | d | c | a | b | d |
| 10 | F | b | C | b | e | c | a | b | d |

Particular compounds of the present invention include one or more of the following:
(4S)-4-({4-[(3-chloro-2-fluorophenyl)aninolquinazolin-7-yl}oxy)-N,N,1-trimethyl-L-propionamide;
(4S)-4-({4-[(3-chloro-2-fluorophenyl)amino]quinazolin-7-yl}oxy)-1-methyl-L-propionamide;
(4S)-4-({4-[(4-cyano-2-fluorophenyl)amino]-7-methoxyquinazolin-6-yl}oxy)-N,N1-trimethyl-D-propionamide;
(4S)-4-({4-[(3-chloro-4-cyanophenyl)amino]-7-methoxyquinazolin-6-yl}oxy)-N,N,1-trimethyl-D-propionamide;
(4S)-4-[(4-{[3-chloro-4-(trifluoromethyl)phenyl]amino}-7-methoxyquinazolin-6-yl)oxy]-N,N,1-trimethyl-D-propionamide;
(4S)-4-({4-[(5-chloropyridin-3-yl)amino]-7-methoxyquinazolin-6-yl}oxy)-N,N,1-trimethyl-D-propionamide;
(4S)-4-({4-[(2-fluoro-4-methylphenyl)amino]-7-methoxyquinazolin-6-yl}oxy)-N,N,1-trimethyl-D-propionamide;
(4S)-4-({4-[(3-chloro-4-fluorophenylamino]-7-methoxyquinazolin-6-yl}oxy)-N,N,1-trimethyl-D-prolinamide;
(4S)-4({4-[(2-fluoro-4-hydroxyphenyl)amino]-7-methoxyquinazolin-6-yl}oxy)-N,N,1-trimethyl-D-prolinamide;
(4S)-4-({4-[(2,4-difluorophenyl)amino]-7-methoxyquinazolin-6-yl}oxy)-N,N,1-trimethyl-D-prolamide;
(4S)-4-({4-[(2,5-difluorophenyl)amino]-7-methoxyquinazolin-6-yl}oxy)-N,N,1-trimethyl-D-prolinamide;
(4S)-4-({4-[(5-chloro-2-fluorophenyl)amino]-7-methoxyquinazolin-6-yl}oxy)-N,N,1-trimethyl-D-prolinamide;
(4S)-4-({4-[(4-chloro-2-fluorophenyl)amino]-7-methoxyquinazolin-6-yl}oxy)-N,N,1-trimethyl-D-prolinamide;
(4S)-4-({4-[(5-chloro-2-hydroxyphenyl)amino]-7-methoxyquinazolin-6-yl}oxy)-N,N,1-trimethyl-D-prolinamide;
(4S)-4({4-[(3-chloro-4-methoxyphenyl)amino]-7-methoxyquinazolin-6-yl}oxy)-N,N,1-trimethyl-D-prolinamide;
(4S)-4-[(4-{[2-(aminosulfonyl)-5-chlorophenyl]amino}-7-methoxyquinazolin-6-yl)oxy]-N,N,1-trimethyl-D-prolinamide;
(4S)-4({7-methoxy-4-[(2,3,4-trifluorophenyl)amino]quinazolin-6-yl}oxy)-N,N,1-trimethyl-D-prolinamide;
(4S)-4-[(4-{[2-fluoro-5-(trifluoromethyl)phenyl]amino}-7-methoxyquinazolin-6-yl)oxy]-N,N,1-trimethyl-D-prolinamide;
(4S)-4-[(4-{[2-fluoro-3-(trifluoromethyl)phenyl]amino}-7-methoxyquinazolin-6-yl)oxy]-N,N,1-trimethyl-D-prolinamide;
(4S)-4-({4-[(3-chloro-2-methoxyphenyl)amino]-7-methoxyquinazolin-6-yl}oxy)-N,N,1-trimethyl-D-prolinamide;
(4S)-4-({4-[(3-chloro-2-methylphenyl)amino]-7-methoxyquinazolin-6-yl}oxy)-N,N,1-trimethyl-D-prolinamide;
(4S)-4-({4-[(3-chloro-4hydroxyphenyl)amino]-7-methoxyquinazolin-6-yl}oxy)-N,N,1-trimethyl-D-prolinamide;
(4S)-4-({4-[(3-ethynylphenyl)amino]-7-methoxyquinazolin-6-yl}oxy)-N,N,1-trimethyl-D-prolinamide;
(4S)-4-({4-[(3-cyanophenylamino]-7-methoxyquazolin-6-yl}oxy)-N,N,1-trimethyl-D-prolinamide;
(4S)-4-{[4-(1H-indol-5-ylamino)-7-methoxyquinazolin-6-yl]oxy}-N,N,1-trimethyl-D-prolinamide;
(4S)-4-({4-[(3-chloro-1H-indol-5-yl)amino]-7-methoxyquinazolin-6-yl)oxy}-N,N,1-trimethyl-D-prolinamide;
(4S)-4-({4-[(3-chloro-2-fluorophenyl)amino]-7-methoxyquinazolin-6-yl}oxy)-N-cyclopropyl-1-methyl-D-prolinamide;
(4S)-4-({4-[(3-chloro-2-fluorophenyl)amino]-7-methoxyquinazolin-6-yl}oxy)-N-(cyclopropylmethyl)-1-methyl-D-prolinamide;
(4S)-4-({4-[(3-chloro-2-fluorophenyl)amino]-7-methoxyquinazolin-6-yl}oxy)-N-(2-methoxyethyl)-1-methyl-D-prolinamide;
(4S)-4-({4-[(3-chloro-2-fluorophenyl)amino]-7-methoxyquinazolin-6-yl}oxy)-N-cyclopentyl-1-methyl-D-prolinamide;
(4S)-4-({4-[(3-chloro-2-fluorophenyl)amino]-7-methoxyquinazolin-6-yl}oxy)-N-cyclopentylmethyl-1 -methyl-D-prolinamide;
(4S)-4-({4-[(3-chloro-2-fluorophenyl)amino]-7-methoxyquinazolin-6-yl}oxy)-N-(2-methoxyethyl)-N,1-dimethyl-D-prolinamide;
(4S)-4-({4-[(3-chloro-2-fluorophenyl)amino]-7-methoxyquinazolin-6-yl}oxy)-N-methoxy-1-methyl-D-prolinamide;
(4S)-4-({4-[(3-chloro-2-fluorophenyl)amino]-7-methoxyquinazolin-6-yl}oxy)-N-cyclohexyl-1methyl-D-prolinamide;
(4S)-4-({4-[(3-chloro-2-fluorophenyl)amino]-7-methoxyquinazolin-6-yl}oxy)-1-methyl-N-(tetrahydro-2H-pyran-4-yl)-D-prolinamide,; and
(4S)-4-({4-[(3-chloro-4-fluorophenyl)amino]-6-methoxyquinazolin-7-yl}oxy)-N,N,1-trimethyl-L-prolinamide;
and pharmaceutically-acceptable salts thereof.

Further particular compounds of the present invention include one or more of the following:
(4S)-4-({4-[(3-chloro-2-fluorophenyl)amino]-7-methoxyquinazolin-6-yl}oxy)-N-[(1R)-1-(hydroxymethyl)-3-methylbutyl]-1-methyl-D-prolinamide;
(4S)-4-({4-[(3-chloro-2-fluorophenyl)amino]-7-methoxyquinazolin-6-yl}oxy)-N-[(1S)-1-(hydroxymethyl)-3-methylbutyl]-1-methyl-D-prolinamide;
(4S)-4-({4-[(3-chloro-2-fluorophenyl)amino]-7-methoxyquinazolin-6-yl}oxy)-N-(3-furylmethyl)-1-methyl-D-prolinamide;
(4S)-4-({4-[(3-chloro-2-fluorophenyl)amino]-7-methoxyquinazolin-6-yl}oxy)-N-(2-furylmethyl)-1-methyl-D-prolinamide;
(4S)-4-({4-[(3-chloro-2-fluorophenyl)amino]-7-methoxyquinazolin-6-yl}oxy)-1-methyl-N-[(5-methylisoxazol-3-yl)methyl]-D-prolinamide;
(4S)-4-({4-[(3-chloro-2-fluorophenyl)amino]-7-methoxyquinazolin-6-yl}oxy)-N-[2-(1H-imidazol-1-yl)ethyl]-1-methyl-D-prolinamide;
(2S)-1-[(4S)-4-({4-[(3-chloro-2-fluorophenyl)amino]-7-methoxyquinazolin-6-yl}oxy)-1-methyl-D-propyl]azetidine-2-carboxamide;
(4S)-4-({4-[(3-chloro-2-fluorophenyl)amino]-7-methoxyquinazolin-6-yl}oxy)-N-[(2R)-2,3-dihydroxypropyl]-1-methyl-D-prolinamide;
(4S)-4-({4-[(3-chloro-2-fluorophenyl)amino]-7-methoxyquinazolin-6-yl}oxy)-1-methyl-N-(1-methyl-1H-pyrazol-5-yl)-D-prolinamide;
(4S)-4-({4-[(3-chloro-2-fluorophenyl)amino]-7-methoxyquinazolin-6-yl}oxy)-1-methyl-N-3-thienyl-D-prolinamide; and
(4S)-4-({4-[(3-chloro-2-fluorophenyl)amino]-7-methoxyquinazolin-6-yl}oxy)-1-methyl-N-(3-methyl-1H-pyrazol-5-yl)-D-prolinamide;
methyl (4S)-4-({4-[(3-chloro-2-fluorophenyl)amino]-7-methoxyquinazolin-6-yl}oxy)-1-methyl-D-propyl-L-serinate;
(4S)-4-({4-[(3-chloro-2-fluorophenyl)amino]-7-methoxyquinazolin-6-yl}oxy)-N-(2-hydroxy-1,1-dimethylethyl)-1-methyl-D-prolinamide;
(4S)-4-({4-[(3-chloro-2-fluorophenyl)amino]-7-methoxyquinazolin-6-yl}oxy)-1-methyl-D-prolinamide;
(4S)-N-[2-(acetylamino)ethyl]-4-({4-[(3-chloro-2-fluorophenyl)amino]-7-methoxyquinazolin-6-yl}oxy)-1-methyl-D-prolinamide;
(4S)-4-({4-[(3-chloro-2-fluorophenyl)amino]-7-methoxyquinazolin-6-yl}oxy)-N-[(3S, 4R)-4-hydroxytetrahydrofuran-3-yl]-1-methyl-D-prolinamide;
(4S)-4-({4-[(3-chloro-2-fluorophenyl)amino]-7-methoxyquinazolin-6-yl}oxy)-N-[1-(hydroxymethyl)cyclopentyl]-1-methyl-D-prolinamide;
(4S)-4-({4-[(3-chloro-2-fluorophenyl)amino]-7-methoxyquinazolin-6-yl}oxy)-N-[(1-(hydroxymethyl)-2-methylpropyl]-1-methyl-D-prolinamide;
(4S)-4({4-[(3-chloro-2-fluorophenyl)amino]-7-methoxyquinazolin-6-yl}oxy)-N-[2-(1H-imidazol-4-yl)ethyl]-1-methyl-D-prolamide;
(4S)-4-({4-[(3-chloro-2-fluorophenyl)amino]-7-methoxyquinazolin-6-yl}oxy)-N-(2-methoxy-1-methylethyl)-1-methyl-D-prolinamide;
(4S)-4-({4-[(3-chloro-2-fluorophenyl)amino]-7-methoxyquinazolin-6-yl}oxy)-1-methyl-N-(2,2,2-trifluoroethyl)-D-prolinamide;
(4S)-N-allyl-4-({4-[(3-chloro-2-fluorophenyl)amino]-7-methoxyquinazolin-6-yl}oxy)-1-methyl-D-prolinamide;
(4S)-4-({4-[(3-chloro-2-fluorophenyl)amino]-7-methoxyquinazolin-6-yl}oxy)-N-(2-ethoxyethyl)-1-methyl-D-prolinamide;
(4S)-4-({4-[(3-chloro-2-fluorophenyl)amino]-7-methoxyquinazolin-6-yl}oxy)-N-(4-hydroxycyclohexyl)-1-methyl-D-prolinamide;
(4S)-4-({4-[(3-chloro-2-fluorophenyl)amino]-7-methoxyquinazolin-6-yl}oxy)-1-methyl-N-(2-methylpropyl-2-en-1-yl)-D-prolinamide;
(4S)-4-({4[(3-chloro-2-fluorophenyl)amino]-7-methoxyquinazolin-6-yl}oxy)-N-[(1S)-1-(hydroxymethyl)propyl]-1-methyl-D-prolinamide;
(4S)-4-({4-[(3-chloro-2-fluorophenyl)amino]-7-methoxyquinazolin-6-yl}oxy)-N-[(2S)-2,3-dihydroxypropyl]-1-methyl-D-prolinamide;
(4S)-4-({4-[(3-chloro-2-fluorophenyl)amino]-7-methoxyquinazolin-6-yl}oxy)-N-(1H-imidazol-2-ylmethyl)-1-methyl-D-prolinamide;
(4S)-4-({4-[(3-chloro-2-fluorophenyl)amino]-7-methoxyquinazolin-6-yl}oxy)-N-[-(2-(2-furyl)ethyl]-1-methyl-D-prolinamide;
(4S)-4-({4-[(3-chloro-2-fluorophenyl)amino]-7-methoxyquinazolin-6-yl}oxy)-1-methyl-N-(tetrahydro-2H-pyran-4-ylmethyl)-D-prolinamide;
(4S)-4-({4-[(3-chloro-2-fluorophenyl)amino]-7-methoxyquinazolin-6-yl}oxy)-N-[(1S)-2-hydroxy-1-methylethyl]-1-methyl-D-prolinamide;
(4S)-4-({4-[(3-chloro-2-fluorophenyl)amino]-7-methoxyquinazolin-6-yl}oxy)-N-[(1R)-2-hydroxy-1-methylethyl]-1-methyl-D-prolinamide;
(4S)-4-({4-[(3-chloro-2-fluorophenyl)amino]-7-methoxyquinazolin-6-yl}oxy)-N-[(2R)-2-hydroxypropyl]-1-methyl-D-prolinamide;
(4S)-4-({4-[(3-chloro-2-fluorophenyl)amino]-7-methoxyquinazolin-6-yl}oxy)-N-[(2S)-2-hydroxypropyl]-1-methyl-D-prolinamide;
(4S)-4-({4-[(3-chloro-2-fluorophenyl)amino]-7-methoxyquinazolin-6-yl}oxy)-1-methyl-N-[(2R)-tetrahydrofuran-2-ylmethyl]-D-prolinamide;
(4S)-4-({4-[(3-chloro-2-fluorophenyl)amino]-7-methoxyquinazolin-6-yl}oxy)-1-methyl-N-[(2S)-tetrahydrofuran-2-ylmethyl]-D-prolinamide
N-(3-chloro-2-fluorophenyl)-7-methoxy-6-{[(3S,5R)-1-methyl-5-(pyrrolidin-1-ylcarbonyl)pyrrolidin-3-yl]oxy}quinazolin-4-amine;
N-(3-chloro-2-fluorophenyl)-7-methoxy-6-({(3S,5R)-1-methyl-5-[(4-methylpiperazin-1-yl)carbonyl]pyrrolidin-3-yl}oxy)quinazolin-4-amine
6-{[(3S,5R)-5-(azetidin-1-ylcarbonyl)-1-methylpyrrolidin-3-yl]oxy}-N-(3-chloro-2-fluorophenyl)-7-methoxyquinazolin-4-amine;
(4S)-4-({4-[(3-chloro-2-fluorophenyl)amino]-7-methoxyquinazolin-6-yl}oxy)-N-(cyanomethyl)-N,1-dimethyl-D-prolinamide;
(4S)-4-({4-[(3-chloro-2-fluorophenyl)amino]-7-methoxyquinazolin-6-yl}oxy)-N-(cyanomethyl)-1-methyl-D-prolinamide;
(4S)-4-({4-[(3-chloro-2-fluorophenyl)amino]-7-methoxyquinazolin-6-yl}oxy)-N,1-dimethyl-N-[(2S)-2-pyrrolidin-1-ylpropyl]-D-prolinamide;
(4S)-4-({4-[(3-chloro-2-fluorophenyl)amino]-7-methoxyquinazolin-6-yl}oxy)-N-[(1R)-2-hydroxy-1-methylethyl]-N,1-dimethyl-D-prolinamide;
(4S)-4-({4-[(3-chloro-2-fluorophenyl)amino]-7-methoxyquinazolin-6-yl}oxy)-N,1-dimethyl-N-(1-methylpiperidin-4-yl)-D-prolinamide;
(4S)-4-({4-[(3-chloro-2-fluorophenyl)amino]-7-methoxylquinazolin-6-yl}oxy)-N,1-dimethyl-N-(tetrahydro-2H-pyran-4-yl)-D-prolinamide;
(4R)-4-({4-[(3-chloro-2-fluorophenyl)amino]-7-methoxyquinazolin-6-yl}oxy)-1-methyl-N-prop-2-yn-1-yl-L-prolinamide;
1-[[(2S,4R)-4-[[4-[(3-chloro-2-fluorophenyl)amino]-7-methoxy-6-quinazolinyl]oxy]-1-methyl-2-pyrrolidinyl]carbonyl]-3-pyrroline;
(4R)-4-({4-[(3-chloro-2-fluorophenyl)amino]-7-methoxyquinazolin-6-yl}oxy)-N-(cyanomethyl)-1-methyl-L-prolinamide;
(4R)-4({4-[(3-chloro-2-fluorophenyl)amino]-7-methoxyquinazolin-6-yl}oxy)-N-(2-cyanoethyl)-1-methyl-L-prolinamide;
(4R)-4-({4-[(3-chloro-2-fluorophenyl)amino]-7-methoxyquinazolin-6-yl}oxy)-N-(cyanomethyl)-N,1-dimethyl-L-prolinamide;
(4R)-4-({4-[(3-chloro-2-fluorophenyl)amino]-7-methoxyquinazolin-6-yl}oxy)-N-(2-methoxyethyl)-1-methyl-L-prolinamide;
(4R)-4-({4-[(3-chloro-2-fluorophenyl)amino]-7-methoxyquinazolin-6-yl}oxy)-N-cyclopropyl-1-methyl-L-prolinamide;
(4R)-4-({4-[(3-chloro-2-fluorophenyl)amino]-7-methoxyquinazolin-6-yl}oxy)-N-cyclopentyl-1-methyl-L-prolinamide;
N-(3-chloro-2-fluorophenyl)-7-methoxy-6-({(3R,5S)-1-methyl-5-[(4-methylpiperazin-1-yl)carbonyl]pyrrolidin-3-yl}oxy)quinazolin-4-amine;
(3S)-1-[(4R)-4-({4-[(3-chloro-2-fluorophenyl)amino]-7-methoxyquinazolin-yl}oxy)-1-methyl-L-propyl]pyrrolidin-3-ol
(4R)-4-({4-[(3-chloro-2-fluorophenyl)amino]-7-methoxyquinazolin-6-yl}oxy)-N-(cyclopropylmethyl)-1-methyl-L-prolinamide;
(4R)-4-({4-[(3-chloro-2-fluorophenyl)amino]-7-methoxyquinazolin-6-yl}oxy)-N-cyclohexyl-N,1-dimethyl-L-prolinamide;
(4R)-4-({-[(3-chloro-2-fluorophenyl)amino]-7-methoxyquinazolin-6-yl}oxy)-1-methyl-N-(tetrahydro-2H-pyran-4-yl)-L-prolinamide;
N-(3-chloro-2-fluorophenyl)-7-methoxy-6-{[(3R,5S)-1-methyl-5-(pyrrolidin-1-ylcarbonyl)pyrrolidin-3-yl]oxy }quinazolin-4-amine;
(4R)-4-({4-[(3-chloro-2-fluorophenyl)amino]-7-methoxyquinazolin-6-yl}oxy)-N-(2-hydroxyethyl)-N,1-dimethyl-L-prolinamide;
(4R)-4({4-[(3-chloro-2-fluorophenyl)amino]-7-methoxyquinazolin-6-yl}oxy)-N-[2-(dimethylamino)ethyl]-1-methyl-L-prolinamide;
(4R)-4-({4-[(3-chloro-2-fluorophenyl)amino]-7-methoxyquinazolin-6-yl}oxy)-N,1-dimethyl-N-(1-methylpiperidin-4-yl)-L-prolinamide;
6-({(3R,5S)-5-[(4-acetylpiperazin-1-yl)carbonyl]-1-methylpyrrolidin-3-yl}oxy)-N-(3-chloro-2-fluorophenyl)-7-methoxyquinazolin-4-amine;
1-[(4R)-4({4[(3-chloro-2-fluorophenyl)amino]-7-methoxyquinazolin-6-yl}oxy)-1-methyl-L-prolyl]piperidin-4-ol;
(4R)-4({4-[(3-chloro-2-fluorophenyl)amino]-7-methoxyquinazolin-6-yl}oxy)-N-(2-methoxyethyl)-N,1-dimethyl-L-prolinamide;
(4R)-4-({4-[(3-chloro-2-fluorophenyl)amino]-7-methoxyquinazolin-6yl}oxy)-N-cyclohexyl-1-methyl-L-prolinamide;
(4S)-4({4-[(3-chloro-2-fluorophenyl)amino]-7-methoxyquinazolin-6-yl}oxy)-N-cyclopropyl-1-methyl-L-prolinamide;
(4S)-4-({4-[(3-chloro-2-fluorophenyl)amino]-7-methoxyquinazolin-6-yl}oxy)-N-(2-methoxyethyl)-1-methyl-L-prolinamide;
(4S)-4-({4-[(3-chloro-2-fluorophenyl)amino]-7-methoxyquinazolin-6-yl}oxy)-N-cyclohexyl-N,1-dimethyl-L-prolinamide;
(4S)-4-({4-[(3-chloro-2-fluorophenyl)amino]-7-methoxyquinazolin-6-yl}oxy)-1-methyl-N-(tetrahydro-2H-pyran-4-yl)-L-prolinamide;
(4S)-4-({4-[(3-chloro-2-fluorophenyl)amino]-7-methoxyquinazolin-6-yl}oxy)-N-(2-methoxyethyl)-N,1-dimethyl-L-prolinamide;
(4S)-4-({4-[(3-chloro-2-fluorophenyl)amino]-7-methoxyquinazolin-6-yl}oxy)-N,1-dimethyl-N-(1-methylpiperidin-4-yl)-L-prolinamide;
(4S)-4-({4-[(3-chloro-2-fluorophenyl)amino]-7-methoxyquinazolin-6-yl}oxy)-N-cyclopentyl-1-methyl-L-prolinamide;
(4S)-4-({4-[(3-chloro-2-fluorophenyl)amino]-7-methoxyquinazolin-6-yl}oxy)-N-methoxy-1-methyl-L-prolinamide;
(4S)-4-({4-[(3-chloro-2-fluorophenyl)amino]-7-methoxyquinazolin-6-yl}oxy)-N-(cyclopropylmethyl)-1-methyl-L-prolinamide;
(4S)-4-({4-[(3-chloro-2-fluorophenyl)amino]-7-methoxyquinazolin-6-yl}oxy)-N-cyclohexyl-1-methyl-L-prolinamide;
and pharmaceutically-acceptable salts thereof.

A further particular compound of the invention is:
(4S)-4-({4-[(3-chloro-2-fluorophenyl)amino]quinazolin-7-yl)oxy)-1-methyl-L-prolinamide trifluoroacetic acid salt.
   Synthesis of Quinazoline Derivatives of the Formula I

A further aspect the present invention provides a process for preparing a quinazoline derivative of Formula I or a pharmaceutically-acceptable salt thereof. It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein

A suitable protecting group for an amino or alkylamino group is, for example, an acyl group, for example an alkanoyl group such as acetyl, an alkoxycarbonyl group, for example a methoxycarbonyl ethoxycarbonyl or t-butoxycarbonyl group, an arylmethoxycarbonyl group, for example benzyloxycarbonyl or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.
Resins may also be used as a protecting group.

The protecting groups may be removed at any convenient stage in the synthesis using conventional techniques well known in the chemical art.

A quinazoline derivative of the Formula I, or a pharmaceutically-acceptable salt thereof, may be prepared by any process known to be applicable to the preparation of chemically-related compounds. Such processes, when used to prepare a quinazoline derivative of the Formula I, or a pharmaceutically-acceptable salt thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March). The preparation of such starting materials is described within the accompanying non-limiting Examples. Alternatively, necessary starting materials are obtainable by analogous procedures to those illustrated which are within the ordinary skill of an organic chemist. Information on the preparation of necessary starting materials or related compounds (which may be adapted to form necessary starting materials) may also be found in the following Patent and Application Publications, the contents of the relevant process sections of which are hereby incorporated herein by reference: WO94/27965, WO 95/03283, WO 96/33977, WO 96/33978, WO 96/33979, WO 96/33980, WO 96/33981, WO 97/30034, WO 97/38994, WO01/66099, U.S. Pat. No. 5,252,586, EP 520 722, EP 566 226, EP 602 851 and EP 635 507.

The present invention also provides that quinazoline derivatives of the Formula I, or pharmaceutically acceptable salts thereof, can be prepared by a process (a) to (j) as follows (wherein the variables are as defined above unless otherwise stated):
Process (a) By reacting a Compound of the Formula II: 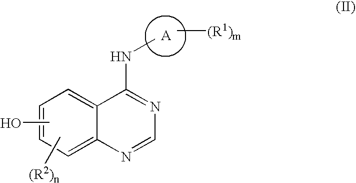
wherein R¹, R², A, M and N have any of the meanings defined hereinbefore except that any functional group is protected if necessary,
with a compound of the Formula III: 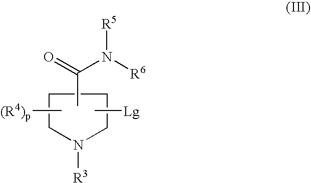
   wherein R³, R⁴, R⁵, R⁶ and p have any of the meanings defined hereinbefore except that any functional group is protected if necessary and Lg is a displaceable group, wherein the reaction is conveniently performed in the presence of a suitable base,

and whereafter any protecting group that is present is removed by conventional means.

A convenient displaceable group Lg is, for example, a halogeno, alkanesulfonyloxy or arylsulfonyloxy group, for example a chloro, bromo, methanesulfonyloxy, 4-nitrobenzenesulfonyloxy or toluene-4-sulfonyloxy group (suitably a methanesulfonyloxy, A nitrobenzenesulfonyloxy or toluene-4-sulfonyloxy group).

The reaction is advantageously carried out in the presence of base. A suitable base is, for example, an organic amine base such as, for example, pyridine, 2,6-lutidine, collidine, 4-dimethylaminopyridine, triethylamine, N-methylmorpholine or diazabicyclo[5.4.0]undec-7-ene, or for example, an alkali metal or alkaline earth metal carbonate or hydroxide, for example sodium carbonate, potassium carbonate, cesium carbonate, calcium carbonate, sodium hydroxide or potassium hydroxide. Alternatively such a base is, for example, an alkali metal hydride, for example sodium hydride, an alkali metal or alkaline earth metal amide, for example sodium amide or sodium bis(trimethylsilyl)amide, or a sufficiently basic alkali metal halide, for example cesium fluoride or sodium iodide. The reaction is suitably effected in the presence of au inert solvent or diluent, for example an alkanol or ester such as methanol, ethanol, 2-propanol or ethyl acetate, a halogenated solvent such as methylene chloride, trichloromethane or carbon tetrachloride, an ether such as tetrahydrofuran or 1,4-dioxan, an aromatic hydrocarbon solvent such as toluene, or (suitably) a dipolar aprotic solvent such as N,N-dimethylformamide, N,N-dimethylacetamide, N-methylpyrrolidin-2-one or dimethylsulfoxide. The reaction is conveniently effected at a temperature in the range, for example, 10 to 150° C. (or the boiling point of the solvent), suitably in the range 20 to 90° C.
Process (b) By modifying a substituent in or introducing a substituent into another quinazoline derivative of Formula I or a pharmaceutically acceptable salt thereof, as hereinbefore defined except that any functional group is protected if necessary, and whereafter any protecting group that is present is removed by conventional means.

Methods for converting substituents into other substituents are known in the art. For example an alkylthio group may be oxidised to an alkylsulfinyl or alkylsulfonyl group, a cyano group reduced to an amino group, a nitro group reduced to an amino group, a hydroxy group alkylated to a methoxy group, a bromo group converted to an alkylthio group or an amino group may be acylated to give an alkanoylamino group (for example by reaction with a suitable acid chloride or acid anhydride). In addition, an R¹ group may be halogenated by reacting it with halogenating agent. For example, a compound of the formula (I) wherein R¹ contains an alkyl group of alkylene group may be chlorinated by reacting it with N-chlorosuccinimide using conditions known in the art. Conveniently, one R¹group may be converted into another R¹ group as a final step in the preparation of a compound of the Formula I. It is also possible to introduce a substituent onto the pyrrole group as a final step in the preparation of a compound of the Formula I.
Process (c) By removal of a protecting group from a quinazoline derivative of Formula I, or a pharmaceutically acceptable salt thereof.

Suitable methods for removal of protecting groups are well known and are discussed herein.

Suitable protecting groups for an amino group are, for example, any of the protecting groups disclosed hereinbefore for an amino group. Suitable methods for the cleavage of such amino protecting groups are also disclosed hereinbefore. In particular, a suitable protecting group is a lower alkoxycarbonyl group such as a tert-butoxycarbonyl group which may be cleaved under conventional reaction conditions such as under acid-catalysed hydrolysis, for example in the presence of trifluoroacetic acid.
Process (d) By reacting a compound of the Formula II as hereinbefore defined with a compound of the Formula III as defined hereinbefore except Lg is OH under Mitsunobu conditions, and whereafter any protecting group that is present is removed by conventional means.

Suitable Mitsunobu conditions include, for example, reaction in the presence of a suitable tertiary phosphine and a di-alkylazodicarboxylate in an organic solvent such as THF, or suitably dichloromethane and in the temperature range 0C - 60° C., but suitably at ambient temperature. A suitable tertiary phosphine includes for example tri-n-butylphosphine or suitably tri-phenylphosphine. A suitable di-alkylazodicarboxylate includes for example diethyl azodicarboxylate (DEAD) or suitably di-tert-butyl azodicarboxylate. Details of Mitsunobu reactions are contained in Tet. Letts., 31, 699, (1990); The Mitsunobu Reaction, D. L. Hughes, Organic Reactions, 1992, Vol. 42, 335-656 and Progress in the Mitsunobu Reaction, D. L. Hughes, Organic Preparations and Procedures International, 1996, Vol. 28, 127-164.
Process (e) For the preparation of those compounds of the Formula I wherein R⁴ is a hydroxy group by the cleavage of a quinazoline derivative of the Formula I wherein R⁴ is a (1-4C)alkoxy group.

The cleavage reaction may conveniently be carried out by any of the many procedures known for such a transformation. The cleavage reaction of a compound of the Formula I wherein R⁴ is a (1-6C)alkoxy group may be carried out, for example, by treatment of the quinazoline derivative with an alkali metal (1-6C)alkylsulfide such as sodium ethanethiolate or, for example, by treatment with an alkali metal diarylphosphide such as lithium diphenylphosphide. Alternatively the cleavage reaction may conveniently be carried out, for example, by treatment of the quinazoline derivative with a boron or aluminum trihalide such as boron tribromide, or by reaction with an organic or inorganic acid, for example trifluoroacetic acid. L-Methionine/methanesulphonic acid is preferred. Such reactions are suitably carried out in the presence of a suitable inert solvent or diluent as defined hereinbefore. A preferred cleavage reaction is the treatment of a quinazoline derivative of the Formula I with pyridine hydrocbloride. The cleavage reactions are suitably carried out at a temperature in the range, for example, of from 10 to 150° C., for example from 25 to 80° C.
Process (f) For the preparation of those compounds of the Formula I wherein R⁴ is (1-4C) alkoxy, by the reaction of a compound of the Formula IV: 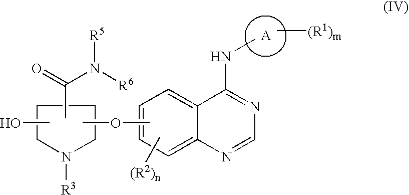
   with a compound of the formula (1-4C)alkyl-Lg, wherein Lg is a displaceable group, wherein the reaction is conveniently performed in the presence of a suitable base;

and whereafter any protecting group that is present is removed by conventional means. Suitable displaceable groups, Lg, are as hereinbefore defined for process a, for example chloro or bromo. The reaction is suitably performed in the presence of a suitable base. Suitable solvents, diluents and bases include, for example those hereinbefore described in relation to process (a).
Process (g)

For the preparation of those compounds of the Formula I wherein R¹, R², R⁴ or R⁶ contain a (1-6C)alkoxy or substituted (1-6C)alkoxy group or a (1-6C)alkylamino or substituted (1-6C)alkylamino group, the alkylation, conveniently in the presence of a suitable base as defined hereinbefore for process a, of a quinazoline derivative of the Formula I wherein R¹, R², R⁴ or R⁶ contain a hydroxy group or a primary or secondary amino group as appropriate. Alkylation may also be used to convert a compound or intermediate wherein R³ is hydrogen to the corresponding compound wherein R³ is alkyl or substituted-alkyl.

A suitable alkylating agent is, for example, any agent known in the art for the alkylation of hydroxy to alkoxy or substituted alkoxy, or for the alkylation of amino to alkylamino or substituted alkylamino, for example an alkyl or substituted alkyl halide, for example a (1-6C)alkyl chloride, bromide or iodide or a substituted (1-6C)alkyl chloride, bromide or iodide, conveniently in the presence of a suitable base as defined hereinbefore, in a suitable inert solvent or diluent as defined hereinbefore and at a temperature in the range, for example, 10 to 140° C., conveniently at or near ambient temperature. An analogous procedure may be used to introduce optionally substituted (2-6C)alkanoyloxy, (2-6C)alkanoylamino and (1-6C)alkanesulfonylamino groups as appropriate.

Conveniently for the production of those compounds of the Formula I wherein R¹ contains a (1-6C)alkylamino or substituted (1-6C)alkylamino group or R³ is converted from hydrogen to alkyl or substituted-alkyl, a reductive amination reaction may be employed using formaldehyde or paraformaldehyde, or a (2-6C)alkanolaldehyde (for example acetaldehyde or propionaldehyde). For example, for the production of those compounds of the Formula I wherein R¹contains an N-methyl group or for the conversion of R³ from hydrogen to an alkyl or substituted-alkyl group, the corresponding compound containing a N—H group may be reacted with formaldehyde in the presence of a suitable reducing agent. A suitable reducing agent is, for example, a hydride reducing agent, for example formic acid, an alkali metal aluminum hydride such as lithium aluminum hydride, or, suitably, an alkali metal borohydride such as sodium borohydride, sodium cyanoborohydride, sodium triethylborohydride, sodium trimethoxyborohydride and sodium triacetoxyborohydride. The reaction is conveniently performed in a suitable inert solvent or diluent, for example tetrahydrofuran and diethyl ether for the more powerful reducing agents such as lithium aluminum hydride, and, for example, methylene chloride or a protic solvent such as methanol and ethanol for the less powerful reducing agents such as sodium triacetoxyborohydride and sodium cyanoborohydride. When the reducing agent is formic acid the reaction is conveniently carried out using an aqueous solution of the formic acid. The reaction is performed at a temperature in the range, for example, 10 to 100° C., such as 70 to 90° C. or, conveniently, at or near ambient temperature. Conveniently, when the reducing agent is formic acid, protecting groups such as tert-butoxycarbonyl on the NH group to be alkylated (for example present from the synthesis of the starting material) may be removed in-situ during the reaction.
Process (h)

By reacting a compound of the formula (V) or reactive derivative thereof 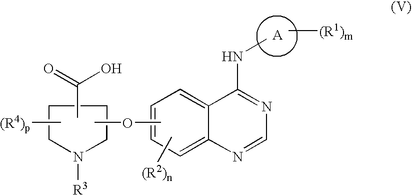
with a compound of the formula HNR⁵R⁶ or a suitable salt in the presence of a suitable base and in an inert solvent.

The coupling reaction is conveniently carried out in the presence of a suitable coupling agent, such as a carbodiimide, or a suitable peptide coupling agent, for example O-(7-azabenzotriazol-1-yl)-N,N,N′,N′-tetramethyluroniumhexafluoro-phosphate (HATU) or a carbodiimide such as dicyclohexylcarbodiimide, optionally in the presence of a catalyst such as dimethylaminopyridine or 4-pyrrolidinopyridine.

The coupling reaction is conveniently carried out in the presence of a suitable base. A suitable base is, for example, an organic amine base such as, for example, pyridine, 2,6-lutidine, collidine, 4-dimethylaminopyridine, triethylamine, di-isopropylethylamine, N-methylmorpholine or diazabicyclo[5.4.0)undec-7-ene, or, for example, an alkali or alkaline earth metal carbonate, for example sodium carbonate, potassium carbonate, cesium carbonate or calcium carbonate.

The reaction is conveniently carried out in the presence of a suitable inert solvent or diluent, for example an ester such as or ethyl acetate, a halogenated solvent such as methylene chloride, chloroform or carbon tetrachloride, an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulfoxide. The reaction is conveniently carried out at a temperature in the range, for example, from 0 to 120° C., conveniently at or near ambient temperature.

A “reactive derivative” of the acid of the formula (V) is a carboxylic acid derivative that will react with an amine of formula (III) to give the corresponding amide. A suitable reactive derivative of a carboxylic acid of the formula (V) is, for example, an acyl halide, for example an acyl chloride formed by the reaction of the acid and an inorganic acid chloride, for example thionyl chloride; a mixed anhydride, for example an anhydride formed by the reaction of the acid and a chloroformate such as isobutyl chloroformate; an active ester, for example an ester formed by the reaction of the acid and a phenol such as pentafluorophenol, an ester such as pentafluorophenyl trifluoroacetate or an alcohol such as methanol, ethanol, isopropanol, butanol or N-hydroxybenzotriazole; or an acyl azide, for example an azide formed by the reaction of the acid and azide such as diphenylphosphinyl azide; an acyl cyanide, for example a cyanide formed by the reaction of an acid and a cyanide such as diethylphosphoryl cyanide. The reaction of such reactive derivatives of carboxylic acid with amines is well known in the art, for example they may be reacted in the presence of a base, such as those described above, and in a suitable solvent, such as those described above. The reaction may conveniently be performed at a temperature as described above.
Process (i)

By reacting a compound of the formula VI: 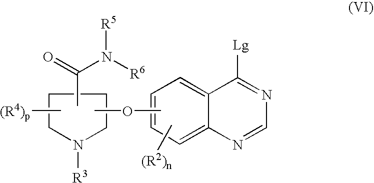
wherein R¹, R², R³, R⁴, R⁵, R⁶, n and p, have any of the meanings defined hereinbefore except that any functional group is protected if necessary and Lg is a displaceable group as hereinbefore defined,
with an aniline of the formula VII: 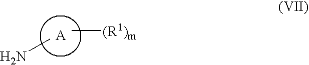
wherein R¹ and m have any of the meanings defined hereinbefore except that any functional group is protected if necessary, and wherein the reaction is conveniently performed in the presence of a suitable acid,

and whereafter any protecting group that is present is removed by conventional means.

Suitable displaceable groups represented by Lg are as hereinbefore defined, in particular halogeno such as chloro.

The reaction is conveniently carried out in the presence of a suitable inert solvent or diluent, for example an alcohol or ester such as, isopropanol or ethyl acetate, a halogenated solvent such as methylene chloride, chloroform or carbon tetrachloride, an ether such as tetrahydrofuran or 1,4-dioxane, an aromatic solvent such as toluene, or a dipolar aprotic solvent such as N,N-dimethylformamide, N,N-dimethylacetamide, N-methylpyrrolidin-2-one acetonitrile or dimethylsulfoxide acetonitrile is favoured. The reaction is conveniently carried out at a temperature in the range, for example, 10 to 250° C., conveniently in the range 40 to 120° C. or where a solvent or diluent is used at the reflux temperature. Conveniently, the compound of formula VI may is reacted with a compound of the formula VII in the presence of a protic solvent such as isopropanol, conveniently in the presence of an acid, for example hydrogen chloride gas in diethyl ether or dioxane, or hydrochloric acid, for example a 4M solution of hydrogen chloride in dioxane, under the conditions described above. Alternatively, this reaction may be conveniently carried out in an aprotic solvent, such as dioxane or a dipolar aprotic solvent such as N,N-diethylacetamide or acetonitrile in the presence of an acid, for example hydrogen chloride gas in diethyl ether or dioxane, or hydrochloric acid. The compound of the formula VI, wherein Lg is halogeno, may be reacted with a compound of the formula VII in the absence of an acid. In this reaction displacement of the halogeno leaving group Lg results in the formation of the acid HLg in-situ and auto-catalysis of the reaction. Conveniently the reaction is carried out in a suitable inert organic solvent, for example isopropanol, dioxane or N,N-dimethylacetamide. Suitable conditions for this reaction are as described above.

Alternatively, the compound of formula VI may is reacted with a compound of the formula VII in the presence of a suitable base. Suitable bases for this reaction are as hereinbefore defined under Process (a). This reaction is conveniently performed in an inert solvent or diluent, for example those mentioned above in relation to this process (i);
Process (j)

Forming the group —CON(R⁵)R⁶ by reacting to the corresponding carboxy compound, wherein any functional groups are protected if necessary, with a primary or secondary amine or a heterocyclic group containing an NH group; and whereafter any protecting group that is present is removed by conventional means.

The coupling reaction is conveniently carried out in the presence of a suitable coupling agent, such as a carbodiimide (for example 1-[3-(Dimethylamino)propyl]-3-ethylcarbodiimide), or a suitable peptide coupling agent, for example O-(7-azabenzotriazol-1-yl)-N,N,N′,N′-tetramethyluronium hexafluoro-phosphate (HATU). The coupling reaction is conveniently carried out in an inert solvent such as, for example, a halogenated solvent such as methylene chloride, or a dipolar aprotic solvent such as N,N-dimethylformamide, N,N-dimethylacetamide, 1-methyl-2-pyrrolidinone. Suitably the coupling reaction is carried out in the presence of a suitable base, such as an organic amine, for example di-isopropylethylamine or 4-dimethylaminopyridine. The coupling reaction is suitable performed at −25° C. to 150° C., conveniently at ambient temperature.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (ie. chemical transformations may be performed upon different intermediates to those associated hereinbefore with a particular reaction).

When a pharmaceutically-acceptable salt of a quinazoline derivative of the Formula I is required, for example an acid-addition salt, it may be obtained by, for example, reaction of said quinazoline derivative with a suitable acid using a conventional procedure. To facilitate isolation of the compound during preparation, the compound may be prepared in the form of a salt that is not a pharmaceutically acceptable salt. The resulting salt can then be modified by conventional techniques to give a pharmaceutically acceptable salt of the compound. Such techniques include, for example ion exchange techniques or re-precipitation of the compound in the presence of a pharmaceutically acceptable counter ion. For example re-precipitation in the presence of a suitable acid such as HCl to give a hydrochloride acid addition salt.

As mentioned hereinbefore some of the compounds according to the present invention may contain one of more chiral centres and may therefore exist as stereoisomers (for example when Q¹ contains a pyrrolidin-3-yl group). Stereoisomers may be separated using conventional techniques, e.g. chromatography or fractional crystallisation. The enantiomers may be isolated by separation of a racemate for example by fractional crystallisation, resolution or HPLC. The diastereomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions which will not cause racemisation or epimerisation, or by derivatisation, with a chiral reagent. Examples of suitable chiral synthesis and separation of isomers are described in the Examples. When a specific stereoisomer is isolated it is suitably isolated substantially free for other stereoisomers, for example containing less than 20%, particularly less than 10% and more particularly less than 5% by weight of other stereoisomers.

In the section above the expression “inert solvent” refers to a solvent which does not react with the starting materials, reagents, intermediates or products in a maimer which adversely affects the yield of the desired product.

Preparation of Starting Materials

Compounds of Formula II are commercially available or may be prepared using conventional techniques or analogous processes to those described in the prior art. In particular those patents and applications listed hereinbefore, such as WO96/15118, WO 01/66099 and EP 566 226. For example, the compounds of Formula Il may be prepared in accordance with Reaction Scheme 1: 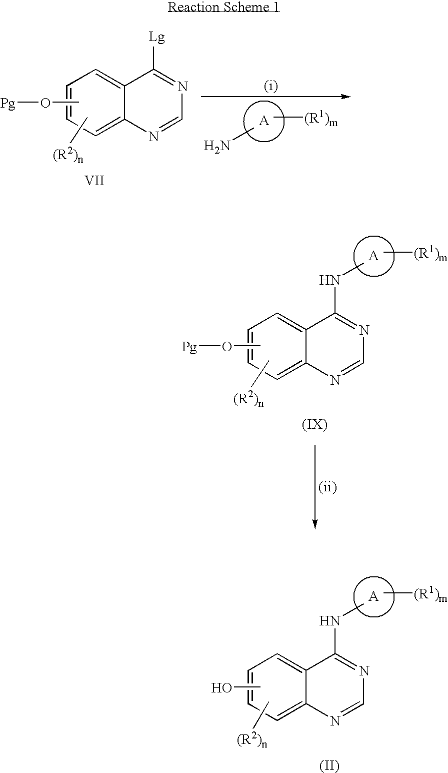
wherein R¹, R², m and n are as hereinbefore defined and Pg is a hydroxy protecting group.
(i) Reaction suitably in an inert protic solvent (such as an alkanol for example iso-propanol), an aprotic solvent (such as dioxane) or a dipolar aprotic solvent (such as N,N-dimethylacetamide) in the presence of an acid, for example hydrogen chloride gas in diethyl dimethylacetamide) in the presence of an acid, for example hydrogen chloride gas in diethyl ether or dioxane, or hydrochloric acid, under analogous conditions to those described above under process (i).

Alternatively the reaction may be carried out in one of the above inert solvents conveniently in the presence of a base, for example potassium carbonate. The above reactions are conveniently carried out at a temperature in the range, for example, 0 to 150° C., suitably at or near the reflux temperature of the reaction solvent.
(ii) Cleavage of Pg may be performed under standard conditions for such reactions. For example when Pg is an alkanoyl group such as acetyl, it may be cleaved by heating in the presence of a methanolic ammonia solution.

Compounds of formula Stat are known or can be prepared using known processes for the preparation of analogous compounds If not commercially available, compounds of the formula (VIE) may be prepared by procedures which are selected from standard chemical techniques, techniques which are analogous to the synthesis of known, structurally similar compounds, or techniques which are analogous to the procedures described in the Examples. For example, standard chemical techniques are as described in Houben Weyl. By way of example the compound of the formula VIII in which R⁴—is methoxy and in the 7-position of the quinolone ring, Lg is chloro and Pg is acetyl may be prepared using the process illustrated in Reaction Scheme 2:

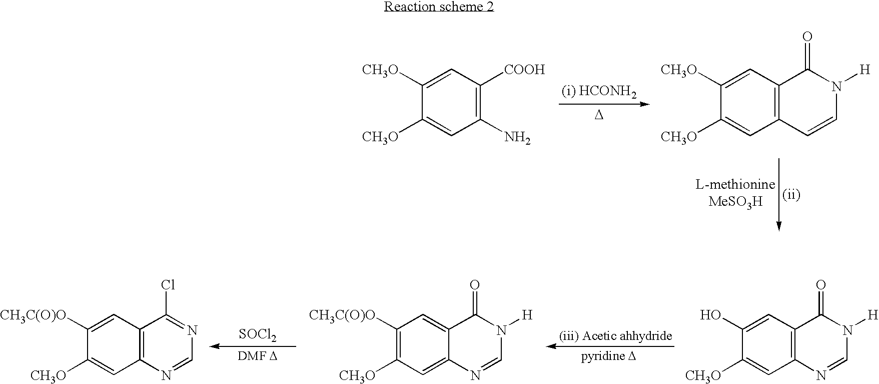

Reaction Scheme 2 may be generalised by the skilled man to apply to compounds within the present specification which are not specifically illustrated (for example to introduce a substituent other than methoxy at the 7-position in the quinazoline ring).

Compounds of the Formula III are commercially available or may be prepared using standard techniques, for example as illustrated in U.S. Pat. No. 5,252,586 and WO 94/27965.

Compounds of the Formula IV may be prepared using process (e) above, starting with a compound prepared, for example using Process (a).

Compounds of the formula V may be prepared by hydrolysing the corresponding carboxylic ester. The carboxylic ester may be formed for example using similar processes to process (a) or process (d) from the appropriate carboxylic ester starting materials.

Compounds of the formula VI may be prepared using conventional methods well known in the art. For example the hydroxy protecting group, Pg, in a compound of the formula VIII as hereinbefore described in Reaction Scheme 1 is removed to give the compound of the formula X: 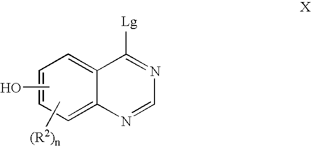
The protecting group Pg may be removed from the compound of formula X using conventional techniques.

The compound of the formula X may then be coupled with a compound of the Formula III as hereinbefore defined using analogous conditions to those described in Process (a) or Process (d).

Certain novel intermediates utilised in the above processes are provided as a further feature of the present invention together with the process for their preparation.

Biological Assays

The following assays may be used to measure the effects of the compounds of the present invention as inhibitors of the erb-tyrosine kinases, as inhibitors in-vitro of the proliferation of KB cells (human naso-pharangeal carcinoma cells) and as inhibitors in vivo on the growth in nude mice of xenografts of LoVo tumour cells (colorectal adenocarcinoma).

a) Protein Tyrosine Kinase Phosphorylation Assays

This test measures the ability of a test compound to inhibit the phosphorylation of a tyrosine containing polypeptide substrate by EGFR tyrosine kinase enzyme.

Recombinant intracellular fragments of EGFR, erbB2 and erbB4 (accession numbers X00588, X03363 and L07868 respectively) were cloned and expressed in the baculovirus/Sf1 system Lysates were prepared from these cells by treatment with ice-cold lysis buffer (20 mM N-2-hydroxyethylpiperizine-N′-2-ethanesulfonic acid (HEPES) pH7.5, 150 mM NaCl, 10% glycerol, 1% Triton X-100, 1.5mM MgCl₂, 1 mM ethylene glycol-bis(β-ammoethyl ether) N′,N′,N′,N′-tetraacetic acid (EGTA), plus protease inhibitors and then cleared by centrifugation.

Constitutive kinase activity of the recombinant protein was determined by its ability to phosphorylate a synthetic peptide (made up of a random co-polymer of Glutamic Acid, Alanine and Tyrosine in the ratio of 6:3:1). Specifically, Maxisorb™ 96-well immunoplates were coated with synthetic peptide (0.2 μg of peptide in a 100 μl phosphate buffered saline (PBS) solution and incubated at 4° C. overnight). Plates were washed in PBS-T (phosphate buffered saline with 0.5% Tween 20) then in 50 mM HEPES pH 7.4 at room temperature to remove any excess unbound synthetic peptide. EGER, ErbB2 or ErbB4 tyrosine kinase activity was assessed by incubation in peptide coated plates for 20 minutes at 22° C. in 100 mM HEPES pH 7.4, adenosine trisphosphate (ATP) at Km concentration for the respective enzyme, 10 mM MnCl₂, 0.1 mM Na₃VO₄, 0.2 mM DL-dithiothreitol (DTT), 0.1% Triton X-100 with test compound in DMSO (final concentration of 2.5%). Reactions were terminated by the removal of the liquid components of the assay followed by washing of the plates with PBS-T.

The immobilised phospho-peptide product of the reaction was detected by immunological methods. Firstly, plates were incubated for 90 minutes at room temperature with ant-phosphotyrosine primary antibodies that were raised in the mouse (4G10 from Upstate Biotechnology). Following extensive washing, plates were treated with Horseradish Peroxidase (HRP) conjugated sheep anti-mouse secondary antibody (NXA931 from Amersham) for 60 minutes at room temperature. After further washing, HRP activity in each well of the plate was measured calorimetrically using 22′-Azino-di-[3-ethylbenzthiazoline sulfonate (6)] diammonium salt crystals (ABTS™ from Roche) as a substrate. Quantification of colour development and thus enzyme activity was achieved by the measurement of absorbance at 405 nm on a Molecular Devices ThermoMax microplate reader. Kinase inhibition for a given compound was expressed as an IC₅₀ value. This was determined by calculation of the concentration of compound that was required to give 50% inbition of phosphorylation in this assay. The range of phosphorylation was calculated from the positive (vehicle plus ATP) and negative (vehicle minus ATP) control values.

b) EGFR Driven KB Cell Proliferation Assay

This assay measures the ability of a test compound to inhibit the proliferation of KB cells (human naso-pharangeal carcinoma obtained from the American Type Culture Collection (ATCC).

KB cells (human naso-pharangeal carcinoma obtained from the ATCC were cultured in Dulbecco's modified Eagle's medium (DMEM) containing 10% foetal calf serum, 2 mM glutamine and non-essential amino acids at 37° C. in a 7.5% CO₂ air incubator. Cells were harvested from the stock flasks using Trypsin/ethylaminediaminetetraacetic acid (EDTA). Cell density was measured using a haemocytometer and viability was calculated using trypan blue solution before being seeded at a density of 1.25×10³ cells per well of a 96 well plate in DMEM containing 2.5% charcoal stripped serum, 1 mM glutamine and non-essential amino acids at 37° C. in 7.5% CO₂ and allowed to settle for 4 hours.

Following adhesion to the plate, the cells are treated with or without EGF (final concentration of 1 ng/ml) and with or without compound at a range of concentrations in dimethylsulfoxide (DMSO) (0.1% fial) before incubation for 4 days. Following the incubation period, cell numbers were determined by addition of 50 μl of 3-(4,5-Dimethylthiazol-2-yl)-2,5-diphenyltetrazolium bromide (MTT) (stock 5 mg/ml) for 2 hours. MTT solution was then tipped off, the plate gently tapped dry and the cells dissolved upon the addition of 100 μl of DMSO.

Absorbance of the solubilised cells was read at 540 nm using a Molecular Devices ThermoMax microplate reader. Inhibition of proliferation was expressed as an IC₅₀ value. This was determined by calculation of the concentration of compound that was required to give 50% inhibition of proliferation. The range of proliferation was calculated from the positive (vehicle plus EGF) and negative (vehicle minus EGF) control values.

c) H16N-2 Cell Proliferation Assay

This assay measures the ability of a test compound to inhibit heregulin β or EGF driven proliferation of H16N-2 cells. These non-neoplastic eptihelial cells respond in a proliferative manner to stimulation with either EGF or heregulin β (Ram, G. R. and Ethier, S. P.(1996) Cell Growth and Differentiation, 7, 551-561) were isolated human mammary tissue (Band, V. and Sager, R. Tumour progression in breast cancer. In: J. S. Rhim and A. Dritschilo (eds.), Neoplastic Transformation in human Cell Culture, pp 169-178. Clifton, N.J.: Humana Press, 1991) and were obtained from the Dana-Farber Cancer Institute, 44 Binney Street, Boston, Mass. 02115.

H16N-2 cells were routinely cultured in culture medium (a 1:1 mix of Gibco F12 and Ham's αMBM media containing 1% foetal calf serum, 10 mM HEPES, 1 μg/ml Insulin, 12.5 ng/ml EGF, 2.8 μM Hydrocortisone, 2 nM Estradiol 5 μM Ascorbic Acid, 10 μg/ml Transferrin, 0.1 mM Phosphoethanolamine, 15 nM Sodium Selenite, 2 mM Glutamine, 10 nM Tri-iodo-thrynoine, 35 μg/ml Bovine pituitary Extract and 0.1 mM Ethanolamine) at 37° C. in a 7.5% CO₂ air incubator. Cells were harvested from the stock flasks using Trypsin/ethylaminediaminetetraacetic acid (EDTA). Cell density was measured using a haemocytometer and viability was calculated using trypan blue solution before being seeded at a density of 1.0×10³ cells per well of a 96 well plate in the above media at 37° C. in 7.5% CO₂ and allowed to settle for 72 hours.

Following this, the cells were starved of serum for 24 hours upon the addition of starvation medium (a 1:1 mix of Gibco F12 and Ham's αMEM media containing, 10 mM HEPES, 2 nM Estradiol, 5 μM Ascorbic Acid, 10 μg/ml Transferrin, 0.1 mM Phosphoethanolamine, 15 nM Sodium Selenite, 2 mM Glutamine, and 0.1 mM Ethanolamine) and incubated at 37° C. in 7.5% CO₂. The cells were then treated with or without compound at a range of concentrations in dimethylsulphoxide (DMSO) (1% final) for two hours before the addition of exogenous ligand (at a final concentration of 100 ng/ml of heregulin or β or 5 ng/ml of EGF) and incubation with both ligand and compound for 4 days at 37° C. in 7.5% CO₂. Following the incubation period, cell numbers were determined by removal of the media by aspiration and incubating with 50 μl of 3-(4,5-Dimethylthiazol-2-yl)-2, 5-diphenyltetrazolium bromide (MTT) (stock 5 mg/ml) for 2 hours. MTT solution was then removed by aspiration, allowed to air dry and the cells dissolved upon the addition of 100 μl of DMSO.

Absorbance of this solubilised cells was read at 540 nm to quantify cell biomass. Inhibition of proliferation was expressed as an IC₅₀ value. This was determined by calculation of the concentration of compound that was required to give 50% inhibition of proliferation. The range of proliferation was calculated from the positive (vehicle plus ligand) and negative (vehicle minus ligand) control values.

d) In vivo Xenograft Assay

This assay measures the ability of a test compound to inhibit the growth of a LoVo tumour (colorectal adenocarcinoma obtained from the ATCC) in Female Swiss athymic mice (Alderley Park, nu/nu genotype).

Female Swiss athymic (nu/nu genotype) mice were bred and maintained in Alderley Park in negative pressure Isolators (PFI Systems Ltd.). Mice were housed in a barrier facility with 12 hr light/dark cycles and provided with sterilised food and water ad libitum. All procedures were performed on mice of at least 8 weeks of age. LoVo tumour cell (colorectal adenocarcinoma obtained from the ATCC) xenografts were established in the hind flank of donor mice by sub cutaneous injections of 1×10⁷ freshly cultured cells in 100 μl of serum free media per animal. On day 5 post-implant, mice were randomised into groups of 7 prior to the treatment with compound or vehicle control that was administered once daily at 0.1 ml/10 g body weight. Tumour volume was assessed twice weekly by bilateral Vernier caliper measurement, using the formula (length×width)×√(length×width)×(π/6), where length was the longest diameter across the tumour, and width was the corresponding perpendicular. Growth inhibition from start of study was calculated by comparison of the mean changes in tumour volume for the control and treated groups, and statistical significance between the two groups was evaluated using a Students t test.

e) hERG-Encoded Potassium Channel Inhibition Assay

This assay determines the ability of a test compound to inhibit the tail current flowing through the human ether-a-go-go -related-gene (hERG)-encoded potassium channel.

Human embryonic kidney (HEK) cells expressing the hERG-encoded channel were grown in Minimum Essential Medium Eagle (EMEM; Sigma-Aldrich catalogue number M2279), supplemented with 10% Foetal Calf Serum (Labtech International; product number 4-101-500), 10% M1 serunmfree supplement (Egg Technologies; product number 70916) and 0.4 mg/ml Geneticin G418 (Sigma-Aldrich; catalogue number G7034). One or two days before each experiment, the cells were detached from the tissue culture flasks with Accutase (TCS Biologicals) using standard tissue culture methods. They were then put onto glass coverslips resting in wells of a 12 well plate and covered with 2 ml of the growing media

For each cell recorded, a glass coverslip containing the cells was placed at the bottom of a Perspex chamber containing bath solution (see below) at room temperature (˜20° C.). This chamber was fixed to the stage of an inverted, phase-contrast microscope. Immediately after placing the coverslip in the chamber, bath solution was perfused into the chamber from a gravity-fed reservoir for 2 minutes at a rate of ˜2 ml/min. After this time, perfusion was stopped.

A patch pipette made from borosilicate glass tubing (GC120F, Harvard Apparatus) using a P-97 micropipette puller (Sutter Instrument Co.) was filled with pipette solution (see hereinafter). The pipette was connected to the headstage of the patch clamp amplifier (Axopatch 200B, Axon Instruments) via a silver/silver chloride wire. The headstage ground was connected to the earth electrode. This consisted of a silver/silver chloride wire embedded in 3% agar made up with 0.85% sodium chloride.

The cell was recorded in the whole cell configuration of the patch clamp technique. Following “break-in”, which was done at a holding potential of −80 mV (set by the amplifier), and appropriate adjustment of series resistance and capacitance controls, electrophysiology software (Clampex, Axon Instruments) was used to set a holding potential (−80 mV) and to deliver a voltage protocol. This protocol was applied every 15 seconds and consisted of a 1 s step to +40 mV followed by a 1 s step to −50 mV. The current response to each imposed voltage protocol was low pass filtered by the amplifier at 1 kHz. The filtered signal was then acquired, on line, by digitising this analogue signal from the amplifier with an analogue to digital converter. The digitised signal was then captured on a computer running Clampex software (Axon Instruments). During the holding potential and the step to +40 mV the current was sampled at 1 kHz. The sampling rate was then set to 5 kHz for the remainder of the voltage protocol.

The compositions, pH and osmolarity of the bath and pipette solution are tabulated below.

| Salt | Pipette (mM) | Bath (mM) |
|---|---|---|
| NaCl | — | 137 |
| KCl | 130 | 4 |
| MgCl₂ | 1 | 1 |
| CaCl₂ | — | 1.8 |
| HEPES | 10 | 10 |
| glucose | — | 10 |
| Na₂ATP | 5 | — |
| EGTA | 5 | — |

| Parameter | Pipette | Bath |
|---|---|---|
| pH | 7.18-7.22 | 7.40 |
| pH adjustment with | 1M KOH | 1M NaOH |
| Osmolarity (mOsm) | 275-285 | 285-295 |

The amplitude of the hERG-encoded potassium channel tail current following the step from +40 mV to −50 mV was recorded on-line by Clampex software (Axon Instruments). Following stabilisation of the tail current amplitude, bath solution containing the vehicle for the test substance was applied to the cell. Providing the vehicle application had no significant effect on tail current amplitude, a cumulative concentration effect curve to the compound was then constructed.

The effect of each concentration of test compound was quantified by expressing the tail current amplitude in the presence of a given concentration of test compound as a percentage of that in the presence of vehicle.

Test compound potency (IC₅₀) was determined by fitting the percentage inhibition values making up the concentration-effect to a four parameter Hi]] equation using a standard data-fitting package. If the level of inhibition seen at the highest test concentration did not exceed 50%, no potency value was produced and a percentage inhibition value at that concentration was quoted.

Although the pharmacological properties of the compounds of the Formula I vary with structural change as expected, in general activity possessed by compounds of the Formula I, may be demonstrated at the following concentrations or doses in one or more of the above tests (a), (b) and (c):

Test (a):—IC₅₀ in the range, for example, 0.001-10 μM;

Test (b):—IC₅₀ in the range, for example, 0.001-10 μM;

Test (c):—IC₅₀ in the range, for example, 0.001-10 μM;

Test (d):—activity in the range, for example, 1-200 mg/kg/day;

No physiologically unacceptable toxicity was observed in Test (c) at the effective dose for compounds tested of the present invention. Accordingly no untoward toxicological effects are expected when a compound of Formula I, or a pharmaceutically-acceptable salt thereof, as defined hereinbefore is administered at the dosage ranges defined hereinafter.

By way of example, using Test (a) (for the inhibition of EGFR tyrosine kinase protein phosphorylation) and Test (b), the KB cell assay described above, representative compounds described in the Examples herein gave the IC₅₀ results shown below in Table B:

TABLE B

| | IC₅₀ (nM) Test (a) | |
|---|---|---|
| | (Inhibition of EGFR | IC₅₀ (nM) Test (b) |
| Example | tyrosine kinase protein | (EGFR driven KB cell |
| (Compound No.) | phosphorylation) | proliferation assay) |
| 11 (13) | 0.008 | 0.144 |
| 11 (14) | 0.010 | 0.139 |
| 13 (3) | 0.012 | 0.063 |

According to a further aspect of the invention there is provided a pharmaceutical composition which comprises a quinazoline derivative of the Formula I, or a pharmaceutically-acceptable thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the conditions, the age and sex of the animal or patient and the route of administration, according to well known principles of medicine.

In using a compound of the Formula I for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration is however preferred, particularly in tablet form. Typically, unit dosage forms win contain about 0.5 mg to 0.5 g of a compound of this invention.

We have found that the compounds of the present invention possess anti-proliferative properties such as anti-cancer properties that are believed to arise from their erbB family receptor tyrosine kinase inhibitory activity, particularly inhibition of the EGF receptor (erbB1) tyrosine kinase. Furthermore, certain of the compounds according to the present invention possess substantially better potency against the EGF receptor tyrosine kinase, than against other tyrosine kinase enzymes, for example erbB2. Such compounds possess sufficient potency against the EGF receptor tyrosine kinase that they may be used in an amount sufficient to inhibit EGF receptor tyrosine kinase whilst demonstrating little, or significantly lower, activity against other tyrosine kinase enzymes such as erbB2. Such compounds are likely to be useful for the selective inhibition of EGF receptor tyrosine kinase and are likely to be useful for the effective treatment of, for example EGF driven tumours.

Accordingly, the compounds of the present invention are expected to be useful in the treatment of diseases or medical conditions mediated alone or in part by erbB receptor tyrosine kinases (especially EGF receptor tyrosine kinase), i.e. the compounds may be used to produce an erbB receptor tyrosine kinase inhibitory effect in a warmblooded animal in need of such treatment. Thus the compounds of the present invention provide a method for the treatment of malignant cells characterised by inhibition of one or more of the erbB family of receptor tyrosine kinases. Particularly the compounds of the invention may be used to produce an anti-proliferative and/or pro-apoptotic and/or anti-invasive effect mediated alone or in part by the inhibition of erbB receptor tyrosine kinases. Particularly, the compounds of the present invention are expected to be useful in the prevention or treatment of those tumours that are sensitive to inhibition of one or more of the erbB receptor tyrosine kinases, such as EGF and/or erbB2 and/or erbB4 receptor tyrosine kinases (especially BGF receptor tyro sine kinase) that are involved in the signal transduction steps which drive proliferation and survival of these tumour cells. Accordingly the compounds of the present invention are expected to be useful in the treatment of psoriasis, benign prostatic hyperplasia (BPH), atherosclerosis and restenosis and/or cancer by providing an anti-proliferative effect, particularly in the treatment of erbB receptor tyrosine kinase sensitive cancers. Such benign or malignant tumours may affect any tissue and include non-solid tumours such as leukemia, multiple myeloma or lymphoma, and also solid tumours, for example bile duct, bone, bladder, brain/CNS, breast, colorectal, endometrial gastric, head and neck, hepatic, lung, neuronal, oesophageal, ovarian, pancreatic, prostate, renal, skin, testicular, thyroid, uterine and vulval cancers.

According to this aspect of the invention there is provided a compound of the Formula I, or a pharmaceutically acceptable salt thereof, for use as a medicament.

According to a further aspect of the invention there is provided a compound of the Formula I, or a pharmaceutically acceptable salt thereof, for use in the production of an anti-proliferative effect in a warm-blooded animal such as man.

Thus according to this aspect of the invention there is provided the use of a quinazoline derivative of the Formula I, or a pharmaceutically-acceptable salt thereof, as defined hereinbefore in the manufacture of a medicament for use in the production of an anti-proliferative effect in a warm-blooded animal such as man.

According to a further feature of this aspect of the invention there is provided a method for producing an anti-proliferative effect in a warm-blooded animal, such as man, in need of such treatment which comprises administering to said animal an effective amount of a quinazoline derivative of the Formula I, or a pharmaceutically acceptable salt thereof, as hereinbefore defined.

According to a further aspect of the invention there is provided the use of a quinazoline derivative of the Formula I, or a pharmaceutically-acceptable salt thereof, as defined hereinbefore in the manufacture of a medicament for use in the prevention or treatment of those tumours which are sensitive to inhibition of erbB receptor tyrosine kinases, such as EGFR and/or erbB2 and/or erbB4 (especially EGFR), that are involved in the signal transduction steps which lead to the proliferation of tumour cells.

According to a further feature of this aspect of the invention there is provided a method for the prevention or treatment of those tumours which are sensitive to inhibition of one or more of the erbB family of receptor tyrosine kinases, such as EGFR and/or erbB2 and/or erbB4 (especially EGFR), that are involved in the signal transduction steps which lead to the proliferation and/or survival of tumour cells which comprises administering to said animal an effective amount of a quinazoline derivative of the Formula I, or a pharmaceutically-acceptable salt thereof, as defined hereinbefore.

According to a further feature of this aspect of the invention there is provided a compound of the Formula I, or a pharmaceutically acceptable salt thereof, for use in the prevention or treatment of those tumours which are sensitive to inhibition of erbB receptor tyrosine kinases, such as EGFR and/or erbB2 and/or erbB4 (especially EGFR), that are involved in the signal transduction steps which lead to the proliferation of tumour cells.

According to a further aspect of the invention there is provided the use of a quinazoline derivative of the Formula I, or a pharmaceutically-acceptable salt thereof, as defined hereinbefore in the manufacture of a medicament for use in providing a EGFR and/or erbB2 and/or erbB4 (especially a EGER) tyrosine kinase inhibitory effect.

According to a further feature of this aspect of the invention there is provided a method for providing a EGFR and/or an erbB2 and or an erbB4 (especially a EGFR) tyrosine kinase inhibitory effect which comprises administering to said animal an effective amount of a quinazoline derivative of the Formula I, or a pharmaceutically-acceptable salt thereof, as defined hereinbefore.

According to a further feature of this aspect of the invention there is provided a compound of the Formula I, or a pharmaceutically acceptable salt thereof, for use in providing a EGFR and/or erbB2 and/or erbB4 (especially a EGFR) tyrosine kinase inhibitory effect.

According to a further feature of the present invention there is provided the use of a quinazoline derivative of the Formula I, or a pharmaceutically-acceptable salt thereof, as defined hereinbefore in the manufacture of a medicament for use in providing a selective EGFR tyrosine kinase inhibitory effect.

According to a further feature of this aspect of the invention there is provided a method for providing a selective EGFR tyrosine kinase inhibitory effect which comprises administering to said animal an effective amount of a quinazoline derivative of the Formula I, or a pharmaceutically-acceptable salt thereof, as defined hereinbefore.

According to a further feature of this aspect of the invention there is provided a compound of the Formula I, or a pharmaceutically acceptable salt thereof, for use in providing a selective EGER tyrosine kinase inhibitory effect.

By “a selective EGFR kinase inhibitory effect” is meant that the quinazoline derivative of Formula I is more potent against EGF receptor tyrosine kinase than it is against other kinases. In particular some of the compounds according to the invention are more potent against EGF receptor kinase than it is against other tyrosine kinases such as other erbB receptor tyrosine kinases such erbB2. For example a selective EGFR kinase inhibitor according to the invention is at least 5 times, preferably at least 10 times more potent against erbB2 receptor tyrosine kinase driven proliferation than it is against BGFR tyrosine kinase driven proliferation, as determined from the relative IC₅₀ values in a suitable assay (for example the H116N-2 assay described above).

According to a further aspect of the present invention there is provided the use of a quinazoline derivative of the Formula I, or a pharmaceutically-acceptable salt thereof, as defined hereinbefore in the manufacture of a medicament for use in the treatment of a cancer (for example a cancer selected from leukemia, multiple myeloma, lymphoma, bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancer).

According to a further feature of this aspect of the invention there is provided a method for treating a cancer (for example a cancer selected from leukemia, multiple myeloma, lymphoma, bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancer) in a warmblooded animal, such as man, in need of such treatment, which comprises administering to said animal an effective amount of a quinazoline derivative of the Formula I, or a pharmaceutically-acceptable salt thereof, as defined hereinbefore.

According to a further aspect of the invention there is provided a compound of the Formula I, or a pharmaceutically acceptable salt thereof, for use in the treatment of a cancer (for example selected from leukemia, multiple myeloma, lymphoma, bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancer).

As mentioned above the size of the dose required for the therapeutic or prophylactic treatment of a particular disease will necessarily be varied depending upon, amongst other things, the host treated, the route of administration and the severity of the illness being treated.

The anti-proliferative treatment defined hereinbefore may be applied as a sole therapy or may involve, in addition to the quinazoline derivative of the invention, conventional surgery or radiotherapy or chemotherapy. Such chemotherapy may include one or more of the following categories of anti-tumour agents :
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremnifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marinastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™ ] and the anti-erbb1 antibody cetuximab [C225]), farnesyl transferase inhibitors, tyrosine kinase inhibitors and serine/threonine kinase inhibitors, for example other inhibitors of the epidermal growth factor family (for example EGFR family tyro sine kinase inhibitors such as N-(3-chloro-4-fluorophenyl)-7-methoxy-6-(3-morpholinopropoxy)quinazolin-4amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a pharmaceutical product comprising a quinazoline derivative of the Formula I as defined hereinbefore and an additional anti-tumour agent as defined hereinbefore for the conjoint treatment of cancer.

Although the compounds of the Formula I are primarily of value as therapeutic agents for use in warm-blooded animals (including man), they are also useful whenever it is required to inhibit the effects of the erbB receptor tyrosine protein kinases. Thus, they are useful as pharmacological standards for use in the development of new biological tests and in the search for new pharmacological agents.

The invention will now be illustrated by the following examples in which, unless stated otherwise:
(i) temperatures are given in degrees Celsius (° C); operations were carried out at room or ambient temperature, that is, at a temperature in the range of 18-25° C.;
(ii) organic solutions were dried over anhydrous magnesium sulf ate; evaporation of solvent was carried out using a rotary evaporator under reduced pressure (600-4000 Pascals; 4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and/or analytical LCMS, and reaction times are given for illustration only;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (UMS) as an internal standard, determined at 300 MHz or 400 MHz using perdeuterio dimethyl sulfoxide (DMSO-d₆) as solvent unless otherwise indicated; the following abbreviations have been used: s, singlet; d, doublet; t, triplet; q, quartet; m, multiplet; b, broad;
(viii) chemical symbols have their usual meanings; SI units and symbols are used;
(ix) solvent ratios are given in volume:volume (v/v) terms; and
(x) mass spectra (MS) were run using a Waters or Micromass electrospray LC-MS in positive or negative ion mode; values for m/z are given; generally, only ions which indicate the parent mass are reported; and unless otherwise stated, the mass ion quoted is (MH)⁺;
(xi) where compounds were purified using Mass-Triggered Preparative LCMS, one of the following sets of conditions was utilised:
LCMS Conditions 1
   Column: Waters ‘Xterra’ (5 microns silica, 19 mm diameter, 100 mm length) Solvent flow rate: 25 ml/min;

Solvent composition: the solvent contained water (A), acetonitrile (B) and 36% ammonia in acetonitrile (C); over a 7.5 minute run cycle the proportion of A in the solvent gradually reduces, the proportion of B gradually increases and the proportion of C remains constant; details of the solvent composition at 0, 1 and 7.5 minutes are shown in Table C below:

TABLE C

| Time (mins) | A (%) | B (%) | C (%) |
|---|---|---|---|
| 0 | 94 | 1 | 5 |
| 1 | 55 | 40 | 5 |
| 7.50 | 20 | 75 | 5 |

[C is added at a constant flow of 5% of the total solvent flow rate];
   LCMS Conditions 2
Column: Phenomenex Synergi Max -RP (50×2.0 mm, 4 μm, 80 A pore size);
Injection Volume: 5μL, a partial loop fill of a 50 μL loop using 2.5 μL air gaps;
Solvent flow rate: 1.1 ml/min;

Solvent composition: the solvent contained water (A), acetonitrile (B) and 50:50 acetonitrile:water, 1% v/v formic acid (C); over an initial 4 minute period of the run cycle the proportion of A in the solvent is reduced, the proportion of B is increased and the proportion of C remains constant; details of the solvent composition at 0, 4 minutes are shown in Table D below:

TABLE D

| Time (mins) | A (%) | B (%) | C (%) |
|---|---|---|---|
| 0 | 95 | 0 | 5 |
| 4 | 57.5 | 37.5 | 5 |

Instrumentation: Waters alliance 2790 quaternary pump and autosampler; Waters ZMD single quadrupole Mass Spectrometer and Waters 996 PDA (Scanning 190-320 nm, displaying 254 nm only);

Operating system. Masslynx 3.5 running Openlynx on Windows NT 4.0 LC; the retention times (in minutes) and the set of conditions utilized are stated below for the exemplified compounds that were purified by Mass-Triggered Preparative LCMS;
(xii) unless stated otherwise compounds containing an asynmetrically substituted carbon and/or sulfur atom have not been resolved;
(xiii) where a synthesis is described as being analogous to that described in a previous example the amounts used are the millimolar ratio equivalents to those used in the previous example;
(xiv) the following abbreviations have been used:
DMF N,N-dimethylformide;
DMA N,N-dimethylacetamide;
THF tetrahydrofuran;
DIPEA diisopropylethylamine
HATU O-(7-azabenzotiiazol-1-yl)-N,N,N′,N′-tetramethyluronium hexafluorophosphate
xv) where a synthesis is described as leading to an acid addition salt (e.g. HCl salt), the specific stoichiometry of the salt was not confirmed.

### EXAMPLE 1

### (4S)-4-({4-[(3-Chloro-2-fluorophenyl)amino]-7-methoxyquinazolin-6-yl}oxy)-N-cyclolpropyl-1-methyl-D-prolinamide

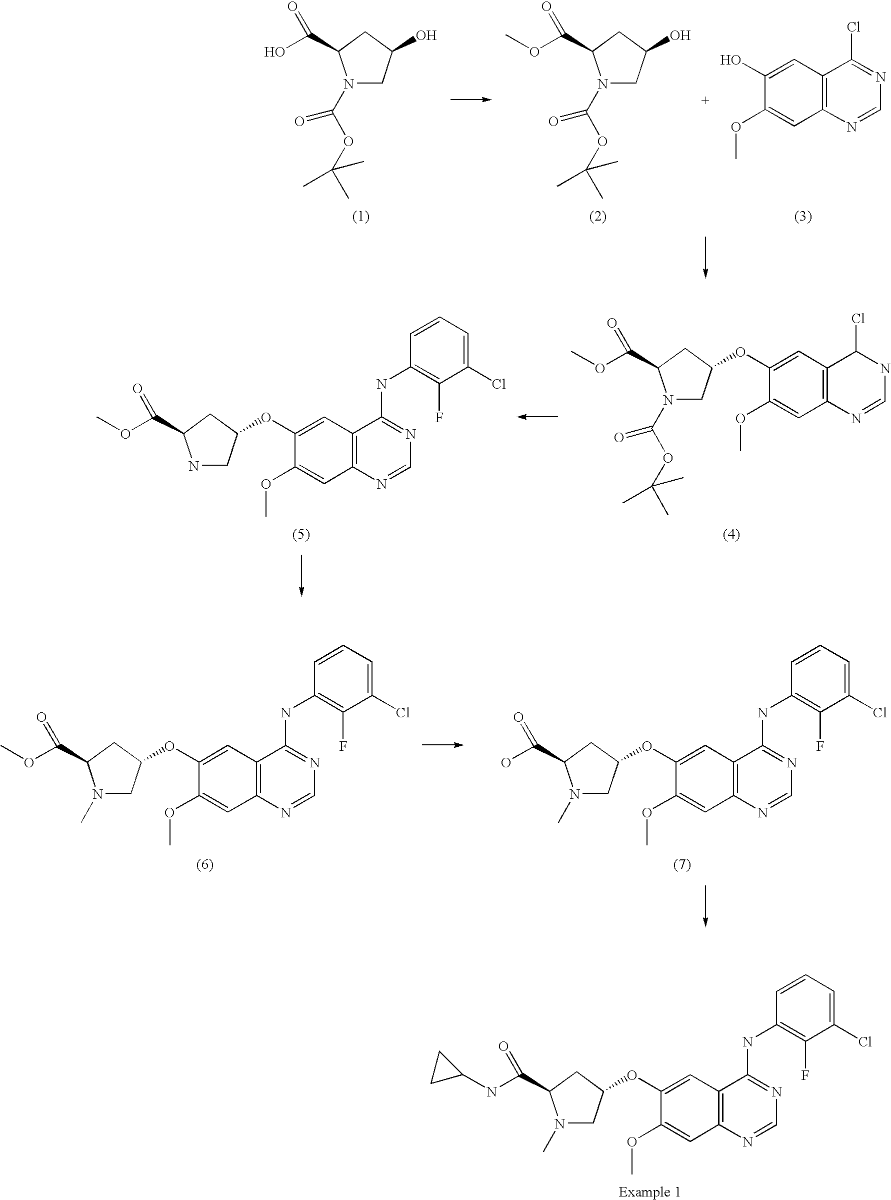

HATU (0.23 g) was added to an agitated solution of (4S)-4-({4-[(3-chloro-2-fluorophenyl)amino]-7-methoxyquinazolin-6-yl}oxy)-1-methyl-D-proline (7) (0.18 g), cyclopropylamine (34.4 mg) and DIPEA (156 mg) in methylene chloride (5 ml). After 16 hrs the reaction mixture was reduced in vacuo. The residues were re-dissolved in methylene chloride and washed with sodium hydroxide solution (2M) and water. The organic phase was then purified by column chromatography on silica eluting with increasingly polar mixtures of methanol/methylene chloride (0/100-10/90). The fractions containing the desired product were combined and evaporated to a foam which was triturated with diethylether to give the title compound as a white solid. (0.15 g). ¹H NMR Spectrum: (DMSO d₆) 0.40-0.48 (m, 2H), 0.57-0.64 (m, 2H), 2.05-2.14 (m, 1H), 2.28 (s, 3H), 2.33-2.45 (m, 1H), 2.48-2.56 (m, 1H+DMSO), 2.61-2.70 (m, 1H), 3.08 (t, 1H), 3.64 (dd, 1H), 3.94 (s, 3H), 5.06 (m, 1H), 7.20 (s, 1H), 7.28 (t, 1H), 7.44-7.56 (m, 2H), 7.65 (s, 1H), 7.87 (d, 1H), 8.35 (s, 1H), 9.63 (s, 1H); Mass Spectrum: (M+H)⁺ 486.44

The starting material 1,2-Pyrrolidinedicarboxylic acid, 4-hydroxy-, 1-(1,1-dimethylethyl) 2-methyl ester, (2R,4R) (2) was prepared as follows:

1-[3-(Dimethylamino)propyl]-3-ethylcarbodiimide hydrochloride (14.73 g) was added to a stirred suspension of 1,2-pyrrolidinedicarboxylic acid, 4-hydroxy-, 1-(1,1-dimethylethyl)ester, (2R,4R) (1) (13.65 g), dimethylaminopyridine (21.65 g) and methanol (5.67 g) in methylene chloride (400 ml) and the reaction mixture was stirred at room temperature for 16 hours. The reaction mixture was washed with citric acid (1.0 M), saturated aqueous sodium bicarbonate solution and saturated brine, dried over MgSO₄, filtered and evaporated. The residues were then purified by column chromatography on silica eluting with increasingly polar mixtures of methanol/methylene chloride (1/99-5/95). The desired product fractions were combined and evaporated to give 1,2-pyrrolidinedicarboxylic acid, 4-hydroxy-, 1-(1,1-dimethylethyl)2-methyl ester, (2R,4R) (2) as a white crystalline solid, (5.9 g). ¹H NMR Spectrum: (DMSO d₆) 1.32+1.38 (2s, 9H), 1.76-1.87 (m, 1H), 2.24-2.28 (m, 1H), 3.06-3.15 (m, 1H), 3.42-3.51 (m, 1H), 3.60+3.63 (2s, 3H), 4.15-4.24 (m, 2H), 4.92-5.00 (m, 1H).

Starting material 1,2-Pyrrolidinedicarboxylic acid, 4-hydroxy-1-(1,1-dimethylethyl)ester, (2R,4R), (1), (Boc-D-cis-hyp-OH) is commercially available

Starting material (3) was prepared as follows:

6-Acetoxy-4-chloro-7-methoxyquinazoline, (Example 25-5 of in WO01/66099;10.0 g, 39.6 mmole) was added in portions to a stirred 7N methanolic ammonia solution (220 ml) cooled to 10° C. in an ice/water bath. After stirring for one hour the precipitate was filtered, washed with diethylether and dried thoroughly under high vacuum to give 4-chloro-7-methoxyquinazolin-6-ol (3) (5.65 g, 67.8%); ¹H NMR Spectrum: (DMSO d₆) 3.96 (s, 3H); 7.25 (s, 1H); 7.31 (s, 1H); 8.68 (s, 1H); Mass Spectrum: (M+H)⁺ 211.

The starting material (4) was prepared as follows:

Di-ethyl azodicarboxylate (5.71 g) was added slowly to a stirred suspension of 1,2-pyrrolidinedicarboxylic acid, 4-hydroxy-, 1-(1,1-dimethylethyl)2-methyl ester, (2R,4R) (2) (5.9 g), 4-chloro-7-methoxyquinazolin-6-ol (3) (4.6 g) and triphenylphosphine(8.6 g) in methylene chloride (400 ml)at 25° C. under an atmosphere of nitrogen and the reaction mixture was stirred for 2 hours. The reaction mixture was then evaporated to ½ volume and purified by column chromatography on silica eluting with increasingly polar mixtures of methanol/methylene chloride (1/99-3/97). The desired product fractions were combined and evaporated to give 1-tert-butyl 2-methyl(2R,4S)-4-[(4-chloro-7-methoxyquinazolin-6-yl)oxy]pyrrolidine-1,2-dicarboxylate (4) as a pale yellow gum

This was used in the preparation of (5) without further purification.

The starting material methyl(4S)-4-({4-[(3-chloro-2-fluorophenyl)amino]-7-methoxyquinazolin-6-yl}oxy)-D-prolinate hydrochloride (5) was prepared as follows:

4.0M HCl in Dioxane (15 ml) was added to a suspension of 1-tert-butyl 2-methyl(2R,4S)-4-[(4-chloro-7-methoxyquinazolin-6-yl)oxy]pyrrolidine-1,2-dicarboxylate (4) and 3-chloro-2-fluoroaniline (2.89 g) in acetonitrile (400 ml) and the reaction mixture was stirred and heated at 70° C. for 3 hours. The resulting precipitate was filtered hot and washed with acetonitrile and diethylether and dried under vacuum to give methyl(4S)-4-({4-[(3-chloro-2-fluorophenyl)amino]-7-methoxyquinazolin-6-yl}oxy)-D-prolinate hydrochloride (5) as an off-white solid, (6.3 g). ¹H NMR Spectrum: (DMSO d₆) 2.46-2.60 (m, 2H), 3.37-3.46 (m, 1H), 3.71 (s, 3H), 3.89-3.98 (m, 4H), 4.53 (t, 1H), 5.42 (m, 1H), 7.29 (t, 1H), 7.38-7.48 (m, 2H), 7.55 (t, 1H), 8.64 (s, 1H), 8.75 (s, 1H), 12.28 (bs, 1H); Mass Spectrum: (M+H)⁺ 446.96

Compound (6) was prepared as follows:

Methyl(4S)-4-({4-[(3-chloro-2-fluorophenyl)amino]-7-methoxyquinazolin-6-yl}oxy)-D-prolinate hydrochloride (5) (6.3 g), paraformaldehyde (3.9 g), sodium cyanoborohydride (3.28 g) and magnesium sulphate (3.13 g) were suspended in methanol (50 ml) and heated to 45° C. for 4 hours under an atmosphere of nitrogen. The reaction mixture was filtered, evaporated and partitioned between ethylacetate and saturated aqueous sodium bicarbonate solution. The organics were then washed with saturated brine, dried over MgSO₄, filtered and evaporated. The residues were then purified by column chromatography on silica eluting with increasingly polar mixtures of methanol/methylene chloride (2-3%) to give methyl(4S)-4-({4-[(3-chloro-2-fluorophenyl)amino]-7-methoxyquinazolin-6-yl}oxy)-1-methyl-D-prolinate (6) as a yellow foam,(4.19 g). ¹H NMR Spectrum: (DMSO d₆) 2.14-2.23 (m, 1H), 2.34 (s, 3H), 2.53-2.60 (m, 2H), 3.36 (t, 1H), 3.61 (dd, 1H), 3.66 (s, 3H), 3.94 (s, 3H), 5.08 (m, 1H), 7.20 (s, 1H), 7.27 (t, 1H), 7.44-7.54 (m, 2H), 7.68 (s, 1H), 8.36 (s, 1H), 9.62 (s, 1H); Mass Spectrum: (M+H)⁺ 460.9

Compound (7) was prepared as follows:

Sodium hydroxide 2M (7 ml) was added to a stirred solution of methyl(4S)-4-({4-[(3-chloro-2-fluorophenyl)amino]-7-methoxyquinazolin-6-yl}oxy)-1-methyl-D-prolinate (4.18 g) in methanol (20 ml) and THF (10 ml) at 25° C. and the reaction mixture was stirred for 4 hours. The reaction mixture was evaporated and the residue re-dissolved in water. The pH of this solution was then adjusted to 6 by the dropwise addition of 2M HCl (aq) to give (4S)-4-({4-[(3-chloro-2-fluorophenyl)amino]-7-methoxyquinazolin-6-yl}oxy)-1-methyl-D-proline (7) as a pale yellow solid which was filtered and washed with water and dried, (3.5 g). ¹H NMR Spectrum: (DMSO d₆) 2.21-2.31 (m, 1H), 2.35-2.49 (m, 1H), 2.50 (s, 3H), 2.78 (dd, 1H), 3.42 (t, 1H), 3.77 (dd, 1H), 3.94 (s, 3H), 5.08 (m, 1H), 7.20 (s, 1H), 7.27 (t, 1H), 7.44-7.54 (m, 2H), 7.74 (s, 1H), 8.37 (s, 1H), 9.75 (s, 1H); Mass Spectrum: (M+H)⁺ 446.9

### EXAMPLE 2

The following compounds were all made from Compound (7) in a similar manner to that of Example 1 from the appropriate amine hydrochlroride.

TABLE 1

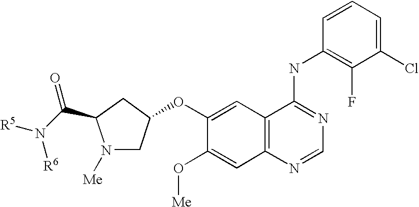

| Compound | R⁵ | R⁶ | Footnote |
|---|---|---|---|
| 1 | H | cyclopropylmethyl | a |
| 2 | H | CH₃OCH₂CH₂— | b |
| 3 | H | cyclopentylmethyl | c |
| 4 | Me | CH₃OCH₂CH₂ | d |
| 5 | H | —OMe | e |
| 6 | H | cyclohexyl | f |
| 7 | H | tetrahydro-2H-pyran-4-yl | g |

a ¹H NMR Spectrum: (DMSO d₆) 0.14-0.20 (m, 2H), 0.37-0.44 (m, 2H), 0.94 (m, 1H), 2.10-2.21 (m, 1H), 2.34 (s, 3H), 2.31-2.44 (m, 1H), 2.56 (dd, 1H), 2.95-3.03 (m, 2H), 3.14 (t, 1H), 3.69 (dd, 1H), 3.94 (s, 3H), 5.08 (m, 1H), 7.23 (s, 1H), 7.29 (t, 1H), 7.45-7.56 (m, 2H), 7.68 (s, 1H), 7.88 (t, 1H), 8.38 (s, 1H), 9.64 (s, 1H); Mass Spectrum: (M + H)⁺ 500.48.
b ¹H NMR Spectrum: (DMSO d₆) 2.10-2.19 (m, 1H), 2.29-2.40 (m, 1H), 2.31 (s, 3H), 2.48-2.58 (m, 1H + DMSO), 3.13 (t,1H), 3.21-3.29 (m, 2H), 3.24 (s, 3H), 3.34-3.38 (m, 2H), 3.68 (dd, 1H), 3.94 (s, 3H), 5.06 (m, 1H), 7.20 (s, 1H), 7.27 (t, 1H), 7.44-7.55 (m, 2H), 7.68 (s, 1H), 7.82 (t, 1H), 8.36 (s, 1H), 9.62 (s, 1H); Mass Spectrum: (M + H)⁺ 504.51.
c ¹H NMR Spectrum: (DMSO d₆) 1.32-1.68 (m, 6H), 1.74-1.86 (m, 2H), 2.06-2.15 (m, 1H), 2.30 (s, 3H), 2.33-2.44 (m, 1H), 2.49-2.57 (m, 1H + DMSO), 3.13 (t, 1H), 3.66 (dd, 1H), 3.94 (s, 3H), 4.02 (q, 1H), 5.08 (m, 1H), 7.20 (s, 1H), 7.29 (t, 1H), 7.45-7.56 (m, 2H), 7.67 (s, 1H), 7.74 (d, 1H), 8.36 (s, 1H), 9.62 (s, 1H); Mass Spectrum: (M + H)⁺ 514.54.
d ¹H NMR Spectrum: (DMSO d₆) 2.07∫2.18(m, 1H), 2.27-2.34 (m, 3H), 2.56-2.67 (m, 1H), 2.83-2.90(m, 4H), 3.21-3.26 (d, 3H), 3.40-3.48 (m, 3H), 3.54-3.88 (m, 3H), 3.93 (s, 3H), 5.10 (m, 1H), 7.20 (s, 1H), 7.27 (t, 1H), 7.44-7.55 (m, 2H), 7.68 (d, 1H), 8.36 (s, 1H), 9.65 (s, 1H); Mass Spectrum: (M + H)⁺ 518.56
e ¹H NMR Spectrum: (DMSO d₆) 2.08-2.17 (m, 1H), 2.33 (s, 3H), 2.40-2.64 (m, 2H + DMSO), 3.08∫3.16 (m, 1H), 3.58-3.70 (m, 1H), 3.60 (s, 3H), 3.94 (s, 3H), 5.08 (m, 1H), 7.20 (s, 1H), 7.27 (t, 1H), 7.44-7.55 (m, 2H), 7.68 (d, 1H), 8.36 (s, 1H), 9.68 (s, 1H), 11.23 (s, 1H); Mass Spectrum: (M + H)⁺ 475.99
f ¹H NMR Spectrum: (DMSO d₆) 1.08-1.34 (m, 6H), 1.60-1.77 (m, 4H), 2.07-2.16 (m 1H), 2.30 (s, 3H), 2.25-2.58 (m, 2H + DMSO), 3.12 (t, 1H), 3.51-3.60 (m, 1H), 3.68 (dd, 1H), 3.92 (s, 3H), 5.08 (m, 1H), 7.20 (s, 1H)(, 7.27 (t, 1H), 7.44-7.55 (m, 2H), 7.61 (d, 1H), 7.67 (s, 1H), 8.37 (s, 1H), 9.64 (s, 1H); Mass Spectrum: (M + H)⁺ 528.63
g ¹H NMR Spectrum: (DMSO d₆) 1.37-1.53 (m, 2H), 1.61-1.70 (m, 2H), 2.08-2.17 (m, 1H), 2.30 (s, 3H), 2.34-2.59 (m, 2H + DMSO), 3.12 (t, 1H), 3.31-3.40 (m, 2H), 3.67 )dd, 1H), 3.75-3.85 (m, 3H), 3.94 (s, 3H), 5.06 (m, 1H), 7.20 (s, 1H), 7.27 (t, 1H), 7.44-7.55 (m, 2H), 7.68 (s, 1H), 7.78 (d, 1H), 8.36 (s, 1H), 9.63 (s, 1H); Mass Spectrum: (M + H)⁺ 530.59.

### EXAMPLE 3

### (4S)-4-({4-[(3-Chloro-4-fluorophenyl)amino]-6-methoxyquinazolin-7-yl}oxy)-N,N,1-trimethyl-L-prolinamide

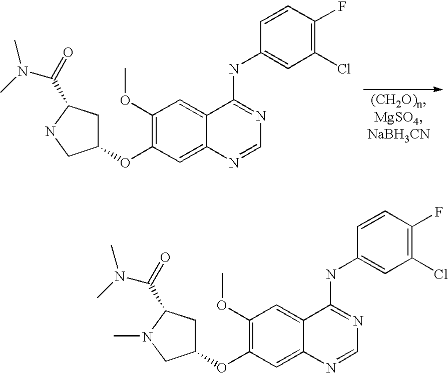

(4S)-4-({4-[(3-Chloro-4-fluorophenyl)amino]-6-methoxyquinazolin-7-yl}oxy)-N,N-dimethyl-L-prolinamide (50 mg, 0.11 mmol) was dissolved in methanol (5 ml) and magnesium sulphate (26 mg, 0.22 mmol), paraformaldehyde (33 mg, 1.09 mmol) and sodium cyanoborohydride (27 mg, 0.44 mmol) added. The mixture was heated at 50° C. for 2 h, cooled, filtered and concentrated under reduced pressure. Column chromatography of the residue (5% 7N ammonia in methanol/dichloromethane) gave (4S)-4-({4-[(3-chloro-4-fluorophenyl)amino]-6-methoxyquinazolin-7-yl}oxy)-N,N,1-trimethyl-L-prolinamide (44 mg, 85%) as a white solid.

¹H NMR spectrum: (DMSO d₆) 1.82 (m, 1H); 2.23 (s, 3H); 2.60 (m, 1H); 2.83 (m, 4H); 3.08 (s, 3H); 3.22 (m, 2H); 3.97 (s, 3H); 5.07 (m, 1H); 7.09 (s, 1H); 7.45 (t, 1H); 7.82 (m, 2H); 8.14 (s, 1H); 8.50 (s, 1H); 9.55 (s, 1H); Mass Spectrum: (M+H)⁺ 474

The starting material was prepared as follows:

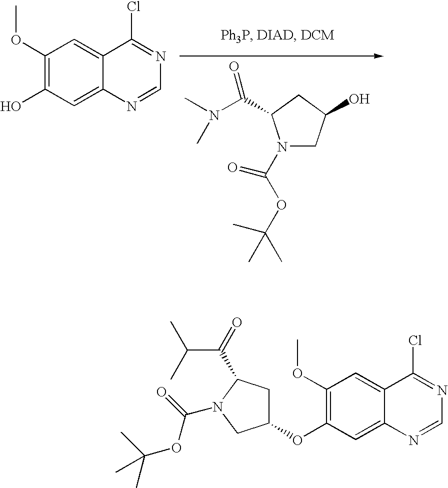

4-Chloro-6-methoxyquinazolin-7-ol (171 mg, 0.81 mmol), (2S,4R)-2-dimethylcarbamoyl-4-hydroxy-pyrrolidine-1-carboxylic add tert-butyl ester (250 mg, 0.97 mmol) and triphenylphosphine (255 mg, 0.97 mmol) were stirred in dichloromethane (12 ml) and diisopropyl azodicarboxylate (191 μl, 0.97 mmol) added. The mixture was stirred at room temperature over night and then concentrated under reduced pressure. Column chromatography eluting with 1:1 ethyl acetate/isohexane gave (2S,4S)-4-(4-chloro-6-methoxy-quinazolin-7-yloxy)-2-dimethylcarbamoyl-pyrrolidine-1-carboxylic acid tert-butyl ester (121 mg, 33%).

¹H NMR spectrum: (DMSO d⁶) 1.35 (m, 9H); 1.87 (m, 1H); 2.85 (m, 3H); 3.00 (m, 4H); 3.39 (m, 1H); 3.95 (s, 3H); 4.08 (m, 1H); 4.65 (m, 1H); 5.27 (m, 1H); 7.40 (s, 1H); 7.55 (s, 1H): 8.89 (s, 1H); Mass Spectrum: (M+Na)⁺ 473.

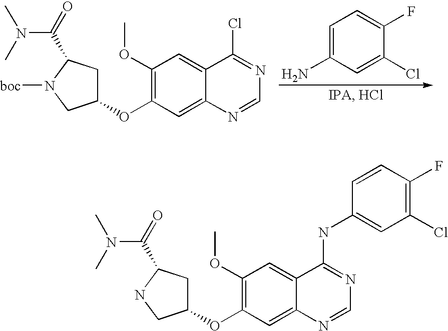

2S,4S)-4-(4-Chloro-6-methoxy-quinazolin-7-yloxy)-2-dimethylcarbamoyl-pyrrolidine-1-carboxylic acid tert-butyl ester (120 mg, 0.27 mmol) and 3-chloro-4-fluoroaniline (46 mg, 0.32 mmol) were stirred in isopropanol (7.5 ml) and hydrogen chloride (80 μl of a 4M solution in dioxane, 0.32 mmol) was added. The mixture was heated at reflux for 2 h, cooled and the solid filtered off. This was dissolved in methanol, absorbed onto an Isolute® SCX column washed with methanol and eluted with 7N ammonia in methanol. These were then evaporated and the residues purified by flash chromatography eluting with increasingly polar mixtures of 10% methanol/dichloromethane to 10% 7N ammonia in methanol/dichloromethane to give (4S)-4-({4-[(3-chloro-4-fluorophenyl)amino]-6-methoxyquinazolin-7-yl}oxy)-N,N-dimethyl-L-prolinamide (56 mg, 48%) as a white solid.

¹H NMR spectrum: (DMSO d₆) 1.68 (m, 1H); 2.71 (m, 1H); 2.85 (s, 3H); 2.95 (dd, 1H); 3.00 (s, 3H); 3.25 (d, 1H); 3.95 (m, 4H); 5.07 (m, 1H); 7.15 (s, 1H); 7.45 (t, 1H); 7.82 (m, 2H); 8.13 (m, 1H); 8.50 (s, 1H); 9.55 (s, 1H); Mass Spectrum: (M+H)⁺ 460

### EXAMPLE 4

### (4S)-4-({4-[(4-Cyano-2-fluorophenyl)amino]-7-methoxyquinazolin-6-yl}oxy)-N,N,1-trimethyl-D-prolinamide

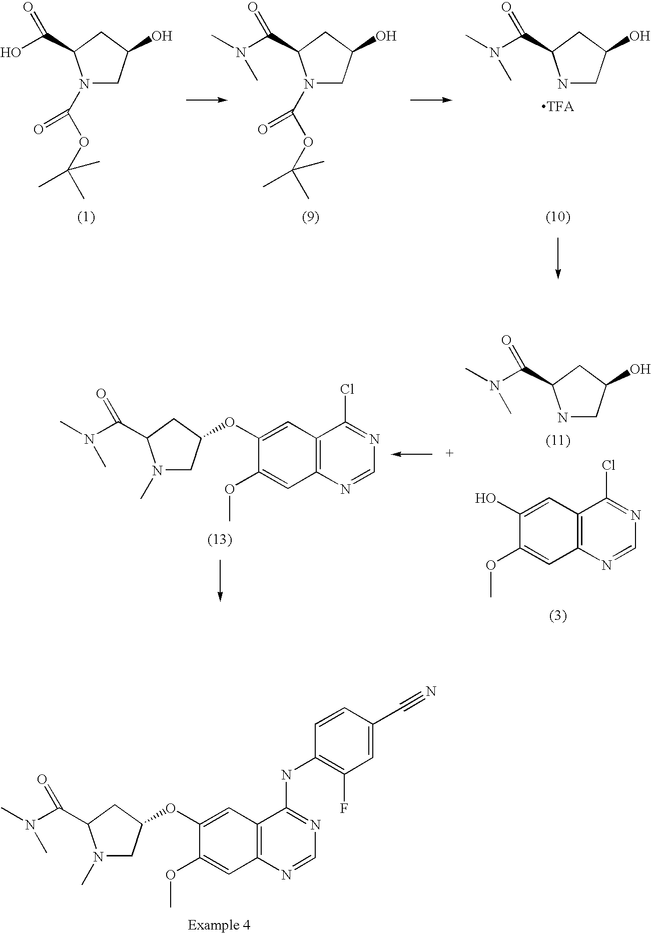

HCl in Dioxan (4.0M, 0.4 ml) was added to a stirred solution of (13) (200 mg) 4-amino-3-fluorobenzonitrile (83 mg) in acetonitrile (10 ml) and the reaction mixture was heated at 65° C. for 3 hours.

The resulting precipitate was filtered, washed with acetonitrile then diethyl ether and dried under vacuum to give the title compound as a beige powder solid HCl salt, (210 mg). ¹H NMR Spectrum: (DMSO d₆+CD₃CO₂D) 2.55-2.69 (m, 1H), 2.75-2.88 (m, 1H), 2.92 (s, 3H), 2.95 (s, 3H), 2.98 (s, 3H), 3.50-3.57 (m, 1H), 4.01 (s, 3H), 4.29 (dd, 1H), 4.94 (dd, 1H), 5.47 (m, 1H), 7.47 (s, 1H), 7.74-7.86 (m, 2H), 8.04 (d, 1H), 8.66 (m, 1), 8.86 (s, 1H); Mass Spectrum: (M+H)+465.

The starting material 1-pyrrolidinecarboxylic acid, 2-[(dimethylamino)carbonyl]-4-hydroxy-, 1,1-dimethylethyl ester, (2R,4R)— (9) was prepared as follows:

1-[3-(Dimethylamino)propyl]-3-ethylcarbodiimide hydrochloride (25.53 g) was added to a stirred suspension of Boc-D-cis-hyp-OH, 1,2-pyrrolidinedicarboxylic acid, 4-hydroxy-1-(1,1-dimethylethyl)ester, (2R,4R) (1) (22.0 g), dimethylaminopyridine (58.11 g) and dimethylamine hydrochloride (15.3 g) in methylene chloride (600 ml) and the reaction mixture was stirred at room temperature for 16 hours. The reaction mixture was washed with citric acid (1.0 M), saturated aqueous sodium bicarbonate solution and saturated brine, dried over magnesium sulphate and filtered. The organic phase was then purified by column chromatography on silica eluting with increasingly polar mixtures of methanol/methylene chloride (1/99-5/95). The desired product fractions were combined and evaporated to give (9) as a white crystalline solid, (16.95 g). ¹H NMR Spectrum: (DMSO d₆) 1.30+1.38 (2s, 9H), 1.50-1.61 (m, 1H), 2.31-2.42 (m, 1H), 2.83+3.02 (2 m, 6H), 3.08-3.14 (m, 1H), 3.46-3.58 (m, 1H), 4.09-4.18 (m, 1H), 4.53-4.61 (m, 1H), 5.15-5.22 (m, 1H).

Starting material Boc-D-cis-hyp-OH (1), 1,2-pyrrolidinedicarboxylic acid, 4-hydroxy-1-(1,1-dimethylethyl)ester, (2R,4R) is commercially available

The starting material (10) was prepared as follows:

TFA (20 ml) was added to a stirred solution of 1-pyrrolidinecarboxylic acid, 2-[(dimethylamino)carbonyl]-4-hydroxy-, 1,1-dimethylethyl ester, (2R,4R)— (9) (5 g) in methylene chloride (20 ml) at 25° C. under an atmosphere of nitrogen and the reaction mixture was stirred for 1.5 hours. The reaction mixture was then reduced in vacuo and triturated with diethyl ether to give the TFA salt of (4R)-4-hydroxy-N,N-dimethyl-D-prolinamide (10) as a white solid. (4.83 g) ¹H NMR Spectrum: (DMSO d₆) 1.68-1.77 (m, 1H), 2.56-2.67 (m, 1H), 2.90 (s, 3H), 2.95 (s, 3H), 3.16-3.20 (m, 2H), 4.37 (m, 1H), 4.52-4.58 (m, 1H), 5.32 (m, 1H).

The starting material (4R)-4-hydroxy-N,N,1-trimethyl-D-prolinamide (4) was prepared as follows:

Platinum oxide was added to a solution of (4R)-4-hydroxy-N,N-dimethyl-D-prolinamide (10) (4.83 g) in formaldehyde (37 wt % solution in water) (3 g), water (16 ml) and acetic acid (30 ml) under an atmosphere of nitrogen. The reaction was then purged with hydrogen and stirred vigorously for 16 hours. The reaction mixture was filtered through celite and reduced in vacuo. The residue was dissolved in methylene chloride and dried over magnesium sulphate. Potassium carbonate (7 g) was added and the mixture stirred for one hour. The crudes were filtered and purified by column chromatography on silica eluting with increasingly polar mixtures of methanol (saturated with ammonia)/methylene chloride (5/95-15/85). The fractions containing the desired product were combined and reduced in vacuo to give (4R)-4-hydroxy-N,N1-trimethyl-D-prolinamide (11) as a colourless oil. (2.57 g). ¹H NMR Spectrum: (DMSO d₆) 1.51-1.60 (m, 1H), 2.17 (s, 3H), 2.28-2.39 (m, 2H), 2.79-2.86 (m, 4H), 3.06 (s, 3H), 3.17 (t, 1H), 4.10-4.19 (1H), 4.80 (d, 1H).

The starting material (4S)-4-[(4-chloro-7-methoxyquinazolin-6-yl)oxy]-N,N,1-trimethyl-D-prolinamide (13) was prepared as follows:

Di-ethyl azodicarboxylate (1.38 g) was added slowly to a stirred suspension of (4R)-4-hydroxy-N,N,1-trimethyl-D-prolinamide (11)(1.0 g), 4-chloro-7-methoxyquinazolin-6-ol (3)(1.11 g) and triphenylphosphine(2.07 g) in methylene chloride (60 ml)at 25° C. under an atmosphere of nitrogen and the reaction mixture was stirred for 2 hours. The reaction mixture was then reduced in vacuo to ½ volume, applied to a silica flash column and eluted with increasingly polar mixtures of methanol/methylene chloride (5/95-10/90). The fractions containing the desired product were combined and evaporated to give (4S)-4-[(4-chloro-7-methoxyquinazolin-6-yl)oxy]-N,N,1-trimethyl-D-prolinamide (13) as a colourless foam (1.6 g).

¹H NMR Spectrum: (DMSO d₆) 2.09-2.20 (m, 1H), 2.25 (s, 3H), 2.28-2.40 (m, 1H), 2.60 (m, 1H), 2.81 (s, 3H), 3.02 (s, 3H), 3.52 (m, 1H), 3.74 (m, 1H), 3.98 (s, 3H), 5.12-5.20 (m, 1H), 7.28 (s, 1H), 7.41 (s, 1H), 8.83 (s, 1H); Mass Spectrum: (M+H)⁺ 365.4

### EXAMPLE 5

The compounds in Table 2 were made in an analogous manner to that of Example 4, all HCl salts.

TABLE 2

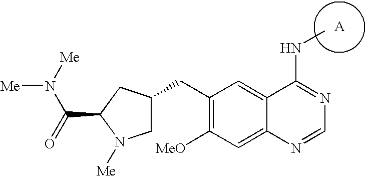

| Compound | A | Footnote |
|---|---|---|
| 1 | 3-chloro-4-cynanophenyl | a |
| 2 | 3-chloro-4-(trifluoromethyl)phenyl | b |
| 3 | 5-chloropyridin-3-yl | c |

a ¹H NMR Spectrum: (DMSO d₆) 2.54-2.66 (m, 1H), 2.70-2.86 (m, 1H), 2.94 (s, 3H), 2.96 (s, 3H), 2.98 (s, 3H), 3.49-3.57 (m, 1H), 4.00 (s, 3H), 4.26 (dd, 1H), 4.95 (t, 1H), 5.63 (m, 1H), 7.48 (s, 1H), 8.00 (d, 1H), 8.32 (d, 1H), 8.57 (s, 1H), 8.90 (m, 2H), 12.00 (s, 1H); Mass Spectrum: (M + H)⁺ 481.06.
b ¹H NMR Spectrum: (DMSO d₆) 2.55-2.68 (m, 1H), 2.75-2.84 (m, 1H), 2.94 (s, 3H), 2.95 (s, 3H), 2.98 (s, 3H), 3.49-3.58 (m, 1H), 4.01 (s, 3H), 4.27 (dd, 1H), 4.95 (t, 1H), 5.61 (m, 1H), 7.53 (s, 1H), 7.92 (d, 1H), 8.21 (d, 1H), 8.44 (s, 1H), 8.94 (s, 1H), 8.98 (s ,1H), 12.30 (s, 1H); Mass Spectrum: (M + H)⁺ 524.04.
c ¹H NMR Spectrum: (DMSO d₆) 2.58-2.80 (m, 2H), 2.90 (s, 3H), 2.92 (s, 3H), 2.96 (s, 3H), 3.45-3.55 (m, 1H), 3.99 (s, 3H), 4.26 (dd, 1H), 4.92 (t, 1H), 5.57 (m, 1H), 7.54 (s, 1H), 8.48-8.51 (m, 2H), 8.92 (s, 1H), 9.01 (s, 1H), 9.10 (s, 1H), 12.66 (s, 1H); Mass Spectrum: (M + H)⁺ 457.05

### EXAMPLE 6

### (4S)-4-({4-[(2-Fluoro-4-methylphenyl)amino]-7-methoxyquinazolin-6-yl}oxy)-N,N,1-trimethyl-D-prolinamide

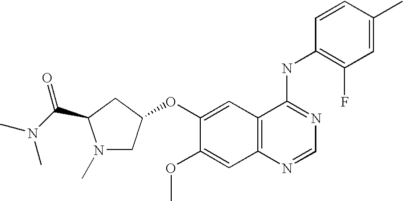

HCl in Dioxan (4.0M, 0.4 ml) was added to a stirred solution of (13) (200 mg) 2-fluoro-4methylaniline (76 mg) in acetonitrile (10 ml) and the reaction mixture was heated at 65° C. for 3 hours.

The reaction mixture was reduced in vacuo and the residue dissolved in Methanol saturated with ammonia (7N). The solution was then reduced in vacuo and the residue dissolved in methylene chloride and washed with saturated aqueous sodium bicarbonate solution and saturated brine. The organic phase was then purified by column chromatography on silica eluting with increasingly polar mixtures of methanol/methylene chloride (0/100-10/90). The desired product fractions were combined and reduced in vacuo and triturated with diethyl ether to give (4S)-4-({4-[(2-fluoro-4-methylphenyl)amino]-7-methoxyquinazolin-6-yl}oxy)-N,N,1-trimethyl-D-prolinamide as a white solid (175 mg). ¹H NMR Spectrum: (DMSO d₆) 2.05-2.18 (m, 1H), 2.30 (s, 3H), 2.35 (s, 3H), 2.44-2.53 (m, 2H+DMSO), 2.83 (s, 3H), 3.04 (s, 3H), 3.59-3.77 (m, 2H), 3.94 (s, 3H), 5.08 (m, 1H), 7.02-7.19 (m, 3H), 7.31-7.40 (m, 1H), 7.68 (s, 1H), 8.28 (s, 1H), 9.43 (s, 1H); Mass Spectrum: (M+H)⁺ 454.

### EXAMPLE 7

The following compounds were made in an analogous manner to that of Example 6.

TABLE 3

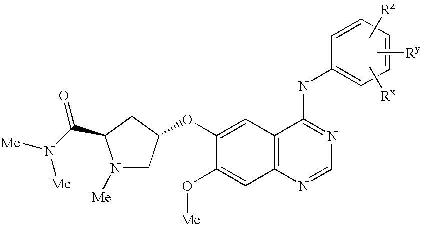

| Compound | R^{x} | R^{y} | R^{z} | Footnotes |
|---|---|---|---|---|
| 1 | 3-Cl | 4-F | H | a |
| 2 | 2-F | 4-OH | H | b |
| 3 | 2-F | 4-F | H | c |
| 4 | 2-F | 5-F | H | d |
| 5 | 2-F | 5-Cl | H | e |
| 6 | 2-F | 4-Cl | H | f |
| 7 | 2-OH | 5-Cl | H | g |
| 8 | 3-Cl | 4-OMe | H | h |
| 9 | 2-SO₂NH₂ | 5-Cl | H | i |
| 10 | 2-F | 3-F | 4-F | j |
| 11 | 2-F | 5-CF₃ | H | k |
| 12 | 2-F | 3-CF₃ | H | l |
| 13 | 2-OMe | 3-Cl | H | m |
| 14 | 2-Me | 3-Cl | H | n |
| 15 | 3-Cl | 4-OH | H | o |
| 16 | 3-CN | H | H | p |
| 17 | *(3)-NCH═ | ═CH-(4) | H | q |

*R^{x} and R^{y} together with the carbon atoms in the 3- and 4-position of the phenyl ring, to which they are attached, form a pyrrole ring.
a ¹H NMR Spectrum: (DMSO d₆) 2.07-2.16 (m, 1H), 2.29 (s, 3H), 2.42-2.58 (m, 2H + DMSO), 2.84 (s, 3H), 3.04 (s, 3H), 3.62 (dd, 1H), 3.71 (t, 1H), 3.93 (s, 3H), 5.12 (m, 1H), 7.20 (s, 1H), 7.43 (t, 1H), 7.69-7.76 (m, 2H), 8.05 (dd, 1H), 8.48 (s, 1H), 9.55 (s, 1H); Mass Spectrum: (M + H)⁺ 474.
b ¹H NMR Spectrum: (DMSO d₆) 2.05-2.15 (m, 1H), 2.28 (s, 3H), 2.46-2.59 (m, 2H + DMSO), 2.83 (s, 3H), 3.04 (s, 3H), 3.61 (dd, 1H), 3.70 (t, 1H), 3.91 (s, 3H), 5.07 (m, 1H), 6.61-6.70 (m, 2H), 7.15 (s, 1H), 7.21 (t, 1H), 7.65 (s, 1H), 8.26 (s, 1H), 9.28 (s, 1H), 9.83 (s, 1H); Mass Spectrum: (M + H)⁺ 456.16
c ¹H NMR Spectrum: (DMSO d₆) 2.06-2.16 (m, 1H), 2.28 (s, 3H), 2.43-2.60 (m, 2H + DMSO), 2.84 (s, 3H), 3.05 (s, 3H), 3.61 (dd, 1H), 3.71 (t, 1H), 3.92 (s, 3H), 5.08 (m, 1H), 7.14 (t, 1H), 7.18 (s, 1H), 7.35 (t, 1H), 7.54 (m, 1H), 7.68 (s, 1H), 8.31 (s, 1H), 9.48 (s, 1H); Mass Spectrum: (M + H)⁺ 458.09
d ¹H NMR Spectrum: (DMSO d₆) 2.05-2.16 (m, 1H), 2.28 (s, 3H), 2.47-2.60 (m, 2H + DMSO), 2.84 (s, 3H), 3.04 (s, 3H), 3.62 (dd, 1H), 3.72 (t, 1H), 3.93 (s, 3H), 5.10 (m, 1H), 7.13 (m, 1H), 7.20 (s, 1H), 7.35 (m, 1H), 7.50 (m, 1H), 7.70 (s, 1H), 8.38 (s, 1H), 9.57 (s, 1H); Mass Spectrum: (M + H)⁺ 458.17
e ¹H NMR Spectrum: (DMSO d₆) 2.08-2.17 (m, 1H), 2.29 (s, 3H), 2.46-2.60 (m, 2H + DMSO), 2.85 (s, 3H), 3.05 (s, 3H), 3.62 (dd, 1H), 3.71 (t, 1H), 3.94 (s, 3H), 5.09 (m, 1H), 7.20 (s, 1H), 7.32-7.40 (m, 2H), 7.65-7.70 (m, 2H), 8.39 (s, 1H), 9.59 (s, 1H); Mass Spectrum: (M + H)⁺ 474.11
f ¹H NMR Spectrum: (DMSO d₆) 2.07-2.17 (m, 1H), 2.29 (s, 3H), 2.42-2.59 (m, 2H + DMSO), 2.83 (s, 3H), 3.04 (s, 3H), 3.61 (dd, 1H), 3.71 (t, 1H), 3.92 (s, 3H), 5.09 (m, 1H), 7.19 (s, 1H), 7.30-7.34 (m, 1H), 7.50-7.59 (m, 2H), 7.68 (s, 1H), 8.33 (s, 1H), 9.56 (s, 1H); Mass Spectrum: (M + H)⁺ 474.10
g ¹H NMR Spectrum: (DMSO d₆) 2.07-2.18 (m, 1H), 2.29 (s, 3H), 2.40-2.50 (m, 2H + DMSO), 2.83 (s, 3H), 3.04 (s, 3H), 3.57-3.75 (m, 2H), 3.93 (s, 3H), 5.11 (m, 1H), 6.94 (d, 1H), 7.10 (d, 1H), 7.18 (s, 1H), 7.54 (s, 1H), 7.69 (s, 1H), 8.35 (s, 1H), 9.22 (s, 1H), 10.04 (s, 1H); Mass Spectrum: (M + H)⁺ 472.06
h ¹H NMR Spectrum: (DMSO d₆) 2.07-2.16 (m, 1H), 2.28 (s, 3H), 2.40-2.59 (m, 2H + DMSO), 2.86 (s, 3H), 3.04 (s, 3H), 3.59-3.72 (m, 2H), 3.87 (s, 3H), 3.93 (s, 3H), 5.12 (m, 1H), 7.15-7.20 (m, 2H), 7.64 (d, 1H), 7.76 (s, 1H), 7.84 (s, 1H), 8.40 (s, 1H), 9.43 (s, 1H); Mass Spectrum: (M + H)⁺ 486.08
i ¹H NMR Spectrum: (DMSO d₆) 2.15-2.24 (m, 1H), 2.32 (s, 3H), 2.40-2.56 (m, 1H + DMSO), 2.60-2.68 (m, 1H), 2.84 (s, 3H), 3.06 (s, 3H), 3.63-3.78 (m, 2H), 3.96 (s, 3H), 5.01 (m, 1H), 7.23 (m, 1H), 7.30-7.37 (m, 2H), 7.83-7.95 (m, 2H), 8.67 (s, 1H), 9.01 (s, 1H); Mass Spectrum: (M + H)⁺ 535.03
j ¹H NMR Spectrum: (DMSO d₆) 2.08-2.17 (m, 1H), 2.28 (s, 3H), 2.42-2.61 (m, 2H + DMSO), 2.83 (s, 3H), 3.05 (s, 3H), 3.62 (dd, 1H), 3.72 (t, 1H), 3.93 (s, 3H), 5.08 (m, 1H), 7.20 (s, 1H), 7.32-7.40 (m, 2H), 7.67 (s, 1H), 8.35 (s, 1H), 9.68 (s, 1H); Mass Spectrum: (M + H)⁺ 476.08
k ¹H NMR Spectrum: (DMSO d₆) 2.09-2.19 (m, 1H), 2.30 (s, 3H), 2.42-2.61 (m, 2H + DMSO), 2.84 (s, 3H), 3.05 (s, 3H), 3.62 (dd, 1H), 3.72 (t, 1H), 3.94 (s, 3H), 5.09 (m, 1H), 7.21 (s, 1H), 7.55 (t, 1H), 7.65-7.71 (m, 2H), 7.95 (d, 1H), 8.36 (s, 1H), 9.69 (s, 1H); Mass Spectrum: (M + H)⁺ 508.07
l ¹H NMR Spectrum: (DMSO d₆) 2.08-2.18 (m, 1H), 2.28 (s, 3H), 2.40-2.60 (m, 2H + DMSO), 2.84 (s, 3H), 3.04 (s, 3H), 3.61 (dd, 1H), 3.71 (t, 1H), 3.93 (s, 3H), 5.09 (m, 1H), 7.20 (s, 1H), 7.45 (t, 1H), 7.65 (t, 1H), 7.70 (s, 1H), 7.91 (t, 1H), 8.35 (s, 1H), 9.68 (s, 1H); Mass Spectrum: (M + H)⁺ 508.06
m ¹H NMR Spectrum: (DMSO d₆) 2.07-2.17 (m, 1H), 2.28 (s, 3H), 2.42-2.60 (m, 2H + DMSO), 2.83 (s, 3H), 3.04 (s, 3H), 3.57-3.75 (m, 2H), 3.68 (s, 3H), 3.92 (s, 3H), 5.12 (m, 1H), 7.14-7.20 (m, 2H), 7.36 (d, 1H), 7.53 (d, 1H), 7.73 (s, 1H), 8.34 (s, 1H), 9.42 (s, 1H); Mass Spectrum: (M + H)⁺ 486.07
n ¹H NMR Spectrum: (DMSO d₆) 2.08-2.15 (m, 1H), 2.17 (s, 3H), 2.29 (s, 3H), 2.44-2.54 (m, 1H + DMSO), 2.57 (dd, 1H), 2.83 (s, 3H), 3.04 (s, 3H), 3.61 (dd, 1H), 3.72 (t, 1H), 3.93 (s, 3H), 5.08 (m, 1H), 7.16 (s, 1H), 7.25-7.30 (m, 2H), 7.35-7.40 (m, 1H), 7.68 (s, 1H), 8.26 (s, 1H), 9.57 (s, 1H); Mass Spectrum: (M + H)⁺ 470.09
o ¹H NMR Spectrum: (DMSO d₆) 2.07-2.15 (m, 1H), 2.29 (s, 3H), 2.40-2.58 (m, 2H + DMSO), 2.84 (s, 3H), 3.04 (s, 3H), 3.59-3.72 (m, 2H), 3.92 (s, 3H), 5.11 (m, 1H), 6.98 (d, 1H), 7.16 (s, 1H), 7.44 (dd, 1H), 7.69 (s, 1H), 7.71 (d, 1H), 8.38 (s, 1H), 9.37 (s, 1H), 9.94 (s, 1H); Mass Spectrum: (M + H)⁺ 472.06
p ¹H NMR Spectrum: (DMSO d₆) 2.08-2.18 (m, 1H), 2.29 (s, 3H), 2.41-2.58 (m, 2H + DMSO), 2.83 (s, 3H), 3.04 (s, 3H), 3.60-3.74 (m, 2H), 3.93 (s, 3H), 4.18 (s, 1H), 5.14 (m, 1H), 7.17-7.23 (m, 2H), 7.40 (t, 1H), 7.72 (s, 1H), 7.83 (d, 1H), 7.92 (s, 1H), 8.35 (s, 1H), 9.68 (s, 1H); Mass Spectrum: (M + H)⁺ 446.12
q ¹H NMR Spectrum: (DMSO d₆) 2.06-2.17 (m, 1H), 2.28 (s, 3H), 2.40-2.58 (m, 2H + DMSO), 2.83 (s, 3H), 3.04 (s, 3H), 3.60-3.73 (m, 2H), 3.92 (s, 3H), 5.12 (m, 1H), 6.42 (s, 1H), 7.13 (s, 1H), 7.26-7.40 (m, 3H), 7.76 (d, 2H), 8.31 (s, 1H), 9.46 (s, 1H), 11.04 (s, 1H); Mass Spectrum: (M + H)⁺ 461.12

### EXAMPLE 8

### (4S)-4-({4-[(3-Chloro-1H-indol-5-yl)amino]-7-methoxyquinazolin-6-yl}oxy)-N,N,1-trimethyl-D-prolinamide

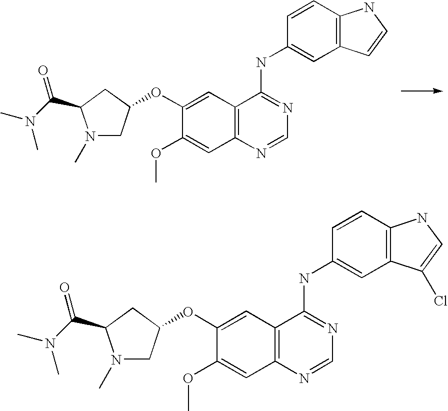

N-Chlorosuccinimide (22 mg) was added to a stirred solution of (4S)-4-{[4-(1H-indol-5-ylamino)-7-methoxyquinazolin-6-yl]oxy}-N,N,1-trimethyl-D-prolinamide (Table 3, Compound 17), (77 mg) in DMF (5 ml) at room temperature under an atmosphere of nitrogen and the reaction mixture was stirred for 1 hour.

The reaction mixture was quenched with water and extracted with ethyl acetate. The organics were then adsorbed onto silica and then purified by column chromatography eluting with increasingly polar mixtures of methanol/methylene chloride (0/100-10/90). The desired product fractions were combined, evaporated and triturated with diethyl ether to give (4S)-4-({4-[(3-chloro-1H-indol-5-yl)amino]-7-methoxyquinazolin-6-yl}oxy)-N,N,1-trimethyl-D-prolinamide as a cream coloured solid (25 mg). ¹H NMR Spectrum: (DMSO d₆) 2.08-2.17 (m, 1H), 2.29 (s, 3H), 2.40-2.59 (m, 2H+DMSO), 2.85 (s, 3H), 3.05 (s, 3H), 3.60-3.73 (m, 2H), 3.92 (s, 3H), 5.14(m, 1H), 7.16 (s, 1H), 7.41 (d, 1H), 7.48-7.52 (m, 2H), 7.75-7.77 (m, 2H), 8.36 (s, 1H), 9.52 (s, 1H), 11.32 (s, 1H); Mass Spectrum: (M+H)⁺ 495.12

### EXAMPLE 9

### (4S)-4-({4-[(3-Chloro-2-fluorophenyl)amino]quinazolin-7-yl}oxy)-N,N,1-trimethyl-L-prolinamide

### EXAMPLE 9

### (4S)-4-({4-[(3-Chloro-2-fluorophenyl)amino]quinazolin-7-yl}oxy)-N,N,1-trimethyl-L-prolinamide

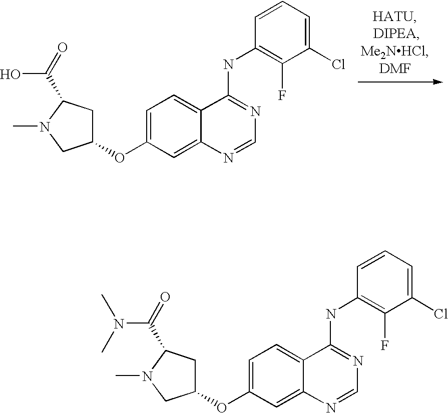

(4S)-4-({4-[(3-chloro-2-fluorophenyl)amino]quinazolin-7-yl}oxy)-1-methyl-L-proline (150 mg, 0.36 mmol), diisopropylethylamine (0.31 ml, 1.80 mmol) and O-(7-azabenzotriazol-1-yl)-N,N,N′,N′-tetramethyluronium hexafluorophosphate (205 mg, 0.54 mmol) were dissolved in N,N-dimethylformamide (2 ml) and dimethylamine hydrochloride (44 mg, 0.54 mmol) added. The mixture was stirred at room temperature for 1.5 h and then concentrated under reduced pressure. The residue was purified by column chromatography on silica eluting with methanol/dichloromethane (5/95) to give (4S)-4-({4-[(3-chloro-2-fluorophenyl)amino]quinazolin-7-yl}oxy)-N,N,1-trimethyl-L-prolinamide (92 mg, 58%) as a white solid. ¹H NMR spectrum: (DMSO d₆) 1.81 (m, 1H); 2.21 (s, 3H); 2.59 (dd, 1H); 2.81 (m, 4H); 3.07 (s, 3H); 3.21 (m, 2H); 5.07 (s, 1H); 7.08 (m, 1H); 7.24 (m, 2H); 7.49 (m, 2H); 8.34 (d, 1H); 8.42 (s, 1H); 9.80 (s, 1H). Mass Spectrum: (MH)⁺ 444.

The starting material was prepared as follows:

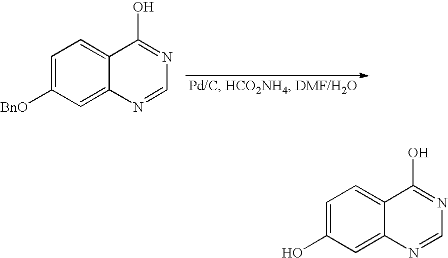

7-(Benzyloxy)quinazolin-4-ol (2.5 g, 9.91 mmol) was suspended in N,N-dimethylformamide (40 ml) and the system purged with nitrogen gas. 10% Palladium on carbon (0.63 g, 25% by mass) and ammonium formate (6.2 g, 99.1 mmol) in water (5 ml) were added. The mixture was stirred at room temperature for 2 h, filtered and concentrated under reduced pressure. The residue was suspended in water, filtered and dried to give quinazoline-4,7-diol (1.08 g, 67%) as a white solid.

¹H NMR spectrum: (DMSO d₆) 6.95 (m, 2H); 7.95 (m, 2H); 10.42 (brs, 1H); 11.90 (brs, 1H); Mass Spectrum: (MH)⁺ 163.

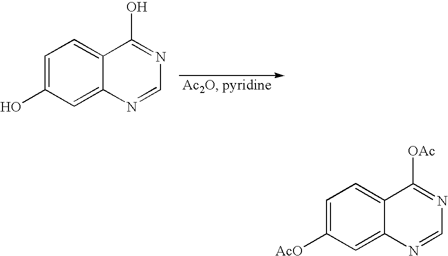

Quinazoline-4,7-diol (1.0 g, 6.17 mmol) was suspended in acetic anhydride (8 ml) and pyridine (1.1 ml, 1.42 mmol) added. The mixture was heated at reflux for 2.5 h, cooled and carefully poured onto ice/water and stirred for 1 h The solid was filtered off and dried to give quinazoline-4,7-diyl diacetate (1 g, 79%).¹H NMR spectrum: (DMSO d₆) 2.33 (s, 3H); 2.74 (s, 3H); 7.39 (dd, 1H); 7.49 (d, 1H); 8.26 (d, 1H); 8.62 (s, 1H).

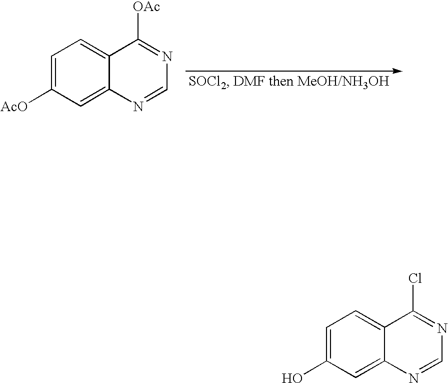

Quinazoline-4,7-diyl diacetate (1.0 g, 4.89 mmol) and N,N-dimethylformamide (a few drops) were heated in thionyl chloride (12 ml) at reflux for 3 h. The mixture was cooled, concentrated under reduced pressure and the system azeotroped with toluene. The residue was dissolved in dichloromethane (6 ml) and carefully added to methanol (8 ml) and concentrated aqueous ammonia solution (1.5 ml) and stirred for 2 h. The mixture was concentrated under reduced pressure and the solid suspended in water, filtered and dried to give 4-chloroquinazolin-7-ol (655 mg, 74%) as a white solid. ¹H NMR spectrum (DMSO d₆) 7.23 (d, 1H); 7.37 (dd, 1H); 8.12 (d, 1H); 8.87 (s, 1H); 11.22 (brs, 1H); Mass Spectrum: (MH)⁺ 181.

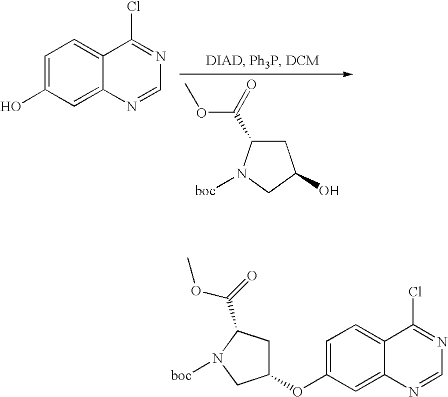

4-Chloroquinazolin-7-ol (0.64 g, 3.54 mmol), (2S,4R)-4-hydroxy-pyrrolidine-1,2-dicarboxylic acid 1-tert-butyl ester 2-methyl ester (1.04 g, 4.25 mmol) and triphenylphosphine (1.11 g, 4.25 mmol) were stirred in dichloromethane (30 ml) and diisopropyl azodicarboxylate (0.84 ml, 4.25 mmol) was slowly added. The mixture was stirred at room temperature for 1.75 h and then concentrated under reduced pressure. The residue was purified by column chromatography on silica eluting with increasingly polar mixtures of isohexane/ethyl acetate (2/1 to 1/1) to give (2S,4S)-4-(4-chloro-quinazolin-7-yloxy)-pyrrolidine-1,2-dicarboxylic acid 1-tert-butyl ester 2-methyl ester (1.09 g, 75%) as a viscous oil. ¹H NMR spectrum (DMSO d₆) 1.37 (m, 9H); 2.29 (d, 1H); 2.71 (m, 1H); 3.53 (m, 1H); 3.63 (m 3H); 3.87 (m, 1H); 4.47 (m, 1H); 5.35 (m, 1H); 7.32 (m, 1H); 7.46 (s, 1H); 8.17 (d, 1H); 8.98 (s, 1H); Mass Spectrum: (MH)⁺ 408.

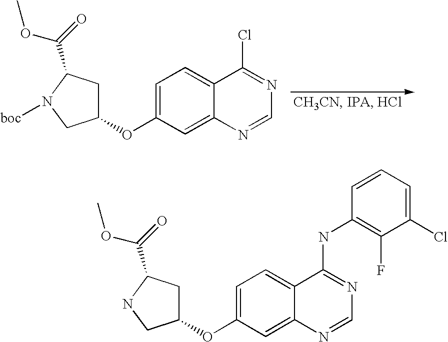

(2S,4S)-4-(4-Chloro-quinazolin-7-yloxy)-pyrrolidine-1,2-dicarboxylic acid 1-tert-butyl ester 2-methyl ester (1.0 g, 2.45 mmol) and 3-chloro-2-fluoroaniline (323 μl, 2.94 mmol) were stirred in acetonitrile (25 ml) and hydrogen chloride (736 μl of a 4M solution in dioxane, 2.94 mmol) was added. The mixture was heated at reflux for 2 h, cooled and concentrated under reduced pressure. The residue was dissolved in methanol, absorbed onto and Isolute® SCX column, washed with methanol and eluted with 7N ammonia in methanol Appropriate fractions were combined and evaporated and the crudes purified by column chromatography on silica eluting with 7N ammonia in methanol/dichloromethane (2/98) to give methyl(4S)-4-({4-[(3-chloro-2-fluorophenyl)amino]quinazolin-7-yl}oxy)-L-prolinate (811 mg, 79%) as a white solid. ¹H NMR spectrum: (DMSO d₆) 2.09 (m, 1H); 2.54 (m, 1H); 2.80 (brs, 1H); 3.15 (m, 2H); 3.64 (s, 3H); 3.81 (dd, 1H); 5.11 (m, 1H); 7.16 (m, 2H); 7.28 (t, 1H); 7.51 (m, 2H); 8.35 (d, 1H); 8.44 (s, 1H); 7.80 (s, 1H); Mass Spectrum: (MH)⁺ 417.

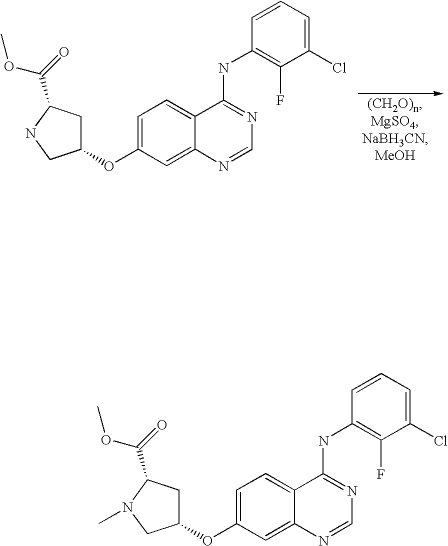

Methyl(4S)-4-({4-[(3-chloro-2-fluorophenyl)amino]quinazolin-7-yl}oxy)-L-prolinate (50 mg, 1.20 mmol) was dissolved in methanol (20 ml) and magnesium sulphate (289 mg, 2.40 mmol), paraformaldehyde (360 mg, 12.0 mmol) and sodium cyanoborohydride (302 mg, 4.80 mmol) added. The mixture was heated at 50° C. for 2.5 h, cooled, filtered and evaporated. The crudes were purified by column chromatography on silica eluting with methanol/dichloromethane (2/98) to give methyl(4S)-4-({4-[(3-chloro-2-fluorophenyl)amino]quinazolin-7-yl}oxy)-1-methyl-L-prolinate (328 mg, 63%) as a white solid.

¹H NMR spectrum (DMSO d₆) 2.08 (m, 1H); 2.38 (s, 3H); 2.75 (dd, 1H); 2.86 (m, 1H); 3.13 (t, 1H); 3.26 (d, 1H); 3.71 (s, 3H); 5.15 (m, 1H); 7.15 (d, 1H); 7.28 (dd, 1H); 7.34 (t, 1H); 7.56 (m, 2H); 8.41 (d, 1H); 8.50 (s, 1H); 9.85 (s, 1H); Mass Spectrum: (MH)⁺ 431.

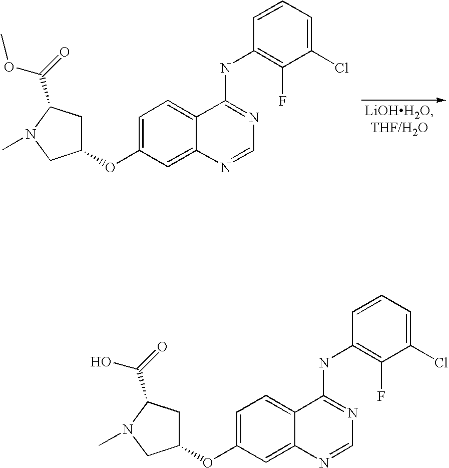

Methyl(4S)-4-({4-[(3-chloro-2-fluorophenyl)amino]quinazolin-7-yl}oxy)-1-methyl-L-prolinate (325 mg, 0.75 mmol) was dissolved in tetrahydrofuran (6 ml) and water (3 ml) and lithium hydroxide monohydrate (158 mg, 3.77 mmol) added. The mixture was stirred at room temperature for 2 h, neutralised with hydrogen chloride (0.95 ml of a 4M solution in dioxane, 3.77 mmol) and concentrated under reduced pressure. The residue was dissolved in methanol, absorbed onto an Isolute® SCX column, washed with methanol and eluted with 7N ammonia in methanol. Fractions containing the desired product were combined and evaporated to give (4S)-4-({4-[(3-chloro-2-fluorophenyl)amino]quinazolin-7-yl}oxy)-1-methyl-L-proline (318 mg, 100%) as a white solid.

¹H NMR spectrum: (DMSO d₆) 2.00 (m, 1H); 2.35 (s, 3H); 2.66 (m, 2H); 2.82 (t, 1H); 3.24 (d, 1H); 5.03 (m, 1H); 7.06 (m, 1H); 7.17 (dd, 1H); 7.25 (dt, 1H); 7.48 (m, 2H); 8.38 (m, 2H); 10.00 (brs, 1H); Mass Spectrum: (MH)⁺ 417.

### EXAMPLE 10

### (4S)-4-({4-[(3-Chloro-2-fluorophenyl)amino]quinazolin-7-yl}oxy)-1-methyl-L-prolinamide triflouroacetic acid salt

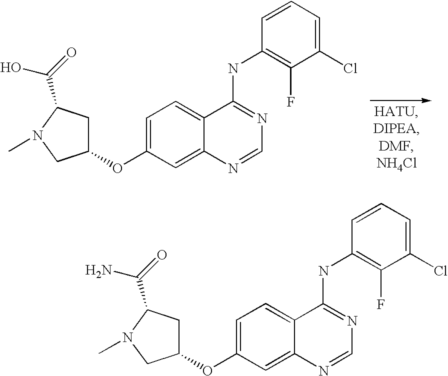

(4S)-4-({4-[(3-chloro-2-fluorophenyl)amino]quinazolin-7-yl}oxy)-1-methyl-L-proline (150 mg, 0.36 mmol), diisopropylethylamine (0.31 ml, 1.80 mmol) and O-(7-azabenzotriazol-1-yl)-N,N,N′,N′-tetramethyluronium hexafluorophosphate (205 mg, 0.54 mmol) were dissolved in N,N-dimethylformamide (2 ml) and ammonium chloride (29 mg, 0.54 mmol) added. The mixture was stirred at room temperature for 1.5 h and then concentrated under reduced pressure. The residues were purified by column chromatography on silica eluting with methanol/dichloromethane(5/95) followed by reverse phase preparative HPLC to give (4S)-4-({4-[(3-chloro-2-fluorophenyl)amino]quinazolin-7-yl}oxy)-1-methyl-L-prolinamide triflouroacetic acid salt (92 mg, 58%) as a white solid. ¹H NMR spectrum (DMSO d₆) 1.96 (m, 1H); 2.36 (s, 3H); 2.72 (dd, 1H); 2.81 (m, 2H); 3.23 (s, 1H); 3.32 (d, 1H); 5.16 (s, 1H); 7.15 (m, 2H); 7.26 (m, 2H); 7.33 (t, 1H); 7.56 (m, 2H); 8.41 (d, 1H); 8.49 (s, 1H); 9.85 (s, 1H); Mass Spectrum: (MH)⁺ 416.

### EXAMPLE 11

4S)-4-({4-[(3-chloro-2-fluorophenyl)amino]-7-methoxyquinazolin-6-yl}oxy)-1-methyl-D-proline (prepared as described in Example 1) was coupled with an appropriate amine to give the following compounds analogously as for the equivalent step in example 1.

### Compound 11-1

### (4S)-4-({4-[(3-chloro-2-fluorophenyl)amino]-7-methoxyquinazolin-6-yl}oxy)-N-[(1S)-1-(hydroxymethyl)-3-methylbutyl]-1-methyl-D-prolinamide

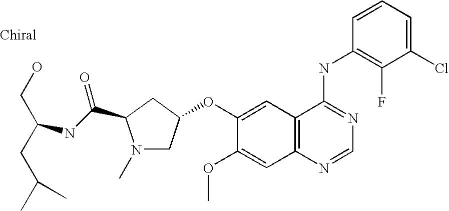

NMR Spectrum: (DMSO d₆+CD₃CO₂D) 0.80-0.90 (m, 6H), 1.25-1.35 (t, 2H), 1.48-1.60 (m, 1H), 2.30-2.40 (m, 2H), 2.46-2.57 (m, 3H+DMSO), 2.81-2.90 (m, 1H), 3.26-3.39 (m, 2H), 3.46-3.55 (m, 1H), 3.80-3.98 (m, 2H), 3.96 (s, 3H), 5.14 (m, 1H), 7.21-7.31 (m, 2H), 7.42-7.55 (m, 2H), 7.64 (s, 1H), 8.38 (s, 1H); Mass Spectrum: (M+H)⁺ 546.

### Compound 11-2

### (4S)-4-({4-[(3-chloro-2-fluorophenyl)amino]-7-methoxyquinazolin-6-yl}oxy)-N-(2-furylmethyl)-1-methyl-D-prolinamide

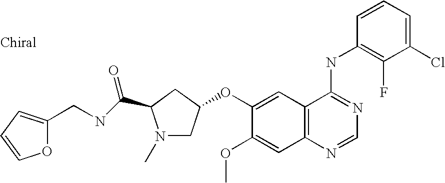

NMR Spec: (DMSO d₆) 2.09-2.20 (m, 1H), 2.30 (s, 3H), 2.24-2.60 (m, 2H+DMSO), 3.10 (t, 1H), 3.68 (m, 1H), 3.94 (s, 3H), 4.30 (d, 2H), 5.07 (m, 1H), 6.20 (s, 1H), 6.36 (s, 1H), 7.20 (s, 1H), 7.27 (t, 1H), 7.44-7.56 (m, 3H), 7.68 (s, 1H), 8.30 (t, 1H), 8.36 (s, 1H), 9.63 (s, 1H); Mass Spectrum: (M+H)⁺ 526.

### Compound 11-3

### (4S)-4-({4-[(3-chloro-2-fluorophenyl)amino]-7-methoxyquinazolin-6-yl}oxy)-1-methyl-N-[(5-methylisoxazol-3-yl)methyl]-D-prolinamide

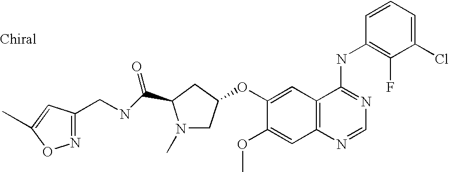

NMR Spectrum: DMSO d₆) 2.11-2.21 (m, 1H), 2.32 (s, 3H), 2.34 (s, 3H), 2.26-2.59 (m, 2H+DMSO), 3.17 (t, 1H), 3.69 (dd, 1H), 3.94 (s, 3H), 4.30 (t, 2H), 5.08 (m, 1H), 6.08 (s, 1H), 7.20 (s, 1H), 7.27 (t, 1H), 7.44-7.55 (m, 2H), 7.68 (s, 1H), 8.35 (s, 1H), 8.42 (t, 1H), 9.62 (s, 1H); Mass Spectrum: (M+H)⁺ 541.

### Compound 11-4

### (4S)-4-({4-[(3-chloro-2-fluorophenyl)amino]-7-methoxyquinazolin-6-yl}oxy)-N-[2-(1H-imidazol-1-yl)ethyl]-1-methyl-D-prolinamide

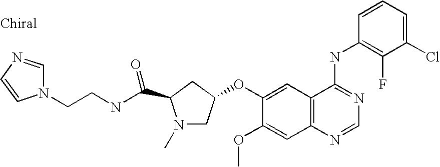

NMR Spectrum: DMSO d₆+CD₃CO₂D) 2.11-2.20 (m, 2), 2.28 (s, 3H), 2.60 (dd, 1H), 3.13 (t, 1H), 3.34-3.56 (m, 2H), 3.66 (dd, 1H), 3.94 (s, 3H), 4.12 (t, 2H), 5.00 (m, 1H), 6.97 (s, 1H), 7.17-7.29 (m, 3H), 7.40-7.52 (m, 2H), 7.66 (s, 1H), 7.81 (s, 1H), 8.35 (s, 1H); Mass Spectrum: (M+H)⁺ 540.

### Compound 11-5

### (2S)-1-[(4S)-4-({4-[(3-chloro-2-fluorophenyl)amino]-7-methoxyquinazolin-6-yl}oxy)-1-methyl-D-prolyl]azetidine-2-carboxamide

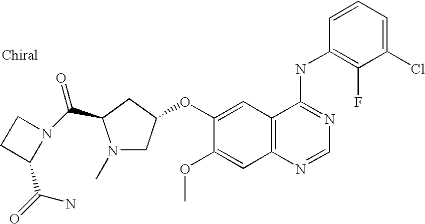

NMR Spectrum: (DMSO d₆+CD₃CO₂D) 1.91-2.00 (1H), 2.06-2.14 (m, 1H), 2.26-2.61 (m, 6H), 3.16 (t, 1H), 3.57-3.71 (m, 1H), 3.79 (t, 1H), 3.88 (s, 3H), 4.09-4.21 (m, 1H), 4.54+4.88 (m, 1H), 5.08 (m, 1H), 7.18 (s, 1H), 7.23 (t, 1H), 7.40-7.52 (m, 2H), 7.67 (d, 1H), 8.33 (s, 1H); Mass Spectrum: (M+H)⁺ 529.

### Compound 11-6

### (4S)-4-({4-[(3-chloro-2-fluorophenyl)amino]-7-methoxyquinazolin-6-yl}oxy)-N-[(2R)-2,3-dihydroxypropyl]-1-methyl-D-prolinamide

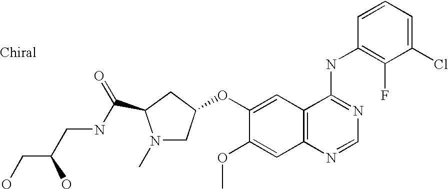

NMR Spectrum: (DMSO d₆+CD₃CO₂D) 2.17-2.29 (m, 1H), 2.29-2.52 (m, 1H), 2.38 (s, 3H), 2.56-2.65 (m, 1H), 2.96-3.05 (m, 1H), 3.18-3.34 (m, 4H), 3.44-3.53 (m, 1H), 3.74 (m, 1H), 3.93 (s, 3H), 5.07 (m, 1H), 7.20 (s, 1H), 7.27 (t, 1H), 7.42-7.54 (m, 2H), 7.68 (s, 1H), 8.35 (s, 1H); Mass Spectrum: (M+H)⁺ 520.

### Compound 11-7

### (4s)-4-({4-[(3-chloro-2-fluorophenyl)amino]-7-methoxyquinazolin-6-yl}oxy)-1-methyl-N-(1-methyl-1H-pyrazol-5-yl)-D-prolinamide

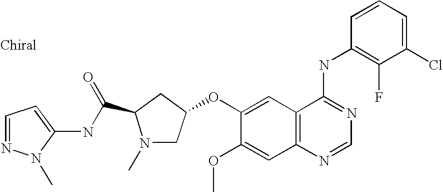

NMR Spectrum: (DMSO d₆) 2.25-2.34 (m, 1H), 2.39-2.52 (m, 1H), 2.42 (s, 3H), 2.63 (dd, 1H) 3.39-3.48 (m, 1H), 3.65 (s, 3H), 3.74 (dd, 1H), 3.95 (s, 3H), 5.13 (m, 1H), 6.18 (s, 1H), 7.21-7.33 (m, 3H), 7.45-7.55 (m, 2H), 7.70 (s, 1H), 8.38 (s, 1H), 9.63 (s, 1H), 9.90 (s, 1H); Mass Spectrum: (M+H)⁺ 526.

### Compound 11-8

### (4S)-4-({cube root}4-[(3-chloro-2-fluorophenyl)amino]-7-methoxyquinazolin-6-yl}oxy)-1-methyl-N-3-thienyl-D-prolinamide

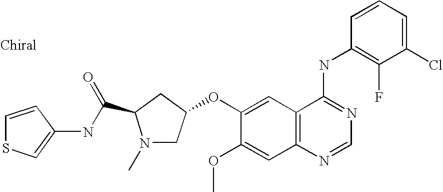

NMR Spectrum: (DMSO d₆) 2.20-2.30 (m, 1H), 2.37 (s, 3H), 2.42-2.55 (m, 1H+DMSO), 2.62 (dd, 1H) 3.26-3.37 (m, 1H+H₂O), 3.72 (dd, 1H), 3.95 (s, 3H), 5.11 (m, 1H), 7.18-7.30 (m, 3H), 7.40-7.54 (m, 3H), 7.59 (d, 1H), 7.68 (s, 1H), 8.35 (s 1H), 9.62 (s, 1H), 10.26 (s, 1H); Mass Spectrum: (M+H)⁺ 528.

### Compound 11-9

### (4S)-4-({4-[(3-chloro-2-fluorophenyl)amino]-7-methoxyquinazolin-6-yl}oxy)-1-methyl-N-(3-methyl-1H-pyrazol-5-yl)-D-prolinamide

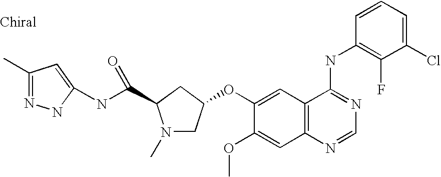

NMR Spectrum: (DMSO d₆) 2.14-2.56 (m, 2H+DMSO), 2.16 (s, 3H), 2.36 (s, 3H), 2.59-2.68 (m, 1H), 3.24-3.45 (m, 1H+H₂O), 3.67-3.75 (m, 1H), 3.93 (s, 3H), 5.11 (m, 1H), 6.29 (s, 1H), 7.21 (s, 1H), 7.28 (t, 1H), 7.44-7.53 (m, 2H), 7.69 (s, 1H), 8.33 (s, 1H), 9.62 (s, 1H); Mass Spectrum: (M+H)⁺ 526.

### Compound 11-10

### methyl(4S)-4-({4-[(3-chloro-2-fluorophenyl)amino]-7-methoxyquinazolin-6-yl}oxy)-1-methyl-D-prolyl-L-serinate

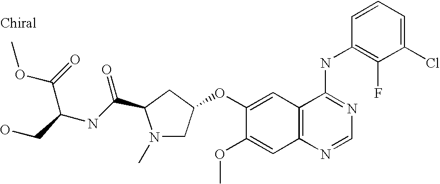

NMR Spectrum: (DMSO d₆+CD₃OOD) 2.18-2.28 (m, 1H), 2.33-2.44 (m, 4H), 2.58-2.65 (m, 1H), 3.25-3.33 (m, 1H), 3.60-3.68 (m, 4H), 3.71-3.80 (m, 2H), 3.93 (s, 3H), 4.36-4.40 (m, 1H), 5.05-5.13 (m, 1H), 7.20-7.30 (m, 2H), 7.42-7.55 (m, 2H), 7.70 (s, 1H), 8.36 (s, 1H); Mass Spectrum: (M+H)⁺ 548.

### Compound 11-11

### (4S)-4-({4-[(3-chloro-2-fluorophenyl)amino]-7-methoxyquinazolin-6-yl}oxy)-N-(2-hydroxy-1,1-dimethylethyl)-1-methyl-D-prolinamide

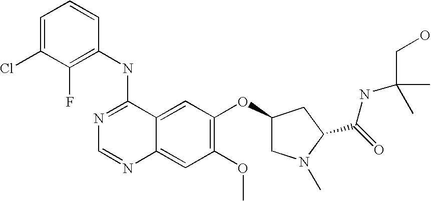

¹H NMR Spectrum: ¹H NMR (500 MHz, DMSO) δ 1.15 (d, 6H), 2.08-2.15 (m, 1H), 2.20-2.25 (m, 1H), 2.27 (s, 3H), 2.98 (t, 1H), 3.17-3.34 (m, 3H), 3.61-3.66 (m, 1), 3.87 (s, 3H), 4.87 (t, 1H), 4.99 (s, 1H), 7.16 (d, 2H), 7.21 (t, 1H), 7.41 (t, 1H), 7.46 (t, 1H), 7.63 (s, 1H), 8.31 (s, 1H), 9.55 (s, 1H).

Mass Spectrum: (M+H)⁺ 518.

### Compound 11-12

### (4S)-4-({4-[(3-chloro-2-fluorophenyl)amino]-7-methoxyquinazolin-6-yl}oxy)-1-methyl-D-prolylglycinamide

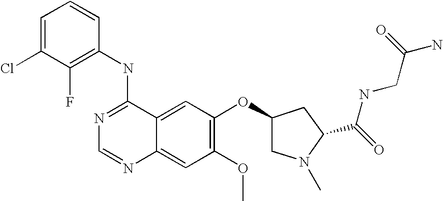

¹H NMR Spectrum: (500 MHz, DMSO) 2.09-2.16 (m, 1H), 2.30 (s, 3H), 3.09 (t, 1H), 3.18-3.23 (m, 2H), 3.56-3.70 (m, 3H), 3.88 (s, 3H), 4.99 (s, 1H), 6.93 (s, 1H), 7.15 (s, 1H), 7.19-7.26 (m, 2H), 7.41 (t, 1H), 7.45 (t, 1H), 7.63 (s, 1H), 7.82 (t, 1H), 8.30 (s, 1H), 9.56 (s, 1H)

Mass Spectrum: (M+H)⁺ 503.

### Compound 11-13

### (4S)—N-[2-(acetylamino)ethyl]-4-({4-[(3-chloro-2-fluorophenyl)amino]-7-methoxyquinazolin-6-yl}oxy)-1-methyl-D-prolinamide

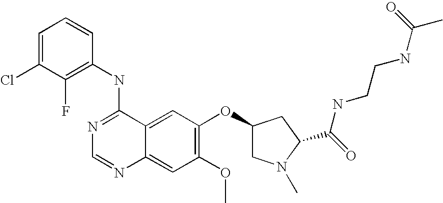

¹H NMR Spectrum: (500 MHz, DMSO) 1.72 (s, 3H), 2.05-2.13 (m, 1H), 2.21-2.34 (m, 4H), 2.43-2.51 (m, 1H), 2.99-3.13 (m, 5H), 3.58-3.63 (m, 1H), 3.88 (s, 3H), 4.97 (s, 1H), 7.15 (s, 1H), 7.21 (t, 1H), 7.38-7.48 (m, 2H), 7.62 (s, 1H), 7.75-7.82 (m, 2H), 8.30 (s, 1H), 9.58 (s, 1H); Mass Spectrum: (M+H)⁺ 531.

### Compound 11-14

### (4S)-4-({4-[(3-chloro-2-fluorophenyl)amino]-7-methoxyquinazolin-6-yl}oxy)-N-[(3S,4R)-4-hydroxytetrahydrofuran-3-yl]-1-methyl-D-prolinamide

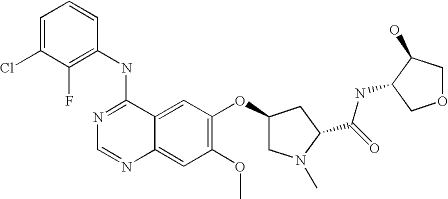

Mass Spectrum: (M+H)⁺ 532; Retention time 2.5 minutes (LCMS Conditions 1—see paragraph (xi) above).

### Compound 11-15

### (4S)-4-({4-[(3-chloro-2-fluorophenyl)amino]-7-methoxyquinazolin-6-yl}oxy)-N-[1-hydroxymethyl)cyclopentyl]-1-methyl-D-prolinamide

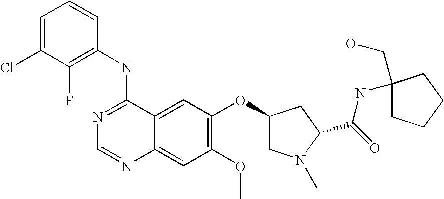

Mass Spectrum: (M+H)⁺ 544; Retention time 2.9 minutes (LCMS Conditions 1—see paragraph (xi) above).

### Compound 11-16

### (4S)-4-({4-[(3-chloro-2-fluorophenyl)amino]-7-methoxyquinazolin-6-yl}oxy)-N-[(1S) -1-(hydroxymethyl)-2-methylpropyl]-1-methyl-D-prolinamide

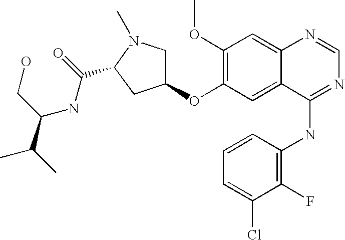

¹H NMR Spectrum: (500 MHz, DMSO) 0.74-0.83 (m, 6H), 1.74-1.83 (m, 1H), 2.09-2.17 (m, 1H), 2.23-2.31 (m, 4H), 2.40-2.50 (m, 1H), 3.11 (t, 1H), 3.29-3.41 (m, 2H), 3.48-3.56 (m, 1H), 3.63-3.68 (m, 1H), 3.88 (s, 3H), 4.53 (s, 1H), 5.00 (s, 1H), 7.15 (s, 1H), 7.21 (t, 1H), 7.31 (d, 1H), 7.41 (t, 1H), 7.46 (t, 1H), 7.64 (s, 1H), 8.31 (s, 1H), 9.56 (s, 1H); Mass Spectrum: (M+H)⁺ 532.

### Compound 11-17

### (4S)-4-({4-[(3-chloro-2-fluorophenyl)amino]-7-methoxyquinazolin-6-yl}oxy)-N-[2-(1H-imidazol-4-yl)ethyl]-1-methyl-D-prolinamide

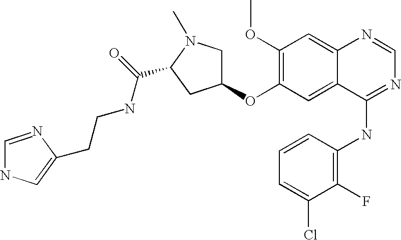

Mass Spectrum: (M+H)⁺ 540; Retention time 2.6 minutes (LCMS Conditions 1—see paragraph (xi) above).

### Compound 11-18

### (4S)-4-({4-[(3-chloro-2-fluorophenyl)amino]-7-methoxyquinazolin-6-yl}oxy)-N-(2-methoxy-1-methylethyl)-1-methyl-D-prolinamide

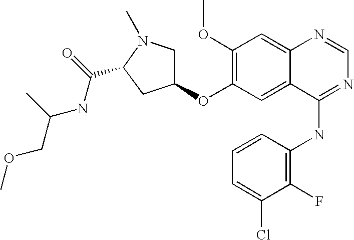

Mass Spectrum: (M+H)⁺ 518; Retention time 2.8 minutes (LCMS Conditions 1—see paragraph (xi) above).

### Compound 11-19

### (4S)-4-({4-[(3-chloro-2-fluorophenyl)amino]-7-methoxyquinazolin-6-yl}oxy)-1-methyl-N-(2,2,2-trifluoroethyl)-D-prolinamide

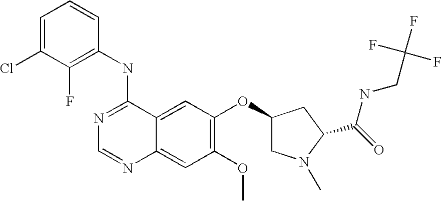

Mass Spectrum: (M+H)⁺ 528; Retention time 3.0 minutes (LCMS Conditions 1—see paragraph (xi) above).

### Compound 11-20

### (4S)—N-allyl-4-({4-[(3-chloro-2-fluorophenyl)amino]-7-methoxyquinazolin-6-yl}oxy)-1-methyl-D-prolinamide

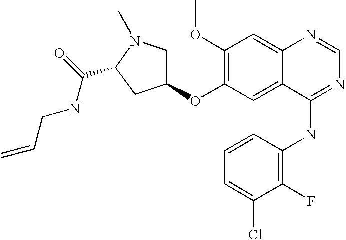

¹H NMR Spectrum: (500 MHz, DMSO) 2.06-2.14 (m, 1H), 2.24-2.35 (m, 4H), 2.40-2.51 (m, 1H), 3.10 (t, 1H), 3.59-3.69 (m, 3H), 3.88 (s, 3H), 4.96-5.08 (m, 3H), 5.70-5.79 (m, 1H), 7.15 (s, 1H), 7.21 (t, 1H), 7.41 (t, 1H), 7.46 (t, 1H), 7.62 (s, 1H), 7.93 (t, 1H), 8.30 (s, 1H), 9.55 (s, 1H); Mass Spectrum: (M+H)⁺ 486.

### Compound 11-21

### (4S)-4-({4-[(3-chloro-2-fluorophenyl)amino]-7-methoxyquinazolin-6-yl}oxy)-N-(2-ethoxyethyl)-1-methyl-D-prolinamide

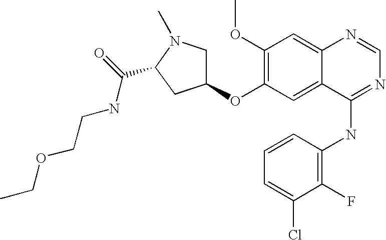

Mass Spectrum: (M+H)⁺ 518; Retention time 2.8 minutes (LCMS Conditions 1—see paragraph (xi) above).

### Compound 11-22

### (4S)-4-({4-[(3-chloro-2-fluorophenyl)amino]-7-methoxyquinazolin-6-yl}oxy)-N-(4-hydroxycyclohexyl)-1-methyl-D-prolinamide

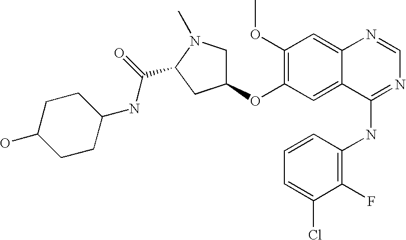

Mass Spectrum: (M+H)⁺ 544; Retention time 2.6 minutes (LCMS Conditions 1—see paragraph (xi) above).

### Compound 11-23

### (4S)-4-({4-[(3-chloro-2-fluorophenyl)amino]-7-methoxyquinazolin-6-yl}oxy)-1-methyl-N-(2-methylprop-2-en-1-yl)-D-prolinamide

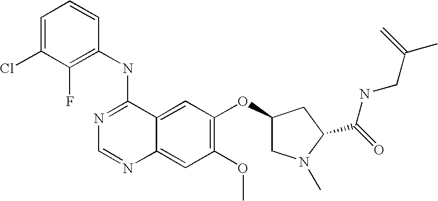

Mass Spectrum: (M+H)⁺ 500; Retention time 3.0 minutes (LCMS Conditions 1—see paragraph (xi) above).

### Compound 11-24

### (4S)-4-({4-[(3-chloro-2-fluorophenyl)amino]-7-methoxyquinazolin-6-yl}oxy)-N-[(1S)-1-(hydroxymethyl)propyl]-1-methyl-D-prolinamide

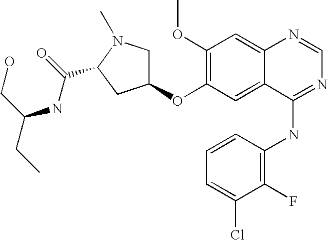

1H NMR (500 MHz, DMSO) 0.76 (t, 3H), 1.22-1.34 (m, 1H), 1.46-1.56 (m, 1H), 2.06-2.15 (m, 1H), 2.23-2.32 (m, 4H), 2.40-2.49 (m, 1H), 3.08 (t, 1H), 3.17-3.27 (m, 1H), 3.30-3.36 (m, 1H), 3.54-3.66 (m, 2H), 3.88 (s, 3H), 4.56 (t, 1H), 5.00 (s, 1H), 7.15 (s, 1H), 7.21 t, 1H), 7.37 (d, 1H), 7.41 (t, 1H), 7.46 (t, 1H), 7.63 (s, 1H), 8.30 (s, 1H), 9.56 (s, 1H)

Mass Spectrum: (M+H)⁺ 518.

### Compound 11-25

### (4S) -4-({4-[(3-chloro-2-fluorophenyl)amino]-7-methoxyquinazolin-6-yl}oxy)-N-[(2S)-2,3-dihydroxypropyl]-1-methyl-D-prolinamide

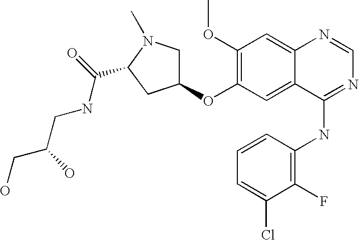

Mass Spectrum: (M+H)⁺ 520; Retention time 2.44 minutes (LCMS Conditions 1—see paragraph (xi) above).

### Compound 11-26

### (4S)-4-({4-[(3-chloro-2-fluorophenyl)amino]-7-methoxyquinazolin-6-yl}oxy)-N-(1H-imidazol-2-ylmethyl)-1-methyl-D-prolinamide

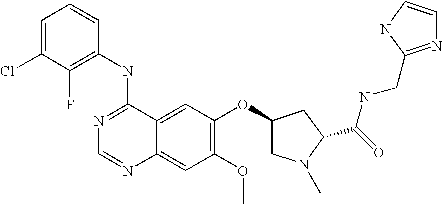

Mass Spectrum: (M+H)⁺ 526; Retention time 2.6 minutes (LCMS Conditions 1—see paragraph (xi) above).

### Compound 11-27

### (4S)-4-({4-[(3-chloro-2-fluorophenyl)amino]-7-methoxyquinazolin-6-yl}oxy)-N-[2-(2-furyl)ethyl]-1-methyl-D-prolinamide

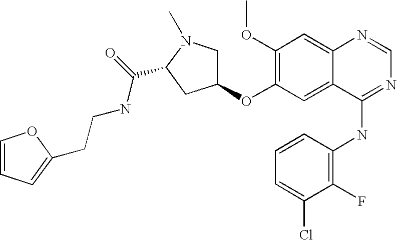

Mass Spectrum: (M+H)⁺ 540; Retention time 3.0 minutes (LCMS Conditions 1—see paragraph (xi) above).

### Compound 11-28

### (4S)-4-({4-[(3-chloro-2-fluorophenyl)amino]-7-methoxyquinazolin-6-yl}oxy)-1-methyl-N-(tetrahydro-2H-pyran-4-ylmethyl)-D-prolinamide

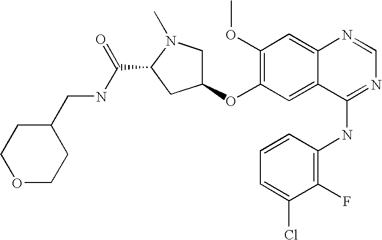

Mass Spectrum: (M+H)⁺ 544; Retention time 2.7 minutes (LCMS Conditions 1—see paragraph (xi) above).

### Compound 11-29

### (4S)-4-({4-[(3-chloro-2-fluorophenyl)amino]-7-methoxyquinazolin-6-yl}oxy)-N-[(1S)-2-hydroxy-1-methylethyl]-1-methyl-D-prolinamide

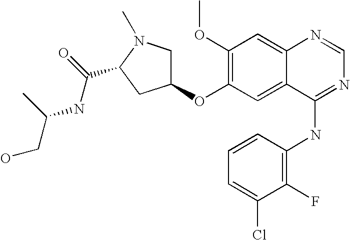

Mass Spectrum: (M+H)⁺ 504; Retention time 2.6 minutes (LCMS Conditions 1—see paragraph (xi) above).

### Compound 11-30

### (4S)-4-({4-[(3-chloro-2-fluorophenyl)amino]-7-methoxyquinazolin-6-yl}oxy)-N-[(1R)-2-hydroxy-1-methylethyl]-1-methyl-D-prolinamide

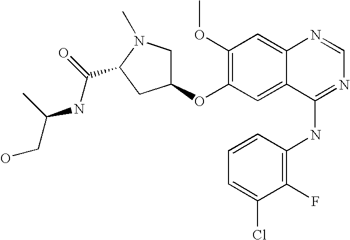

1H NMR Spectrum: (500 MHz, DMSO) 0.98 (d, 3H), 2.06-2.13 (m, 1H), 2.23-2.31 (m, 4H), 2.41-2.50 (m, 1H), 3.05 (t, 1H), 3.16-3.33 (m, 2H), 3.58-3.64 (m, 1H), 3.70-3.77 (m, 1H), 3.88 (s, 3H), 4.62 (t, 1H), 5.00 (s, 1H), 7.15 (s, 1H), 7.21 (t, 1H), 7.38-7.48 (m, 3H), 8.30 (s, 1H), 8.30 (s, 1H), 9.55 (s, 1H); Mass Spectrum: (M+H)⁺ 504.

### Compound 11-31

### (4S)-4-({4-[(3-chloro-2-fluorophenyl)amino]-7-methoxyquinazolin-6-yl}oxy)-N-[(2R)-2-hydroxypropyl]-1-methyl-D-prolinamide

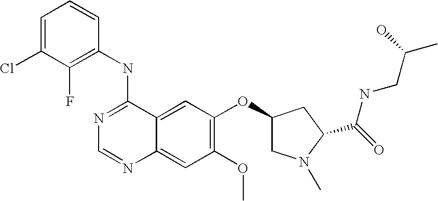

Mass Spectrum: (M+H)⁺ 504; Retention time 2.6 minutes (LCMS Conditions 1—see paragraph (xi) above).

### Compound 11-32

### (4S)-4-({4-[(3-chloro-2-fluorophenyl)amino]-7-methoxyquinazolin-6-yl}oxy)-N-[(2S)-2-hydroxypropyl]-1-methyl-D-prolinamide

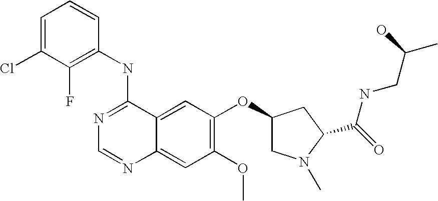

Mass Spectrum: (M+H)⁺ 504; Retention time 2.6 minutes (LCMS Conditions 1—see paragraph (xi) above).

### Compound 11-33

### (4S)-4-({4-[(3-chloro-2-fluorophenyl)amino]-7-methoxyquinazolin-6-yl}oxy)-1-methyl-N-[(2R)-tetrahydrofuran-2-ylmethyl]-D-prolinamide

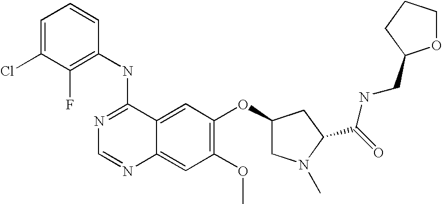

Mass Spectrum: (M+H)⁺ 530; Retention time 2.8 minutes (LCMS Conditions 1—see paragraph (xi) above).

### Compound 11-34

### (4)-4-({4-[(3-chloro-2-fluorophenyl)amino]-7-methoxyquinazolin-6-yl}oxy)-1-methyl-N-[(2S)-tetrahydrofuran-2-ylmethyl]-D-prolinamide

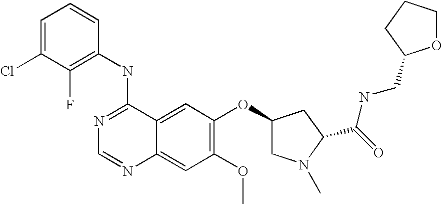

Mass Spectrum: (M+H)⁺ 530; Retention time 2.8 minutes (LCMS Conditions 1—see paragraph (xi) above).

### Compound 11-35

### N-(3-chloro-2-fluorophenyl)-7-methoxy-6-{[(3S,5R)-1-methyl-5-(pyrrolidin-1-ylcarbonyl)pyrrolidin-3-yl]oxy}quinazolin-4-amine

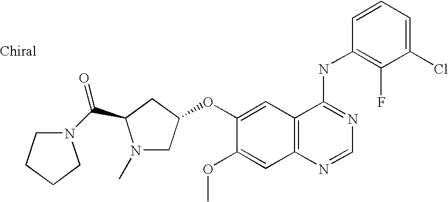

NMR Spectrum: (DMSO d₆) 1.69-1.80 (m, 2H), 1.80-1.91 (m, 2H), 2.08-2.17 (m, 1H), 2.31 (s, 3H), 2.51-2.60 (m, 2H), 3.25-3.37 (m, 1H+H2O), 3.42-3.58 (m, 4H), 3.65 (dd, 1H), 3.94 (s, 3H), 5.10 (m, 1H), 7.20 (s, 1H), 7.27 (t, 1H), 7.44-7.55 (m, 2H), 7.68 (s, 1H), 8.35 (s, 1H), 9.67 (s, 1H); Mass Spectrum: (M+H)⁺ 500.

### Compound 11-36

### N-(3-chloro-2-fluorophenyl)-7-methoxy-6-({(3S,5R)-1-methyl-5-[(4-methylpiperazin-1yl)carbonyl]pyrrolidin-3-yl}oxy)quinazolin-4-amine

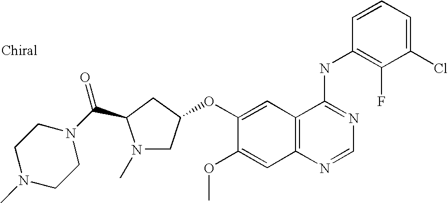

NMR Spectrum: (DMSO d₆) 2.07-2.60 (m, 7H+DMSO), 2.17 (s, 3H), 2.30 (s, 3H), 3.44-3.76 (m, 6H), 3.93 (s, 3H), 5.09 (m, 1H), 7.20 (s, 1H), 7.27 (t, 1H), 7.44-7.55 (m, 2H), 7.68 (d, 1H), 8.35 (s, 1H), 9.65 (s, 1H); Mass Spectrum: (M+H)⁺ 529.

### Compound 11-37

### 6-{[(3S,5R)-5-(azetidin-1-ylcarbonyl)-1-methylpyrrolidin-3-yl]oxy}-N-(3-chloro-2-fluorophenyl)-7-methoxyquinazolin-4-amine

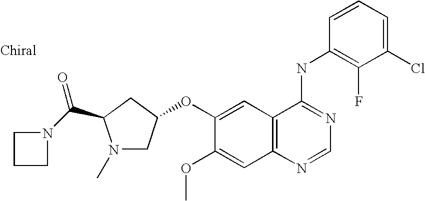

¹H NMR Spectrum: (300 MHz, DMSO) 2.10-2.24 (m, 4H), 2.33 (s, 3H), 2.44-2.59 (m, 2H), 3.62-3.72 (m, 1H), 3.80-3.93 (m, 2H), 3.93 (s, 3H), 4.13-4.32 (m, 2H), 5.08 (s, 1H), 7.20 (s, 1H), 7.27 (t, 1H), 7.40-7.57 (m, 2H), 8.36 (s, 1H), 8.36 (s, 1H), 9.65 (s, 1H); Mass Spectrum: (M+H)⁺ 486.

### Compound 11-38

### (4s)-4-{(4-[(3-chloro-2-fluorophenyl)amino]-7-methoxyquinazolin-6-yl}oxy)-N-(cyanomethyl)-N,1-dimethyl-D-prolinamide

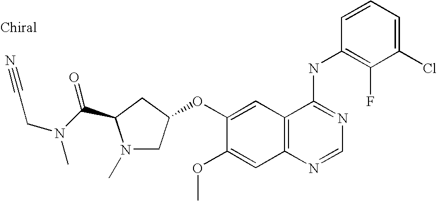

¹H NMR Spectrum: (300 MHz, DMSO) 2.13-2.27 (m, 1H), 2.31 (s, 3H), 2.43-2.65 (m, 2H), 2.91 (s, 3H)*, 3.15 (s, 3H)*, 3.56-3.67 (m, 1H), 3.79 (t, 1H), 3.93 (s, 3H), 4.39 (s, 2H)**, 4.78 (s, 2H)**, 5.10 (s, 1H), 7.20 (s, 1H), 7.27 (t, 1H), 7.43-7.55 (m, 2H), 7.69 (s, 1H), 8.36 (s, 1H), 9.65 (s, 1H); Mass Spectrum: (M+H)⁺ 499.

* and **rotameric signals

### Compound 11-39

### (4S)-4-({4-[(3-chloro-2-fluorophenyl)amino]-7-methoxyquinazolin-6-yl}oxy)-N-(cyanomethyl)-1-methyl-D-prolinamide

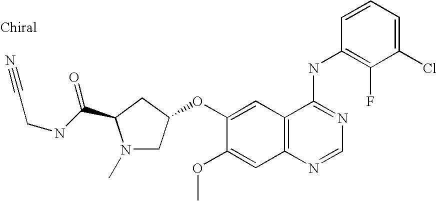

¹H NMR Spectrum: (300 MHz, DMSO) 2.13-2.26 (m, 1H), 2.26-2.45 (m, 1H), 2.32 (s, 3H), 2.58 (dd, 1H), 3.20 (t, 1H), 3.67 (dd, 1H), 3.93 (s, 3H), 4.13 (d, 2H), 5.05 (s, 1H), 7.21 (s, 1H), 7.27 (t, 1H), 7.41-7.56 (m, 2H), 7.67 (s, 1H), 8.36 (s, 1H), 8.60 (t, 1H), 9.63 (s, 1H); Mass Spectrum: (M+H)⁺485.

### Compound 11-40

### (4S)-4-({4-[(3-chloro-2-fluorophenyl)amino]-7-methoxyquinazolin-6-yl}oxy)-N,1-dimethyl-N-[(2S)-2-pyrrolidin-1-ylpropyl]-D-prolinamide

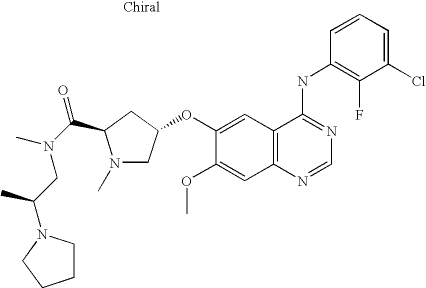

¹H NMR Spectrum: (DMSO d₆+CD₃CO₂D) 1.15 (d, 3H), 1.75-1.89 (m, 4H+CH₃COOH), 2.16-2.28 (m, 1H), 2.30 (s, 3H)*, 2.35 (s, 3H)*, 2.40-2.57 (m, 1H+DMSO), 2.60-2.69 (m, 1H), 2.82 (s, 3H)**, 2.97-3.23 (m, 4H), 3.07 (s, 3H)**, 3.39-3.54(m, 2H), 3.59-3.70 (m, 2H), 3.74-3.83 (m, 1H), 3.92 (s, 3H), 5.11 (m, 1H), 7.18-7.30 (t, 1H), 7.21 (s, 1H), 7.42-7.55 (m, 2H), 7.68-7.75 (m, 1H), 8.35 (s, 1H); Mass Spectrum: (M+H)⁺ 571.

* rotameric signals

** rotameric signals

### Compound 11-41

### (4S) -4-({4-[(3-chloro-2-fluorophenyl)amino]-7-methoxyquinazolin-6-yl}oxy)-N-[(1R)-2-hydroxy-1-methylethyl]-N,1-dimethyl-D-prolinamide

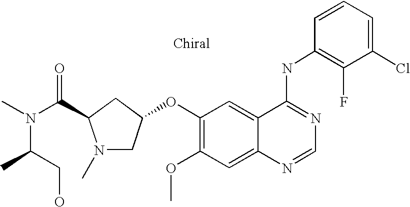

¹H NMR Spectrum: (DMSO d₆+CD₃CO₂D) 0.95-1.12 (m, 3H), 2.17-2.30 (m, 1H), 2.36-2.56 (m, 1H+DMSO), 2.40 (s, 3H), 2.63-2.78 (m, 1H), 2.68 (s, 3H)*, 2.87 (s, 3H)*, 3.29-3.42 (m, 2H), 3.64-3.82 (m, 1H), 3.87-4.11(m, 2H)**, 3.93 (s, 3H), 4.54 (m, 2H)**, 5.10 (m, 1H), 7.18-7.30 (t, 1H), 7.22 (s, 1H), 7.39-7.57 (m, 2H), 7.70 (s, 1H), 8.36 (s, 1H);

Mass Spectrum: (M+H)⁺ 518.

* rotameric signals

** rotameric signals

### Compound 11-42

### (4S)-4-({4-[(3-chloro-2-fluorophenyl)amino]-7-methoxyquinazolin-6-yl}oxy)-N,1-dimethyl-N-(1-methylpiperidin-4-yl)-D-prolinamide

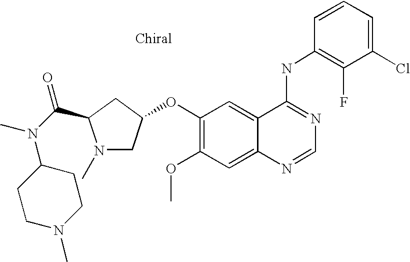

¹H NMR Spectrum: (DMSO d₆+CD₃CO₂D) 1.49-1.78 (m, 2H), 1.81-2.11 (m, 2H+CH₃COOH), 2.13-2.29 (m, 1H), 2.32 (s, 3H), 2.41-2.94 (m, 4H+DMSO), 2.49 (s, 3H), 2.70 (s, 3H)*, 2.88 (s, 3H)*, 3.14-3.28 (m, 2H), 3.59-3.70 (m, 1H), 3.73-3.85 (m, 1H), 3.92 (s, 3H), 3.98-4.11 (m, 1H)**, 4.37-4.50 (m, 1H)**, 5.12 (m, 1H), 7.18-7.31 (t, 1H), 7.20 (s, 1H), 7.38-7.57 (m, 2H), 7.73 (s, 1H), 8.35 (s, 1H); Mass Spectrum: (M+H)⁺ 557.

* rotameric signals

** rotameric signals

### Compound 11-43

### (4S)-4-({4-[(3-chloro-2-fluorophenyl)amino]-7-methoxyquinazolin-6-yl}oxy)-N,1-dimethyl-N-(tetrahydro-2H-pyran-4-yl)-D-prolinamide

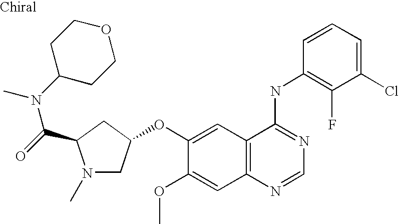

¹H NMR Spectrum: (DMSO d₆+CD₃CO₂D) 1.35-1.55 (m, 2H), 1.55-1.95 (m, 2H+CH₃COOH), 2.29-2.65 (m, 1H+DMSO), 2.70-2.90 (m, 4H), 2.97 (s, 3H), 3.25-3.45 (m, 3H), 3.45-3.60 (m, 1H), 3.60-3.75 (m, 1H)*, 3.75-4.05 (m, 2H), 3.92 (s, 3H), 4.10-4.28 (m, 1H), 4.33-4.53 (m, 1H)*, 4.85 (m, 1H)**, 5.00 (m, 1H)**, 7.24 (dd, 1H), 7.30 (s, 1H), 7.39-7.56 (m, 2H), 7.70-7.80 (m, 1H), 8.43 (s, 1H); Mass Spectrum: (M+H)⁺ 544.

* rotameric signals

** rotameric signals

### EXAMPLE 12

### (4R)-4-({4-[(3-chloro-2-fluorophenyl)amino]-7-methoxyquinazolin-6-yl}oxy)-1-methyl-N-prop-2-yn-1-yl-L-prolinamide

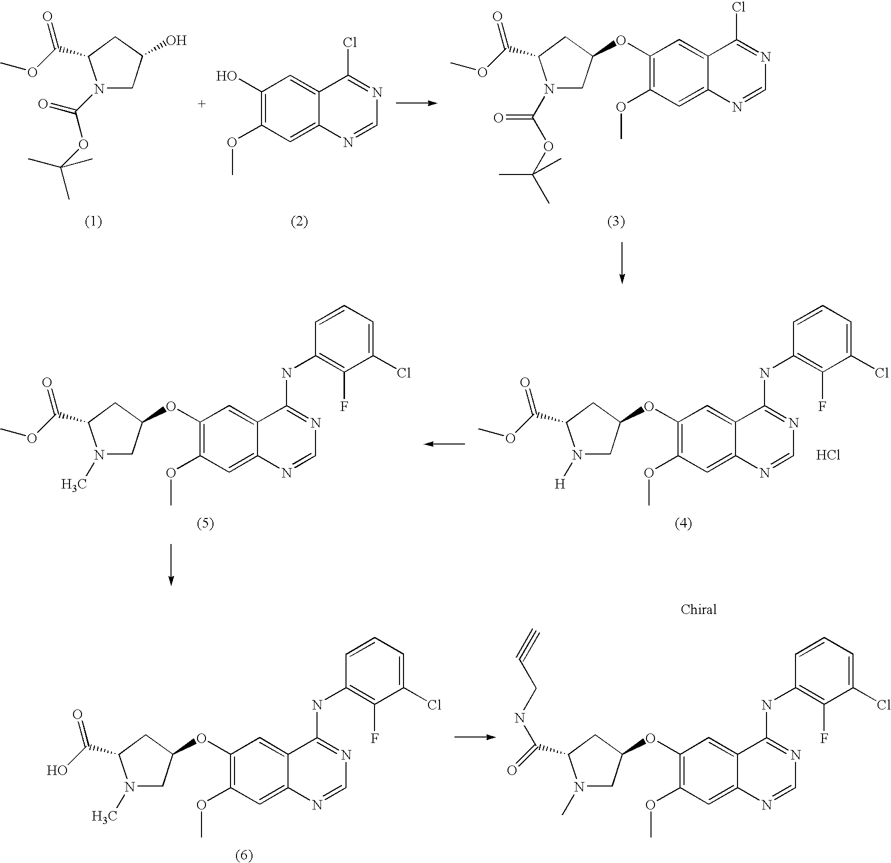

### Example 12

HATU (0.34 g) was added to an agitated solution of (4R)-4-({4-[(3-chloro-2-fluorophenyl)amino]-7-methoxyquinazolin-6-yl}oxy)-1-methyl-L-proline (0.2 g), propargylamine (49.3 mg) and DIPEA (231 mg) in dimethylacetamide (10 ml). The mixture was stirred at 50° C. for 10 minutes then allowed to stand at room temperature overnight. The reaction mixture was reduced in vacuo. The residues were re-dissolved in methylene chloride and washed with sodium hydroxide solution (2M) and water. The organic phase was then purified by column chromatography on silica eluting with increasingly polar mixtures of methanol/methylene chloride (0/100-12/88). The fractions containing the desired product were combined and evaporated to a foam which was triturated with diethylether to give the title compound as a white solid. (0.067 g). ¹H NMR Spectrum: (DMSO d₆) 2.08-2.22 (m, 1H), 2.25-2.62 (m, 2H+DMSO), 2.31 (s, 3H), 3.06 (s, 1H), 3.15 (t, 1H), 3.62-3.72 (m, 1H), 3.78-4.02 (m, 2H), 3.93 (s, 3H), 5.06 (m, 1H), 7.16-7.32 (m, 1H), 7.21 (s, 1H), 7.43-7.56 (m, 2H), 7.67 (s, 1H), 8.28 (m, 1H), 8.36 (s, 1H), 9.63 (s, 1H); Mass Spectrum: (M+H)⁺ 484.

The starting material was prepared as follows:

1-tert-butyl 2-methyl(2S,4S)-4-hydroxypyrrolidine-1,2-dicarboxylate (1), (Boc-cis-Hyp-OMe) is commercially available.

Di-ethyl azodicarboxylate (12.4 g) was added slowly to a stirred suspension of 1-tert-butyl 2-methyl(2S,4S)-4-hydroxypyrrolidine-1,2-dicarboxylate (1) (17.46 g), 4-chloro-7-methoxyquinazolin-6-ol (2) (10 g) [prepared as described in Example 1 above (compound 3)] and triphenylphosphine(18.67 g) in methylene chloride (300 ml) at 25° C. under an atmosphere of nitrogen and the reaction mixture was stirred for 1 hours. The reaction mixture was then evaporated to ½ volume and purified by column chromatography on silica eluting with increasingly polar mixtures of methanol/methylene chloride (0/100-3.5/96.5). The desired product fractions were combined and evaporated to give 1-tert-butyl 2-methyl(2S,4R)-4-[(4-chloro-7-methoxyquinazolin-6-yl)oxy]pyrrolidine-1,2-dicarboxylate(3) as a pale yellow foam Mass Spectrums: (M+H)⁺ 438.

This was used in the preparation of (4) without further purification.

The starting material (4) was prepared as follows:

4.0M HCl in Dioxane (39.2 ml) was added to a suspension of 1-tert-butyl 2-methyl (2S,4R)-4-[(4-chloro-7-methoxyquinazolin-6-yl)oxy]pyrrolidine-1,2-dicarboxylate (3) and 3-chloro-2-fluoroaniline (7.61 g) in acetonitrile (300 ml) and the reaction mixture was stirred and heated at 50° C. for 1 hours. The resulting precipitate was filtered hot and washed with acetonitrile and diethylether and dried under vacuum to give methyl(4R)-4-({4-[(3-chloro-2-fluorophenyl)amino]-7-methoxyquinazolin-6-yl}oxy)-L-prolinate hydrochloride (4) as an off-white solid, (23.05 g). ¹H NMR Spectrum: (DMSO d₆) 2.46-2.74 (m, 2H), 3.24-3.68 (m, 1H), 3.78 (s, 3H), 3.95-4.07 (m, 1H), 4.00 (s, 3H), 4.61 (t, 1H), 5.50 (m, 1H), 7.35 (t, 1H), 7.47-7.57 (m, 1H), 7.49 (s, 1H), 7.63 (t, 1H), 8.73 (s, 1H), 8.83 (s, 1H), 12.38 (bs, 1H):Mass Spectrum: (M+H)⁺ 447.

Methyl(4R)-4-({4-[(3-chloro-2-fluorophenyl)amino]-7-methoxyquinazolin-6-yl}oxy)-L-prolinate hydrochloride (4) (22.9 g), paraformaldehyde (14.25 g), sodium cyanoborohydride (11.97 g) and magnesium sulphate (11.4 g) were suspended in methanol (600 ml) and heated at 45° C. for 3 hours under an atmosphere of nitrogen. The reaction mixture was filtered, evaporated and partitioned between ethylacetate and saturated aqueous sodium bicarbonate solution. The organics were then washed with saturated brine, dried over MgSO₄, filtered and evaporated. The residues were then purified by column chromatography on silica eluting with increasingly polar mixtures of methanol/methylene chloride (0/100-10/90) to give methyl(4R)-4-({4-[(3-chloro-2-fluorophenyl)amino]-7-methoxyquinazolin-6-yl}oxy)-1-methyl-L-prolinate (5) as a yellow solid,(14.87 g). ¹H NMR Spectrum: (DMSO d₆) 2.13-2.25 (m, 1H), 2.34 (s, 3H), 2.46-2.61 (m, 2H+DMSO), 3.37 (t, 1H), 3.57-3.69 (m, 1H), 3.66 (s, 3H), 3.93 (s, 3H), 5.08 (m, 1H), 7.21 (s, 1H), 7.23-7.31 (t, 1H), 7.43-7.58 (m, 2H), 7.69 (s, 1h), 8.37 (s, 1H), 9.62 (s, 1H); Mass Spectrum: (M+H)⁺ 461.

Sodium hydroxide 2M (24.2 ml) was added to a stirred solution of methyl(4R)-4-({4-[(3-chloro-2-fluorophenyl)amino]-7-methoxyquinazolin-6-yl}oxy)-1-methyl-L-prolinate (5) (14.87 g) in methanol (100 ml) at 25° C. and the reaction mixture was stirred for 1 hour. The reaction mixture was evaporated and the residue re-dissolved in water. The pH of this solution was then adjusted to 6 by the dropwise addition of 2M HCl (aq) to give (4R)-4-({4-[(3-chloro-2-fluorophenyl)amino]-7-methoxyquinazolin-6-yl}oxy)-1-methyl-L-proline (6) as a pale yellow solid which was filtered and washed with water and dried, (11.5 g). ¹H NMR Spectrum: (DMSO d₆) 2.19-2.32 (m, 1H), 2.40-2.60 (m, 4H+DMSO), 3.75 (dd, 1H), 3.40 (t, 1H), 3.69-3.80 (dd, 1H), 3.93 (s, 3H), 5.09 (m, 1H), 7.17-7.33 (t, 1H), 7.21 (s, 1H), 7.41-7.58 (m, 2H), 7.72 (s, 1H), 8.37 (s, 1H), 9.69 (s, 1H); Mass Spectrum: (M+H)⁺ 447.

### EXAMPLE 13

The following compounds were prepared by coupling (4R)-4-({4-[(3-chloro-2-fluorophenyl)amino]-7-methoxyquinazolin-6-yl}oxy)-1-methyl-L-proline (intermediate (6) described in Example 12 above) with an appropriate amine using the same methodology as described in example 1.

### Compound 13-1

### N-(3-chloro-2-fluorophenyl)-6-{[(3R,5S)-5-(2,5-dihydro-1H-pyrrol-1-ylcarbonyl)-1-methylpyrrolidin-3-yl]oxy}-7-methoxyquinazolin-4-amine

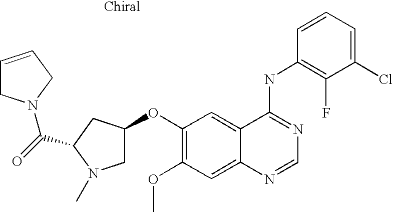

¹H NMR Spectrum: (DMSO d₆) 2.08-2.18 (m, 1H), 2.28 (s, 3H), 2.42-2.59 (m, 2H+DMSO), 3.45-3.55 (m, 1H), 3.57-3.65 (m, 1H), 3.90 (s, 3H), 3.94-4.16 (m, 2H+ethyl acetate), 4.22-4.42 (m, 2H), 5.08 (m, 1H), 5.80-5.90 (m, 2H), 7.17 (s, 1H), 7.24 (t, 1H), 7.40-7.52 (m, 2H), 7.65 (s, 1H), 8.33 (s, 1H), 9.62 (s, 1H); Mass Spectrum: (M+H)⁺ 498.

### Compound 13-2

### (4R)-4-({4-[(3-chloro-2-fluorophenyl)amino]-7-methoxyquinazolin-6-yl}oxy)-N-(cyanomethyl)-1-methyl-L-prolinamide

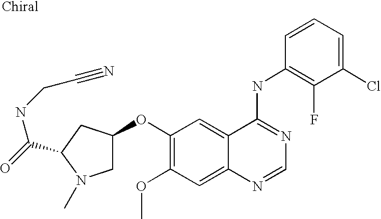

¹H NMR Spectrum: (DMSO d₆) 2.14-2.27 (m, 1H), 2.29-2.45 (m, 1H), 2.32 (s, 3H), 2.58 (dd, 1H), 3.17-3.32 (m, 1H+H₂O), 3.63-3.72 (m, 1H), 3.94 (s, 3H), 4.09-4.16 (m, 2H), 5.06 (m, 1H), 7.21 (s, 1H), 7.27 (t, 1H), 7.43-7.57 (m, 2H), 7.67 (s, 1H), 8.36 (s, 1H), 8.60 (m, 1H), 9.62 (s, 1H); Mass Spectrum: (M+H)⁺ 485.

### Compound 13-3

### (4R)-4-({4-[(3-chloro-2-fluorophenyl)amino]-7-methoxyquinazolin-6-yl}oxy)-N-(2-cyanomethyl)-1-methyl-L-prolinamide

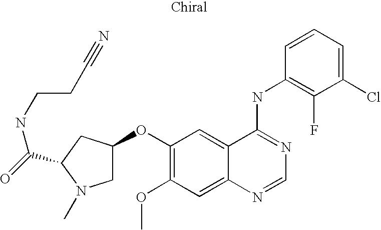

^{H} NMR Spectrum: (DMSO d₆+CD₃CO₂D) 2.17-2.45 (m, 2H), 2.38 (s, 3H), 2.58-2.70 (m, 3H), 3.19-3.28 (m, 1H), 3.28-3.42 (m, 2H), 3.66-3.80 (m, 1H), 3.92 (s, 3H), 5.07 (m, 1H), 7.19-7.29 (m, 2H), 7.38-7.55 (m, 2H), 7.68 (s, 1H), 8.35 (s, 1H); Mass Spectrum: (M+H)⁺ 499.

### Compound 13-4

### (4R)-4-({4-[(3-chloro-2-fluorophenyl)amino]-7-methoxyquinazolin-6-yl}oxy)-N-(cyanomethyl)-N,1-dimethyl-L-prolinamide

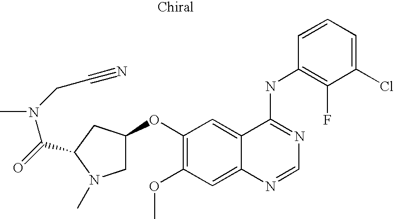

¹H NMR Spectrum: (DMSO d₆+CD₃CO₂D) 2.35-2.58 (m, 5H+DMSO), 2.88-2.97 (m, 1H), 3.12 (s, 3H), 3.76-3.84 (m, 1H), 3.91 (s, 3H), 4.18 (t, 1H), 4.39 (s, 2H)*, 4.69 (s 2H)*, 5.15 (m, 1H), 7.23 (m, 1H), 7.25 (s, 1H), 7.38-7.52 (m, 2H), 7.70 (s, 1H), 8.36 (s, 1H); Mass Spectrum: (M+H)⁺ 499.

* rotameric protons

### Compound 13-5

### (4R)-4-({4-[(3-chloro-2-fluorophenyl)amino]-7-methoxyquinazolin-6-yl}oxy)-N-(2-methoxyethyl)-1-methyl-L-prolinamide

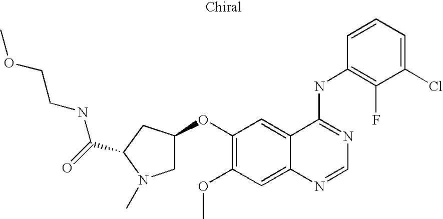

¹H NMR Spectrum: (DMSO d₆+CD₃CO₂D) 2.17-2.43 (m, 2H), 2.39 (s, 3H), 2.64-2.73 (m, 1H), 3.14-3.38 (m, 5H), 3.20 (s, 3H), 3.69-3.79 (m, 1H), 3.90 (s, 3H), 5.05 (m, 1H), 7.16-7.28 (m, 1H), 7.19 (s, 1H), 7.38-7.53 (m, 2H), 7.65 (s, 1H), 8.34 (s, 1H); Mass Spectrum: (M+H)⁺ 504.

### Compound 13-6

### (4R)-4-({4-[(3-chloro-2-fluorophenyl)amino]-7-methoxyquinazolin-6-yl}oxy)-N-cyclopropyl-1-methyl-L-prolinamide

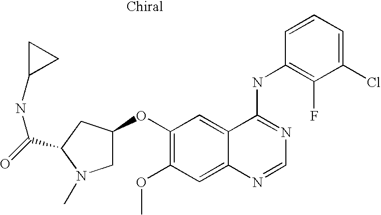

¹H NMR Spectrum: (DMSO d₆+CD₃CO₂D) 0.002-0.08 (m, 2H), 0.16-0.27 (m, 2H), 1.82-2.06 (m, 2H), 2.04 (s, 3H), 2.21-2.31 (m, 1H), 2.37 (dd, 1H), 2.12-3.03 (m, 1H), 3.34-3.44 (m, 1H), 3.51 (s, 3H), 4.68 (m, 1H), 6.78-6.88 (m, 1H), 6.98-7.14 (m, 2H), 7.26 (s, 1H), 7.95 (s, 1H); Mass Spectrum: (M+H)⁺ 486.

### Compound 13-7

### (4R)-4-({4-[(3-chloro-2-fluorophenyl)amino]-7-methoxyquinazolin-6-yl}oxy)-N-cyclopentyl-1-methyl-L-prolinamide

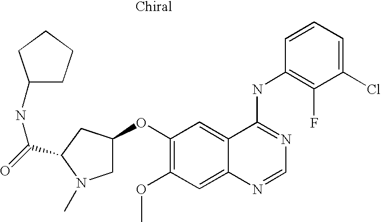

¹H NMR Spectrum: (DMSO d₆) 1.30-1.70 (m, 6H), 1.72-1.88 (m, 2H), 2.05-2.15 (m, 1H), 2.29 (s, 3H), 2.31-2.44 (m, 1H), 2.46-2.57 (m, 1H+DMSO), 3.11 (t, 1H), 3.65 (dd, 1H), 3.93 (s, 3H), 3.97-4.09 (m, 1H), 5.06 (m, 1H), 7.20 (s, 1H), 7.27 (t, 1H), 7.44-7.54 (m, 2H), 7.67 (s, 1H), 7.72 (d, 1H), 8.37 (s, 1H), 9.65 (s, 1H); Mass Spectrum: (M+H)⁺ 514.

### Compound 13-8

### N-(3-chloro-2-fluorophenyl)-7-methoxy-6-({(3R,5S)-1-methyl-5-[(4-methylpiperazin-1-yl)carbonyl[pyrrolidin-3-yl}oxy)quinazolin-4-amine

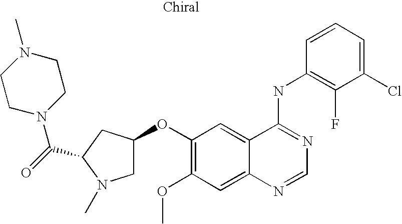

¹H NMR Spectrum: (DMSO d₆+CD₃CO₂D) 2.12-2.29 (m, 1H), 2.30 (s, 3H), 2.31-2.57 (m, 8H+DMSO), 2.85 (dd, 1H), 3.26-3.62 (m, 4H), 3.64-3.71 (m, 1H), 3.83 (t, 1H), 3.92 (s, 3H), 5.10 (m, 1H), 7.18-7.30 (m, 1H), 7.22 (s, 1H), 7.40-7.54 (m, 2H), 7.68 (s, 1H), 8.35 (s, 1H); Mass Spectrum: (M+H)⁺ 529.

### Compound 13-9

### (3S)-1-[(4R)-4-({4-[(3-chloro-2-fluorophenyl)amino]-7-methoxyquinazolin-6-yl}oxy)-1-methyl-L-prolyl]pyrrolidin-3-ol

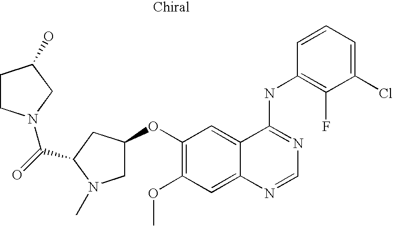

¹H NMR Spectrum: (DMSO+CD₃CO₂D) 1.69-1.99 (m, 2H+CH₃COOH), 2.40-2.59 (m, 2H+DMSO), 2.64 (d, 3H), 2.98-3.11 (m, 1H), 3.24-3.54 (m, 4H)*, 3.61-3.72 (m, 1H)*, 3.87-3.97 (m, 1H), 3.92 (s, 3H), 4.05-4.33 (m, 2H), 5.16 (m, 1H), 7.19-7.27 (m, 2H), 7.37-7.53 (m, 2H), 7.70 (s, 1H), 8.35 (s, 1H); Mass Spectrum: (M+H)⁺ 516.

* rotameric signals

### Compound 13-10

### (4R)-4-({4-[(3-chloro-2-fluorophenyl)amino]-7-methoxyquinazolin-6-yl}oxy)-N-(cyclopropylmethyl)-1-methyl-L-Prolinamide

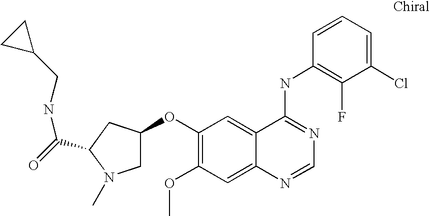

¹H NMR Spectrum: (DMSO d₆) 0.21-0.28 (m, 2H), 0.41-0.51 (m, 2H), 0.93-1.06 (m, 1H), 2.16-2.26 (m, 1H), 2.36-2.50 (m, 1H), 2.39 (s, 3H), 2.54-2.65 (m, 1H+DMSO), 3.00-3.08 (m, 2H), 3.20 (t, 1H), 3.70-3.79 (m, 1H), 4.00 (s, 3H), 5.12 (m, 1H), 7.28 (s, 1H), 7.34 (t, 1H), 7.50-7.62 (m, 2H), 7.74 (s, 1H), 7.91-7.98 (m, 1H), 8.44 (s, 1H), 9.70 (s, 1H); Mass Spectrum: (M+H)⁺ 500.

### Compound 13-11

### (4R)-4-({4-[(3-chloro-2-fluorophenyl)amino]-7-methoxyquinazolin-6-yl}oxy)-N-cyclohexyl-N,1-dimethyl-L-prolinamide

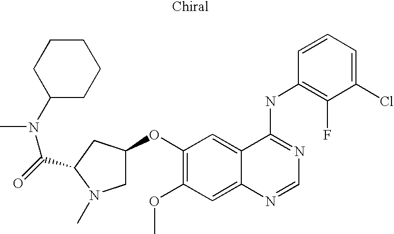

¹H NMR Spectrum (DMSO d₆) 1.19-1.63 (m, 8H), 1.67-1.80 (m, 2H), 2.05-2.17 (m, 1H), 2.28 (s, 3H), 2.41-2.54 (m, 1H+DMSO), 2.54-2.63 (m, 1H), 2.70 (s, 3H)*, 2.88 (s, 3H)*, 3.56-3.65 (m, 1H), 3.67-3.80 (m, 1H), 3.93 (s, 3H), 4.20-4.34 (m, 1H), 5.10 (m, 1H), 7.20 (s, 1H), 7.27 (t, 1H), 7.43-7.54 (m, 2H), 7.68 (m, 1H), 8.36 (s, 1H), 9.66 (s, 1H); Mass Spectrum: (M+H)⁺ 542.

* rotameric signals

### Compound 13-12

### (4R)-4-({4-[(3-chloro-2-fluorophenyl)amino]-7-methoxyquinazolin-6-yl}oxy)-1-methyl-N-(tetrahydro-2-pyran-4-yl)-L-prolinamide

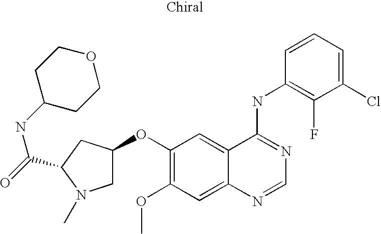

¹H NMR Spectrum: (DMSO d₆) 1.37-1.54 (m, 2H), 1.60-1.74 (m, 2H), 2.08-2.20 (m, 1H), 2.24-2.44 (m, 1H), 2.31 (s, 3H), 2.47-2.62 (m, 1H+DMSO), 3.08-3.20 (m, 1H), 3.24-3.40 (m, 2H+H₂O), 3.63-3.72 (m, 1H), 3.75-3.86 (m, 3H), 3.93 (s, 3H), 5.07 (m, 1H), 7.20 (s, 1H), 7.27 (t, 1H), 7.45-7.55 (m, 2H), 7.67 (s, 1H), 7.80 (d, 1H), 8.36 (s, 1H), 9.63 (s, 1H); Mass Spectrum: (M+H)⁺ 530.

### Compound 13-13

### N-(3-chloro-2-fluorophenyl)-7-methoxy-6-{[(3R,5S)-1-methyl-5-(pyrrolidin-1-ylcarbonyl)pyrrolidin-3-yl]oxy}quinazolin-4-amine

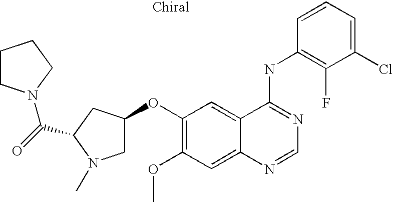

¹H NMR Spectrum: (DMSO d₆+CD₃CO₂D) 1.73-1.90 (m, 5H+CH₃COOH), 2.38-2.60 (m, 2H+DMSO), 2.71 (s, 3H), 3.16 (dd, 1H), 3.26-3.43 (m, 3H), 3.50-3.59 (m, 1H), 3.91 (s, 3H), 3.95-4.04 (m, 1H), 4.26 (t, 1H), 5.18 (m, 1H), 7.22 (t, 1H), 7.27 (s, 1H), 7.37-7.53 (m, 2H), 7.71 (s, 1H), 8.36 (s, 1H); Mass Spectrum: (M+H)⁺ 500.

### Compound 13-14

### (4R)-4-({4-[(3-chloro-2-fluorophenyl)amino]-7-methoxyquinazolin-6-yl}oxy)-N-(2-hydroxyethyl)-N,1-dimethyl-L-prolinamide

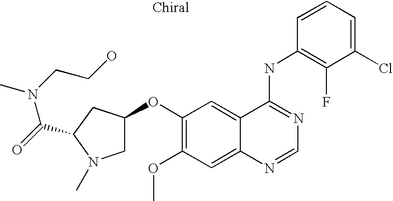

¹H NMR Spectrum: (DMSO ₆+CD₃CO₂D) 2.13-2.28 (m, 1H), 2.36 (s, 3H), 2.45-2.59 (m, 1H+DMSO), 2.65-2.76 (m, 1H), 2.85 (s, 3H)*, 3.08 (s, 3H)*, 3.31-3.57 (m, 4H), 3.64-3.76 (m, 1H), 3.80-4.02 (m, 1H), 3.93 (s, 3H), 5.12 (m, 1H), 7.18-7.30 (t, 1H), 7.22 (s, 1H), 7.40-7.55 (m, 2H), 7.69 (s, 1H), 8.36 (s, 1H); Mass Spectrum: (M+H)⁺ 50

* rotameric signals

### Compound 13-15

### (4R)-4-({4-[(3-chloro-2-fluorophenyl)amino]-7-methoxyquinazolin-6-yl}oxy)-N-[2-dimethylamino)ethyl]-1-methyl-L-prolinamide

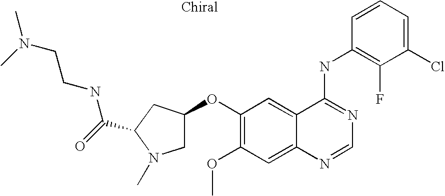

¹H NMR Spectrum: (DMSO d₆+CD₃CO₂D) 2.13-2.24 (m, 1H), 2.29-2.44 (m, 1H), 2.32 (s, 3H), 2.46-2.53 (s, 6H), 2.54-2.66 (m, 1H+DMSO), 2.89-3.00 (m, 2H), 3.10-3.20 (m, 1H), 3.30-3.44 (m, 2H),), 3.67 (dd, 1H), 3.91 (s, 3H), 5.05 (m, 1H), 7.17-2.29 (t, 1H), 7.22 (s, 1H), 7.40-7.55 (m, 2H), 7.70 (s, 1H), 8.35 (s, 1H); Mass Spectrum: (M+H)⁺ 517.

### Compound 13-16

### (4R)-4-({4-[(3-chloro-2-fluorophenyl)amino]-7-methoxyquinazolin-6-yl}oxy)-N,1-dimethyl-N-(1-methylpiperidin-4-yl)-L-prolinamide

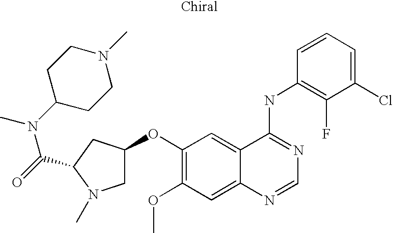

Mass Spectrum: (M+H)⁺ 557; Retention time 1.05 minutes (LCMS Conditions 2—see paragraph (xi) above).

### Compound 13-17

### 6({(3R,5S)-5-[(4-acetylpiperazin-1-yl)carbonyl]-1-methylpyrrolidin-3-yl}oxy)-N-(3-chloro-2-fluorophenyl)-7-methoxyquinazolin-4-amine

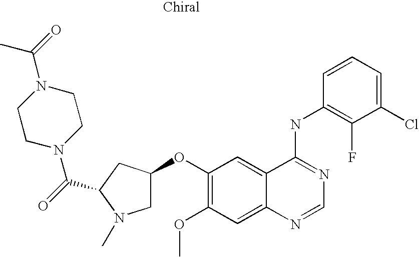

¹H NMR Spectrum: (DMSO d₆+CD₃CO₂D) 1.99 (s, 3H), 2.16-2.28 (m, 1H), 2.38 (s, 3H), 2.45-2.58 (m, 1H+DMSO), 2.63-2.73 (m, 1H), 3.34-3.64 (m, 8H), 3.70 (dd, 1H), 3.82-3.96 (m, 1H), 3.92 (s, 3H), 5.11 (m, 1H), 7.19-7.29 (t, 1H), 7.21 (s, 1H), 7.42-7.55 (m, 2H), 7.69 (s, 1H), 8.36 (s, 1H); Mass Spectrum: (M+H)⁺ 557.

### Compound 13-18

### 1-[(4R)-4-({4-[(3-chloro-2-fluorophenyl)amino]-7-methoxyquinazolin-6-yl}oxy)-1-methyl-L-prolyl]piperidin-4-ol

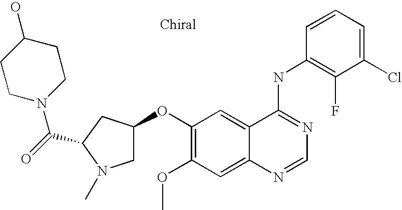

¹H NMR Spectrum: (DMSO d₆+CD₃CO₂D) 1.18-1.43 (m, 2H), 1.64-1.82 (m, 2H), 2.15-2.29 (m, 1H), 2.39 (s, 3H), 2.43-2.57 (m, 1H+DMSO), 2.63-2.76 (m, 1H), 2.98-3.12 (m, 1H), 3.16-3.28 (m, 1H), 3.63-3.98 (m, 5H), 3.92 (s, 3H), 5.11 (m, 1H), 7.19-7.29 (t, 1H), 7.21 (s, 1H), 7.40-7.55 (m, 2H), 7.69 (s, 1H), 8.35 (s, 1H); Mass Spectrum: (M+H)⁺ 530.

### Compound 13-19

### (4R)-4-({4-[(3-chloro-2-fluorophenyl)amino]-7-methoxyquinazolin-6-yl}oxy)-N-(2-methoxyethyl)-N,1-dimethyl-L-prolinamide

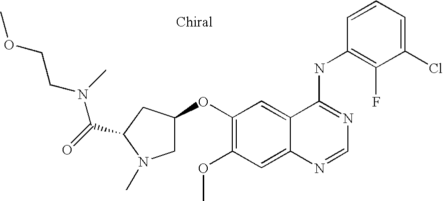

¹H NMR Spectrum: (DMSO d₆+CD₃CO₂D) 2.42-2.55 (m, 1H+DMSO), 2.60 (s, 3H), 2.84-2.91 (m, 1H), 2.89 (s, 3H)*, 3.00-3.15 (m, 1H), 3.04 (s, 3H)*, 3.23 (s, 3H)**, 3.24 (s, 3H)**, 3.40-3.56 (m, 4H), 3.86-4.02 (m, 1H), 3.93 (s, 3H), 4.26-4.42 (m, 1H), 5.18 (m, 1H), 7.20-7.30 (t, 1H), 7.24 (s, 1H), 7.41-7.55 (m, 2H), 7.75-7.78 (m, 1H), 8.37 (s, 1H); Mass Spectrum: (M+H)⁺ 518.

* rotameric signals

** rotameric signals

### Compound 13-20

### (4R)-4-({4-[(3-chloro-2-fluorophenyl)amino]-7-methoxyquinazolin-6-yl}oxy)-N-cyclohexyl-1-methyl-L-prolinamide

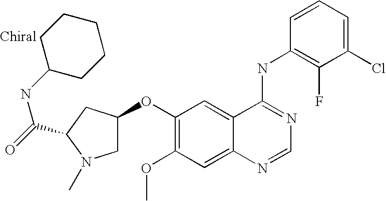

¹H NMR Spectrum: (DMSO d₆) 1.05-1.32 (m, 5H), 1.49-1.58 (m, 1H), 1.60-1.76 (m, 4H), 2.06-2.16 (m, 1H), 2.23-2.42 (m, 1H), 2.29 (s, 3H), 2.46-2.56 (m, 1H+DMSO), 3.11 (t, 1H), 3.51-3.59, (m, 1H), 3.65 (dd, 1H), 3.93 (s, 3H), 5.06 (m, 1H), 7.20 (s, 1H), 7.27 (t, 1H), 7.44-7.54 (m, 2H), 7.61 (d, 1H), 7.67 (s, 1H), 8.37 (s, 1H), 9.63 (s, 1H); Mass Spectrum: (M+H)⁺ 528.

### EXAMPLE 14

### (4S)-4-({4-[(3-chloro-2-fluorophenyl)amino]-7-methoxyquinazolin-6-yl}oxy)-N-cyclopropyl-1-methyl-L-prolinamide

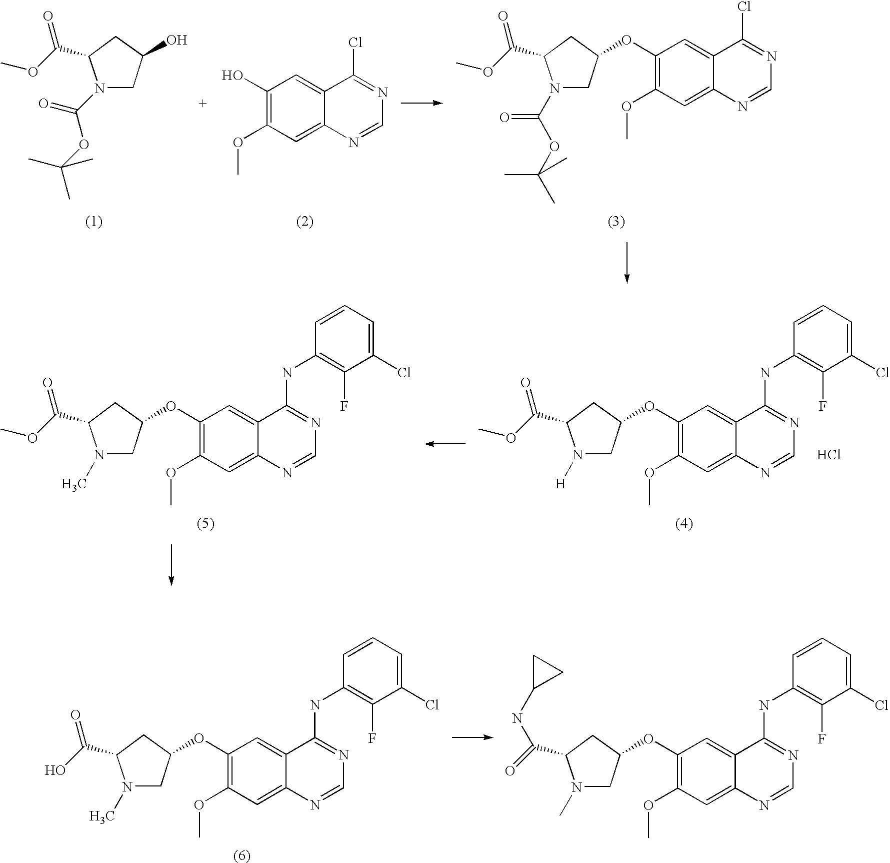

HATU (0.34 g) was added to stirred solution of (4S)-4-({4-[(3-chloro-2-fluorophenyl)amino]-7-methoxyquinazolin-6-yl}oxy)-1-methyl-L-proline (0.15 g), cyclopropylamine (38 mg) and DIPEA (0.17 ml) in dimethylacetamide (5.25 ml). The mixture was stirred at room temperature overnight. The reaction mixture was partitioned between saturated aqueous sodium bicarbonate and ethyl acetate (×3). Combined organics were dried over magnesium sulphate, filtered and evaporated. The residues were re-dissolved in methylene chloride and washed with sodium hydroxide solution (2M) and water. The crudes were then purified by column chromatography on silica eluting with methylene chloride/7N ammonia solution in methanol (96/4). Fractions containing the desired product were then combined and evaporated. This was triturated with a mixture of methylene chloride (3 ml)/isohexane (15 ml) to give (4S)-4-({4-[(3-chloro-2-fluorophenyl)amino]-7-methoxyquinazolin-6-yl}oxy)-N-cyclopropyl-1-methyl-L-prolinamide as a white solid which was collected by filtration and dried(0.085 g); ¹H NMR Spectrum: (DMSO d₆) 0.48 (m, 2H), 0.64 (m, 2H), 1.92 (m, 1H), 2.29 (s, 3H), 2.69(m, 2H), 2.84 (m, 2H), 3.30 (m, 1H), 3.96 (s, 3H), 5.02 (m, 1H), 7.23 (s, 1H), 7.31 (m, 1H), 7.53 (m, 2H), 7.66 (s, 1H), 7.70 (m, 1H), 8.40 (s, 1H), 9.58 (br s, 1H); Mass Spectrum: (M+H)⁺ 486.

Starting material 1-tert-butyl 2-methyl(2S,4R)-4-hydroxypyrrolidine-1,2-dicarboxylate (1) is commercially available.

Starting material (3) was prepared as follows:

1-tert-butyl 2-methyl(2S,4R)-4-hydroxypyrrolidine-1,2-dicarboxylate (1) was coupled with 4-chloro-7-methoxyquinazolin-6-ol (2) analogously as for the equivalent step in example 1 to give 1-tert-butyl 2-methyl(2S,4S)-4-[(4-chloro-7-methoxyquinazolin-6-yl)oxy]pyrrolidine-1,2-dicarboxylate (3). Mass Spectrum: (M+H)⁺ 438.

This was used in the preparation of (4) without further purification.

The starting material (4) was prepared as follows:

1-tert-butyl 2-methyl(2S,4S)-4-[(4-chloro-7-methoxyquinazolin-6-yl)oxy]pyrrolidine-1,2-dicarboxylate (3) was coupled with 3-chloro-2-fluoroaniline analogously as for the equivalent step in example 1 to give methyl(4S)-4-({4-[(3-chloro-2-fluorophenyl)amino]-7-methoxyquinazolin-6-yl}oxy)-L-prolinate hydrochloride (4). ¹H NMR Spectrum: (DMSO d₆) 2.50-2.67 (m, 1H), 2.70-2.85 (m, 1H), 3.55 (d, 1H), 3.75 (s, 3H), 3.88 (dd, 1H), 3.98 (s, 3H), 4.75 (dd, 1H), 5.50 (m, 1H), 7.35 (t, 1H), 7.45 (s, 1H), 7.53 (t, 1H), 7.63 (t, 1H), 8.73 (s, 1H), 8.81 (s, 1H), 12.36 (bs, 1H); Mass Spectrum: (M+H)⁺ 447.

Compound (5) was prepared as follows:

Methyl(4S)-4-({4-[(3-chloro-2-fluorophenyl)amino]-7-methoxyquinazolin-6-yl}oxy)-L-prolinate hydrochloride (4) (7.5 g), paraformaldehyde (4.66 g), sodium cyanoborohydride (3.91 g) and magnesium sulphate (3.72 g) were suspended in methanol (75 ml) and heated to 40° C. overnight. The reaction mixture was filtered, evaporated and partitioned between methylene chloride and saturated aqueous sodium bicarbonate solution. The organics were then washed with saturated brine, dried over MgSO₄, filtered and evaporated. The residues were then purified by column chromatography on silica eluting with methylene chloride/7N ammonia solution in methanol (98/2) to give methyl(4S)-4-({4-[(3-chloro-2-fluorophenyl)amino]-7-methoxyquinazolin-6-yl}oxy)-1-methyl-L-prolinate as a yellow solid, (3.66 g). ¹H NMR Spectrum: (DMSO d₆) 1.89-2.08 (m, 1H), 2.30 (s, 3H), 2.60-2.80 (m, 1H), 2.80-3.00 (m, 1H), 3.00-3.13 (m, 1H), 3.23 (m, 1H), 3.65 (s, 3H), 3.93 (s, 3H), 5.00(m, 1H), 7.20 (s, 1H), 7.27 (dd, 1H), 7.40-7.58 (m, 2H), 7.60 (s, 1H), 8.37 (s, 1H), 9.58 (s, 1H); Mass Spectrum: (M+H)⁺ 461.

Compound (6) was prepared as follows:

Sodium hydroxide 2M (6 ml) was added to a stirred solution of methyl(4S)-4-({4-[(3-chloro-2-fluorophenyl)amino]-7-methoxyquinazolin-6-yl}oxy)-1-methyl-L-prolinate (5) (3.66 g) in a mixture of methanol (24 ml) and tetrahydrofuran. The reaction mixture was stirred at 40° C. for 1.5 hours. The reaction mixture was evaporated and the residue re-dissolved in water. The pH of this solution was then adjusted to 6 by the dropwise addition of 2M HCl (aq) to give (4S)-4-({4-[(3-chloro-2-fluorophenyl)amino]-7-methoxyquinazolin-6-yl}oxy)-1-methyl-L-proline (6) as a yellow solid which was filtered, washed with water and dried, (2.95 g). ¹H NMR Spectrum: (DMSO d₆) 1.9-2.15 (m, 1H), 2.49 (s, 3H+DMSO), 2.80-3.05 (m, 2H) 3.20 (t, 1H, 3.42 (d, 1H), 3.93 (s, 3H), 5.05(m, 1H), 7.20 (s, 1H), 7.26 (dd, 1), 7.40-7.60 (m, 2H), 7.68 (s, 1H), 8.37 (s, 1H), 9.70 (brs, 1H); Mass Spectrum: (M+H)⁺ 447.

### EXAMPLE 15

The following compounds were made using the same methodology as described above by coupling (4S)-4-({4-[(3-chloro-2-fluorophenyl)amino]-7-methoxyquinazolin-6-yl}oxy)-1-methyl-L-proline with the appropriate amine.

### Compound 15-1

### (4S)-4-({4-[(3-chloro-2-fluorophenyl)amino]-7-methoxyquinazolin-6-yl}oxy)-N-(2-methoxyethyl)-1-methyl-L-prolinamide

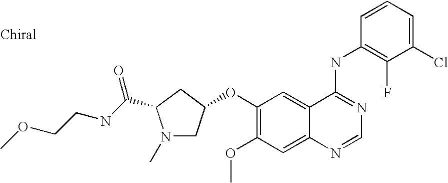

¹H NMR Spectrum: (DMSO d₆) 1.89 (m, 1H), 2.30(s, 3H), 2.72(m, 2H), 2.89 (m, 2H), 3.25 (s, 3), 3.36 (m, 4H), 3.96 (s, 3H), 5.03 (m, 1H), 7.22 (s, 1H), 7.31 (m, 1H), 7.51 (m, 2H), 7.66 (s, 1H), 7.75 (m, 1H), 8.38 (s, 1H), 9.58 (brs, 1H); Mass Spectrum: (M+H)⁺ 504.

### Compound 15-2

### (4S)-4-({4-[(3-chloro-2-fluorophenyl)amino]-7-methoxyquinazolin-6-yl}oxy)-N-cyclohexyl-N,1-dimethyl-L-prolinamide

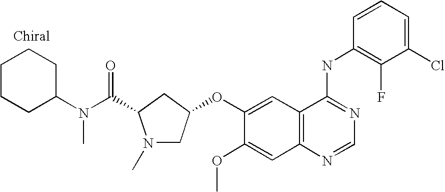

¹H NMR Spectrum: (DMSO d₆ 373K) 1.14 (m, 1H), 1.37 (m, 2H), 1.57 (m, 4H), 1.79 (m, 2H), 2.06 (m, 1H), 2.38 (s, 3H), 2.72 (m, 2H), 2.87 (m, 4H), 3.40 (m, 1H), 3.48 (m, 1H), 3.97 (s, 3H), 4.20 (n, 1H), 5.08 (m, 1H), 7.25 (m, 2H), 7.41 (m, 1H), 7.61 (m, 1H), 7.76 (s, 1H), 8.39 (s, 1H), 9.25 (br s, 1H); Mass Spectrum: (M+H)⁺ 542.

### Compound 15-3

### N-(3-chloro-2-fluorophenyl)-7-methoxy-6-({(3S,5S)-1-methyl-5-[(4-methylpiperazin-1-yl)carbonyl]pyrrolidin-3-yl}oxy)quinazolin-4-amine

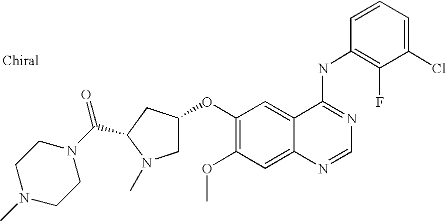

¹H NMR Spectrum: (DMSO d₆ 373K) 2.05 (m, 1H), 2.21 (s, 3H), 2.32 (m, 5H), 2.66 (m, 1H), 2.82 (m, 1H), 3.30 (m, 4H), 3.68 (m, 4H), 3.97 (s, 3H), 5.10 (m, 1H), 7.29 (m, 2H), 7.45 (m, 1H), 7.61 (m, 1H), 7.70 (s, 1H), 8.40 (s, 1H), 9.25 (br s, 1H); Mass Spectrum: (M+H)⁺ 529.

### Compound 15-4

### (4S)-4-({4-[(3-chloro-2-fluorophenyl)amino]-7-methoxyquinazolin-6-yl}oxy)-1-methyl-N-(tetrahydro-2H-pyran-4-yl)-L-prolinamide

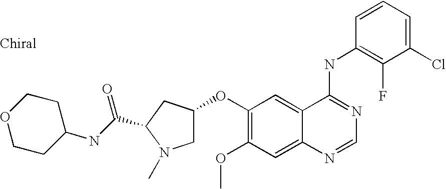

¹H NMR Spectrum: (DMSO d₆ 373K) 1.56 (m, 2H), 1.79 (m, 2H), 2.03 (m, 1H), 2.40 (s, 3H), 2.80 (m, 2H), 2.93 (m, 1H), 3.41 (m, 3H), 3.87 (m, 3H), 3.99 (s, 3H), 5.08 (m, 1H), 7.27 (m, 2H), 7.41 (m, 1H), 7.48 (m, 1H), 7.62 (m, 1H), 7.72 (s, 1H), 8.40 (s, 1H), 9.27 (br s, 1H); Mass Spectrum: (M+H)⁺ 530.

### Compound 15-5

### N-(3-chloro-2-fluorophenyl)-7-methoxy-6-{[(3S,5S)-1-methyl-5-(pyrrolidin-1-ylcarbonyl)pyrrolidin-3-yl]oxy}quinazolin-4-amine

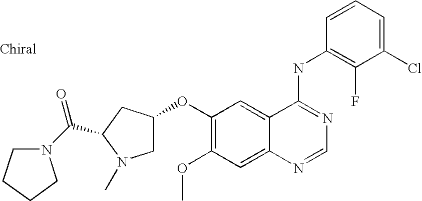

¹H NMR Spectrum: (DMSO d₆) 1.75 (m, 2H), 1.87 (m, 3H), 2.28 (s, 3H), 2.68 (m, 1H), 2.89 (m, 1H), 3.22 (m, 4H), 3.56 (m, 2H), 3.96 (s, 3H), 5.03 (m, 1H), 7.22 (s, 1H), 7.29 (m, 1H), 7.50 (m, 2H), 7.66 (s, 1H), 8.38 (s, 1H), 9.57 (brs, 1H); Mass Spectrum: (M+H)⁺ 500.

### Compound 15-6

### (4S)-4-({4-[(3-chloro-2-fluorophenyl)amino]-7-methoxyquinazolin-6-yl}oxy)-N-(2-methoxyethyl)-N,1-dimethyl-L-prolinamide

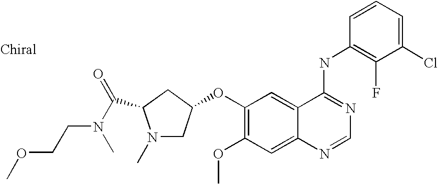

¹H NMR Spectrum: (DMSO d₆ 373K) 2.07 (n, 1H), 2.32 (s, 3H), 2.78 (m, 2H), 3.02 (m, 2H), 3.29 (s, 3H), 3.32-3.65 (m, 7H), 3.93 (s, 3H), 5.07 (m, 1H), 7.21 (s, 1H), 7.25 (m, 1H), 7.40 (m, 1H), 7.59 (m, 1H), 7.70 (s, 1H), 8.38 (s, 1H), 9.23 (brs, 1H); Mass Spectrum:(M+H)⁺ 518.

### Compound 15-7

### (4S)-4-({4-[(3-chloro-2-fluorophenyl)amino]-7-methoxyquinazolin-6-yl}oxy)-N,1-dimethyl-N-(1-methylpiperidin-4-yl)-L-prolinamide

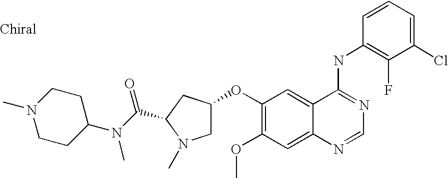

1H NMR Spectrum: (DMSO d₆) 1.40 (m, 2H), 1.62-2.08 (m, 6H), 2.18 (s, 3H), 2.27 (s, 3H), 2.58-2.98 (m, 6H), 3.27 (m, 2H), 3.98 (s, 3H), 4.21 (m, 1H), 5.02 (m, 1H), 7.21 (s, 1H), 7.29 (m, 1H), 7.50 (m, 1H), 7.57 (m, 1H), 7.64 (m, 1H), 8.39 (s, 1H), 9.58 (brs, 1H); Mass Spectrum: (M+H)⁺ 557.

### Compound 15-8

### (4S)-4-({4-[(3-chloro-2-fluorophenyl)amino]-7-methoxyquinazolin-6-yl}oxy)-N-cyclopentyl-1-methyl-L-prolinamide

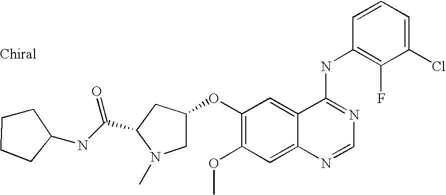

¹H NMR Spectrum: (DMSO d₆) 1.23-1.93 (m, 9H), 2.31 (s, 3H), 2.72 (m, 1H), 2.89 (m, 2H), 3.32 (m, 1H), 3.92 (s, 3H), 4.02 (m, 1H), 5.02 (m, 1H), 7.20 (s, 1H), 7.29 (m, 1H), 7.50 (m, 3H), 7.62 (s, 1H), 8.37 (s, 1H), 9.47 (brs, 1H); Mass Spectrum: (M+H)⁺ 514.

### Compound 15-9

### (4S)-4-({4-[(3-chloro-2-fluorophenyl)amino]-7-methoxyquinazolin-6-yl}oxy)-N-methoxy-1-methyl-L-prolinamide

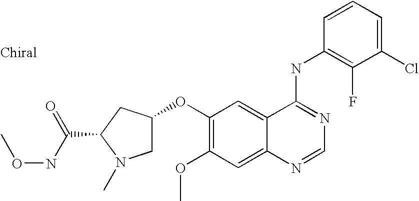

¹H NMR Spectrum: (DMSO d₆ 373K) 2.07 (m, 1H), 2.32 (s, 3H), 2.45 (m, 1H), 2.72 (m, 2H), 3.29 (m, 1H), 3.63 (s, 3H), 3.97 (s, 3H), 5.03 (m, 1H), 7.22 (s, 1H), 7.25 (m, 1H), 7.41 (m, 1H), 7.58 (m, 1H), 7.69 (s, 1H), 8.38 (s, 1H), 9.22 (br s, 1H), 10.56 (br s, 1H); Mass Spectrum: (M+H)⁺ 476.

### Compound 15-10

### (4S)-4-({4-[(3-chloro-2-fluorophenyl)amino]-7-methoxyquinazolin-6-yl}oxy)-N-(cyclopropylmethyl)-1-methyl-L-prolinamide

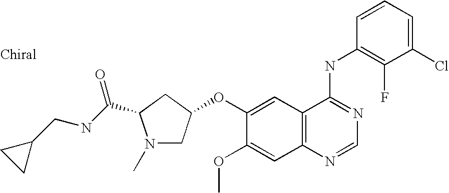

¹H NMR Spectrum: (DMSO d₆) 0.10-0.20 (m, 2H), 0.30-0.40 (m, 2H), 0.85-1.00 (m, 1H) 1.80-1.95 (m, 1H), 2.30 (s, 3H), 2.70 (dd, 1H), 2.80-3.05 (m, 4H), 3.15 (d, 1H), 3.92 (s, 3H), 5.00 (m, 1H), 7.20 (s, 1H), 7.27 (t, 1H), 7.45-7.55 (m, 2H), 7.63 (s, 1H), 7.75 (t, 1H), 8.36 (s, 1H), 9.25 (brs, 1H); Mass Spectrum: (M+H)⁺ 499.5.

### Compound 15-11

### (4S)-4-({4-[(3-chloro-2-fluorophenyl)amino]-7-methoxyquinazolin-6-yl}oxy)-N-cyclohexyl-1-methyl-L-prolinamide

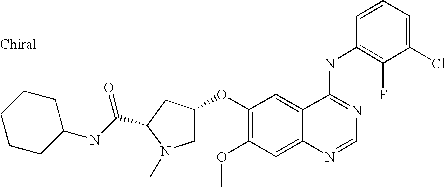

¹H NMR Spectrum: (DMSO d₆ 300K) 1.15-1.40 (m, 5H), 1.54-1.62 (m, 1H), 1.62-1.85 (m, 4H), 1.97-2.04 (m, 1H), 2.35 (s, 3H), 2.70-2.82 (m, 2H), 2.85-3.05 (m, 1+H2O), 3.36 (d, 1H), 3.50-3.65 (m, 1H), 3.95 (s, 3H), 5.03 (m, 1H), 7.15-7.32 (m, 3H), 7.40 (t, 1H), 7.59 (t, 1H), 7.68 (s, 1H), 8.35 (s, 1H), 9.22 (brs, 1H); Mass Spectrum: (M+H)⁺ 528.

1. A quinazoline derivative of the Formula (I): 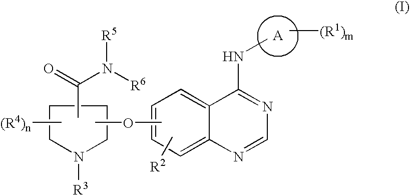
wherein:
either R² is in the 6-position and the substituted-pyrrolidinyloxy group is in the 7-position of the quinazoline ring or R² is in the 7-position and the substituted-pyrrolidinyloxy group is in the 6-position of the quinazoline ring;
A is phenyl or pyridyl;
each R¹ is a substituent on a ring carbon atom in ring A and is independently selected from halogeno, cyano, nitro, hydroxy, carboxy, trifluoromethyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkoxycarbonyl, ureido, N-(1-6C)alkylureido, N,N-di-[(1-6C)alkyl]ureido, —NR^{a}R^{b}, —SO₂NR^{a}R^{b} and a group of the formula —CONR^{a}R^{b} (wherein R^{a} is hydrogen or (1-6C)alkyl and R^{b} selected from hydrogen, (1-6C)alkyl, phenyl, benzyl, heterocyclyl, heterocyclyl(1-3C)alkyl, heteroaryl, heteroaryl(1-3C)alkyl, (3-7)cycloalkyl and (3-7)cycloalkyl(1-3C)alkyl wherein any alkyl, heterocyclyl, heteroaryl and cycloalkyl groups in R^{a} and R^{b} are optionally substituted by 1, 2 or 3 substituents selected from (1-4C)alkyl, halogeno, hydroxy and (1-4C)alkoxy;
or R^{a} and R^{b} together with the nitrogen atom to which they are attached form a 4, 5 or 6-membered ring which optionally contains an additional ring heteroatom selected from nitrogen, oxygen and sulphur and which is optionally substituted by 1 or 2 substituents on an available ring carbon atom, independently selected from halogeno, hydroxy, (1-4C)alkyl and (1-3C)alkylenedioxy and optionally substituted on any available ring nitrogen by a substituent selected from (1-4C)alkyl and (2-4C)alkanoyl (provided the ring is not thereby quaternised), and wherein any (1-4C)alkyl or (2-4C)alkanoyl group present as a substituent on the ring formed by R^{a} and R^{b} together with the nitrogen atom to which they are attached is optionally substituted by 1, 2 or 3 substituents independently selected from halogeno, hydroxyl, (1-4C)alkyl and (1-4C)alkoxy;
or, when two R¹ groups are attached to adjacent carbon atoms, they may, together with the carbon atoms to which they are attached, form a pyrrole ring, wherein the pyrrole ring is optionally substituted by 1 or 2 substituents independently selected from (1-6C)alkyl, halogeno, cyano, nitro, hydroxy, amino, carbamoyl, sulfamoyl and trifluoromethyl;
or, when two R¹ groups are attached to adjacent carbon atoms, they may, together form a (1-3C)alkylenedioxy group [—O(CH₂)₁₋₃O];
m is 0, 1, 2 or 3; each R² is selected from hydrogen, (1-6C)alkyl, (3-6C)cycloalkyl, (3-6C)cycloalkyl(1-3C)alkyl and a group of the formula R⁷O—, wherein R⁷ is (1-6C)alkyl optionally substituted by 1, 2 or 3 substituents independently selected from hydroxy and a group of the formula R⁸O— (wherein R⁸ is (1-3C)alkyl);
R³ is selected from hydrogen, (1-6C)alkyl, (3-6C)cycloalkyl, (3-6C)cycloalkyl(1-3C)alkyl, (1-6C)alkylthio, (1-6C)alkylsulfinyl, (1-6C)alkylsulfonyl, (2-6C)alkanoyl, carbamoyl(1-6C)alkyl, N-(1-6C)alkylcarbamoyl(1-6C)alkyl, N,N-di-[(1-6C)alkyl]carbamoyl(1-6C)alkyl, sulfamoyl(1-6C)alkyl, N-(1-6C)alkylsulfamoyl(1-6C)alkyl, N,N-di-[(1-6C)alkyl]sulfamoyl(1-6C)alkyl and (2-6C)alkanoyl(1-6C)alkyl,
and wherein any (1-6C)alkyl or (2-6C)alkanoyl group within R³ is optionally substituted by 1, 2 or 3 substituents independently selected from halogeno, hydroxy and (1-6C)alkyl and/or optionally a substituent selected from cyano, nitro, (2-8C)alkenyl, (2-8C)alkynyl, (1-6C)alkoxy and NR^{c}R^{d}, wherein R^{c} is hydrogen or (1-4C)alkyl and R^{d} is hydrogen or (1-4C)alkyl and wherein any (1-4C)alkyl in R^{c} or R^{d} is optionally substituted by 1, 2 or 3 substituents independently selected from halogeno and hydroxy and/or optionally a substituent selected from cyano, nitro and (1-4C)alkoxy,
or R^{c} and R^{d} together with the nitrogen atom to which they are attached form a 4, 5 or 6 membered ring which optionally contains an additional ring heteroatom selected from nitrogen, oxygen and sulphur and which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised),
and wherein any (1-4C)alkyl or (2-4C)alkanoyl group present as a substituent on the ring formed by R^{c} and R^{d} together with the nitrogen atom to which they are attached is optionally substituted by 1, 2 or 3 substituents independently selected from halogeno and hydroxy and/or optionally a substituent selected from (1-4C)alkyl and (1-4C)alkoxy;
each R⁴ is independently selected from (1-4C)alkyl, (1-4C)alkoxy, cyano, halogeno, hydroxyl and oxo;
n is 0, 1 or 2;
R⁵ is hydrogen or (1-6C)alkyl;
R⁶ is selected from hydrogen, (1-6C)alkyl, (2-6C)alkenyl, (2-6C)alkynyl, (1-6C)alkoxy, (3-7)cycloalkyl, (1-6C)alkylsulfonyl, heterocyclyl, heteroaryl, (3-7)cycloalkyl(1-3C)alkyl, (3-7)heterocyclyl(1-3C)alkyl and heteroaryl(1-3C)alkyl,
and wherein any (1-3C)alkyl, (1-6C)alkyl, (3-7)cycloalkyl, heteroaryl or heterocyclyl group within R⁵ or R⁶ is optionally substituted (on any available carbon atoms) by 1, 2 or 3 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heterocyclyl group within R⁶ is optionally substituted on any available ring nitrogen (provided the ring is not thereby quaternised) by (1-4C)alkyl or (2-4C)alkanoyl, or R⁵ and R⁶ together with the nitrogen atom to which they are attached form a 4, 5 or 6 membered ring which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised),
and wherein any (1-4C)alkyl or (2-4C)alkanoyl group present as a substituent on the ring formed by R⁵ and R⁶ together with the nitrogen atom to which they are attached is optionally substituted by 1, 2 or 3 substituents independently selected from halogeno and hydroxy and/or optionally a substituent selected from (1-4C)alkyl and (1-4C)alkoxy;
provided that when the pyrrolidinyloxy group is linked to the 6-position of the quinazoline ring, m is 2 and substituents R¹ are both halogeno and attached to the 2- and 3-positions of the ring A, then R⁶ is selected from substituted-(1-6C)alkyl (wherein substituted-(1-6C)alkyl is (1-6C)alkyl substituted by 1, 2 or 3 substituents independently selected from halogeno, hydroxy(1-6C)alkyl, (1-6C)alkoxycarbonyl, carbamoyl, (2-6C)alkanoylamino, (1-6C)alkylamino, di-[(1-6C)alkyl]amino and hydroxy and/or optionally a substituent selected from oxo, cyano, nitro and (1-4C)alkoxy), (2-6C)alkenyl, (2-6C)alkynyl, (1-6C)alkoxy, (3-7)cycloalkyl, (1-6C)alkylsulfonyl, (3-7)heterocyclyl, heteroaryl, (3-7)cycloalkyl(1-6C)alkyl, (3-7)heterocyclyl(1-6C)alkyl and heteroaryl(1-6C)alkyl,
and wherein any (3-7)cycloalkyl, heteroaryl or (3-7)heterocyclyl group within R⁶ is optionally substituted (on any available carbon atoms) by 1, 2 or 3 substituents independently selected from halogeno, hydroxy, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heteroaryl or heterocyclyl group within R⁶ is optionally substituted on any available ring nitrogen (provided the ring is not thereby quaternised) by (1-4C)alkyl or (2-4C)alkanoyl, or
R⁵ and R⁶ together with the nitrogen atom to which they are attached form a 4, 5 or 6 membered ring which contains one or two nitrogen atoms as the only hetero atoms present in the ring and which is optionally substituted on an available ring carbon atom by 1 or 2 substituents independently selected from hydroxy, carbamoyl, (1-4C)alkyl, and
(1-3C)alkylenedioxy; and wherein any 4, 5 or 6 membered heterocyclic ring formed by R⁵ and R⁶ is optionally substituted on any available ring nitrogen (provided the ring is not thereby quaternised) by (1-4C)alkyl or (2-4C)alkanoyl;
or a pharmaceutically-acceptable salt thereof.

2. A quinazoline derivative according to claim 1, wherein R⁵ is hydrogen or (1-6C)alkyl and R⁶ is selected from hydrogen, (1-6C)alkyl, (2-6C)alkenyl, (2-6C)alkynyl, (1-6C)alkoxy, (3-7)cycloalkyl, (1-6C)alkylsulfonyl, heterocyclyl, heteroaryl, (3-7)cycloalkyl(1-3C)alkyl, (3-7)heterocyclyl(1-3C)alkyl and heteroaryl(1-3C)alkyl,
and wherein any (1-3C)alkyl, (1-6C)alkyl, (3-7)cycloalkyl, heteroaryl or heterocyclyl group within R⁵ or R⁶ is optionally substituted (on any available carbon atoms) by 1, 2 or 3 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heterocyclyl group within R⁶ is optionally substituted on any available ring nitrogen (provided the ring is not thereby quaternised) by (1-4C)alkyl or (2-4C)alkanoyl, or R⁵ and R⁶ together with the nitrogen atom to which they are attached form a 4, 5 or 6 membered ring which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised),
and wherein any (1-4C)alkyl or (2-4C)alkanoyl group present as a substituent on the ring formed by R⁵ and R⁶ together with the nitrogen atom to which they are attached is optionally substituted by 1, 2 or 3 substituents independently selected from halogeno and hydroxy and/or optionally a substituent selected from (1-4C)alkyl and (1-4C)alkoxy;
provided that when the pyrrolidinyloxy group is linked to the 6-position of the quinazoline ring, m is 2 and substituents R¹ are both halogeno and attached to the 2- and 3-positions of the ring A, then R⁶ is selected from substituted-(1-6C)alkyl (wherein substituted-(1-6C)alkyl is (1-6C)alkyl substituted by 1, 2 or 3 substituents independently selected from (1-6C)alkoxycarbonyl, carbamoyl, (2-6C)alkanoylamino, and oxo or a (1-6C)alkoxycarbonyl together with a hydroxy group), (1-6C)alkoxy, (1-6C)alkylsulfonyl, (3-7)heterocyclyl (wherein the heterocyclyl is carbon linked), heteroaryl, (3-7)heterocyclyl(1-6C)alkyl (wherein the heterocyclyl is carbon linked to the (1-6C)alkyl moiety) and heteroaryl(1-6C)alkyl, and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy, and wherein any heteroaryl or heterocyclyl group within R⁶ is optionally substituted on any available ring nitrogen (provided the ring is not thereby quaternised) by (1-4C)alkyl or (2-4C)alkanoyl, or
R⁵ and R⁶ together with the nitrogen atom to which they are attached form a 4, 5 or 6 membered ring which contains one or two nitrogen atoms as the only hetero atoms present in the ring and which is substituted on an available ring carbon atom by 1 or 2 substituents independently selected from carbamoyl and (1-3C)alkylenedioxy.

3. A quinazoline derivative according to claim 1 or claim 2, wherein R⁵ is hydrogen, methyl, ethyl propyl, isopropyl or isobutyl and R⁶ is selected from hydrogen, methyl, ethyl propyl, isopropyl, isobutyl, vinyl, isopropenyl, allyl, but-2-enyl ethynyl, 2-propynyl, butynyl, methoxy, ethoxy propoxy,isopropoxy, cyclopropyl, cyclopentyl, cyclohexyl, azetidinyl, oxazepanyl, pyrrolinyl, pyrrolidinyl, morpholinyl, tetrahydro-1,4thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl, tetrahydrothiopyranyl, thiomorpholinyl, pyrazolyl, thienyl, oxazolyl, isoxazolyl, imidazolyl, pyridinyl, pyridazinyl, pyrazinyl, pyrimidyl, furanyl, pyrazolyl, thiazolyl, isothiazolyl, thiadiazolyl, cyclopropylmethyl, cyclopentylmethyl, cyclohexylmethyl, 2-cyclopropylethyl, 2-cyclopentylethyl, 2-cyclohexylethyl, azetidinylmethyl, oxazepanylmethyl, pyrrolinylmethyl, pyrrolidinylmethyl, morpholinylmethyl, tetrahydro-1,4-thiazinylmethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl, tetrahydrothienylmethyl, tetrahydrothiopyranylmethyl, thiomorpholinylmethyl, pyrazolylmethyl, thienylmethyl, oxazolylmethyl, isoxazolylmethyl, imidazolylmethyl, pyridinylmethyl, pyridazininylmethyl, pyrazinylmethyl, pyrimidylmethyl, furanylmethyl, pyrazolylmethyl, thiazolylmethyl, isothiazolylmethyl, thiadiazolylmethyl, 2-(azetidinyl)ethyl, 2-(oxazepanyl)ethyl, 2-(pyrrolinyl)ethyl, 2-(pyrrolidinyl)ethyl, 2-(morpholinyl)ethyl, 2-(tetrahydro-1,4-thiazinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrothienyl)ethyl, 2-(tetrahydrothiopyranyl)ethyl, 2-(thiomorpholinyl)ethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl, 2-(isothiazolyl)ethyl and 2-(thiadiazolyl)ethyl,
and wherein any alkyl, cycloalkyl, heteroaryl or heterocyclyl group within R⁵ or R⁶ is optionally substituted (on any available carbon atoms) by 1 or 2 substituents independently selected from fluoro, chloro, bromo, hydroxymethyl, 2-hydroxyethyl, methoxycarbonyl ethoxycarbonyl, carbamoyl, acetamido, propionamido and hydroxy and/or optionally a substituent selected from oxo, cyano, methoxy and ethoxy,
and wherein any heterocyclyl group within R⁶ is optionally substituted on any available ring nitrogen (provided the ring is not thereby quaternised) by methyl, ethyl, acetyl or propionyl, or R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, bromo, hydroxy, methyl, ethyl and propylenedioxy, and optionally substituted on any available ring nitrogen by a substituent selected from methyl, ethyl, acetyl and propionyl (provided the ring is not thereby quaternised),
and wherein any alkyl or alkanoyl group present as a substituent on the ring formed by R⁵ and R⁶ together with the nitrogen atom to which they are attached is optionally substituted by 1 or 2 substituents independently selected from fluoro, chloro, bromo and hydroxy and/or optionally a substituent selected from methyl, ethyl, methoxy and ethoxy;
provided that when the pyrrolidinyloxy group is linked to the 6-position of the quinazoline ring, m is 2 and substituents R¹ are both halogeno and attached to the 2- and 3-positions of the ring A, then R⁶ is selected from substituted-methyl, substituted-ethyl substituted-propyl, substituted-isopropyl, substituted-isobutyl (wherein the substituted groups are substituted by 1 or 2 substituents independently selected from methoxycarbonyl, ethoxycarbonyl, carbamoyl, acetamido, propionamido and oxo or a methoxycarbonyl group together with a hydroxy group or an ethoxycarbonyl group together with a hydroxy group) methoxy, ethoxy, propoxy, isopropoxy,
a carbon linked heterocyclyl group selected from azetidinyl, oxazepanyl, pyrrolinyl, pyrrolidinyl, morpholinyl, tetrahydrofuranyl, tetrahydro-1,4thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl, tetrahydropyranyl, tetrahydrothiopyranyl, thiomorpholinyl
a heteroaryl group selected from pyrazolyl, thienyl, oxazolyl, isoxazolyl, imidazolyl, pyridinyl, pyridazinyl, pyrazinyl, pyrimidyl, furanyl, thiazolyl, isothiazolyl, thiadiazolyl,
a (3-7)heterocyclyl(1-6C)alkyl group (wherein the heterocyclyl is carbon linked to the (1-6C)alkyl moiety) selected from azetidinylmethyl, oxazepanylmethyl, pyrrolinylmethyl, pyrrolidinylmethyl, morpholinylmethyl, tetrahydro-1,4-thiazinylmethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl, tetrahydrofuranylmethyl, tetrahydrothienylmethyl, tetrahydropyranylmethyl, tetrahydrothiopyranylmethyl, thiomorpholinylmethyl, 2-(azetidinyl)ethyl, 2-(oxazepanyl)ethyl, 2-(pyrrolinyl)ethyl, 2-(pyrrolidinyl)ethyl, 2-(morpholinyl)ethyl, 2-(tetrahydro-1,4-thiazinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrofuranyl)ethyl, 2-(tetrahydrothienyl)ethyl, 2-(tetrahydropyranyl)ethyl, 2-(tetrahydrothiopyranyl)ethyl, 2-(thiomorpholinyl)ethyl,
a heteroaryl(1-6C)alkyl group selected from pyrazolylmethyl, thienylmethyl, oxazolylmethyl, isoxazolylmethyl, imidazolylmethyl, pyridinylmethyl, pyridazinylmethyl, pyrazinylmethyl, pyrimidylmethyl, furanylmethyl, pyrazolylmethyl, thiazolylmethyl, isothiazolylmethyl, thiadiazolylmethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl, 2-(isothiazolyl)ethyl and 2-(thiadiazolyl)ethyl,
and wherein any heteroaryl or heterocyclyl group within R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido, propionamido and hydroxy and/or optionally a substituent selected from oxo, cyano, methoxy and ethoxy,
and wherein any heteroaryl or heterocyclyl group within R⁶ is optionally substituted on any available ring nitrogen (provided the ring is not thereby quaternised) by methyl, ethyl, acetyl or propionyl
or R⁵ and R⁶ together with the nitrogen atom to which they are attached form an azetidin-1-yl ring substituted carbamoyl or (1-3C)alkylenedioxy.

4. A quinazoline derivative according to any one of claims 1 to 3, wherein R⁵ is hydrogen, methyl or ethyl and R⁶ is selected from hydrogen, methyl, ethyl, propyl, isopropyl, isobutyl, vinyl, isoprop-2-enyl, allyl, but-2-enyl ethynyl, 2-prop-2-ynyl, but-3-ynyl, methoxy, ethoxy, cyclopropyl, cyclopentyl, cyclohexyl, azetidinyl, pyrrolinyl, pyrrolidinyl, morpholinyl, piperidinyl, piperazinyl, tetrahydropyridinyl, thiomorpholinyl, 1,2,3,6-tetrahydropyridin-1-yl, pyrazolyl, thienyl, oxazolyl, isoxazolyl, imidazolyl, pyridinyl, pyridazinyl, pyrazinyl, pyrimidyl, furanyl, pyrazolyl, thiazolyl, isothiazolyl, cyclopropylmethyl, cyclopentylmethyl, cyclohexylmethyl, 2-cyclopropylethyl, 2-cyclopentylethyl, 2-cyclohexylethyl, azetidinylmethyl, pyrrolinylmethyl, pyrrolidinylmethyl, morpholinylmethyl, piperidinylmethyl, piperazinylmethyl, tetrahydropyridinylmethyl, thiomorpholinylmethyl, pyrazolylmethyl, thienylmethyl, oxazolylmethyl, isoxazolylmethyl, imidazolylmethyl, pyridinylmethyl, pyridazinylmethyl, pyrazinylmethyl, pyrimidylmethyl, furanylmethyl, pyrazolylmethyl, thiazolylmethyl, isothiazolylmethyl, 2-(azetidinyl)ethyl2-(pyrrolinyl)ethyl, 2-(pyrrolidinyl)ethyl, 2-(morpholinyl)ethyl, 2-(piperidinyl)ethyl, 2-(piperazinyl)ethyl, 2-(tetrahydropyridinyl)ethyl, 2-(thiomorpholinyl)ethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl)ethyl,
and wherein any alkyl, cycloalkyl, heteroaryl or heterocyclyl group within R⁵ or R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido and hydroxy and/or optionally a substituent selected from oxo, cyano, methoxy, and ethoxy,
and wherein any heterocyclyl group within R⁶ is optionally substituted on any available ring nitrogen (provided the ring is not thereby quaternised) by methyl, ethyl, acetyl or propionyl, or R⁵ and R⁶ together with the nitrogen atom to which they are attached form a azetidin-1-yl, pyrrolin-1-yl, pyrrolidin-1-yl, piperidino, morpholino or piperazino ring which is optionally substituted by 1 or 2 substituents on an available ring carbon atom, independently selected from fluoro, chloro, hydroxy, methyl, ethyl and propylenedioxy, and optionally substituted on any available ring nitrogen by a substituent selected from methyl, ethyl, acetyl and propionyl (provided the ring is not thereby quaternised),
and wherein any alkyl or alkanoyl group present as a substituent on the ring formed by R⁵ and R⁶ together with the nitrogen atom to which they are attached is optionally substituted by 1 or 2 substituents independently selected from fluoro, chloro and hydroxy and/or optionally a substituent selected from methyl, ethyl methoxy and ethoxy;
provided that when the pyrrolidinyloxy group is linked to the 6-position of the quinazoline ring, m is 2 and substituents R¹ are both halogeno and attached to the 2- and 3-positions of the ring A, then R⁶ is selected from substituted-methyl, substituted-ethyl substituted-propyl, substituted-isopropyl, substituted-isobutyl, (wherein the substituted groups are substituted by 1 or 2 substituents independently selected from methoxycarbonyl, ethoxycarbonyl, carbamoyl, acetamido and oxo or a methoxycarbonyl group together with a hydroxy group), methoxy, ethoxy,
a carbon linked heterocyclyl group selected from azetidinyl, pyrrolinyl, pyrrolidinyl, morpholinyl, tetrahydrofuranyl, piperidinyl, piperazinyl, tetrahydropyridinyl, tetrahydropyranyl, thiomorpholinyl,
a heteroaryl group selected from pyrazolyl, thienyl, oxazolyl, isoxazolyl, imidazolyl, pyridinyl, pyridazinyl, pyrazinyl, pyrimidyl, furanyl, pyrazolyl, thiazolyl, isothiazolyl,
a (3-7)heterocyclyl(1-6C)alkyl group (wherein the heterocyclyl is carbon linked to the (1-6C)alkyl moiety) selected from azetidinylmethyl, pyrrolinylmethyl, pyrrolidinylmethyl, morpholinylmethyl, piperidinylmethyl, piperazinylmethyl, tetrahydrofuranylmethyl, tetrahydropyranylmethyl, tetrahydropyridinylmethyl, thiomorpholinylmethyl, 2-(azetidinyl)ethyl, 2-(pyrrolinyl)ethyl, 2-(pyrrolidinyl)ethyl, 2-(morpholinyl)ethyl, 2-(piperidinyl)ethyl, 2-(piperazinyl)ethyl, 2-(tetrahydrofuranyl)ethyl, 2-(tetrahydropyranyl)methyl, 2-(tetrahydropyridinyl)ethyl, 2-(thiomorpholinyl)ethyl,
a heteroaryl(1-6C)alkyl group selected from pyrazolylmethyl, thienylmethyl, oxazolylmethyl, isoxazolylmethyl, imidazolylmethyl, pyridinylmethyl, pyridazinylmethyl, pyrazinylmethyl, pyrimidylmethyl, furanylmethyl, pyrazolylmethyl, thiazolylmethyl, isothiazolylmethyl, 2-(pyrazolyl)ethyl, 2-(thienyl)ethyl, 2-(oxazolyl)ethyl, 2-(isoxazolyl)ethyl, 2-(imidazolyl)ethyl, 2-(pyridinyl)ethyl, 2-(pyridazinyl)ethyl, 2-(pyrazinyl)ethyl, 2-(pyrimidyl)ethyl, 2-(furanyl)ethyl, 2-(pyrazolyl)ethyl, 2-(thiazolyl)ethyl and 2-(isothiazolyl)ethyl,
and wherein any heteroaryl or heterocyclyl group within R⁶ is optionally substituted (on any available carbon atoms) by 1 or 2 substituents independently selected from fluoro, chloro, bromo, hydroxymethyl, 2-hydroxyethyl, methoxycarbonyl, ethoxycarbonyl, carbamoyl, acetamido and hydroxy and/or optionally a substituent selected from oxo, cyano, methoxy and ethoxy,
and wherein any heteroaryl or heterocyclyl group within R⁶ is optionally substituted on any available ring nitrogen (provided the ring is not thereby quaternised) by methyl, ethyl, acetyl or propionyl;
or R⁵ and R⁶ together with the nitrogen atom to which they are attached form an azetidin-1-yl ring substituted by a carbamoyl group.

5. A quinazoline derivative according to any one of claims 1 to 4, wherein R⁵ is hydrogen or methyl and R⁶ is selected from hydrogen, methyl, ethyl, propyl, isopropyl, vinyl, isoprop-2-enyl, allyl, but-2-enyl ethynyl, 2-propynyl, but-3-ynyl, methoxy, cyclopropyl, cyclopentyl, 1-(hydroxymethyl)cyclopentyl, cyclohexyl, 4-hydroxycyclohexyl, cyclopropylmethyl, cyclopentylmethyl, methoxymethyl, 2-(methoxy)ethyl, 2-(ethoxy)ethyl, carbamoylmethyl, 2-(acetyl)ethyl, cyanomethyl, 2-(cyano)ethyl, 2,3-dihydroxypropyl, 2-(hydroxyl)-1,1-dimethylethyl, 2,2,2-trifluoroethyl, 1-(ethoxycarbonyl)-2-hydroxyethyl, 2-acetamido)ethyl, tetrahydrofuran-2-ylmethyl, imidazol-2-ylmethyl, 1-methylpyrazol-5-yl, 1-methylpyrazol-5-yl, 3-methylpyrazol-5-yl, imidazol-1-ylmethyl, 2-(imidazol-1-yl)ethyl, furan-2-ylmethyl,2-(furan-2-yl)ethyl, 5-methylisoxazol-3-ylmethyl, thien-3yl, morpholino, piperidin-4-yl, 1-methylpiperidin-4-yl, tetrahydro-2H-pyran-4-yl and 3-oxotetrahydrofuran-4-yl,
or R⁵ and R⁶ together with the nitrogen atom to which they are attached form a 3-hydroxyazetidin-1-yl, 2-carbamoylazetidin-1-yl, pyrrolin-1-yl, pyrrolidin-1-yl, 3-hydroxy, pyrrolidin-1-yl, piperidino, morpholino or piperazino group;
provided that when the pyrrolidinyloxy group is linked to the 6-position of the quinazoline ring, m is 2 and substituents R¹ are both halogeno and attached to the 2- and 3-positions of the ring A, then R⁶ is selected from methoxy, carbamoylmethyl, 2-(hydroxy)-1-(methoxycarbonyl)ethyl, 1-(ethoxycarbonyl)-2-hydroxyethyl, 2-(acetamido)ethyl, piperidin-4-yl, 1-methylpiperidin-4yl, tetrahydropyran-4-yl, 4-hydroxytetrahydrofuran-3-yl, 3-oxotetrahydrofuran-4-yl, 1-methylpyrazol-5-yl, thien-3yl, 3-methylpyrazol-5-yl, tetrahydrofuran-2-ylmethyl, tetrahydropyran-4-ylmethyl, furan-2-ylmethyl, 2-(furan-2-yl)ethyl, imidazol-1-ylmethyl, imidazol-2-ylmethyl, imidazol-2-ylmethyl, 2-(imidazol-1-yl)ethyl, 2-(imidazol-4-yl)ethyl and 5-methylisoxazol-3-ylmethyl or R⁵ and R⁶ together with the nitrogen atom to which they are attached form an azetidinyl substituted in the 2 position by a carbamoyl group.

6. A quinazoline derivative according to claim 1 or claim 2, wherein R⁵ is hydrogen or (1-6C)alkyl and R⁶ is selected from hydrogen, (1-6C)alkyl, (2-6C)alkenyl, (2-6C)alkynyl, (1-6C)alkoxy, (3-7)cycloalkyl, (1-6C)alkylsulfonyl, heterocyclyl, heteroaryl, (3-7)cycloalkyl(1-3C)alkyl, (3-7)heterocyclyl(1-3C)alkyl and heteroaryl(1-3C)alkyl, and wherein any (1-3C)alkyl, (1-6C)alkyl, (3-7)cycloalkyl, heteroaryl or heterocyclyl group within R⁵ or R⁶ is optionally substituted (on any available carbon atoms) by 1, 2 or 3 substituents independently selected from halogeno, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy,
and wherein any heterocyclyl group within R⁶ is optionally substituted on any available ring nitrogen (provided the ring is not thereby quaternised) by (1-4C)alkyl or (2-4C)alkanoyl, or R⁵ and R⁶ together with the nitrogen atom to which they are attached form a 4, 5 or 6 membered ring which is optionally substituted by 1 or 2 substituents on an available ring carbon atom, independently selected from halogeno, hydroxy, (1-4C)alkyl and (1-3C)alkylenedioxy, and optionally substituted on any available ring nitrogen by a substituent selected from (1-4C)alkyl and (2-4C)alkanoyl (provided the ring is not thereby quaternised),
and wherein any (1-4C)alkyl or (2-4C)alkanoyl group present as a substituent on the ring formed by R⁵ and R⁶ together with the nitrogen atom to which they are attached is optionally substituted by 1, 2 or 3 substituents independently selected from halogeno and hydroxy and/or optionally a substituent selected from (1-4C)alkyl and (1-4C)alkoxy;
provided that when the pyrrolidinyloxy group is linked to the 6-position of the quinazoline ring, m is 2 and substituents R¹ are both halogeno and attached to the 2- and 3-positions of the ring A, then R⁶ is selected from (3-7)heterocyclyl (wherein heterocyclyl is carbon linked), heteroaryl, (3-7)heterocyclyl(1-6C)alkyl (wherein the heterocyclyl is carbon linked to the (1-6C)alkyl moiety) and heteroaryl(1-6C)alkyl,
and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy, and wherein any heteroaryl or heterocyclyl group within R⁶ is optionally substituted on any available ring nitrogen (provided the ring is not thereby quaternised) by (1-4C)alkyl or (2-4C)alkanoyl.

7. A quinazoline derivative according to anyone of the preceding claims, wherein m is 0, 1, 2 or 3 and R¹ is independently selected from halogeno, cyano, nitro, hydroxy, trifluoromethyl, (1-6C)alkyl, (1-6C)alkoxy, (1-6C)alkylthio, (1-6C)alkylsulfinyl, (1-6C)alkylsulfonyl, ureido, N-(1-6C)alkylureido, N,N-di-[(1-6C)alkyl]ureido, —NR^{a}R^{b}, —SO₂NR^{a}R^{b} and a group of the formula —CONR^{a}R^{b} (wherein R^{a} and R^{b} are as hereinabove defined);
or, when two R¹ groups are attached to adjacent carbon atoms, they may, together with the carbon atoms to which they are attached, form a pyrrole ring, wherein the pyrrole ring is optionally substituted by 1 or 2 substituents independently selected from (1-6C)alkyl, halogeno, cyano, nitro, hydroxy, amino, carbamoyl, sulfamoyl and trifluoromethyl;
or, when two R¹ groups are attached to adjacent carbon atoms, they may, together form a (1-3C)alkylenedioxy group.

8. A quinazoline derivative according to claim 7, wherein m is 0, 1 or 2 and R¹ is independently selected from fluoro, chloro, cyano, trifluoromethyl, methyl, methoxy, methylthio, isobutylthio, sulfamoyl, and a group of the formula —CONR^{a}R^{b} (wherein R^{a} is hydrogen or methyl and R^{b} selected from hydrogen, methyl, ethyl, isobutyl, furanyl, cyclopentyl and cyclohexyl wherein any alkyl, (3-7)cycloalkyl, heteroaryl in R^{a} and R^{b} are optionally substituted by 1 or 2 substituents selected from hydroxy and methoxy;
or R^{a} and R^{b} together with the nitrogen atom to which they are attached form a 1,2,3,6-tetrahydropyridin-1-yl, pyrrolidin-1-yl, piperidino, piperazin-1-yl or morpholino ring, which is optionally substituted by 1 or 2 substituents on an available ring carbon atom, independently selected from hydroxyl and optionally substituted on any available ring nitrogen by a substituent selected from methyl and acetyl (provided the ring is not thereby quaternised), or, when two R¹ groups are attached to adjacent carbon atoms, they may, together with the carbon atoms to which they are attached, form a pyrrole ring, wherein the pyrrole ring is optionally substituted by 1 or 2 substituents independently selected from hydroxy;
or, when two R¹ groups are attached to adjacent carbon atoms, they may, together form a (1-3C)alkylenedioxy group.

9. A quinazoline derivative according to claim 7 or claim 8, wherein m is 2 and R¹ is positioned in the 2- and 3-positions of ring A and R¹ is independently selected from fluoro and chloro.

10. A quinazoline derivative according to any one of the preceding claims, wherein ring A is phenyl or pyrid-3-yl.

11. A quinazoline derivative according to any one of the preceding claims, wherein ring A is phenyl.

12. A quinazoline derivative according to any one of the preceding claims, wherein R² is selected from hydrogen, (1-6C)alkyl and a group of the formula R⁷O—, wherein R⁷ is (1-6C)alkyl optionally substituted by 1 or 2 substituents independently selected from hydroxy and a group of the formula R⁸O— (wherein R⁸ is (1-3C)alkyl).

13. A quinazoline derivative according to any one of the preceding claims, wherein R² is selected from hydrogen, methyl, ethyl and a group of the formula R⁷O—, wherein R⁷ is methyl or ethyl.

14. A quinazoline derivative according to any one of the preceding claims, wherein R² is methoxy.

15. A quinazoline derivative according to any one of claims 1 to 13, wherein R² is hydrogen.

16. A quinazoline derivative according to any one of the preceding claims, wherein R² is in the 6-position and the substituted-pyrrolidinyloxy group is in the 7-position of the quinazoline ring.

17. A quinazoline derivative according to any one of claims 1 to 15, wherein R² is in the 7-position and the substituted-pyrrolidinyloxy group is in the 6-position of the quinazoline ring.

18. A quinazoline derivative according to any one of the preceding claims, wherein R³ is selected from hydrogen, (1-6C)alkyl, (3-6C)cycloalkyl, (3-6C)cycloalkyl(1-3C)alkyl (2-6C)alkanoyl;
and wherein any (1-6C)alkyl or (2-6C)alkanoyl group within R³ is optionally substituted by 1 or 2 substituents independently selected from halogeno, hydroxy and (1-6C)alkyl and/or optionally a substituent selected from cyano, nitro, (2-8C)alkenyl, (2-8C)alkynyl, (1-6C)alkoxy and NR^{c}R^{d}, wherein R^{c} is hydrogen or (1-4C)alkyl and R^{d} is hydrogen or (1-4C)alkyl.

19. A quinazoline derivative according to anyone of the preceding claims, wherein R³ is methyl.

20. A quinazoline derivative according to anyone of the preceding claims, wherein n is 0, 1 or 2 and R⁴ is independently selected from methyl, ethyl, methoxy, ethoxy, hydroxyl and oxo.

21. A quinazoline derivative according to anyone of the preceding claims, wherein n is 0.

22. A quinazoline derivative according to anyone of the preceding claims, wherein the —CONR⁵R⁶ group is in the 2-position of the pyrrolidine ring.

23. A quinazoline derivative according to anyone of the preceding claims, wherein the substituted-quinazolinyloxy group is in the 3-position of the pyrrolidine ring.

24. A quinazoline derivative according to any one of the preceding claims having a structural sub-formula A2 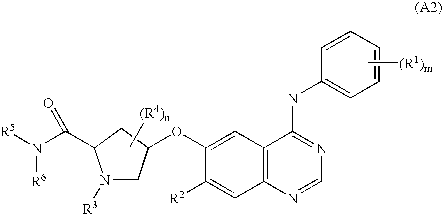
wherein:
m is 2 and R¹ is 2-fluoro and 3-chloro;
R² is methoxy;
R³ is methyl;
n is 0;
and R⁵ is hydrogen or (1-6C)alkyl and R⁶ is selected from substituted-(1-6C)alkyl (wherein substituted-(1-6C)alkyl is (1-6C)alkyl substituted by 1, 2 or 3 substituents independently selected from (1-6C)alkoxycarbonyl, carbamoyl, (2-6C)alkanoylamino, and oxo or a (1-6C)alkoxycarbonyl together with a hydroxy group), (1-6C)alkoxy, (1-6C)alkylsulfonyl, (3-7)heterocyclyl (wherein the heterocyclyl is carbon linked), heteroaryl, (3-7)heterocyclyl(1-6C)alkyl (wherein the heterocyclyl is carbon linked to the (1-6C)alkyl moiety) and heteroaryl(1-6C)alkyl,
and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy, and wherein any heteroaryl or heterocyclyl group within R⁶ is optionally substituted on any available ring nitrogen (provided the ring is not thereby quaternised) by (1-4C)alkyl or (2-4C)alkanoyl, or
R⁵ and R⁶ together with the nitrogen atom to which they are attached form a 4, 5 or 6 membered ring which contains one or two nitrogen atoms as the only heteroatoms present in the ring and which is optionally and which is substituted on an available ring carbon atom by 1 or 2 substituents independently selected from carbamoyl and (1-3C)alkylenedioxy.

25. A quinazoline derivative according to claim 24, wherein R⁶ is selected from (3-7)heterocyclyl (wherein the heterocyclyl is carbon linked), heteroaryl, (3-7)heterocyclyl(1-6C)alkyl (wherein the heterocyclyl is carbon linked to the (1-6C)alkyl moiety) and heteroaryl(1-6C)alkyl,
and wherein any heteroaryl or (3-7)heterocyclyl group within R⁶ is optionally substituted (on any available carbon atoms) by 1, 2 or 3 substituents independently selected from halogeno, (1-6C)alkyl, hydroxy(1-6C)alkyl, (1-6C)alkoxycarbonyl, carbamoyl, (2-6C)alkanoylamino and hydroxy and/or optionally a substituent selected from oxo, cyano, nitro and (1-4C)alkoxy, and wherein any heteroaryl or heterocyclyl group within R⁶ is optionally substituted on any available ring nitrogen (provided the ring is not thereby quaternised) by (1-4C)alkyl or (2-4C)alkanoyl.

26. A quinazoline derivative selected from one or more of the following:
(4S)-4-({4-[(3-chloro-2-fluorophenyl)amino]quinazolin-7-yl}oxy)-N,N,1-trimethyl-L-prolinamide;
(4S)-4-({4-[(3-chloro-2-fluorophenyl)amino]quinazolin-7-yl}oxy)-1-methyl-L-prolinamide;
(4S)-4-({4-[(4-cyano-2-fluorophenyl)amino]-7-methoxyquinazolin-6-yl}oxy)-N,N,1-trimethyl-D-prolinamide;
(4S)-4-({4-[(3-chloro-4-cyanophenyl)amino]-7-methoxyquinazolin-6-yl}oxy)-N,N,1-trimethyl-D-prolinamide;
(4S)-4-[(4-{[3-chloro-4-(trifluoromethyl)phenyl]amino}-7-methoxyquinazolin-6-yl)oxy]-N,N,1-trimethyl-D-prolinamide;
(4S)-4-({4-[(5-chloropyridin-3-yl)amino]-7-methoxyquinazolin-6-yl}oxy)-N,N,1-trimethyl-D-prolinamide;
(4S)-4-({4-[(2-fluoro-4-methylphenyl)amino]-7-methoxyquinazolin-6-yl}oxy)-N,N,1-trimethyl-D-prolinamide;
(4S)-4-({4-[(3-chloro-4-fluorophenyl)amino]-7-methoxyquinazolin-6-yl}oxy)-N,N,1-trimethyl-D-prolinamide;
(4S)-4-({4-[(2-fluoro-4-hydroxyphenyl)amino]-7-methoxyquinazolin-6-yl}oxy)-N,N,1-trimethyl-D-prolinamide;
(4S)-4-({4-[(2,4-difluorophenyl)amino]-7-methoxyquinazolin-6-yl}oxy)-N,N,1-trimethyl-D-prolinamide; (4S)-4-({4-[(2,5-difluorophenyl)amino]-7-methoxyquinazolin-6-yl}oxy)-N,N,1-trimethyl-D-prolinamide;
(4S)-4-({4-[(5-chloro-2-fluorophenyl)amino]-7-methoxyquinazolin-6-yl}oxy)-N,N,1-trimethyl-D-prolinamide;
(4S)-4-({4-[(4chloro-2-fluorophenyl)amino]-7-methoxyquinazolin-6-yl}oxy)-N,N,1-trimethyl-D-prolinamide;
(4S)-4-({4-[(5-chloro-2-hydroxyphenyl)amino]-7-methoxyquinazolin-6-yl}oxy)-N,N,1-trimethyl-D-prolinamide;
(4S)-4-({4-[(3-chloro-4-methoxyphenyl)amino]-7-methoxyquinazolin-6-yl}oxy)-N,N,1-trimethyl-D-prolinamide;
(4S)-4-[(4-{[2-(aminosulfonyl)-5-chlorophenyl]amino}-7-methoxyquinazolin-6-yl)oxy]-N,N,1-trimethyl-D-prolinamide;
(4S)-4-({7-methoxy-4-[(2,3,4-trifluorophenyl)amino]quinazolin-6-yl}oxy)-N,N,1-trimethyl-D-prolinamide;
(4S)-4-[(4-{[2-fluoro-5-(trifluoromethyl)phenyl]amino}-7-methoxyquinazolin-6-yl)oxy]-N,N,1-trimethyl-D-prolinamide;
(4S)-4-[(4-{[2-fluoro-3-(trifluoromethyl)phenyl]amino}-7-methoxyquinazolin-6-yl)oxy]-N,N,1-trimethyl-D-prolinamide;
(4S)-4-({4-[(3-chloro-2-methoxyphenyl)amino]-7-methoxyquinazolin-6-yl}oxy)-N,N,1-trimethyl-D-prolinamide;
(4S)-4-({4-[(3-chloro-2-methylphenyl)amino]-7-methoxyquinazolin-6-yl}oxy)-N,N,1-trimethyl-D-prolinamide;
(4S)-4-({4-[(3-chloro-4-hydroxyphenyl)amino]-7-methoxyquinazolin-6-yl}oxy)-N,N,1-trimethyl-D-prolinamide;
(4S)-4-({4-[(3-ethynylphenyl)amino]-7-methoxyquinazolin-6-yl}oxy)-N,N,1-trimethyl-D-prolinamide;
(4S)-4-({4-[(3-cyanophenyl)amino]-7-methoxyquinazolin-6-yl}oxy)-N,N,1-trimethyl-D-prolinamide;
(4S)-4-{[4-(1H-indol-5-ylamino)-7-methoxyquinazolin-6-yl]oxy}-N,N,1-trimethyl-D-prolinamide;
(4S)-4-({4-[(3-chloro-1H-indol-5-yl)amino]-7-methoxyquinazolin-6-yl}oxy)-N,N,1-trimethyl-D-prolinamide;
(4S)-4-({4-[(3-chloro-2-fluorophenyl)amino]-7-methoxyquinazolin-6-yl}oxy)-N-cyclopropyl-1-methyl-D-prolinamide;
(4S)-4-({4-[(3-chloro-2-fluorophenyl)amino]-7-methoxyquinazolin-6-yl}oxy)-N-(cyclopropylmethyl)-1-methyl-D-prolinamide;
(4S)-4-({4-[(3-chloro-2-fluorophenyl)amino]-7-methoxyquinazolin-6-yl}oxy)-N-(2-methoxyethyl)-1-methyl-D-prolinamide;
(4S)-4-({4-[(3-chloro-2-fluorophenyl)amino]-7-methoxyquinazolin-6-yl}oxy)-N-cyclopentyl-1-methyl-D-prolinamide;
(4S)-4-({4-[(3-chloro-2-fluorophenyl)amino]-7-methoxyquinazolin-6-yl}oxy)-N-cyclopentylmethyl-1-methyl-D-prolinamide;
(4S)-4-({4-[(3-chloro-2-fluorophenyl)amino]-7-methoxyquinazolin-6-yl}oxy)-N-(2-methoxyethyl)-N,1-dimethyl-D-prolinamide;
(4S)-4-({4-[(3-chloro-2-fluorophenyl)amino]-7-methoxyquinazolin-6-yl}oxy)-N-methoxy-1-methyl-D-prolinamide;
(4S)-4-({4-[(3-chloro-2-fluorophenyl)amino]-7-methoxyquinazolin-6-yl}oxy)-N-cyclohexyl-1-methyl-D-prolinamide;
(4S)-4-({4-[(3-chloro-2-fluorophenyl)amino]-7-methoxyquinazolin-6-yl}oxy)-1-methyl-N-(tetrahydro-2H-pyran-4-yl)-D-prolinamide; and
(4S)-4-({4-[(3-chloro-4-fluorophenyl)amino]-6-methoxyquinazolin-7-yl}oxy)-N,N,1-trimethyl-L-prolinamide;
(4S)-4-({4-[(3-chloro-2-fluorophenyl)amino]-7-methoxyquinazolin-6-yl}oxy)-N-[(1R)-1-(hydroxymethyl)-3-methylbutyl]-1-methyl-D-prolinamide;
(4S)-4-({4-[(3-chloro-2-fluorophenyl)amino]-7-methoxyquinazolin-6-yl}oxy)-N-[(1S)-1-(hydroxymethyl)-3-methylbutyl]-1-methyl-D-prolinamide;
(4S)-4-({4-[(3-chloro-2-fluorophenyl)amino]-7-methoxyquinazolin-6-yl}oxy)-N-(3-furylmethyl)-1-methyl-D-prolinamide;
(4S)-4-({4-[(3-chloro-2-fluorophenyl)amino]-7-methoxyquinazolin-6-yl}oxy)-N-(2-furylmethyl)-1-methyl-D-prolinamide;
(4S)-4-({4-[(3-chloro-2-fluorophenyl)amino]-7-methoxyquinazolin-6-yl}oxy)-1-methyl-N-[(5-methylisoxazol-3-yl)methyl]-D-prolinamide;
(4S)-4-({4-[(3-chloro-2-fluorophenyl)amino]-7-methoxyquinazolin-6-yl}oxy)-N-[2-(1H-imidazol-1-yl)ethyl]-1-methyl-D-prolinamide;
(2S)-1-[(4S)-4-({4-[(3-chloro-2-fluorophenyl)amino]-7-methoxyquinazolin-6-yl}oxy)-1-methyl-D-prolyl]azetidine-2-carboxamide;
(4S)-4-({4-[(3-chloro-2-fluorophenyl)amino]-7-methoxyquinazolin-6-yl}oxy)-N-[(2R)-2,3-dihydroxypropyl]-1-methyl-D-prolinamide;
(4S)-4-({4-[(3-chloro-2-fluorophenyl)amino]-7-methoxyquinazolin-6-yl}oxy)-1-methyl-N-(1-methyl-1H-pyrazol-5-yl)-D-prolinamide;
(4S)-4-({4-[(3-chloro-2-fluorophenyl)amino]-7-methoxyquinazolin-6-yl}oxy)-1-methyl-N-3-thienyl-D-prolinamide; and
(4S)-4-({4-[(3-chloro-2-fluorophenyl)amino]-7-methoxyquinazolin-6-yl}oxy)-1-methyl-N-(3-methyl-1H-pyrazol-5-yl)-D-prolinamide;
methyl(4S)-4-({4-[(3-chloro-2-fluorophenyl)amino]-7-methoxyquinazolin-6-yl}oxy)-1-methyl-D-prolyl-L-serinate;
(4S)-4-({4-[(3-chloro-2-fluorophenyl)amino]-7-methoxyquinazolin-6-yl}oxy)-N-(2-hydroxy-1,1-dimethylethyl)-1-methyl-D-prolinamide;
(4S)-4-({4-[(3-chloro-2-fluorophenyl)amino]-7-methoxyquinazolin-6-yl}oxy)-1-methyl-D-prolylglycinamide;
(4S)—N-[2-(acetylamino)ethyl]-4-({4-[(3-chloro-2-fluorophenyl)amino]-7-methoxyquinazolin-6-yl}oxy)-1-methyl-D-prolinamide;
(4S)-4-({4-[(3-chloro-2-fluorophenyl)amino]-7-methoxyquinazolin-6-yl}oxy)-N-[(3S,4R)-4-hydroxytetrahydrofuran-3-yl]-1-methyl-D-prolinamide;
(4S)-4-({4-[(3-chloro-2-fluorophenyl)amino]-7-methoxyquinazolin-6-yl}oxy)-N-[1-(hydroxymethyl)cyclopentyl]-1-methyl-D-prolinamide;
(4S)-4-({4-[(3-chloro-2-fluorophenyl)amino]-7-methoxyquinazolin-6-yl}oxy)-N-[(1S)-1-(hydroxymethyl)-2-methylpropyl]-1-methyl-D-prolinamide;
(4S)-4-({4-[(3-chloro-2-fluorophenyl)amino]-7-methoxyquinazolin-6-yl}oxy)-N-[2-(1H-imidazol-4-yl)ethyl]-1-methyl-D-prolinamide;
(4S)-4-({4-[(3-chloro-2-fluorophenyl)amino]-7-methoxyquinazolin-6-yl}oxy)-N-(2-methoxy-1-methylethyl)-1-methyl-D-prolinamide;
(4S)-4-({4-[(3-chloro-2-fluorophenyl)amino]-7-methoxyquinazolin-6-yl}oxy)-1-methyl-N-(2,2,2-trifluoroethyl)-D-prolinamide;
(4S)—N-({4-[(3-chloro-2-fluorophenyl)amino]-7-methoxyquinazolin-6-yl}oxy)-1-methyl-D-prolinamide (4S)-4-({4-[(3-chloro-2-fluorophenyl)amino]-7-methoxyquinazolin-6-yl}oxy)-N-(2-ethoxyethyl)-1-methyl-D-prolinamide;
(4S)-4-({4-[(3-chloro-2-fluorophenyl)amino]-7-methoxyquinazolin-6-yl}oxy)-N-(4-hydroxycyclohexyl)-1-methyl-D-prolinamide;
(4S)-4-({4-[(3-chloro-2-fluorophenyl)amino]-7-methoxyquinazolin-6-yl}oxy)-1-methyl-N-(2-methylprop-2-en-1-yl)-D-prolinamide;
(4S)-4-({4-[(3-chloro-2-fluorophenyl)amino]-7-methoxyquinazolin-6-yl}oxy)-N-[(1S)-1-(hydroxymethyl)propyl]-1-methyl-D-prolinamide;
(4S)-4-({4-[(3-chloro-2-fluorophenyl)amino]-7-methoxyquinazolin-6-yl}oxy)-N-[(2S)-2,3-dihydroxypropyl]-1-methyl-D-prolinamide;
(4S)-4-({4-[(3-chloro-2-fluorophenyl)amino]-7-methoxyquinazolin-6-yl}oxy)-N-(1H-imidazol-2-ylmethyl)-1-methyl-D-prolinamide;
(4S)-4-({4-[(3-chloro-2-fluorophenyl)amino]-7-methoxyquinazolin-6-yl}oxy)-N-[2-(2-furyl)ethyl]-1-methyl-D-prolinamide;
(4S)-4-({4-[(3-chloro-2-fluorophenyl)amino]-7-methoxyquinazolin-6-yl}oxy)-1-methyl-N-(tetrahydro-2H-pyran-4-ylmethyl)-D-prolinamide;
(4S)-4-({4-[(3-chloro-2-fluorophenyl)amino]-7-methoxyquinazolin-6-yl}oxy)-N-[(1S)-2-hydroxy-1-methylethyl]-1-methyl-D-prolinamide;
(4S)-4-({4[(3-chloro-2-fluorophenyl)amino]-7-methoxyquinazolin-6-yl}oxy)-N-[(1R)-2-hydroxy-1-methylethyl]-1-methyl-D-prolinamide;
(4S)-4-({4-[(3-chloro-2-fluorophenyl)amino]-7-methoxyquinazolin-6-yl}oxy)-N-[(2R)-2-hydroxypropyl]-1-methyl-D-prolinamide;
(4S)-4-({4-[(3-chloro-2-fluorophenyl)amino]-7-methoxyquinazolin-6-yl}oxy)-N-[(2S)-2-hydroxypropyl]-1-methyl-D-prolinamide;
(4S)-4-({4-[(3-chloro-2-fluorophenyl)amino]-7-methoxyquinazolin-6-yl}oxy)-1-methyl-N-[(2R)-tetrahydrofuran-2-ylmethyl]-D-prolinamide;
(4S)-4-({4-[(3-chloro-2-fluorophenyl)amino]-7-methoxyquinazolin-6-yl}oxy)-1-methyl-N-[(2S)-tetrahydrofuran-2-ylmethyl]-D-prolinamide
N-(3-chloro-2-fluorophenyl)-7-methoxy-6-{[(3S,5R)-1-methyl-5-(pyrrolidin-1-ylcarbonyl)pyrrolidin-3-yl}oxy}quinazolin-4-amine;
N-(3-chloro-2-fluorophenyl)-7-methoxy-6-({(3S,5R)-1-methyl-S-[(4methylpiperazin-1-yl)carbonyl}pyrrolidin-3-yl)oxy)quinazolin-4-amine
6-{[(3S,5R)-5-(azetidin-1-ylcarbonyl)-1-methylpyrrolidin-3-yl]oxy}-N-(3-chloro-2-fluorophenyl)-7-methoxyquinazolin-4-amine;
(4S)-4-({4-[(3-chloro-2-fluorophenyl)amino]-7-methoxyquinazolin-6-yl}oxy)-N-(cyanomethyl)-N,1-dimethyl-D-prolinamide;
(4S)-4-({4-[(3-chloro-2-fluorophenyl)amino]-7-methoxyquinazolin-6-yl}oxy)-N-(cyanomethyl)-1-methyl-D-prolinamide;
(4S)-4-({4-[(3-chloro-2-fluorophenyl)amino]-7-methoxyquinazolin-6-yl}oxy)-N,1-dimethyl-N-[(2S)-2-pyrrolidin-1-ylpropyl]-D-prolinamide;
(4S)-4-({4-[(3-chloro-2-fluorophenyl)amino]-7-methoxyquinazolin-6-yl}oxy)-N-[(1R)-2-hydroxy-1-methylethyl]-N,1-dimethyl-D-prolinamide;
(4S)-4-({4-[(3-chloro-2-fluorophenyl)amino]-7-methoxyquinazolin-6-yl}oxy)-N,1-dimethyl-N-(1-methylpiperidin-4yl)-D-prolinamide;
(4S)-4-({4-[(3-chloro-2-fluorophenyl)amino]-7-methoxyquinazolin-6-yl}oxy)-N,1-dimethyl-N-(tetrahydro-2H-pyran-4-yl)-D-prolinamide;
(4R)-4-({4-[(3-chloro-2-fluorophenyl)amino]-7methoxyquinazolin-6-yl}oxy)-1-methyl-N-prop-2-yn-1-yl-L-prolinamide;
1-[[(2S,4R)-4-[[4-[(3-chloro-2-fluorophenyl)amino]-7-methoxy-6-quinazolinyl]oxy]-1-methyl-2-pyrrolidinyl]carbonyl]-3-pyrroline;
(4R)-4-({4-[(3-chloro-2-fluorophenyl)amino]-7-methoxyquinazolin-6-yl}oxy)-N-(cyanomethyl)-1-methyl-L-prolinamide;
(4R)-4-({4-[(3-chloro-2-fluorophenyl)amino]-7-methoxyquinazolin-6-yl}oxy)-N-(2-cyanoethyl)-1-methyl-L-prolinamide;
(4R)-4-({4-[(3-chloro-2-fluorophenyl)amino]-7-methoxyquinazolin-6-yl}oxy)-N-(cyanomethyl)-N,1-dimethyl-L-prolinamide;
(4R)-4-({4-[(3-chloro-2-fluorophenyl)amino]-7-methoxyquinazolin-6-yl}oxy)-N-(2-methoxyethyl)-1-methyl-L-prolinamide;
(4R)-4-({4-[(3-chloro-2-fluorophenyl)amino]-7-methoxyquinazolin-6-yl}oxy)-N-cyclopropyl-1-methyl-L-prolinamide;
(4R)-4-({4-[(3-chloro-2-fluorophenyl)amino]-7-methoxyquinazolin-6-yl}oxy)-N-cyclopentyl-1-methyl-L-prolinamide;
N-(3-chloro-2-fluorophenyl)-7-methoxy-6-({(3R,5S)-1-methyl-5-[(methylpiperazin-1-yl)carbonyl]pyrrolidin-3-yl}oxy)quinazolin-4-amine;
(3S)-1-[(4R)-4-({4-[(3-chloro-2-fluorophenyl)amino]-7-methoxyquinazolin-6-yl}oxy)-1-methyl-L-prolyl]pyrrolidin-3-ol
(4R)-4-({4-[(3-chloro-2-fluorophenyl)amino]-7-methoxyquinazolin-6-yl}oxy)-N-(cyclopropylmethyl)-1-methyl-L-prolinamide;
(4R)-4-({4-[(3-chloro-2-fluorophenyl)amino]-7-methoxyquinazolin-6-yl}oxy)-N-cyclohexyl-N,1-dimethyl-L-prolinamide;
(4R)-4-({4-[(3-chloro-2-fluorophenyl)amino]-7-methoxyquinazolin-6-yl}oxy)-1-methyl-N-(tetrahydro-2H-pyran-4-yl)-L-prolinamide;
N-(3-chloro-2-fluorophenyl)-7-methoxy-6-{[(3R,5S)-1-methyl-5-(pyrrolidin-1-ylcarbonyl)pyrrolidin-3-yl]oxy}quinazolin-4-amine;
(4R)-4-({4-[(3-chloro-2-fluorophenyl)amino]-7-methoxyquinazolin-6-yl}oxy)-N-(2-hydroxyethyl)-N,1-dimethyl-L-prolinamide;
(4R)-4-({4-[(3-chloro-2-fluorophenyl)amino]-7-methoxyquinazolin-6-yl}oxy)-N-[2-(dimethylamino)ethyl]-1-methyl-L-prolinamide;
(4R)-4-({4-[(3-chloro-2-fluorophenyl)amino]-7-methoxyquinazolin-6-yl}oxy)-N,1-dimethyl-N-(1-methylpiperidin-4-yl)-L-prolinamide;
6-({(3R,5S)-5-[(4-acetylpiperazin-1-yl)carbonyl]-1-methylpyrrolidin-3-yl}oxy)-N-(3-chloro-2-fluorophenyl)-7-methoxyquinazolin-4-amine;
1-[(4R)-4-({4[(3-chloro-2-fluorophenyl)amino]-7-methoxyquinazolin-6yl}oxy)-1-methyl-L-prolyl]piperidin-4-ol;
(4R)-4-({4-[(3-chloro-2-fluorophenyl)amino]-7-methoxyquinazolin-6-yl}oxy)-N-(2-methoxyethyl)-N,1-dimethyl-L-prolinamide;
(4R)-4-({4-[(3-chloro-2-fluorophenyl)amino]-7-methoxyquinazolin-6-yl)}oxy)-N-cyclohexyl-1-methyl-L-prolinamide;
(4S)-4-({4-[(3-chloro-2-fluorophenyl)amino]-7-methoxyquinazolin-6-yl}oxy)-N-cyclopropyl-1-methyl-L-prolinamide;
(4S)-4-({4-[(3-chloro-2-fluorophenyl)amino]-7-methoxyquinazolin-6-yl}oxy)-N-(2-methoxyethyl)-1-methyl-L-prolinamide;
(4S)-4-({4-[(3-chloro-2-fluorophenyl)amino]-7-methoxyquinazolin-6-yl}oxy)-N-cyclohexyl-N,1-dimethyl-L-prolinamide;
(4S)-4-({4-[(3-chloro-2-fluorophenyl)amino]-7-methoxyquinazolin-6-yl}oxy)-1-methyl-N-(tetrahydro-2H-pyran-4-yl)-L-prolinamide;
(4S)-4-({4-[(3-chloro-2-fluorophenyl)amino]-7-methoxyquinazolin-6-yl}oxy)-N-(2-methoxyethyl)-N,1-dimethyl-L-prolinamide;
(4S)-4-({4-[(3-chloro-2-fluorophenyl)amino]-7-methoxyquinazolin-6-yl}oxy)-N,1-dimethyl-N-(1-methylpiperidin-4-yl)-L-prolinamide;
(4S)-4-({4-[(3-chloro-2-fluorophenyl)amino]-7-methoxyquinazolin-6-yl}oxy)-N-cyclopentyl-1-methyl-L-prolinamide;
(4S)-4-({4-[(3-chloro-2-fluorophenyl)amino]-7-methoxyquinazolin-6-yl}oxy)-N-methoxy-1-methyl-L-prolinamide;
(4S)-4-({4-[(3-chloro-2-fluorophenyl)amino]-7-methoxyquinazolin-6-yl}oxy)-N-(cyclopropylmethyl)-1-methyl-L-prolinamide;
(4S)-4-({4-[(3-chloro-2-fluorophenyl)amino]-7-methoxyquinazolin-6-yl}oxy)-N-cyclohexyl-1-methyl-L-prolinamide;
and pharmaceutically-acceptable salts thereof.

27. (4S)-4-({4-[(3-chloro-2-fluorophenyl)amino]quinazolin-7-yl}oxy)-1-methyl-L-prolinamide trifluoroacetic acid salt.

28. A pharmaceutical composition which comprises a quinazoline derivative of the Formula I, or a pharmaceutically-acceptable salt or prodrug form thereof, as defined in any one of claims 1 to 25 in association with a pharmaceutically-acceptable diluent or carrier.

29. A quinazoline derivative of the Formula I as defined in any one of claims 1 to 25, or a pharmaceutically acceptable salt or prodrug form thereof, for use as a medicament.

30. The use of a quinazoline derivative of the Formula I, or a pharmaceutically-acceptable salt or prodrug form thereof, as defined in any one of claims 1 to 25 in the manufacture of a medicament for use in the production of an anti-proliferative effect in a warm-blooded animal.

31. A method for producing an anti-proliferative effect in a warm-blooded animal in need of such treatment, which comprises administering to, said animal a quinazoline derivative of the Formula I, or a pharmaceutically acceptable salt or prodrug form thereof, as defined in any one of claims 1 to 25.

32. A process for the preparation of a quinazoline derivative of the Formula I as defined in claim 1 which is selected from one of the following:
Process (a) reacting a compound of the Formula II: 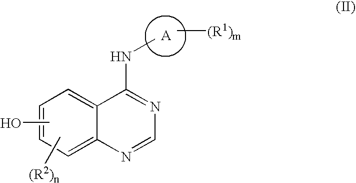
wherein R¹, R², A, m and n have any of the meanings defined in claim 1, except that any functional group is protected if necessary,
with a compound of the Formula III in the presence of a suitable base: 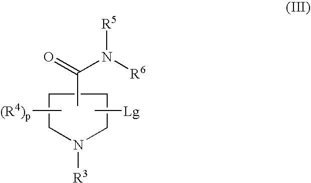
wherein R³, R⁴, R⁵, R⁶ and p have any of the meanings defined in claim 1, except that any functional group is protected if necessary and Lg is a displaceable group,
and whereafter any protecting group that is present is removed;
Process (b) modifying a substituent in, or introducing a substituent into, another quinazoline derivative of Formula I, or a pharmaceutically acceptable salt thereof, as defined in claim 1, except that any functional group is protected if necessary,
and whereafter any protecting group that is present is removed;
Process (c) the removal of a protecting group from a quinazoline derivative of Formula I, or a pharmaceutically acceptable salt thereof, as claimed in claim 1;
Process (d) reacting a compound of the Formula II as defined in reference to process (a) above with a compound of the Formula III as defined in reference to process (a) above, except Lg is OH, under Mitsunobu conditions, and whereafter any protecting group that is present is removed by conventional means;
Process (e) For the preparation of those compounds of the Formula I defined in claim 1 wherein R⁴ is a hydroxy group, by the cleavage of a quinazoline derivative of the Formula I wherein R⁴is a (1-4C)alkoxy group.
Process (f) For the preparation of those compounds of the Formula I defined in claim 1 wherein R⁴ is (1-4C)alkoxy, by the reaction of a compound of the Formula IV: 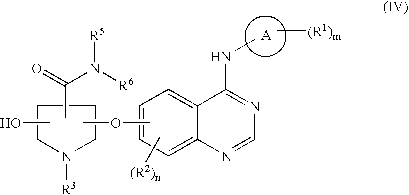
with a compound of the formula (1-4C)alkyl-Lg in the presence of a base, wherein Lg is a displaceable group,
and whereafter any protecting group that is present is removed by conventional means;
Process (g) For the preparation of those compounds of the Formula I defined in claim 1 wherein R¹, R², R⁴ or R⁶ contain a (1-6C)alkoxy or substituted (1-6C)alkoxy group or a (1-6C)alkylamino or substituted (1-6C)alkylamino group, said process comprising the alkylation of a quinazoline derivative of the Formula I wherein R¹, R², R⁴ or R⁶ contain a hydroxy group or a primary or secondary amino group as appropriate;
Process (h) reacting a compound of the formula (V) or reactive derivative thereof 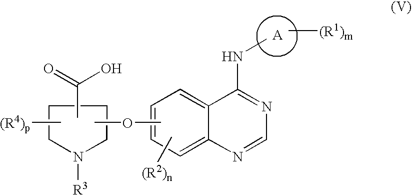
with a compound of the formula HNR⁵R⁶ or a suitable salt thereof in the presence of a base and in an inert solvent;
Process (i) reacting a compound of the formula VI: 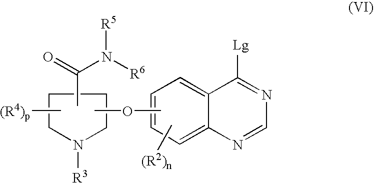
wherein R¹, R², R³, R⁴, R⁵, R⁶, n and p, have any of the meanings defined in claim 1, except that any functional group is protected if necessary, and Lg is a displaceable group as defined in reference to Process (a) above,
with an aniline of the formula VII in the presence of a suitable acid: 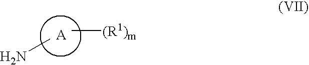
wherein R¹ and m have any of the meanings defined defined in claim 1, except that any functional group is protected if necessary,
Process (j) Forming the group —CON(R⁵)R⁶ by reacting to the corresponding carboxy compound, wherein any functional groups are protected if necessary, with a primary or secondary amine or a heterocyclic group containing an NH group;
and whereafter any protecting group that is present is removed by conventional means.
